(19) United States
(12) Patent Application Publication
(10) Pub. No.: US 2010/0249633 A1
(43) Pub. Date: Sep. 30, 2010

(54) SYSTEMS AND METHODS FOR DETERMINING REGULARITY OF RESPIRATION

(75) Inventors: Amy Droitcour, San Francisco, CA (US); Charles El Hourani, Honolulu, HI (US); Tommy Shing, Honolulu, HI (US); Alex Vergara, Honolulu, HI (US)

Correspondence Address: KNOBBE MARTENS OLSON & BEAR LLP, 2040 MAIN STREET, FOURTEENTH FLOOR, IRVINE, CA, 92614, US

(73) Assignee: KAI MEDICAL, INC., Honolulu, HI (US)

(21) Appl. No.: 12/790,748

(22) Filed: May 28, 2010

Related U.S. Application Data
(62) Division of application No. 12/575,447, filed on Oct. 7, 2009.
Application No. 12/575,447 is a continuation-in-part of application No. 12/418,518, filed on Apr. 3, 2009.
(60) Provisional application No. 61/072,983, filed on Apr. 3, 2008.
(60) Provisional application No. 61/072,982, filed on Apr. 3, 2008.
(60) Provisional application No. 61/123,017, filed on Apr. 3, 2008.
(60) Provisional application No. 61/123,135, filed on Apr. 3, 2008.
(60) Provisional application No. 61/125,021, filed on Apr. 21, 2008.
(60) Provisional application No. 61/125,019, filed on Apr. 21, 2008.
(60) Provisional application No. 61/125,018, filed on Apr. 21, 2008.
(60) Provisional application No. 61/125,023, filed on Apr. 21, 2008.
(60) Provisional application No. 61/125,027, filed on Apr. 21, 2008.
(60) Provisional application No. 61/125,022, filed on Apr. 21, 2008.
(60) Provisional application No. 61/125,020, filed on Apr. 21, 2008.
(60) Provisional application No. 61/125,164, filed on Apr. 22, 2008.
(60) Provisional application No. 61/128,743, filed on May 23, 2008.
(60) Provisional application No. 61/137,519, filed on Jul. 30, 2008.
(60) Provisional application No. 61/137,532, filed on Jul. 30, 2008.
(60) Provisional application No. 61/194,838, filed on Sep. 29, 2008.
(60) Provisional application No. 61/194,836, filed on Sep. 29, 2008.
(60) Provisional application No. 61/194,839, filed on Sep. 29, 2008.
(60) Provisional application No. 61/194,840, filed on Sep. 29, 2008.
(60) Provisional application No. 61/194,848, filed on Sep. 30, 2008.
(60) Provisional application No. 61/196,762, filed on Oct. 17, 2008.
(60) Provisional application No. 61/200,761, filed on Dec. 2, 2008.
(60) Provisional application No. 61/200,876, filed on Dec. 3, 2008.
(60) Provisional application No. 61/141,213, filed on Dec. 29, 2008.
(60) Provisional application No. 61/204,881, filed on Jan. 9, 2009.
(60) Provisional application No. 61/204,880, filed on Jan. 9, 2009.
(60) Provisional application No. 61/206,356, filed on Jan. 30, 2009.
(60) Provisional application No. 61/154,176, filed on Feb. 20, 2009.
(60) Provisional application No. 61/154,728, filed on Feb. 23, 2009.
(60) Provisional application No. 61/154,732, filed on Feb. 23, 2009.
(60) Provisional application No. 61/178,930, filed on May 15, 2009.
(60) Provisional application No. 61/181,289, filed on May 27, 2009.
(60) Provisional application No. 61/184,315, filed on Jun. 5, 2009.
(60) Provisional application No. 61/226,707, filed on Jul. 18, 2009.

(51) Int. Cl. A61B 5/08

(52) U.S. Cl. 600/534

(57) ABSTRACT

A radar-based physiological motion sensor is disclosed. Doppler-shifted signals can be extracted from the signals received by the sensor. The Doppler-shifted signals can be digitized and processed subsequently to extract information related to the cardiopulmonary motion in one or more subjects. The information can include respiratory rates, heart rates, waveforms due to respiratory and cardiac activity, direction of arrival, abnormal or paradoxical breathing, etc. In various embodiments, the extracted information can be displayed on a display.

### CROSS-REFERENCE TO RELATED APPLICATIONS

This application is a divisional of and claims the benefit under 35 U.S.C. §120 of U.S. application Ser. No. 12/575,447 (Atty. Docket No. KSENS.100CP1), filed on Oct. 7, 2009, titled “Non-Contact Physiologic Motion Sensors and Methods For Use;” which is a continuation-in-part of and claims the benefit under 35 U.S.C. §120 of U.S. application Ser. No. 12/418,518 (Atty. Docket No. KSENS.100A), filed on Apr. 3, 2009, titled “Non-Contact Physiologic Motion Sensors and Methods For Use;” which in turn claims the benefit under 35 U.S.C. §119(e) of U.S. Provisional Application No. 61/072,983 (Atty. Docket No. KSENS.021PR), filed on Apr. 3, 2008, titled “Doppler Radar System for Local and Remote Respiration Signals Monitoring”; U.S. Provisional Application No. 61/072,982 (Atty. Docket No. KSENS.023PR), filed on Apr. 3, 2008, titled “Method for Detection of Cessation of Breathing”; U.S. Provisional Application No. 61/123,017 (Atty. Docket No. KSENS.024PR), filed on Apr. 3, 2008, titled “Method for Detection of Motion Interfering with Respiration”; U.S. Provisional Application No. 61/123,135 (Atty. Docket No. KSENS.025PR), filed on Apr. 3, 2008, titled “Method for Detection of Presence of Subject”; U.S. Provisional Application No. 61/125,021 (Atty. Docket No. KSENS.028PR), filed on Apr. 21, 2008, titled “Non-contact Spirometry with a Doppler Radar”; U.S. Provisional Application No. 61/125,019 (Atty. Docket No. KSENS.029PR), filed on Apr. 21, 2008, titled “Monitoring Physical Activity with a Physiologic Monitor”; U.S. Provisional Application No. 61/125,018 (Atty. Docket No. KSENS.030PR), filed on Apr. 21, 2008, titled “Non-contact Method for Calibrating Tidal Volume Measured with Displacement Sensors”; U.S. Provisional Application No. 61/125,023 (Atty. Docket No. KSENS.032PR), filed on Apr. 21, 2008, titled “Use of Empirical Mode Decomposition to Extract Physiological Signals from Motion Measured with a Doppler Radar”; U.S. Provisional Application No. 61/125,027 (Atty. Docket No. KSENS.033PR), filed on Apr. 21, 2008, titled “Use of Direction of Arrival and Empirical Mode Decomposition Algorithms to Isolate and Extract Physiological Motion Measured with a Doppler Radar”; U.S. Provisional Application No. 61/125,022 (Atty. Docket No. KSENS.034PR), filed on Apr. 21, 2008, titled “Data Access Architectures for Doppler Radar Patient Monitoring Systems”; U.S. Provisional Application No. 61/125,020 (Atty. Docket No. KSENS.035PR), filed on Apr. 21, 2008, titled “Use of Direction of Arrival Algorithms to Isolate and Separate Physiological Motion Measured with a Doppler Radar”; U.S. Provisional Application No. 61/125,164 (Atty. Docket No. KSENS.036PR), filed on Apr. 22, 2008, titled “Biometric Signature Collection Using Doppler Radar System”; U.S. Provisional Application No. 61/128,743 (Atty. Docket No. KSENS.037PR), filed on May 23, 2008, titled “Doppler Radar Based Vital Signs Spot Checker”; U.S. Provisional Application No. 61/137,519 (Atty. Docket No. KSENS.039PR), filed on Jul. 30, 2008, titled “Doppler Radar Based Monitoring of Physiological Motion Using Direction of Arrival”; U.S. Provisional Application No. 61/137,532 (Atty. Docket No. KSENS.040PR), filed on Jul. 30, 2008, titled “Doppler Radar Respiration Spot Checker with Narrow Bean Antenna Array”; U.S. Provisional Application No. 61/194,838 (Atty. Docket No. KSENS.041PR), filed on Sep. 29, 2008, titled “Doppler Radar-Based Body Worn Respiration Sensor”; U.S. Provisional Application No. 61/194,836 (Atty. Docket No. KSENS.042PR), filed on Sep. 29, 2008, titled “Wireless Sleep Monitor Utilizing Non-Contact Monitoring of Respiration Motion”; U.S. Provisional Application No. 61/194,839 (Atty. Docket No. KSENS.043PR), filed on Sep. 29, 2008, titled “Continuous Respiratory Rate and Pulse Oximetry Monitoring System”; U.S. Provisional Application No. 61/194,840 (Atty. Docket No. KSENS.044PR), filed on Sep. 29, 2008, titled “Separation of Multiple Targets' Physiological Signals Using Doppler Radar with DOA Processing”; U.S. Provisional Application No. 61/194,848 (Atty. Docket No. KSENS.045PR), filed on Sep. 30, 2008, titled “Detection of Paradoxical Breathing with a Doppler Radar System”; U.S. Provisional Application No. 61/196,762 (Atty. Docket No. KSENS.046PR), filed on Oct. 17, 2008, titled “Monitoring of Chronic Illness Using a Non-contact Respiration Monitor”; U.S. Provisional Application No. 61/200,761 (Atty. Docket No. KSENS.047PR), filed on Dec. 2, 2008, titled “Detection of Paradoxical Breathing with a Paradoxical Breathing Indicator with a Doppler Radar System”; U.S. Provisional Application No. 61/200,876 (Atty. Docket No. KSENS.048PR), filed on Dec. 3, 2008, titled “Doppler Radar Based Monitoring of Physiological Motion Using Direction of Arrival and An Identification Tag”; U.S. Provisional Application No. 61/141,213 (Atty. Docket No. KSENS.049PR), filed on Dec. 29, 2008, titled “A Non-Contact Cardiopulmonary Sensor Device for Medical and Security Applications”; U.S. Provisional Application No. 61/204,881 (Atty. Docket No. KAI-00050), filed on Jan. 9, 2009, titled “Doppler Radar Based Continuous Monitoring of Physiological Motion”; U.S. Provisional Application No. 61/204,880 (Atty. Docket No. KM-00051), filed on Jan. 9, 2009, titled “Doppler Radar Respiration Spot Checker with Narrow Beam Antenna Array”; U.S. Provisional Application No. 61/206,356 (Atty. Docket No. KM-00052), filed on Jan. 30, 2009, titled “Doppler Radar Respiration Spot Check Device with Narrow Beam Antenna Array: Kai Sensors Non-Contact Respiratory Rate Spot Check”; U.S. Provisional Application No. 61/154,176 (Atty. Docket No. KM-00053), filed on Feb. 20, 2009, titled “A Non-Contact Cardiopulmonary Monitoring Device for Medical Imaging System Applications”; U.S. Provisional Application No. 61/154,728 (Atty. Docket No. KAI-00054), filed on Feb. 23, 2009, titled “Doppler Radar-Based Measurement of Vital Signs for Battlefield Triage”; U.S. Provisional Application No. 61/154,732 (Atty. Docket No. KAI-00055), filed on Feb. 23, 2009, titled “Doppler Radar-Based Measurement of Presence and Vital Signs of Subjects for Home Healthcare”. Each of the foregoing applications is incorporated herein by reference in its entirety.

This application is a divisional of and claims the benefit under 35 U.S.C. §120 of U.S. application Ser. No. 12/575,447 (Atty. Docket No. KSENS.100CP1), filed on Oct. 7, 2009, titled “Non-Contact Physiologic Motion Sensors and Methods For Use;” which claims the benefit under 35 U.S.C. §119(e) of U.S. Provisional Application No. 61/178,930 (Atty. Docket No. KAI-00057), filed on May 15, 2009, titled “Aiming or Aligning Methods and Indicator Display for a Doppler Radar System;” U.S. Provisional Application No. 61/181,289 (Atty. Docket No. KAI-00058), filed on May 27, 2009, titled “Intermittent Doppler Radar Respiration Spot Check;” U.S. Provisional Application No. 61/184,315 (Atty. Docket No. KAI-00059), filed on Jun. 5, 2009, titled “Doppler Radar Respiration Spot Check with Automatic Measurement Length;” U.S. Provisional Application No. 61/226,707 (Atty. Docket No. KAI-00060), filed on Jul. 18, 2009, titled “Spiral Antenna for a Contacting Cardiopulmonary Sensor.” Each of the foregoing applications is incorporated herein by reference in its entirety.

### BACKGROUND

1. Field of the Invention

This application in general relates to monitors that can assess the physiological and psychological state of a subject and, in particular, relates to non-contact and radar-based physiologic sensors and their method of use.

2. Description of the Related Art

Motion sensors that can obtain physiological information of a subject, such as respiratory activity, cardiac activity, cardiovascular activity, and cardiopulmonary activity on a continuous or intermittent basis can be useful in various medical applications. Unfortunately, such physiologic activity often occurs in the presence of various other motions, such as, for example, rolling over while sleeping, etc. Thus, data from such motion sensors will typically include desired components corresponding to the physiological activity being measured, and undesired components corresponding to other motions, noise, etc. Existing systems do not adequately separate the desired components from the undesired components.

### SUMMARY

These and other problems are solved by a system that uses a radar-based sensor to sense physiological motion and a processing system that analyzes the data from the radar to distinguish desired data components corresponding to various physiological activity from undesired data components due to other activity, motions, noise, etc. The system can be used to obtain respiratory rate, heart rate, and physiological waveforms including, but not limited to, heart waveforms, pulse waveform, and/or a respiratory waveform. These rates and waveforms can be analyzed to assess various physiological and medical parameters such as, for example, respiratory rates, cardiac rates, respiratory effort, depth of breath, tidal volume, vital signs, medical conditions, psychological state, or location of the subject, etc. These waveforms can also be used to synchronize ventilation or medical imaging with respiratory and/or cardiac motion. The information in these rates and waveforms can be used in many embodiments, including vital signs assessments, apnea monitors, general patient monitoring, neonatal monitoring, burn victim monitoring, home monitoring of the elderly or disabled, triage, chronic illness management, post-surgical monitoring, monitoring of patients during medical imaging scans, disease detection, assessment of psychological state, psychological or psychiatric evaluation, pre-resuscitation assessment, post-resuscitation assessment, and/or lie detection. Various embodiments of the motion sensors can be used in medical applications in various environments including, but not limited to, hospitals, clinics, homes, skilled nursing facilities, assisted living facilities, health kiosks, emergency rooms, emergency transport, patient transport, disaster areas, and battlefields. Various embodiments of the motion sensors can be used for security applications including, but not limited to, security screening at airports, borders, sporting events and other public events, or as a lie detector. Various embodiments of the physiological motion sensors can distinguish valid measurement of heart and respiratory activity from interference, noise, or other motion, and it can provide continuous, point in time, intermittent and/or piecemeal data from which rates, signatures, and key variations can be recognized. Various embodiments of the physiological motion sensor can operate with no contact and work at a distance from a subject. Some embodiments of the physiological motion sensor can also operate when placed on the subject's chest in contact with the body. Various embodiments of the physiological motion sensor can operate on subjects in any position, including lying down, reclined, sitting, or standing. Various embodiments of the physiological motion sensor can operate on subjects from different positions relative to the subject, including from the subject's, from the subject's side, from the subject's back, from above the subject, and from below the subject.

One embodiment includes a method of sensing motion using a motion sensor, the method that includes generating electromagnetic radiation from a source of radiation, wherein the frequency of the electromagnetic radiation is in the radio frequency range, transmitting the electromagnetic radiation towards a subject using one or more transmitters, receiving a radiation scattered at least by the subject using one or more receivers, extracting a Doppler shifted signal from the scattered radiation, transforming the Doppler shifted signal to a digitized motion signal, the digitized motion signal comprising one or more frames, wherein the one or more frames include time sampled quadrature values of the digitized motion signal, demodulating the one or more frames using a demodulation algorithm executed by a processor to isolate a signal corresponding to a physiological movement of the subject or a part of the subject, analyzing the signal to obtain information corresponding to a non-cardiopulmonary motion or other signal interference, processing the signal to obtain information corresponding to the physiological movement of the subject or a part of the subject, substantially separate from the non-cardiopulmonary motion or other signal interference, and communicating the information to an output system that is configured to perform an output action.

In one embodiment, the output system includes a display unit configured to display the information. In one embodiment, the output system includes an audible system that is configured to report information or alerts audibly based on the information. In one embodiment, the output system includes an external medical system that is configured to perform an action based on the information. In one embodiment, the demodulating algorithm includes a linear demodulation algorithm, an arc-based demodulation algorithm or a non-linear demodulation algorithm. In one embodiment, the information is displayed at least alphanumerically, graphically and as a waveform.

In one embodiment, the subject is a human being or an animal and the physiological movement includes at least one of a motion due to respiratory activity of the subject, motion due to a cardiopulmonary activity of the subject, motion due to a cardiac activity of the subject, motion due to a cardiovascular activity of the subject, and motion due to a physical activity of the subject.

In various embodiment the demodulating algorithm includes projecting the signal in a complex plane on a best-fit line, projecting the signal in a complex plane on a principal eigenvector, or aligning a signal arc to a best-fit circle and using the best-fit circle parameters to extract the angular information from the signal arc.

In various embodiment demodulating includes computing in the processor a first set of covariance matrices of a first subset of frames selected from the one or more frames, determining a first A-matrix, wherein the first A-matrix includes a weighted sum of the first set of covariance matrices, determining a first parameter vector corresponding to a first primary value of the first A matrix, storing the first parameter vector in a memory device which is in communication with the processor. In one embodiment, demodulation includes, computing in the processor a second set of covariance matrices of a second subset of frames selected from the one or more frames, determining a second A-matrix, wherein the second A-matrix includes a weighted sum of the second set of covariance matrices, determining a second parameter vector corresponding to a second primary value of the second A-matrix, calculating an inner product of the first parameter vector and the second parameter vector, multiplying the second parameter vector by the sign of the inner product, and projecting the values of the second frame on the second parameter vector to obtain the demodulated signal. In one embodiment, the first primary value includes the largest eigenvalue of the first A-matrix and the first primary vector includes an eigenvector corresponding to the eigenvalue. In one embodiment, the second primary value includes the largest eigenvalue of the second A-matrix and the second primary vector includes an eigenvector corresponding to the eigenvalue.

In one embodiment, the source of radiation includes an oscillator. In one embodiment, the one or more transmitters include one or more antennae. In one embodiment, the one or more receivers include one or more antennae or arrays of antennae. In one embodiment, the transmitting and receiving antennae are the same antennae. In one embodiment, the receiver includes a homodyne receiver. In one embodiment, the receiver includes a heterodyne receiver. In one embodiment, the receiver includes a low-IF receiver configured to transform the Doppler-shifted signal to a Doppler-shifted signal comprising frequencies in a low intermediate frequency range, which is digitized and digitally transformed to a digitized motion signal.

In one embodiment, the processor includes at least one of a digital signal processor, a microprocessor and a computer. In one embodiment, the output system includes a display unit configured to display information regarding the physiological movement of a user at a remote location.

In one embodiment, analyzing the signal includes executing a non-cardiopulmonary motion detection algorithm configured to detect the absence of non-cardiopulmonary motion is detected if the signal includes a single stable source or the presence of non-cardiopulmonary signal if at least the signal is unstable or at least the signal has multiple sources.

In one embodiment, analyzing the signal includes executing a non-cardiopulmonary motion detection algorithm configured to detect the presence of non-cardiopulmonary motion if the signal indicates an excursion larger than the subject's maximum chest excursion from cardiopulmonary activity.

In one embodiment, analyzing the signal includes executing a non-cardiopulmonary motion detection algorithm configured to detect the presence of non-cardiopulmonary motion if a best-fit vector related to linear demodulation changes significantly.

In one embodiment, analyzing the signal includes executing a non-cardiopulmonary motion detection algorithm configured to detect the presence of non-cardiopulmonary motion if a RMS difference between a complex constellation of the signal and a best fit vector related to linear demodulation changes significantly.

In one embodiment, analyzing the signal includes executing a non-cardiopulmonary motion detection algorithm configured to detect the presence of non-cardiopulmonary motion if an origin or radius of a best-fit circle related to arc-based demodulation changes significantly.

In one embodiment, analyzing the signal includes executing a non-cardiopulmonary motion detection algorithm configured to detect the presence of non-cardiopulmonary motion if a RMS difference between a complex constellation of the signal and a best-fit circle related to arc-based demodulation changes significantly.

In one embodiment, analyzing the signal includes executing a non-cardiopulmonary motion detection algorithm by a processor to detect the presence or absence of non-cardiopulmonary motion or other signal interference from the digitized motion signal, wherein the non-cardiopulmonary motion detection algorithm includes a first mode which detects a presence of non-cardiopulmonary motion or other signal interference and a second mode which detects a cessation of non-cardiopulmonary motion or other signal interference.

One embodiment includes communicating information related to a signal quality of a cardiopulmonary motion signal, based on at least one of: a presence of non-cardiopulmonary motion or other signal interference, an absence of non-cardiopulmonary motion or other signal interference, a degree of non-cardiopulmonary motion or other signal interference, an assessment of the signal-to-noise ratio, a detection of low signal power, or a detection of signal clipping or other signal interference, to an output system configured to output the information.

In one embodiment, the first mode includes selecting a first subset of frames from the one or more frames and computing in the processor a first set of covariance matrices of the first subset of frames filtered by a low-pass filter, determining a first A-matrix wherein the A-matrix includes a weighted sum of the first set of covariance matrices, determining a first parameter vector corresponding to a first primary value of the first A matrix, storing the first parameter vector in a memory device which is in communication with the processor. One embodiment further includes computing in the processor a second set of covariance matrices of a second subset of frames filtered by the low-pass filter, determining a second A-matrix, wherein the A-matrix includes a weighted sum value of the second set of covariance matrices, determining a first and a second primary value of the second A-matrix, determining a second parameter vector corresponding to the first primary value of the second A-matrix, calculating an inner product of the first parameter vector and the second parameter vector, calculating a ratio of the first primary value of the second A matrix to the second primary value of the second A matrix, calculating a first energy corresponding to the average energy of a third subset of frames filtered by a high-pass filter and a second energy corresponding to the average energy of a fourth subset of frames filtered by a high-pass filter, and calculating a ratio of the second energy to the first energy. In one embodiment, the first primary value includes the largest eigenvalue of the first A-matrix and the first primary vector includes an eigenvector corresponding to the eigenvalue. In one embodiment, the first primary value of the second A-matrix includes the second largest eigenvalue of the second A-matrix, the second primary value of the second A-matrix includes the largest eigenvalue of the second A-matrix and the second primary vector of the second A-matrix includes an eigenvector corresponding to the first primary value of the second A-matrix.

One embodiment includes computing in the processor a first condition, the first condition being the inner product is less than a first threshold value or the ratio of the first primary value of the second A matrix to the second primary value of the second A matrix is less than a second threshold value or the ratio of the second energy to the first energy is greater than a third threshold value, wherein the presence of non-cardiopulmonary motion or other signal interference is detected if the first condition is true and the ratio of the second energy to the first energy is greater than a fourth threshold value. In one embodiment, the first threshold value is approximately between 0.6 and 1. In one embodiment, the second threshold value is approximately between 4 and 12. In one embodiment, the third threshold value is approximately between 4 and 20. In one embodiment, the fourth threshold value is approximately between 0.1 and 0.8.

In one embodiment, the second mode includes selecting in the processor each and every consecutive subset of frames within a fifth subset of frames, computing in the processor covariance matrices for every subset of frames computing in the processor an A′-matrix for each subset of frames, wherein the A′-matrix is the weighted average of the covariance matrices in the subset, computing in the processor a rho-matrix, wherein each element of the rho-matrix corresponds to a first primary vector of the corresponding A′-matrix, computing the inner product of each pair of primary vectors in the rho-matrix and selecting a minimum absolute value of the inner products, calculating an A matrix which is the sum of the covariance matrices in a sixth subset of frames, determining the first primary value of the A-matrix and the second primary value of the A matrix, calculating the ratio of the first primary value of the A matrix to the second primary value of the A matrix,

One embodiment includes computing in the processor a second condition, the second condition being the minimum absolute value of the inner products is greater than a first threshold value and the ratio of the first primary value to the second primary value is greater than a second threshold value, wherein the cessation of non-cardiopulmonary motion or other signal interference is detected if the second condition is true. In one embodiment, the fifth threshold value is approximately between 0.6 and 1. In one embodiment, the sixth threshold value is approximately between 4 and 12. In one embodiment, the first primary vector includes an eigenvector corresponding to the largest eigenvalue of the corresponding A′-matrix. In one embodiment, the first primary value includes the largest eigenvalue of the A-matrix and the second primary value includes the second largest eigenvalue of the A-matrix. One embodiment includes computing a frame from the one or more frames when the non-cardiopulmonary motion substantially ceased. In one embodiment, one or more frames preceding the frame are discarded.

One embodiment includes a method of estimating the rate of a physiological motion using a motion sensor, generating an electromagnetic radiation from a source of radiation, wherein the frequency of the electromagnetic radiation is in the radio frequency range, transmitting the electromagnetic radiation towards a subject using one or more transmitters, receiving a radiation scattered at least by the subject using one or more receivers, extracting a Doppler shifted signal from the scattered radiation, transforming and digitizing the Doppler shifted signal to a digitized motion signal, the digitized motion signal comprising one or more frames, wherein the one or more frames include time sampled quadrature values of the digitized motion signal, demodulating the one or more frames using a demodulation algorithm executed by a processor to isolate a signal corresponding to a physiological movement of the subject or a part of the subject, executing a non-cardiopulmonary motion detection algorithm by the processor to identify from the digitized motion signal one or more non-cardiopulmonary motion detection events or other signal interference events corresponding to the presence or absence of a non-cardiopulmonary motion or other signal interference, executing by a processor a rate estimation algorithm to estimate a rate of the physiological movement, and providing information related to at least the rate of the physiological movement of the subject or a part of the subject to an output unit that is configured to output the information.

In one embodiment, the rate estimation algorithm includes collecting a plurality of samples from the demodulated frames, identifying one or more samples from the plurality of samples corresponding to non-cardiopulmonary motion detection events and setting to zero the one or more samples from the plurality of samples to obtain at least a first subset of the plurality of samples, and subtracting in the processor a mean of the first subset from the first subset. One embodiment includes calculating in the processor a Fourier transform of the samples included in the first subset to obtain a magnitude spectrum of the samples in the first subset. In one embodiment, the estimated frequency domain rate of the physiological movement corresponds to the largest magnitude component in the spectrum of the samples in the first subset. One embodiment includes identifying either at least three positive zero crossings or at least three negative zero crossings in the first subset, identifying at least a first value for the samples within a first and a second zero crossing, the first value being the largest magnitude positive value or largest magnitude negative value, identifying at least a second value for the samples within a second and a third zero crossing, the second value being the largest magnitude positive value or largest magnitude negative value comparing the first and second values against a threshold value, identifying at least a first breathing event if the first value is greater than a threshold value, identifying at least a second breathing event if the second value is greater than a threshold value, and estimating a time domain respiration rate based on at least the first and second breathing events and the time interval between the first, second and third zero crossings. One embodiment includes calculating in the processor a Fourier transform of the samples included in the first subset to obtain a magnitude spectrum of the samples in the first subset, estimating a frequency domain respiration rate of the physiological movement that corresponds to the largest magnitude spectrum of the samples in the first subset, and comparing the time domain rate and the frequency domain rate to verify an accuracy of the time domain rate and the frequency domain rate.

In one embodiment, the rate estimation algorithm includes identifying at least three consecutive peaks from the plurality of samples, such that a valley is included between two consecutive peaks, and determining a respiration rate based on a number of consecutive peaks detected and the time interval between a first and a last peak.

In one embodiment, the rate estimation algorithm includes identifying at least three consecutive valleys from the plurality of samples, such that a peak is included between two consecutive valleys, and determining a respiration rate based on a number of consecutive valleys detected and the time interval between a first and a last valley. In one embodiment, the rate algorithm selects whether to identify peaks or valleys depending on which occurs first. In one embodiment, the rate estimation algorithm averages the respiration rate based on a number of consecutive peaks and the respiration rate based on a number of consecutive valleys to improve the robustness of the rate estimate.

One embodiment includes a system for sensing a physiological motion including one or more antennas configured to transmit electromagnetic radiation, one or more antennas configured to receive electromagnetic radiation, at least one processor configured to extract information related to cardiopulmonary motion by executing at least one of a demodulation algorithm, a non-cardiopulmonary motion detection algorithm, a rate estimation algorithm, a paradoxical breathing algorithm and a direction of arrival algorithm, and a communications system configured to communicate with an output device, the output device configured to output information related to the cardiopulmonary motion. In one embodiment, a vital signs monitor is configured to monitor at least one of a respiration rate, a heart rate, a depth of breath, respiratory waveform, heart waveform, tidal volume activity and degree of asynchronous breathing in one or more subjects. In one embodiment, an apnea detection system is configured to monitor at least one of a respiration rate, a heart rate, a depth of breath, tidal volume and paradoxical breathing and the presence or absence of breathing in one or more subjects. In one embodiment, a sleep monitor is configured to monitor at least one of a respiration rate, respiratory effort, a heart rate, a depth of breath, tidal volume, paradoxical breathing, activity, position, and physical movement in one or more subjects. In one embodiment, a vital signs measurement system is configured to measure at least one of respiration rate, heart rate, ratio of inhale time to exhale time, tidal volume, and depth of breath in one or more subjects. In one embodiment, a vital signs measurement system is configured to perform a measurement at a point in time or at intermittent points in time.

One embodiment includes a psycho-physiological state monitor configured to monitor at least one of a respiration rate, a heart rate, respiratory waveform, heart waveform, activity, a depth of breath, tidal volume, inhale time, exhale time, and inhale time to exhale time ratio in one or more subjects in response to one or more external stimuli.

In one embodiment, the system sends information to an imaging system, the imaging system configured to image a subject, the information configured to synchronize the imaging system to a physiological motion in the subject.

In one embodiment, the system is configured to send information to a medical device, the information configured to operate the medical device. In one embodiment, the medical device includes a defibrillator. In one embodiment, the system is configured to assess at least one of the presence or absence of respiratory motion and the presence or absence of heart motion.

One embodiment includes a physical activity monitor configured to monitor at least one of a respiration rate, a heart rate, a depth of breath, tidal volume, frequency of non-cardiopulmonary motion, and duration of non-cardiopulmonary motion in one or more subjects. In one embodiment, the weighted sum includes an arithmetic mean. In one embodiment, the medical device includes a ventilator.

One embodiment includes a method of estimating the presence or absence of paradoxical breathing using a motion sensor by generating an electromagnetic radiation from a source of radiation, wherein the frequency of the electromagnetic radiation is in the radio frequency range, transmitting the electromagnetic radiation towards a subject using one or more transmitters, receiving a radiation scattered at least by the subject using one or more receivers, extracting a Doppler shifted signal from the scattered radiation, transforming the Doppler shifted signal to a digitized quadrature motion signal, the digitized quadrature motion signal comprising one or more frames, wherein the one or more frames include time sampled quadrature values of the digitized motion signal, executing a non-cardiopulmonary motion detection algorithm by the processor to identify from the digitized motion signal one or more non-cardiopulmonary motion detection events or other signal interference events corresponding to the presence or absence of a non-cardiopulmonary motion or other signal interference, executing by a processor a paradoxical breathing indication algorithm to estimate the presence or absence of paradoxical breathing, and providing information related to at least the presence, absence, or degree of paradoxical breathing. In one embodiment, the paradoxical breathing indication algorithm includes selecting a subset of the frames, filtering the frames using a low-pass filter, and obtaining a complex constellation plot of the filtered frames.

In one embodiment, an absence of paradoxical breathing is detected if the complex constellation plot is approximately linear, such that the magnitude of a first dimension of the complex constellation plot is greater than a second dimension of the complex constellation plot.

In one embodiment, a presence of paradoxical breathing is detected if the complex constellation plot has a first and a second dimension, such that the first and second dimensions have comparable magnitude.

In one embodiment, a paradoxical factor is calculated to estimate a degree of paradoxical breathing. In one embodiment, the paradoxical factor can be estimated by calculating in the processor a covariance matrix of the subset, calculating a first primary value and a second primary value of the covariance matrix, calculating a first primary vector corresponding to the first primary value and a second primary vector corresponding to the second primary value, projecting the signal on the first primary vector and determining a first amplitude corresponding to the largest peak-to-peak value of the projected signal on the first primary vector, projecting the signal on the second primary vector and determining a second amplitude corresponding to the largest peak-to-peak value of the projected signal on the second primary vector, calculating a first ratio of the first amplitude to the second amplitude, calculating a second ratio of the first primary value to the second primary value, and calculating a product of the first ratio to the second ratio. In one embodiment, the first and second primary value include eigenvalues of the covariance matrix and the first and second primary vectors include eigenvectors corresponding to the first and second primary value.

In one embodiment, the paradoxical indicator is calculated with a cost function performed on the paradoxical factor. In one embodiment, the presence or absence of paradoxical breathing is determined by comparing the output of the cost function to a threshold.

In one embodiment, the paradoxical indicator is analyzed to provide a first indication for absence of paradoxical breathing, a second indication for uncertain results and a third indication for the presence of paradoxical breathing.

One embodiment includes a method of estimating the direction of arrival using a motion sensor by generating an electromagnetic radiation from a source of radiation, wherein the frequency of the electromagnetic radiation is in the radio frequency range, transmitting the electromagnetic radiation towards a subject using one or more transmitters, receiving a radiation scattered at least by the subject using one or more receivers, extracting a Doppler shifted signal from the scattered radiation, transforming the Doppler shifted signal to a digitized quadrature motion signal, the digitized quadrature motion signal comprising one or more frames, wherein the one or more frames include time sampled quadrature values of the digitized motion signal from each receiver, executing by a processor a direction of arrival algorithm to estimate the number of targets and corresponding angles, and providing information corresponding to at least one of the cardiopulmonary movement of one or more subjects or a part of one or more subjects, the number of subjects, and the direction of one or more subjects to an output unit that is configured to output the information. In one embodiment, the direction of arrival algorithm includes filtering a subset of frames selected from the one or more frames using a low pass filter, each frame consisting of signals from a plurality of receive channels in the multiple receive antenna array, calculating the power spectrum density of all the channels for the low pass filtered subset of frames, using the power of the frequency components in the calculated power spectrum density to determine the frequency components that are most likely to contain a cardiopulmonary signals from one or more subjects, identifying the angular direction of each frequency component, identifying at least a first and a second angular direction such that each angular direction is separated from the other angular direction by an angular distance greater than or equal to an angular resolution of the one or more receivers, eliminating one or more angles that are separated by an angular distance less than the angular resolution of the one or more receivers, and generating one or more DOA vectors with unity magnitude for each target in the angular direction, and smoothing the DOA vectors with a weighted average of a current DOA vector and a previous DOA vectors in a buffer. One embodiment further includes separating the signal from each angular direction by steering spatial nulls towards the other angular directions, executing by the processor a non-cardiopulmonary motion detection algorithm to detect a presence or absence of non-cardiopulmonary motion or other signal interference in each separated signal, and executing by the processor a demodulation algorithm to demodulate each of the separated signals, and process each demodulated signal to obtain information corresponding to the cardiopulmonary motion if absence of non-cardiopulmonary motion is detected. One embodiment further includes isolating the signal from the desired subject by steering spatial nulls toward the other angular directions, executing by the processor a non-cardiopulmonary motion detection algorithm to detect a presence or absence of non-cardiopulmonary motion or other signal interference in the isolated signal, and executing by the processor a demodulation algorithm to demodulate the isolated signal, and process the demodulated signal to obtain information corresponding to the subject's cardiopulmonary motion if absence of non-cardiopulmonary motion is detected.

In one embodiment, the direction of arrival algorithm includes filtering a subset of frames selected from the one or more frames using a low pass filter, each frame consisting of signals from a plurality of receive channels included in the multiple receiver antenna array, calculating the power spectrum density of all the channels for the low pass filtered subset of frames, using the power of the frequency components in the calculated power spectrum density to determine the frequency components that are most likely to contain the cardiopulmonary signals from one or more subjects, identifying an angular direction of each frequency component, identifying at least a first and a second angular direction such that each angular direction is separated from the other angular direction by an angular distance greater than or equal to an angular resolution of the multiple receiver antenna array, eliminating one or more angles that are separated by an angular distance less than the angular resolution of the multiple receiver antenna array, generating a DOA vector with unity magnitude for each target in the angular direction, smoothing the DOA vectors with a weighted average of the current DOA vectors and previous DOA vectors in a buffer, repeating the DOA algorithm periodically and updating the DOA vectors, and communicating angles corresponding to the DOA vectors to the output unit.

Disclosed herein is a method of sensing motion using a motion sensor. The method can include the steps of: generating electromagnetic radiation from a source of radiation, wherein the frequency of the electromagnetic radiation is in the radio frequency range; transmitting the electromagnetic radiation towards a subject using one or more transmitters; receiving a radiation scattered at least by the subject using one or more receivers; extracting a Doppler shifted signal from the scattered radiation; transforming the Doppler shifted signal to a digitized motion signal, said digitized motion signal comprising one or more frames, wherein the one or more frames comprise time sampled quadrature values of the digitized motion signal; processing said one or more frames to obtain information corresponding to the cardiopulmonary movement of the subject or a part of the subject, substantially separate from non-cardiopulmonary motion or other signal interference; estimating the subject's depth of breath from the cardiopulmonary movement information; and communicating the information to an output system that is configured to perform an output action.

In some embodiments, estimation of depth of breath comprises: obtaining information about the absolute time-varying chest position by extracting the time-varying phase difference between the transmitted radiation and the received radiation and multiplying by a constant conversion factor; identifying maximum inhale points and maximum exhale points on the absolute time-varying chest position; and determining the difference in position between the maximum inhale points and the maximum exhale points. In some embodiments, the estimation of depth of breath comprises: estimating the center circle on which the samples lie in the complex plan; identifying the endpoints of the arc on which the samples lie; determining the central angle subtended by the arc, and multiplying the angle by a constant conversion factor.

The end-points of the arc can be identified by one or more of: identifying the points of minimal velocity, identifying the center of high-density clusters of samples, or identifying points with large changes in direction. The estimation of depth of breath can also include: counting the number of rotations of the signal around the center in the complex plane; and multiplying this number by a constant conversion factor. In some embodiments, information corresponding to the cardiopulmonary movement of the subject includes one or more of: respiratory rate, pulse rate, inhale time to exhale time ratio, and irregularity of respiration. In some embodiments, the output action includes alarms for one or more of: depth of breath below a threshold, depth of breath above a threshold, depth of breath multiplied by a respiratory rate above a threshold, and a depth of breath multiplied by a respiratory rate below a threshold.

Also disclosed herein is a method of sensing motion using a motion sensor. The method comprises generating electromagnetic radiation from a source of radiation, wherein the frequency of the electromagnetic radiation is in the radio frequency range; transmitting the electromagnetic radiation towards a subject using one or more transmitters; receiving a radiation scattered at least by the subject using one or more receivers; extracting a Doppler shifted signal from the scattered radiation; transforming the Doppler shifted signal to a digitized motion signal, said digitized motion signal comprising one or more frames, wherein the one or more frames comprise time sampled quadrature values of the digitized motion signal; conditioning the digitized motion signal into a conditioned motion signal using a conditioning algorithm executed by a processor to prepare the digitized motion signal for demodulation; demodulating said conditioned motion signal using demodulation algorithms executed by a processor to convert a quadrature digitized motion signal to a motion waveform; processing the motion waveform to obtain information corresponding to the cardiopulmonary movement of the subject or a part of the subject, substantially separate from non-cardiopulmonary motion or other signal interference; and communicating the information to an output system that is configured to perform an output action.

In some embodiments, the conditioning algorithm comprises reducing the signal to no more than about 10, 9, 8, 7, 6, 5, 4, 3, or less points for representation. The points for representation can be selected from, for example, one or more of: end points comprising the extremes of an arc; points of minimum or maximum velocity; points of minimum or maximum acceleration; centers of clusters of high point density; points of largest change in direction; points of largest change in segment length; self-intersection points; points of intersection with a fitted shape; points of intersection with a fitted shape's axis; and the midpoint between other key points. The conditioning algorithm can include smoothing the arc in the complex plane, and/or segmentation of the signal in the complex plane. In some embodiments, segmentation comprises one or more of generating line segments based on a pre-defined number of samples, a fraction of the number of samples in one respiratory cycle, a multiple of the number of samples in one respiratory cycle, and an adaptively set number of samples.

The demodulation algorithm can include identification of a center with a center-find algorithm, setting the center to zero, and performing an arctangent function on the data points. In some embodiments, the center-find algorithm comprises identifying the best-fit circle to the samples through a least-mean-square-error method or a maximum-likelihood-estimator method that defines a circle with geometric or algebraic method. In some embodiments, the center-find algorithm comprises finding using a least-squares method to find the point of intersection between lines perpendicular to segments between data points of the conditioned motion signal. In still other embodiments, the center-find algorithm comprises calculating the geometric center of the data points. The arc can be smoothed by: applying a two-dimensional gradient to the samples in the complex plane; using the gradient peak values to define the arc's trajectory; and adjusting the samples to be along this trajectory. The conditioning algorithm can include using an endpoint-finding algorithm to identify the end-points of the arc; estimating the trajectory of the arc; adjusting the arc's trajectory such that it has the endpoints estimated by the endpoint-finding algorithm; and adjusting the samples to be along the adjusted trajectory. The conditioning algorithm can also include computing a best-fit line in the complex plane repeatedly for subsets, such as small subsets, of consecutive samples. In some embodiments, the demodulation algorithm comprises evaluating the changes in the direction of the best-fit lines and accumulating them.

Also disclosed herein is a method of performing a non-contact, point-in-time measurement of vital signs. The method includes the steps of generating electromagnetic radiation from a source of radiation, wherein the frequency of the electromagnetic radiation is in the radio frequency range; transmitting the electromagnetic radiation towards a subject using one or more transmitters; receiving a radiation scattered at least by the subject using one or more receivers; extracting a Doppler shifted signal from the scattered radiation; transforming the Doppler shifted signal to a digitized motion signal, said digitized motion signal comprising one or more frames, wherein the one or more frames comprise time sampled quadrature values of the digitized motion signal; demodulating said one or more frames using a demodulation algorithm executed by a processor to isolate a signal corresponding to a physiological movement of the subject or a part of the subject; analyzing the signal to obtain information regarding signal quality that flags each frame of the signal as low quality or high quality; processing the signal to obtain information corresponding to the physiological movement of the subject or a part of the subject, substantially separate from said non-cardiopulmonary motion or other signal interference; determining the length of the measurement interval with an interval selection algorithm that utilizes the information regarding signal quality and the information corresponding to the physiological movement of the subject or a part of the subject; and communicating the information to an output system that is configured to perform an output action.

In some embodiments, information regarding signal quality comprises information corresponding to a non-cardiopulmonary motion or other signal interference, and/or information corresponding to an assessment of whether the received signal power is adequate for processing the signal. In some embodiments, the interval selection algorithm extends the interval until at least about 5, 10, 15, 20, 25, 30, 35, 40, 45, 60 seconds, or more of high-quality data is obtained. The time interval could be consecutive. In some embodiments, the interval selection algorithm extends the interval until at least 1, 2, 3, 4, 5, 6, 7, 8, 9, 10, or more complete breaths, which can be consecutive breaths, with high-quality data is obtained. In some embodiments, the interval selection algorithm assesses the irregularity of respiration in at least 5, 10, 15, 20, 25, 30, 35, 40, 45, 60 seconds or more of high-quality data, and if this assessment indicates irregular breathing, extends the measurement until breathing appears to be regular, a periodic pattern repeats, or at least 5, 10, 15, 20, 25, 30, 35, 40, 45, 60 seconds or more has passed and breathing is still irregular and non-periodic. In some embodiments, the interval selection algorithm extends the interval until 15-60 seconds of high-quality data is obtained, and/or about 3-5 complete breaths with high quality data in some embodiments. The interval selection algorithm can have a time-out, such that if the interval extends beyond 10, 20, 30, 40, 50, 60 seconds, or more, or between about 30 seconds and 5 minutes in some embodiments, the device provides an error message, retry message, or error code. In some embodiments, the time-out is determined by other equipment when the device is integrated with another device that performs vital signs measurements. In some embodiments, the time-out occurs at the completion of all the other vital signs measurements.

Also disclosed herein is a system for sensing a physiological motion. The system includes one or more antennas configured to transmit electromagnetic radiation; one or more antennas configured to receive electromagnetic radiation; at least one processor configured to extract information related to cardiopulmonary motion by executing at least one of a demodulation algorithm, a non-cardiopulmonary motion detection algorithm, and a rate estimation algorithm; and a communications system configured to communicate with an output device, said output device configured to output information related to the cardiopulmonary motion.

The device can provide a spot check (point-in-time) measurement of vital signs, which can include, for example, a respiratory rate or heart rate. The source of radiation can be a voltage-controlled oscillator, which is phase-locked to a crystal with a phase-lock loop circuit, such that the frequency of the radiation can be selected within a band, providing a tunable frequency synthesizer and frequency selectivity. In some embodiments, the same antenna is configured to transmit and receive electromagnetic radiation, and the antenna comprises an array of metal elements with an air gap between the elements and the ground plane, The air gap can be between about 0.25 to 1 inch, such as about 0.5 inches in some embodiments. Spread spectrum techniques can be used to introduce a pseudo-random phase noise to the frequency synthesizer utilizing the phase-locked oscillator. In some embodiments, the system includes a direct-conversion receiver with an active I/Q demodulator to provide differential quadrature signals, a fully differential signals conditioning stage with filtering and amplification, and a differential-input analog-to-digital converter. The signal conditioning can provide a DC-coupled signal, and the ADC can be high-resolution, such as 12, 16, 20, 24 bits, or more. The system can be powered by a variety of power sources, such as AC or DC current. In one embodiment, the system is powered through 5V USB bus power. The system can include a radio and processor integrated in the same housing, or as separate modules. The processor can run the algorithms and provides rate and other information to a separate host computer. The host computer can provide a command over a communications interface to initiate measurements. The device can include an integrated light source to provide feedback on the proper aiming of the device. The light source can include, for example, an LED such as a high-intensity directional LED. The integrated light source can illuminate the areas included in the antenna field of view. The system can also include a button that can be used to turn the light source on and off, and/or a display such as an integrated display. The sensor's integrated display can provide instant feedback messages including progress, error messages, retry messages, low-signal information, results, and other information. The system can also include real-time audio feedback, such that if the system is aimed improperly such that the signal power is low, there is an audible indication.

In some embodiments, disclosed is a method of sensing motion using a motion sensor. The method can include the steps of generating electromagnetic radiation from a source of radiation, wherein the frequency of the electromagnetic radiation is in the radio frequency range; transmitting the electromagnetic radiation towards a subject using one or more transmitters; receiving a radiation scattered at least by the subject using one or more receivers; extracting a Doppler shifted signal from the scattered radiation; transforming the Doppler shifted signal to a digitized motion signal, said digitized motion signal comprising one or more frames, wherein the one or more frames comprise time sampled quadrature values of the digitized motion signal; demodulating said one or more frames using a demodulation algorithm executed by a processor to isolate a signal corresponding to a physiological movement of the subject or a part of the subject; analyzing the signal to obtain information corresponding to a non-cardiopulmonary motion or other signal interference; processing the signal to obtain information corresponding to the physiological movement of the subject or a part of the subject, substantially separate from said non-cardiopulmonary motion or other signal interference; estimating point-in time vital signs parameters at a pre-determined intervals; and communicating the information to an output system that is configured to perform an output action.

In some embodiments, the output action comprises the display of a history of point-in-time measurements, including values and times, such that trends can be viewed. Estimating point in time vital signs parameters can comprise determining the length of the measurement interval with a interval selection algorithm that utilizes the information corresponding to a non-cardiopulmonary motion or other signal interference and information corresponding to the physiological movement of the subject or a part of the subject. The pre-determined intervals can be user selectable from a menu of intervals. The pre-determined intervals can be selected by the user with a keypad interface. In some embodiments, an external device controls a device which estimates point-in-time vital signs parameters by sending commands for when to start measurements, in cases wherein the device that estimates point-in-time vital signs does not have interval measurement capability. The external device can be, for example, a computer, a vital signs measurement device, or a patient monitor.

In some embodiments, disclosed herein is a method of estimating the presence or absence of paradoxical breathing using a motion sensor. The method can include the steps of generating an electromagnetic radiation from a source of radiation, wherein the frequency of the electromagnetic radiation is in the radio frequency range; transmitting the electromagnetic radiation towards a subject using one or more transmitters; receiving a radiation scattered at least by the subject using one or more receivers; extracting a Doppler shifted signal from the scattered radiation; transforming the Doppler shifted signal to a digitized quadrature motion signal, said digitized quadrature motion signal comprising one or more frames, wherein the one or more frames comprise time sampled quadrature values of the digitized motion signal; executing a non-cardiopulmonary motion detection algorithm by the processor to identify from the digitized motion signal one or more non-cardiopulmonary motion detection events or other signal interference events corresponding to the presence or absence of a non-cardiopulmonary motion or other signal interference; executing by a processor a paradoxical breathing indication algorithm to estimate the presence or absence of paradoxical breathing; and providing information related to at least the presence, absence, or degree of paradoxical breathing. In some embodiments, the paradoxical breathing indication algorithm comprises: evaluating the distribution of samples in the complex plane and distinguishing an arc or a line from an ellipse, circle, crescent-moon shape, kidney-bean shape, egg shape, figure-8 or ribbon shape, or other shape that is not a line or arc, indicating the absence of paradoxical breathing is a line or arc is detected; and indicating the presence of paradoxical breathing if a shape other than a line or arc is detected.

In some embodiments, the paradoxical breathing indication algorithm comprises comparing the trajectory in the complex plane during inhalation with that during exhalation; indicating the absence of paradoxical breathing if the two are similar; and indicating the presence of paradoxical breathing if the two are significantly different. In other embodiments, the paradoxical breathing indication algorithm comprises: segmenting the shape in the complex plane by determining the best-fit line for each frame (segments of the data); calculating an orientation vector pointing in the direction of movement in the complex plane for every frame; calculating the change in phase between each consecutive orientation vector; determining whether the change in phase between each consecutive orientation vector is positive or negative; indicating the presence of paradoxical breathing if either positive phase change or negative phase change is dominant; and indicating the absence of paradoxical breathing if the phase change is approximately evenly distributed between positive and negative. In some embodiments, the paradoxical breathing indication algorithm comprises fitting the samples in the complex plane to an arc that subtends an angle no greater than a threshold value. The angle could be between 0-180 degrees, such as 90 to 180 degrees, or less than about 180, 170, 160, 150, 140, 130, 120, 110, 100, 90, 80, 70, 60, or less degrees in some embodiments. The threshold can be determined based on information in the patient's medical record. In some embodiments, the paradoxical breathing indication algorithm comprises fitting the samples in the complex plane to an ellipse; determining the eccentricity of the ellipse; indicating the presence of paradoxical breathing if the eccentricity of the ellipse is above a threshold; and indicating the absence of paradoxical breathing if the eccentricity of the ellipse is below a threshold. In some embodiments, comparing the trajectory comprises fitting a circle or an arc to the inhalation samples in the complex plane and to the exhalation samples in the complex plane; and comparing the centers and the radii of the circles for inhalation and exhalation. The paradoxical breathing indication algorithm can also include calculating the area enclosed by a full breathing cycle in the complex plane; indicating the presence of respiration if the area bounded by the points is greater than a threshold; and indicating the absence of respiration if the area bounded by the points is less than a threshold. In some embodiments, the paradoxical breathing indication algorithm includes fitting a circle to the samples in the complex plane from one or more complete breathing cycles; estimating the center of that circle; calculating the distance from each sample to the center of the circle; calculating the variance of the distance from each sample to the center of the circle; indicating the presence of paradoxical breathing if the variance is above a threshold; and indicating the absence of paradoxical breathing if the variance is below a threshold.

Also disclosed herein is a method of determining the regularity of respiration, comprising: processing one or more frames of a respiratory waveform to obtain information regarding the irregularity or regularity of respiration; said respiratory waveform comprising one or more frames, wherein the one or more frames comprise time sampled values of respiratory signals; and communicating the information to an output system that is configured to perform an output action.

The respiratory waveform can be obtained by one of Doppler radar, ultrawideband radar, impedance pneumography, chest straps, airflow measurements, or load cells. Information regarding the irregularity or regularity of respiration includes, for example, assessment of the irregularity of the breath-to-breath interval or respiratory rate; assessment of the irregularity of the amplitude of a breath or the depth of breath; assessment of both irregularity in the amplitude of respiration and irregularity in the breath-to-breath interval; estimation of the cycle length of periodic or Cheyne-Stokes breathing; assessment of the length of apnea in each cycle or the average length of apnea over several cycles; and/or the history of irregularity. The output of the system can be an indication of regularity or irregularity (a binary state); an integrated regularity index that compiles a variety of information about the regularity of respiration into a signal number or a single bar graph; separate indications of the irregularity of the breath-to-breath interval and the irregularity of the depth of breath; or individual indications of several measures of irregularity. In some embodiments, processing one or more frames comprises: performing an auto-correlation function on a subset of frames; identifying whether major peaks are present; identifying the number of samples from the center to major peaks, if they are present; determining whether breathing is regular based on the number of samples to the first major peak and the height of the first major peak; and identifying the second major peak that is not a multiple of the respiratory period as the period of periodic breathing.

The subset of frames can include samples obtained over a time longer than the expected period of respiration. In some embodiments, the subset of frames includes samples obtained over a time longer than the expected cycle period of irregular respiration. The method can also include using a wavelet transform function to create an index of repeating patterns in a respiration signal. In some embodiments, the irregularity of the breath-to-breath interval, or breath duration, is estimated from one or more of the group consisting of: the standard deviation of the breath-to-breath interval, the frequency of apneaic events, the coefficient of variation of the breath-to-breath interval, the standard deviation of the respiratory rate, and the coefficient of variation of the respiratory rate. In some embodiments, the irregularity of the amplitude of a breath or the depth of breath, or breath duration, is estimated from the standard deviation of the breath depth, the coefficient of variation of the breath depth, the standard deviation of the respiratory signal amplitude, or the coefficient of variation of the respiratory signal amplitude. Information regarding the irregularity or regularity of respiration can include assessment of whether irregular breathing is periodic. This assessment can include estimating each breath-to-breath interval, and storing it with the time point at the end of the interval in which it was calculated; interpolating between these breath-to-breath intervals to create a waveform; performing the Fourier transform, performing the autocorrelation function, or calculating the power spectral density of the waveform; determining whether there are significant peaks of the Fourier transform, the autocorrelation function, or the power spectral density of the waveform; and determining that if significant peaks exist, the breathing is irregular and periodic. The assessment can also include interpolating between these breath-to-breath intervals to create a waveform; identifying peaks of the waveform; determining the time between the peaks; calculating the coefficient of variation of the time between the peaks; determining if the coefficient of variation of the time between the peaks is low, the breathing is irregular and periodic; and determining if the coefficient of variation of the time between the peaks is low, the breathing is irregular and is not periodic. In some embodiments, assessment of whether irregular breathing is periodic comprises: identifying apneaic events; determining the time of cessation of apneaic events; estimating the interval between the cessation of each consecutive pair of apneaic events; determining whether the interval between the cessation of each consecutive pair of apneaic events is consistent by calculating the coefficient of variation of the interval between the events by calculating the coefficient of variation; determining if the coefficient of variation is below a threshold, breathing is periodic; and determining if the coefficient of variation is above a threshold, breathing is irregular and not periodic. In some embodiments, assessment of whether irregular breathing is periodic comprises calculating the envelope of the respiratory waveform; performing the Fourier transform, performing the autocorrelation function, or calculating the power spectral density of the waveform; and determining whether there are significant peaks of the Fourier transform, the autocorrelation function, or the power spectral density of the waveform. In some embodiments, the envelope is calculated by interpolating between the peak amplitudes, or squaring the signal and applying a low-pass filter.

The integrated respiratory status index can be a value, that is 0 for regular respiration, and can vary up to 1, 2, 3, 4, 5, or 6, with 1 point added for each of the following: irregular breath-breath interval; irregular breath depths; periodic breath-breath interval; periodic breath depth; periodic breath depth cycle time >60 seconds; periodic breath-breath interval cycle time >60 seconds; periodic breathing includes apnea >20 seconds; non-periodic irregular breathing includes apnea >20 seconds more frequently than once every 10 minutes.

In some embodiments, the integrated respiratory status index is a value that is 0 for regular respiration that increases by one point for each 5, 10, 20, 30%, or more in the coefficient of variation of the breath-to-breath interval and by one point for each 5, 10, 20, 30% or more in the coefficient of variation in the depth of breath.

In some embodiments, information regarding the irregularity or regularity of respiration is assessed by the following algorithms:

(a) Estimate the breath-to-breath interval and the depth of breath for each breath as respiration is processed.

(b) Over an interval of 50 breaths, calculate the mean and standard deviation of the breath-breath interval, and the mean and standard deviation of the depth of breath.

(c) Calculate the coefficient of variation of the breath-to-breath interval and the depth of breath. If neither one is above a threshold, the respiration is considered regular. If the coefficient of variation of either the breath-breath interval or the depth of breath is above a threshold, the respiration is considered irregular, and additional processing is performed. In some embodiments, the threshold is 25%.

(d) If the respiration is irregular, determine whether the cycle time is periodic by interpolating between breath-breath intervals and depth of breath estimates, taking a Fourier transform of each waveform, and determining whether a periodic component exists in either waveform. If a periodic component exists in at least one of the waveforms, the cycle time is periodic. If a periodic component does not exist in either waveform, the cycle time is not periodic.

(e) If the cycle time is not periodic, repeat step (d) with a longer interval of breaths (150 breaths). If the cycle time is still not periodic, skip to step (g).

(f) If the cycle time is periodic, calculate the cycle time finding by peaks in the interpolated breath-breath interval in step (d) and determining the mean time between the peaks. If multiple peaks are not available, extend the interval used for this step.

(g) If the cycle is not periodic, isolate the breath-breath intervals longer than 20 seconds. Calculate the number of these intervals divided by the total time interval used for calculation. Calculate the mean of these apneaic events.

(h) If the cycle is periodic, determine the length of apnea in each period, and average this number to get the average apnea length per cycle.

(i) Display the data. If respiration is regular, indicate that respiration is “regular”. If respiration is irregular, indicate either “periodic—cycle time X” where X is the cycle time or “irregular.” If apneaic events exist, indicate “—average apnea length Y” and, if respiration is not periodic also indicate “—Z apneaic events/minute.”

Also disclosed herein is a method of sensing motion using a motion sensor, the method comprising: generating electromagnetic radiation from a source of radiation, wherein the frequency of the electromagnetic radiation is in the radio frequency range; transmitting the electromagnetic radiation towards a subject using one or more transmitters; receiving a radiation scattered at least by the subject using one or more receivers; extracting a Doppler shifted signal from the scattered radiation; transforming the Doppler shifted signal to a digitized motion signal, said digitized motion signal comprising one or more frames, wherein the one or more frames comprise time sampled quadrature values of the digitized motion signal; processing said one or more frames to obtain information corresponding to the cardiopulmonary movement of the subject or a part of the subject, substantially separate from non-cardiopulmonary motion or other signal interference; estimating the subject's respiratory rate from the cardiopulmonary movement information; and communicating the information to an output system that is configured to perform an output action.

In some embodiments, the respiratory rate is estimated by counting repeating key points, which are points in a respiration cycle that are identifiable using specific algorithms. The key points can include peaks, valleys, zero crossings, points of fastest change, points of no change, and points with the greatest change in direction. In some embodiments, the respiratory rate is determined before demodulation by making key points in the complex plane. The key points can also include points with low velocity in the complex plane or points with high velocity in the complex plane.

The rate of the respiratory signal can be estimated in the time domain by tracking the points where a signal crosses a time-delayed version of itself. The time delay can be adaptively set using the spectrum of the data to provide a delay that is long enough to suppress small variations or noise, and short enough to compare within the same respiratory cycle. The cardiopulmonary movement information can be pre-conditioned before rate estimation by normalizing the envelope of the signal before applying a rate estimation algorithm that utilizes peak-finding. In some embodiments, each breath is identified based on breath characteristics, and breaths that meet the required characteristics are used for rate-finding. Breath characteristics can include the ratio of the duration of an inhale to the ratio of an exhale that must lie within a defined interval, and can include detection of a peak and detection of a valley. The defined interval can be determined based on the patient's height, weight, and other information in the patient's medical chart. The defined interval can also be adaptively determined based on prior observations of the patient. The characteristics can be, for example, the ratio of inhale time to exhale time, the length of pauses in breathing, the ratio of the length of a pause in breathing to the breathing period, the depth of breath, and the inflection points of the breath. The characteristics of the breath can include the mean, variance, and kurtosis of the breath. The characteristics of the breath can also include the coefficients of a wavelet decomposition of the signal or the coefficients of a Fourier transform of the signal. The respiratory signal being considered can have the same characteristics extracted as those in a database of breathing signals, the features from each are compared, and if a match is found, the signal is labeled as a breath. In some embodiments, the cardiopulmonary movement information, if indicated to have irregular or periodic breathing, is separated into at least a first section and a second section in which breaths are similar, such that the rates can be estimated separately for each section. The sections can be separated by, for example, frequency and power, empirical mode decomposition, or wavelet decomposition. The information communicated to an output system can include both rates of the first section and the second section, or a weighted average of the rates based on the length of time of each section.

Various embodiments disclosed herein are directed toward a system for sensing motion using a motion sensor. The system includes one or more sources for generating electromagnetic radiation, wherein the frequency of the generated electromagnetic radiation is in the radio frequency range. The system further includes one or more transmitters that are configured to transmit the generated electromagnetic radiation towards a subject and one or more receivers that are configured to receive a radiation scattered at least by the subject. A Doppler shifted signal is extracted signal extractor from the scattered radiation by a signal extractor. The system further includes a processor that is configured to transform the Doppler shifted signal to a digitized motion signal, the digitized motion signal having one or more frames, wherein the one or more frames comprise time sampled quadrature values of the digitized motion signal. The one or more frames are demodulated using a demodulation algorithm that is executed by a demodulator. The demodulation process results in isolating a signal corresponding to a physiological movement of the subject or a part of the subject. The isolated signal can be analyzed to obtain information corresponding to a non-cardiopulmonary motion or other signal interference. The disclosed system can be configured to process the signal to obtain information corresponding to the physiological movement of the subject or a part of the subject, which is substantially separate from said non-cardiopulmonary motion or other signal interference and estimate point-in time vital signs parameters at pre-determined intervals and communicate the information to an output system that is configured to perform an output action. In various embodiments, the processor can be configured to function as a signal extractor and a demodulator.

Various embodiments disclosed herein describe a system for estimating the presence or absence of paradoxical breathing using a motion sensor. The system includes one or more sources for generating electromagnetic radiation, wherein the frequency of the generated electromagnetic radiation is in the radio frequency range. The system further includes one or more transmitters configured to transmit the generated electromagnetic radiation towards a subject and one or more receivers configured to receive a radiation scattered at least by the subject. A signal extractor is used to extract a Doppler shifted signal from the scattered radiation and transform the Doppler shifted signal to a digitized motion signal using a processor. The digitized motion signal can include one or more frames, wherein the one or more frames comprise time sampled quadrature values of the digitized motion signal. In various embodiments, the processor is configured to execute a non-cardiopulmonary motion detection algorithm to identify from the digitized motion signal one or more non-cardiopulmonary motion detection events or other signal interference events corresponding to the presence or absence of a non-cardiopulmonary motion or other signal interference. The processor can be further configured to execute a paradoxical breathing indication algorithm to estimate the presence or absence of paradoxical breathing. Information related to at least the presence, absence, or degree of paradoxical breathing is provided by the system. In various embodiments, the system can be further configured to process the one or more frames to obtain information corresponding to the cardiopulmonary movement of the subject or a part of the subject, substantially separate from non-cardiopulmonary motion or other signal interference and estimate the subject's depth of breath from the cardiopulmonary motion.

### BRIEF DESCRIPTION OF THE DRAWINGS

FIG. 1A schematically illustrates an embodiment of a physiological motion sensor system comprising radar.

FIGS. 1B-1F illustrates measurements obtained by the system illustrated in FIG. 1A.

FIG. 2 schematically illustrates a block diagram of a radar-based physiological motion sensor system integrated with a remote interface.

FIG. 3 schematically illustrates a block diagram of a system including radar-based physiological motion sensor including an add-on module.

FIG. 4 schematically illustrates the block diagram of a standalone radar-based sensor device configured to communicate with a hospital network.

FIG. 5 schematically illustrates another embodiment of a standalone radar-based sensor device with wireless connectivity.

FIG. 6 schematically illustrates another embodiment of a radar-based physiological motion sensor comprising a processor and a display.

FIGS. 6A-6C schematically illustrate various embodiments of a radar-based physiological motion sensor that is configured to wirelessly communicate with a patient monitor.

FIG. 6D illustrates a block diagram of an embodiment of a system configured as an activity index indicator.

FIG. 6E illustrates a screen shot of a display device that displays the activity index.

FIG. 7 schematically illustrates an embodiment of a radar-based physiological motion sensor comprising a transmitter and multiple receivers.

FIG. 8 illustrates a flowchart of an embodiment of a method configured to perform DC cancellation.

FIGS. 8A and 8B illustrate flowcharts of an embodiment of a method configured to perform DC compensation.

FIG. 8C illustrates the acquired signal fit to a curve or a line.

FIG. 8D illustrates a demodulation algorithm utilizing a circle-find or an arc-find function.

FIGS. 8E-8H illustrate various embodiments of data acquisition systems.

FIG. 8I illustrates the effect of sweeping the frequency of the local oscillator on the DC offset.

FIGS. 8J-8L illustrate various embodiments of the radar sensor including an aiming aid.

FIG. 8M illustrates a schematic for radio frequency tags and a sensor set.

FIG. 8N illustrates a screen shot of a display associated with a continuous vital signs monitor equipped with a tag-based power indicator.

FIG. 9 illustrates an embodiment of a linear demodulation algorithm.

FIG. 9A illustrates the heart trace obtained with a vector locked to the respiration vector.

FIG. 9B illustrates the heart trace obtained with independent vectors.

FIGS. 9C and 9D illustrate embodiments of a demodulation process.

FIGS. 10A-10D illustrate an embodiment of a rate estimation algorithm including frequency domain rate estimation and time domain rate estimation.

FIG. 10E shows the different key points in a respiration cycle.

FIG. 10F illustrates a method to identify the peaks and valleys in a respiration cycle based on the first derivative of the respiration signal.

FIG. 10G illustrates a graph of the signal and the time-delayed version of the signal.

FIG. 10H illustrates a screen shot of an embodiment of a display device associated with a radar based sensor device that is configured to operate in the Auto Mode.

FIG. 10I illustrates an embodiment of an algorithm to assess the regularity of respiration.

FIG. 10J illustrates a system configured to determine the regularity of respiration.

FIGS. 11A and 11B illustrate the phasor diagrams for normal breathing and paradoxical breathing.

FIG. 11C shows an embodiment of a cost function configured to convert the paradoxical factor to a paradoxical indicator.

FIGS. 11D and 11E illustrate the baseband outputs with multi-path delayed signals when the body parts exhibit simultaneous expansion and contraction.

FIGS. 11F and 11G illustrate the baseband outputs with multi-path delayed signals when the body parts exhibit expand or contract with different phase delay.

FIG. 12 illustrates an arc that is fit to the respiratory data.

FIGS. 12A-12D illustrates an embodiment of a method configured to detect non-cardiopulmonary motion.

FIG. 12E illustrates a transition table.

FIG. 12F illustrates a state diagram.

FIG. 13 schematically illustrates a block diagram of an embodiment of a self testing circuit.

FIG. 14 (which consists of 14A and 14B) illustrate an embodiment of a method for separating multiple cardiopulmonary signals.

FIG. 15 illustrates measurements showing the separation of respiratory signals from two targets.

FIG. 16 (which consists of 16A and 16B) illustrate an embodiment algorithm for tracking the direction of one or more cardiopulmonary signals.

FIG. 16BA illustrates a summation pattern and a subtraction pattern of two rectangular patch antennas separated by a half wavelength.

FIG. 16BB illustrates an embodiment of a compact array.

FIGS. 16C-16F illustrate various embodiments of an identification system that is used to provide positive patient identification in conjunction with remote vital signal sensing.

FIG. 16G illustrates a system of enabling positive identification using a tag attached to the patient.

FIG. 16H illustrates an embodiment of a passive transponder RFID technology.

FIG. 16I illustrates an embodiment of a Doppler respiratory and identification reader.

FIG. 16J illustrates an embodiment of a method of identification reading and vital signs signals processing of the sideband signals.

FIG. 17 illustrates an alternate embodiment of the radar-based physiological motion sensor system.

FIG. 18 illustrates an embodiment of the radar-based physiological motion sensor comprising a sensor unit, a computational unit and a display unit.

FIG. 19 illustrates an embodiment of an interface (e.g., a display screen) configured to output cardiopulmonary or cardiovascular related information.

FIG. 20 illustrates a screen shot of a display device showing a respiratory rate.

FIG. 21 illustrates an alternate embodiment of the radar-based physiological motion sensor comprising a sensor unit, a computational unit and a display unit.

FIG. 21A illustrates an embodiment of a system that is powered using a USB interface.

FIGS. 21B-21F illustrate various screen shots of the display associated with an embodiment of a radar based sensor device.

FIG. 22 illustrates an alternate embodiment of the radar-based physiological motion sensor comprising a sensor unit and a processor.

FIG. 23 shows a screen shot of an embodiment of a display device configured to display the respiration signal and the heart signal in addition to other information.

FIG. 24 is a screen shot of a display device or unit illustrating the respiratory rate, activity indicator and position of a sleeping subject.

FIG. 25A shows the application of the system in a hospital environment to measure the respiratory and/or cardiac activity of a patient.

FIG. 25B is a screenshot of the display device illustrated in FIG. 25A.

FIGS. 26A and 26B illustrate screen shots of a display device that can be used for viewing the vital signs provided by the device

FIG. 27 illustrates an embodiment of a DC-cancellation circuit.

FIG. 28 illustrates an embodiment of a method to determine a paradoxical breathing indicator.

FIGS. 29 and 30 are screen shots of a display device configured to display the output from a system configured to detect paradoxical breathing

FIG. 31 illustrates an embodiment of a system including a compact antenna array.

FIG. 32 illustrates an embodiment of a system including two receiving antennas.

FIG. 33 illustrates the screen shot of a display device configured to output cardiopulmonary information of two people after DOA processing separated their respiratory signals.

FIG. 34 illustrates a screen shot of a display device configured to display a respiratory waveform and tidal volume.

FIG. 35 illustrates a screen shot of a display device configured to display the respiratory motion waveforms for two people.

FIG. 36A shows a complex constellation plot of the quadrature phase component and the in-phase component of a signal.

FIG. 36B shows a plot of depth of breath versus time as measured by a radar-based physiological motion sensor and a conventional motion sensor, e.g., chest strap.

FIG. 36C shows a snapshot of a display device illustrating the tidal volume, a waveform corresponding to the respiratory activity and a respiratory rate.

FIG. 37 illustrates a schematic layout of an array element including a transmitting antenna and at least four receiving antennas.

FIGS. 38A-38C illustrate information related to cardiopulmonary activity as measured by a wearable Doppler radar system in contact with a subject.

FIG. 38D illustrate information related to cardiopulmonary activity as measured by a non-contact Doppler radar system.

FIGS. 38E-38J show embodiments of a display device configured to display measurements related to cardiopulmonary activity and indicate presence of a subject.

FIG. 38K illustrates an embodiment of a spiral antenna.

FIG. 38L illustrates the matching property for the spiral antenna.

FIG. 38M illustrates the simulation results of RF signal power.

FIG. 38N illustrates the blood pressures that were measured by an embodiment of the radar based motion sensor.

FIG. 38P illustrates an embodiment of an air gap antenna.

FIG. 38Q illustrates a partial RF circuit that can be mounted on a subject's body.

FIG. 38R illustrates the correlation between the pulse signal from the radar sensor and the mean arterial pressure.

FIGS. 39A and 39B describe embodiments of a network topology of a plurality of clusters including a radar-based physiological motion sensors.

### DETAILED DESCRIPTION

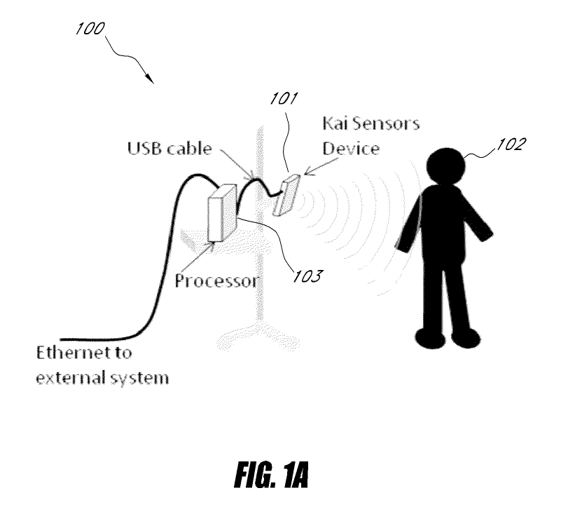

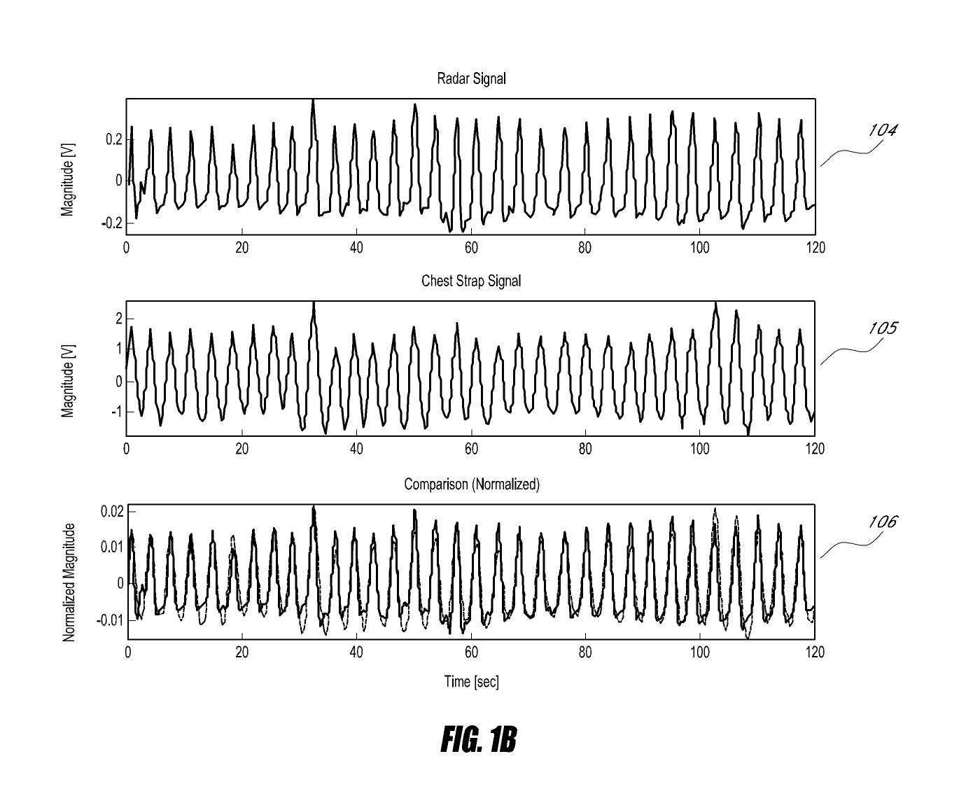

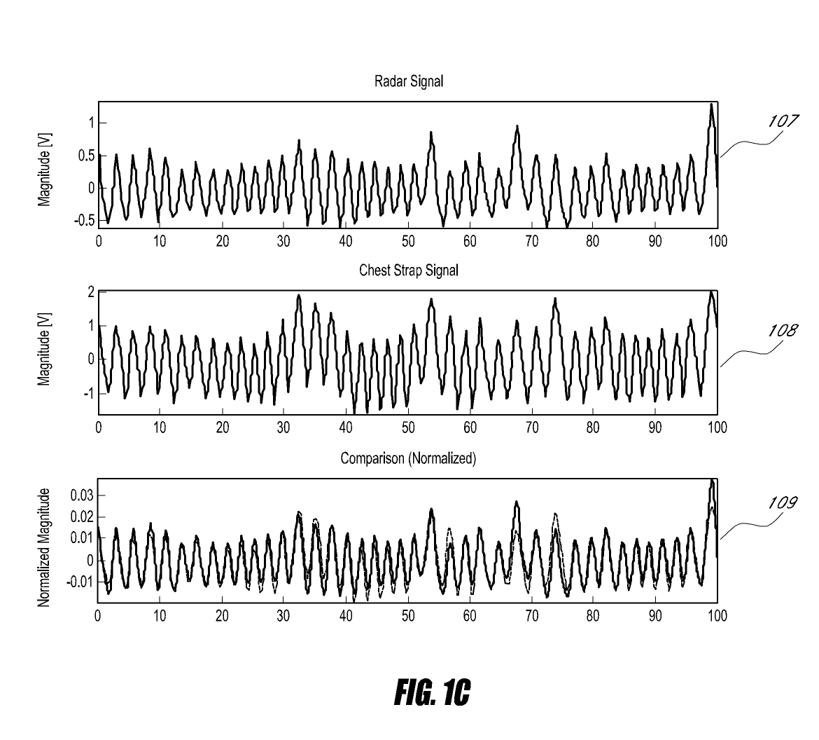

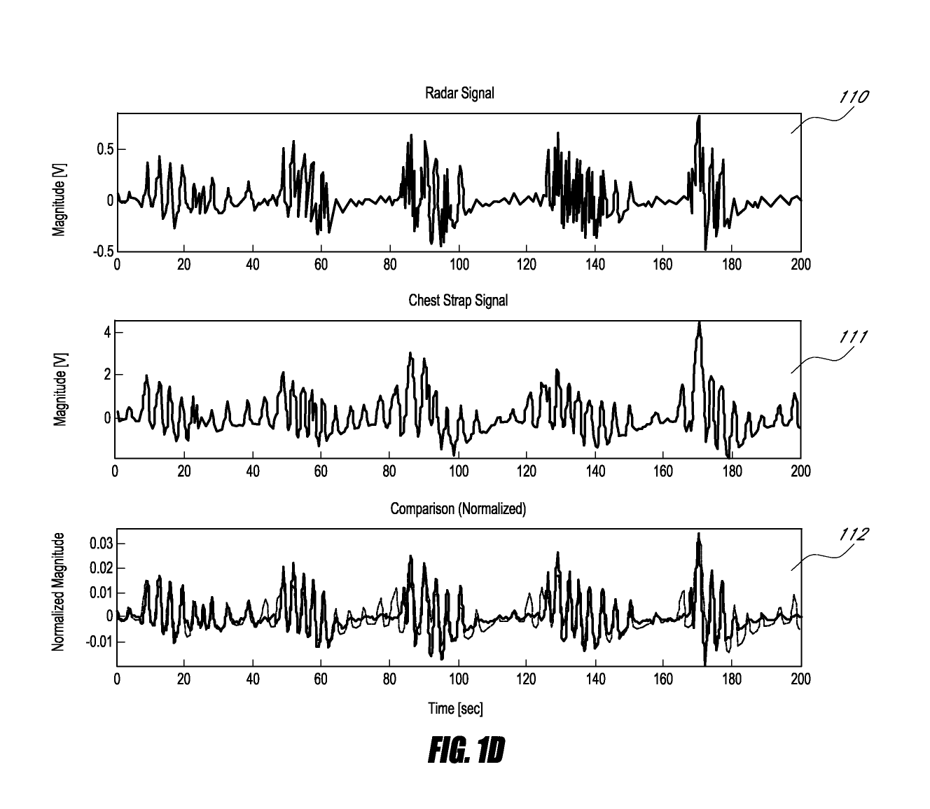

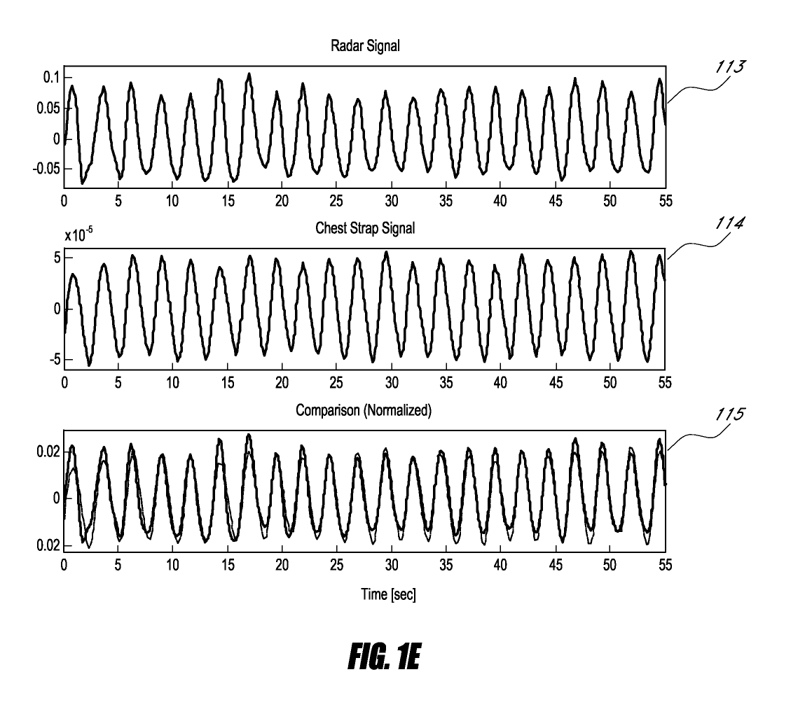

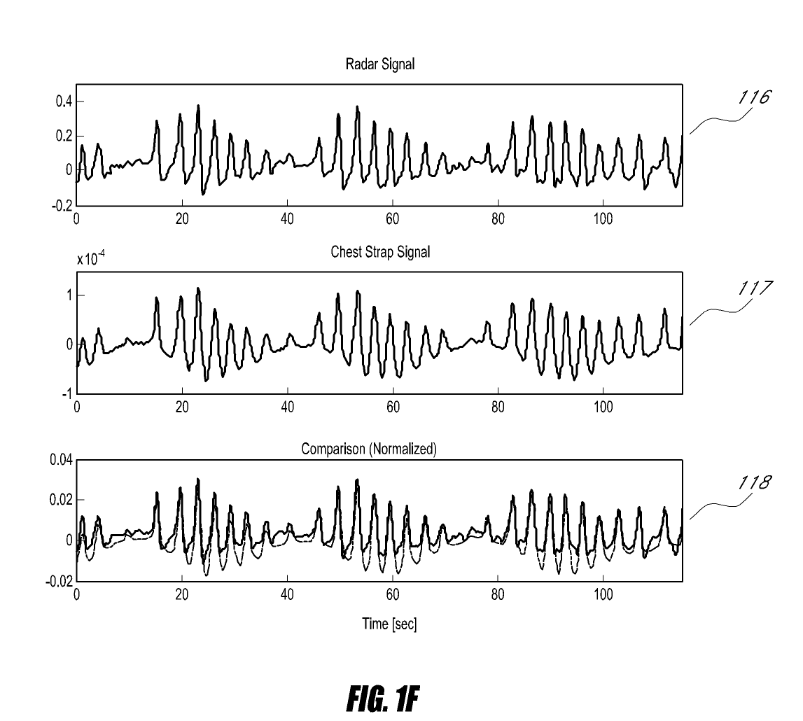

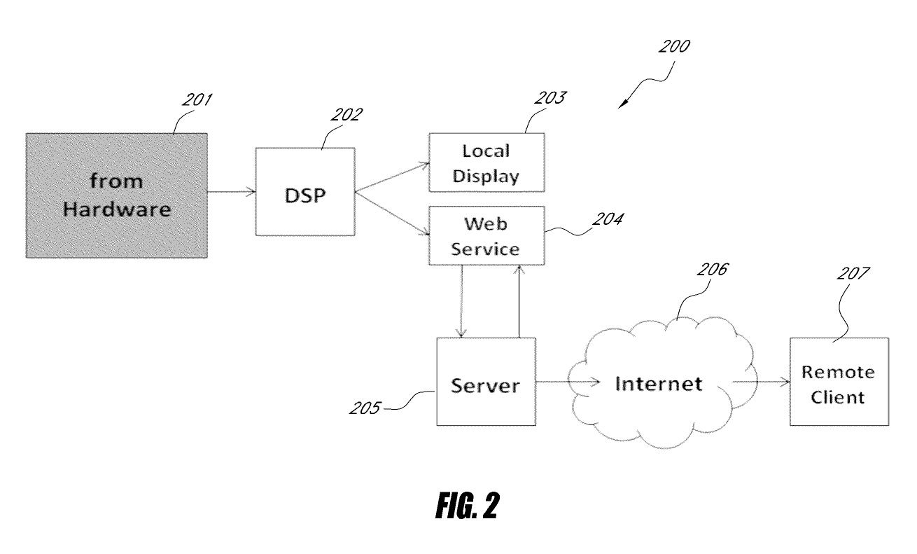

FIG. 1A shows a physiological motion sensor system 100 wherein a radar 101 senses motion and/or physiologic activity of a subject 102. Data from the radar 101 is provided to a processing system 103 that analyzes the radar data to determine various desired physiological parameters and provide output information regarding the physiological parameters to an output system or device configured to perform an output action. In various embodiments, the output device can include a display system configured to display an audible system configured to report information or issue alerts or a medical device configured to perform a function based on the information. The system 100 can further include a communications system configured to communicate using wired or wireless communication links. The communications system can use standard or proprietary protocols. FIG. 1B shows an example of a measurement obtained by the system 100 as displayed on a display unit.

FIGS. 1B-1F illustrate examples of the measurement obtained by the system 100. The measurements can include waveforms due to cardiopulmonary activity of a subject 102 displayed on a display unit.

FIG. 1B illustrates the waveforms obtained by embodiments of the system 100 described above for a 54-year-old male subject with a body mass index (BMI) of 23 with Hypertension and Congestive Heart Failure. Plot 104 of FIG. 1B shows the physiological motion signal (e.g., respiratory rate and the amplitude of respiration) detected by the radar-based physiological motion sensor system. Plot 105 illustrates the physiological motion signal detected by a conventional contact physiological motion sensor (e.g., a chest strap). Plot 106 shows the comparison between the normalized motion signal detected by the radar-based physiological motion sensor and the normalized conventional sensor. Plot 106 shows good correspondence between the two signals.

FIG. 1C illustrates variations in the respiratory rate and the amplitude of respiration obtained by embodiments of the system described above for a 44-year-old male with a BMI of 40, with Diabetes, Hypertension, and CAD. Plot 107 of FIG. 1C shows the physiological motion signal (e.g., respiratory rate and the amplitude of respiration) detected by the radar-based physiological motion sensor system. Plot 108 illustrates the physiological motion signal detected by a conventional contact physiological motion sensor (e.g., a chest strap). Plot 109 shows the comparison between the normalized motion signal detected by the radar-based physiological motion sensor and the normalized conventional sensor. As observed earlier, plot 109 shows good correspondence between the two signals.

FIG. 1D illustrates the physiological motion signal for a 55-year-old male with a BMI of 40, with High Cholesterol, Hypertension, and CAD, while he was snoring. Plot 110 shows the motion signal detected by the radar-based physiological motion sensor and illustrates detection of apnea (cessation of breathing) and variation in the respiration signal baseline. Plot 111 is a corresponding measurement obtained by a conventional monitor while plot 112 illustrates the comparison between the conventional monitor and the system 100.

FIG. 1E illustrates the physiological motion signal for a 59-year-old female with a BMI of 30, with COPD and CHF. Plot 113 shows the measurement obtained by the physiological motion sensor of system 100. Plot 114 shows the corresponding measurement obtained by a conventional sensor and plot 115 shows the comparison between the two measurements.

FIG. 1F illustrates the physiological motion signal for a 57-year-old Female with a BMI of 38, with CHF and CAD. Plot 116 illustrates detection of apnea (cessation of breathing) and variation in the respiration signal baseline for the subject. Plot 117 illustrates a corresponding measurement obtained by a conventional sensor and plot 118 shows the comparison between the two.

In various embodiments, the radar-based physiological sensor can include a user interface to allow a user to enter information or to allow the user to enter commands and/or instructions. In various embodiments, the user interface can include a start button and a stop button as disclosed in U.S. Provisional App. No. 61/128,743 which is incorporated herein in its entirety, said starting and stopping buttons. In various embodiments, the user interface can include a clear button. In various embodiments, the user interface can include additional buttons (e.g., a save button, a print button, etc.) or a keypad.

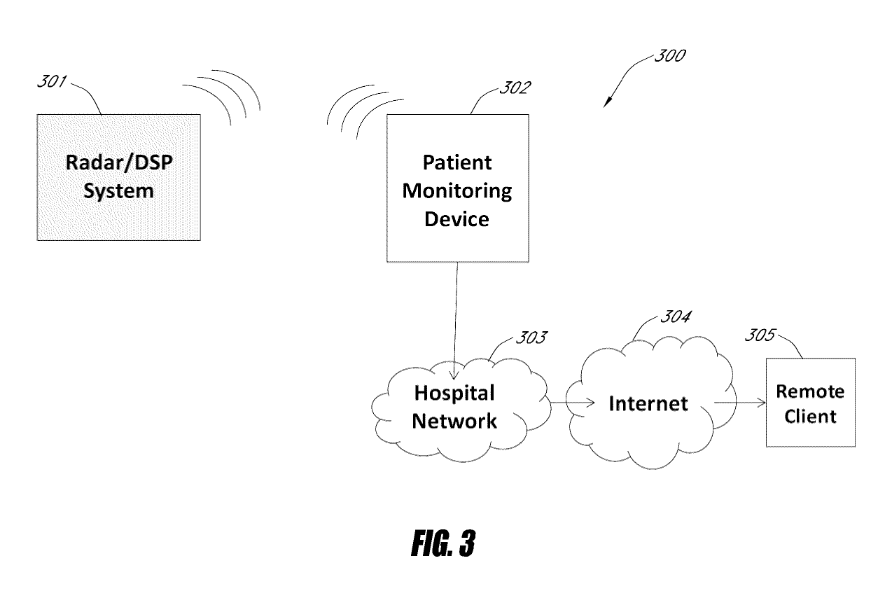

In various embodiments, the system 100 can communicate the information to a remote display and/or a central server or a computer. In some embodiments, SOAP web service can communicate data to a server. From the server, the respiration data can be accessed by a remote client with a browser and an internet connection as disclosed in U.S. Provisional App. No. 61/072,983, which is incorporated herein by reference in its entirety. FIG. 2 illustrates a block diagram of a system integrated with a remote interface 200. The system illustrated in FIG. 2 includes a radar-based physiological sensor 201 in electrical communication with a signal processor 202. The information from the signal processor can be displayed locally on a local display 203 or can be stored in a server 205 over a web service 204. A remote client 207 can access the information stored on the server using the internet 206 or some other communication protocol.

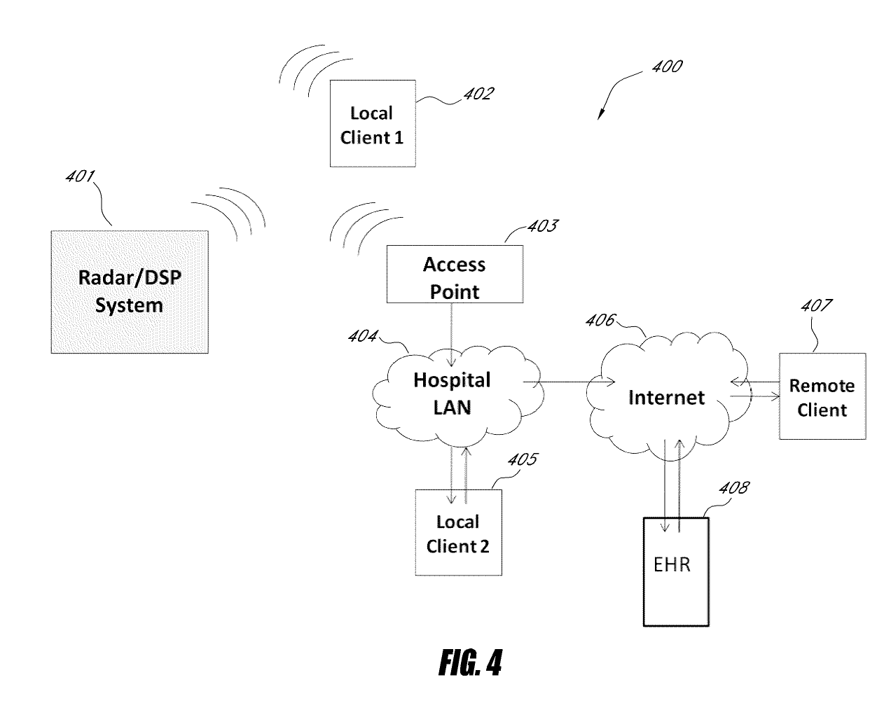

In various embodiments, the system 100 can include an add-on module with wireless connectivity as disclosed in U.S. Provisional App. No. 61/125,022, which is incorporated herein by reference in its entirety. FIG. 3 illustrates a block diagram of a system 300 including radar-based physiological sensor including an add-on module. As illustrated in FIG. 3, the device 301 is networked to a patient monitoring system 302 using a personal area network technology such as Bluetooth, Ultra Wide Band, Wireless USB, etc. The patient monitoring system 302 can display the cardiopulmonary motion information on its local interface and/or forward the data to a remote database over the internet 304 or a hospital network 303 such that it can be accessed by a remote client 305.

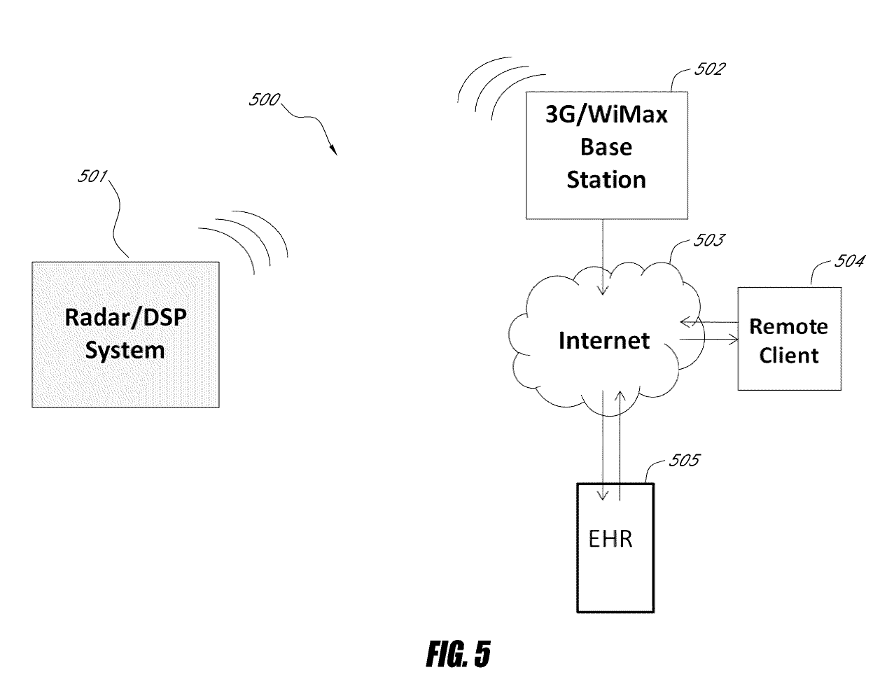

FIG. 4 illustrates the block diagram of a Standalone Device configured to communicate with a hospital network. The system 400 illustrated in FIG. 4 includes a radar-based physiological sensor system 401 similar to the system 100 described above including a digital signal processor. The system 401 is in wireless communication to an access point 403. The radar-based physiological sensor system 401 can communicate information related to the physiological or cardiopulmonary motion to a remote server, connected to the hospital network 404, via the access point 403 using a wireless communication technology such as Bluetooth, Wireless USB, etc. The access point 403 can be connected to the hospital network 404 (e.g., the hospital LAN) over a wired or a wireless network. A local client 402 or 405 can access the information from the system 401 or the server wirelessly or over the hospital network 404. A remote client 407 can also have access to the information over the internet 406. In various embodiments, the information from the system 401 can be communicated to a central database 408 maintaining electronic health records over the internet 406.

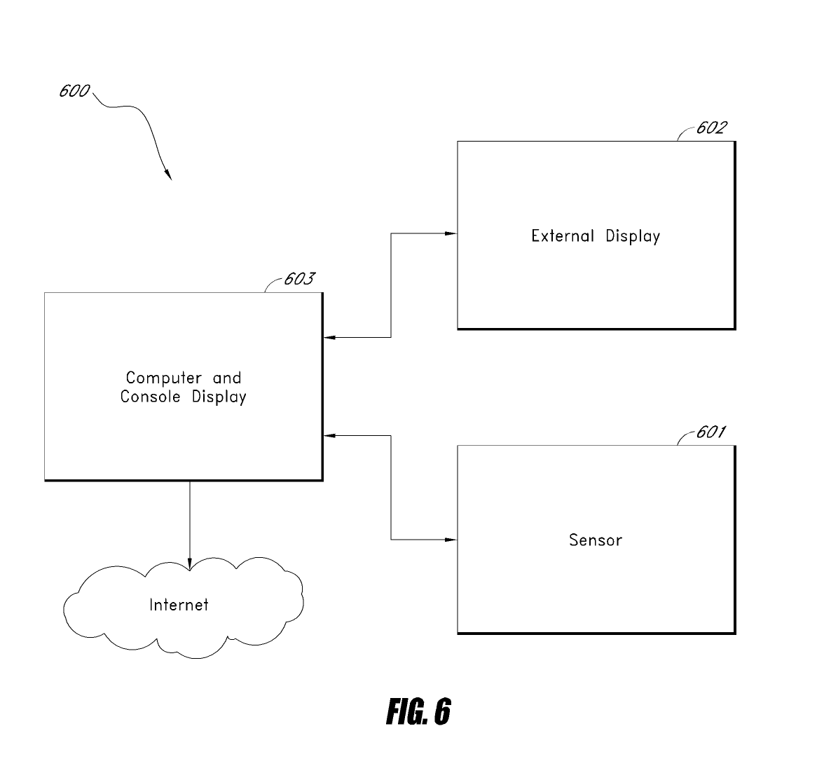

Various embodiments of the system 100 can communicate information using TCP/IP over Ethernet Connectivity or with Serial RS-232 Connectivity. FIG. 5 illustrates another embodiment of a standalone device with wireless connectivity 500 as disclosed in U.S. Provisional App. No. 61/125,022, which is incorporated herein by reference in its entirety. A radar system 501 similar to system 100 described above can use any of several wireless technologies to connect with a central healthcare practitioner's station, a patient information database, and/or an electronic medical record 505. The network can be configured to forward or display the data on PC's, PDA's or medical tablets of a remote client 504 over the internet 503. In a hospital setting, the system 501 can use communication protocols such 802.11 or any other communication protocol the hospital uses for networking. If the system 501 is used in a home or field setting, a 3G cellular or WiMax connection can be used in lieu of a LAN technology to send the data to the electronic health record 505 or a remote client 504 or other databases via the internet 503. In various embodiments, the information sent by the system 501 can be viewed by a healthcare practitioner.

In various embodiments, the device 501 can also be made to conform with the standards set forth by the Continua health alliance by following a scheme such that the device uses Bluetooth or USB to connect with a managing computer which will disseminate the data to a healthcare provider's network for storage or examination.

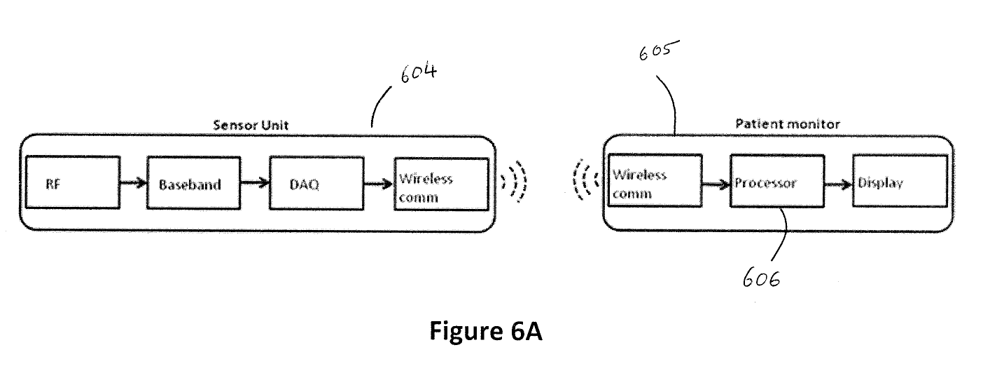

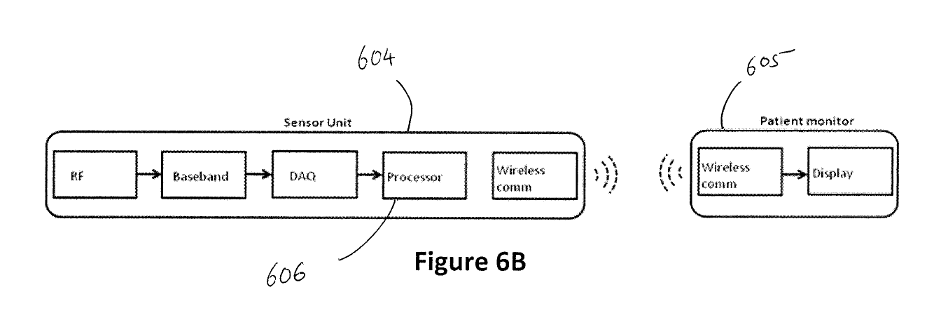

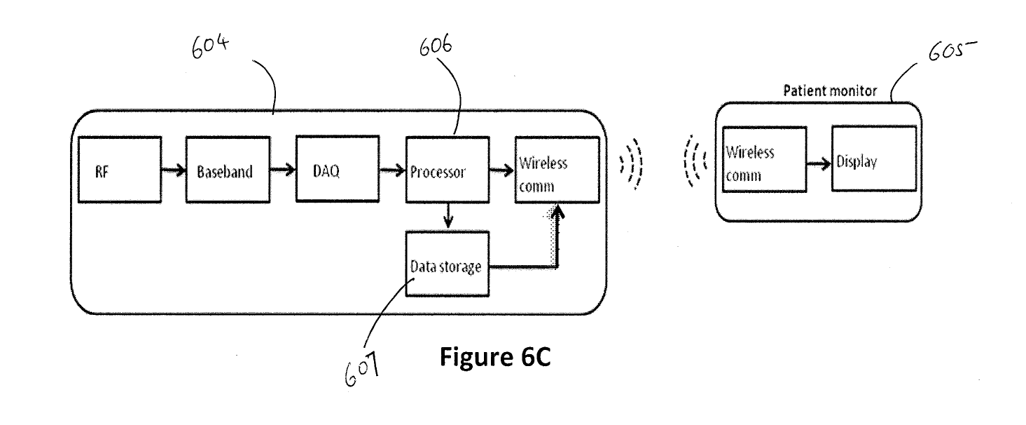

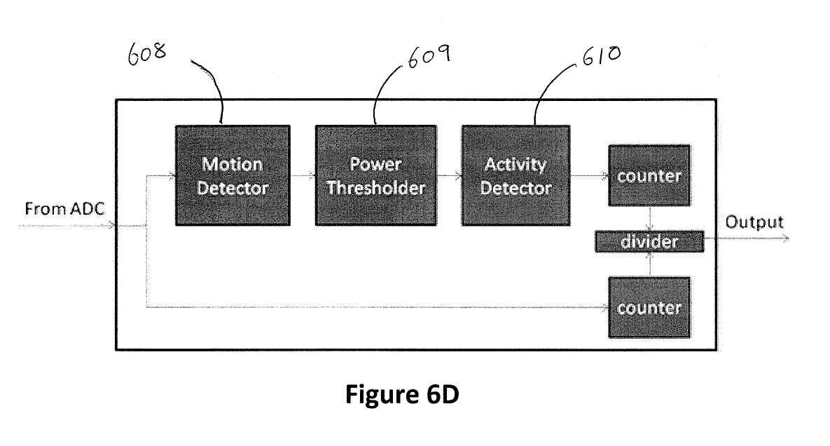

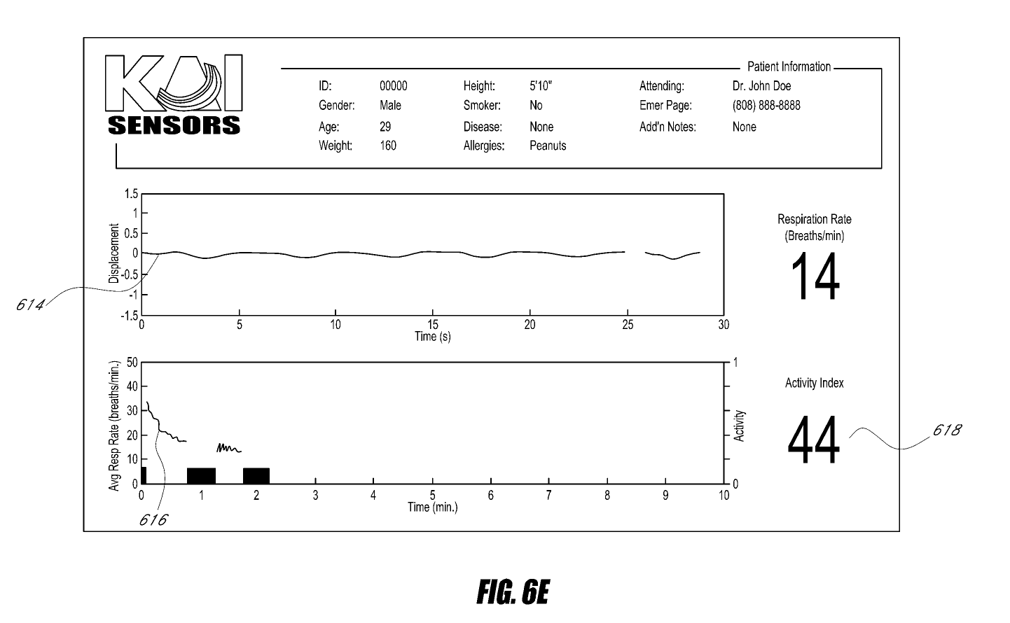

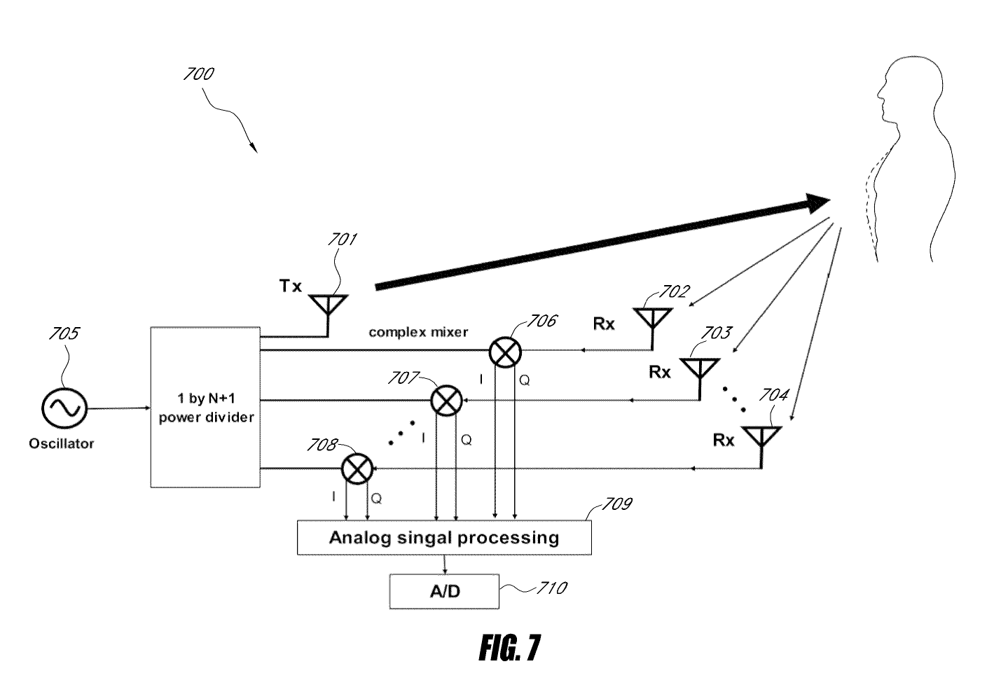

FIG. 6 illustrates a system 600 including a physiological motion sensor 601 similar to system 100 described above in communication with a computer including a console display 603. In some embodiments, the computer 603 can be in communication with an external display 602. In some embodiments, the sensor 601 can communicate information related to the physiological motion to the computer for storage and/or display. A remote client can be able to access the information from the computer over the internet.

Various embodiments of the physiological motion sensor system 100 described herein can be used as continuous monitoring devices and systems. Various embodiments of the system 100 can be used to measure cardiopulmonary motion from a distance ranging from many meters to the point of contact with body. Various embodiments of the system 100 provide physiological waveforms, displays of physiological variables, history plots of physiological variables, indications of signal quality and/or indications of specific conditions. Various embodiments can include physiological waveforms including respiratory waveforms, heart waveforms, and/or pulse waveforms. Various embodiments can include physiological variables including respiratory rate, heart rate, tidal volume, depth of breath, inhale time, exhale time, inhale time to exhale time ratio, airflow rate, heart beat-to-beat interval, and/or heart rate variability. Various embodiments can include indications of signal quality, which can be general such as good quality, or poor quality, or which can be specific, including indication of low signal power, signal interference, non-cardiopulmonary motion, or circuit noise. Indications of specific conditions can include general indications of health, warnings of physiological variables that are outside the normal range, indication of abnormal breathing patterns, or indication of paradoxical breathing.

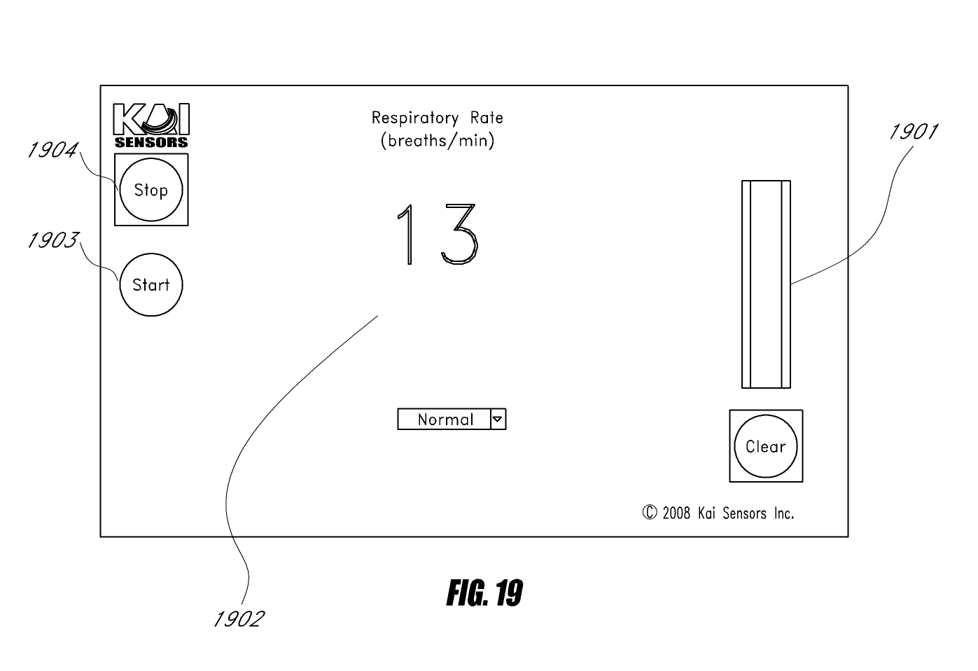

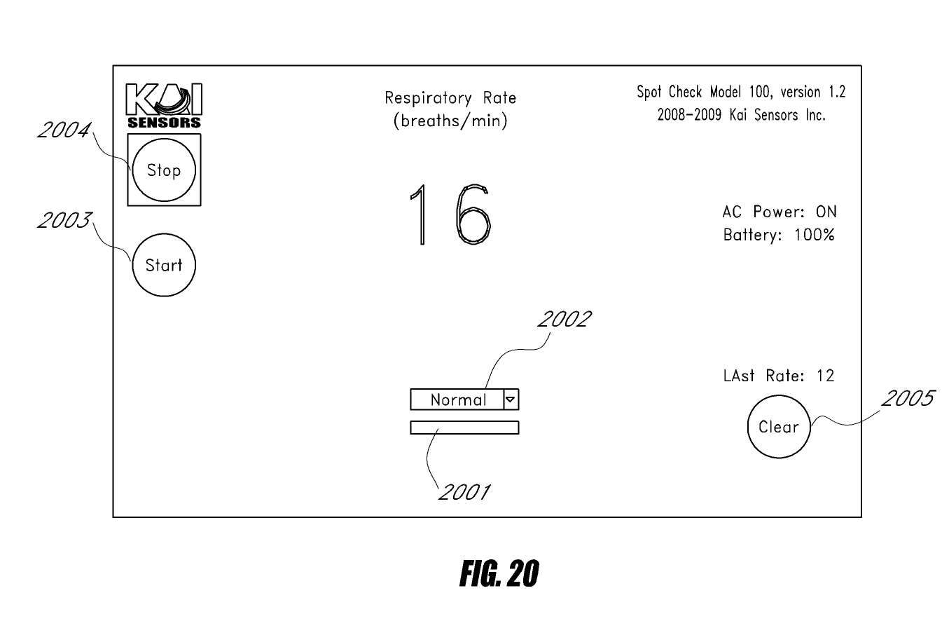

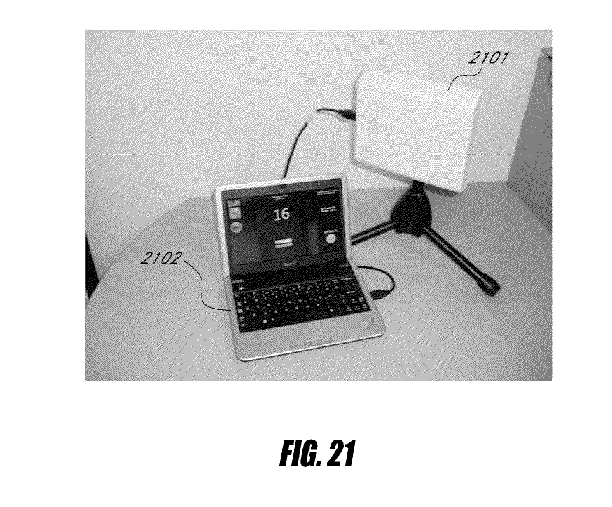

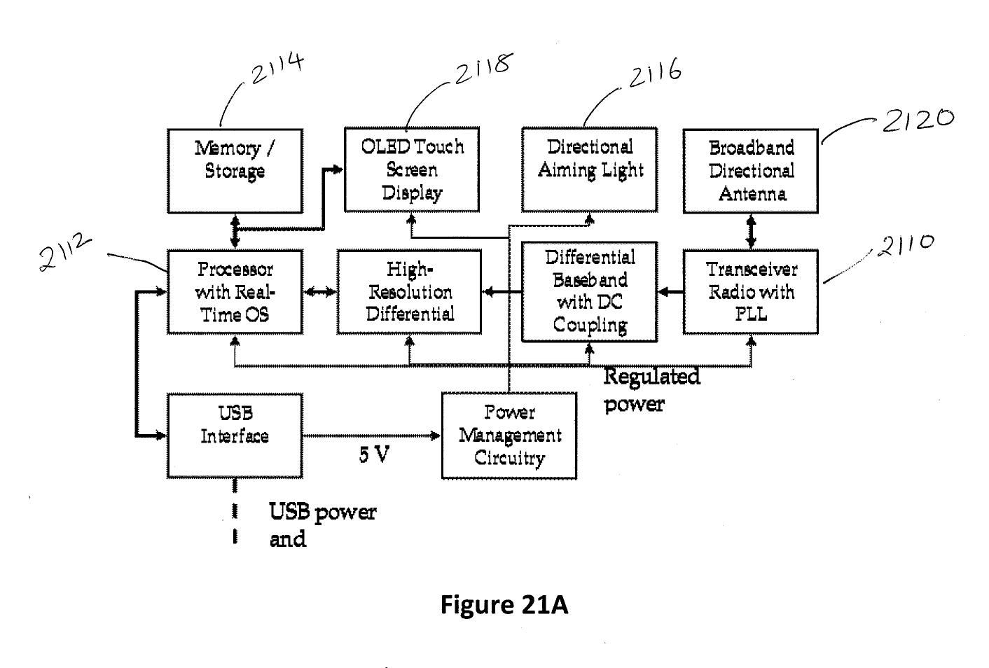

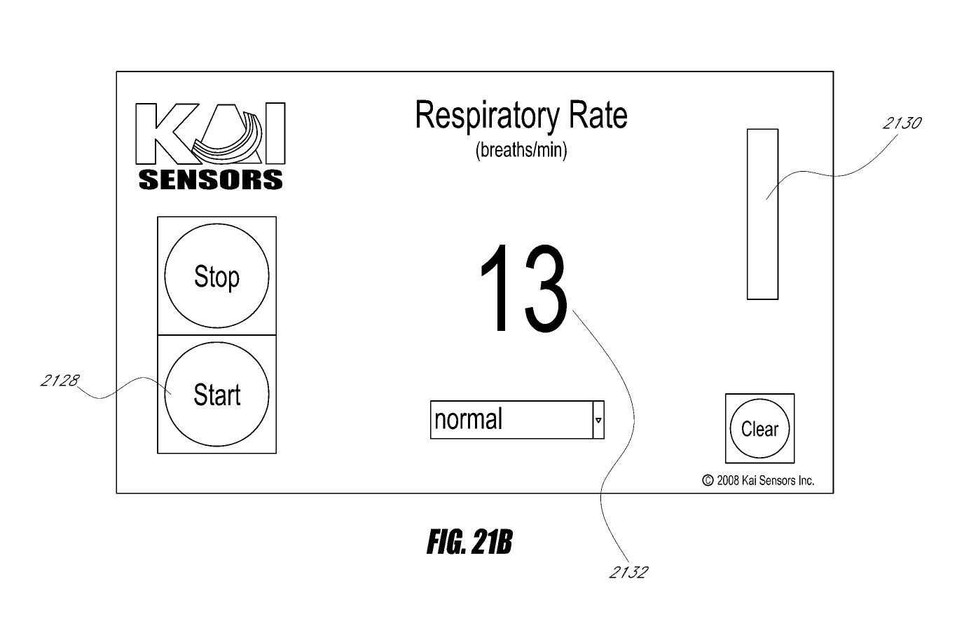

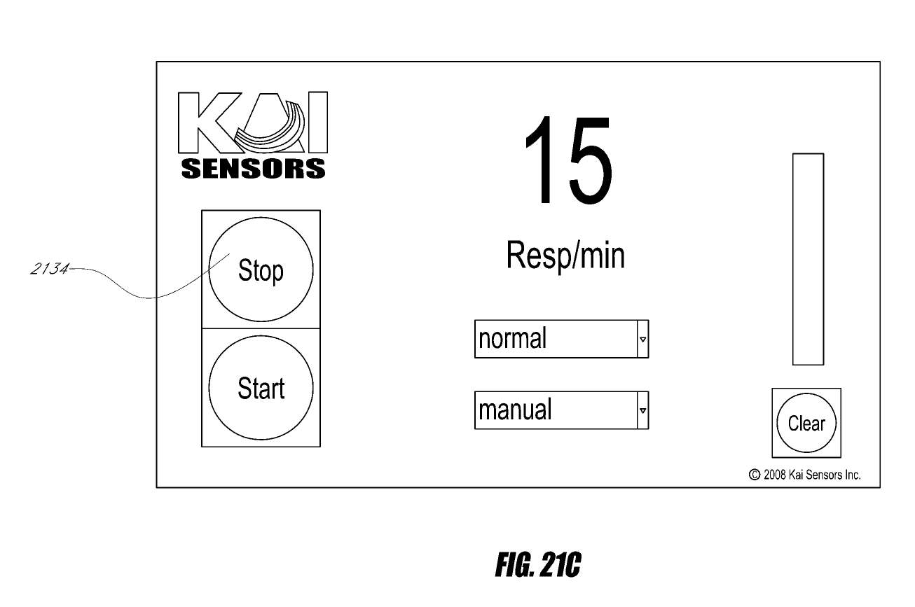

As shown below in FIG. 21, in various embodiments, the continuous vital signs monitor can have a local interface, including buttons and display, and it can have electronic communications to a central monitoring site (such as a central nurse's station) or to a central database (such as an electronic medical record). In various embodiments, the system 100 can be a stand-alone device, or it can be a module integrated in another vital signs monitoring device (e.g., a hospital monitoring system). Various embodiments of the continuous vital signs monitor can be used in the hospital or clinic for general patient monitoring, for monitoring of post-surgical patients, for monitoring of patients receiving pain medications that put them at high risk of respiratory depression, for monitoring patients with respiratory diseases or disorders, for monitoring patients using invasive or non-invasive ventilators, and for monitoring of patients during medical imaging scans as disclosed in U.S. Provisional App. No. 61/154,176 which is incorporated herein by reference in its entirety. Various embodiments of the continuous vital signs monitoring system 100 can be used in pediatric and/or neonatal wards in hospitals.

Various embodiments of the continuous vital signs monitor can be used in the home as disclosed in U.S. Provisional App. No. 61/072,983, which is incorporated herein by reference in its entirety and in U.S. Provisional App. No. 61/196,762 which is incorporated herein by reference in its entirety. Various embodiments of the device can operate locally, remotely or both. Various embodiments of the device can connect to another device, including, but not limited to, a personal health system, another home healthcare device, a personal computer, a mobile phone, a set-top box, or a data aggregator. Various embodiments of the device can connect via a wired or wireless connection to a central station at a remote location (away from the home). In various embodiments, the system 100 can have a local display which displays some or all of the obtained data on the display. In various embodiments, the system 100 can communicate the information to another device in the home, and/or it can communicate the information via a wired or wireless connection to a central database that is remote (e.g., away from the home). In various embodiments, the device can operate with local control, can be controlled by another device via a wired or wireless connection, can operate automatically, or can be controlled by a central system that is remote (e.g., away from the home). In various embodiments, this home device can be used for general vital signs monitoring, or it can be used to monitor chronic illnesses that affect the cardiopulmonary system including, but not limited to, Diabetes, Chronic Obstructive Pulmonary Disease, and Congestive Heart Failure. In various embodiments, the non-contact continuous vital signs monitor can be a module that is integrated into a personal health system or another home healthcare device, sharing its display and communications. Various embodiments of the system 100 can conform to Continua Health Alliance guidelines.

In various embodiments, the continuous vital signs monitor can also be used in a skilled nursing facility, in a similar embodiment to the hospital monitor. Embodiments of this device can be used for general vital signs monitoring of the elderly or ill, and can also be used for early detection of pneumonia. Embodiments of the continuous vital signs monitor can also be used in emergency vehicles (e.g., ambulances, helicopters, etc.) to monitor a patient during emergency transport. Various embodiments of the system 100 can also determine the duration of subject activity or the percentage of time the subject is active. This information can be used to provide an activity index. Changes in the activity index can be used as indicators of a change in health state. In various embodiments, the physiological motion sensor can be used to detect battlefield survivors and monitor their physiological signals as disclosed in U.S. Provisional App. No. 61/001,995 which is incorporated herein by reference in its entirety. In various embodiments, a software based array configuration that is executable by a processor can be applied to Doppler radar to search for survivors in detecting mode, and to track them in target mode by focusing the beam. Survivor location can be determined from DOA processing at dual or multiple frequencies.

As described in more detail below, the system 100 can include algorithms for calculating respiratory rate, accuracy of the respiratory rate, algorithms to recognize inaccurate data, to recognize interfering motion, to recognize electrical signal interference, to recognize electrical noise, to report varying rates, to analyze the regularity or irregularity of the respiratory rate and to signal or alert a user if the respiratory rate is high or low, etc.

As described in more detail below, the system 100 can include hardware and/or software which is executable by a processor to improve signal quality, such as, for example, RF leakage cancellation, DC cancellation, noise cancellation, low IF architecture, homodyne system balancing, etc. Various embodiments of the system 100 described herein can have the capability to discern between cardiopulmonary and other motions. In various embodiments of the system 100, methods and algorithms for motion discrimination and detection can enable increased accuracy of cardiopulmonary data. Various embodiments described herein employ methods of decreasing the delay between the occurrence of an event and the reporting and display of that event by DC cancellation and high speed data acquisition. A low time delay is typically important for applications in which another device uses the reported event to initiate or trigger another action. A low time delay also improves synchronization with other measurements. The respiration or heart waveforms that are generated by the various embodiments described herein can be used to trigger actions by other systems. For example, various embodiments describe triggering medical imaging (e.g., with CT or MRI scans) based on cardiac or respiratory displacement and triggering assistive ventilation based on spontaneous respiratory effort. The respiration or heart waveforms that are generated by the various embodiments described herein can be used to provide physiological synchronization with other systems. For example, various embodiments describe synchronizing cardiopulmonary motion or other motion to medical imaging (e.g., CT scans or MRI) systems, assistive ventilation systems, polygraph systems, security screening systems, biofeedback systems, chronic disease management systems and exercise equipment.

Various embodiments of the system 100 can automatically, using the algorithms related to Direction of Arrival (DOA), track a subject's physiological signals as the subject moves around e.g., up and down in a bed. Various embodiments of the system 100 can automatically, using the algorithms related to DOA, track a subject's location as the subject moves around e.g., up and down in a bed. Various embodiments of the system 100 can be configured to cancel extraneous motion when extracting cardiopulmonary motion which can result in greater accuracy of the readings. Various embodiments of the system 100 can also, using algorithms such as DOA, separate and monitor or measure secondary or multiple cardiopulmonary motion sources (e.g., cardiopulmonary motion of a second or multiple subjects nearby can be reported simultaneously). Various embodiments of the system 100 can also, using algorithms such as DOA, separate and suppress secondary or multiple cardiopulmonary motion sources (e.g., cardiopulmonary motion of a second or multiple subjects nearby can be suppressed such that only the intended subject is measured). Various embodiments of the system 100 can include a radio frequency identification (RFID) tag in conjunction with DOA to ensure tracking of the desired subject.

Various embodiments described herein can use various approaches for motion compensation such as empirical mode decomposition (EMD), suppression of secondary motion sources with direction of arrival (DOA) processing, blind signal separation (BSS), independent component analysis (ICA), and suppression of motion in the direction of high-frequency received signals.

Various embodiments of the system 100 can include radio frequency identification (RFID) tag configured to enable positive identification of a monitored subject. Various embodiments of the system 100 can be adapted to have various sizes, form factors and physical dimensions suitable for including in a bedside unit, a hand held unit, in a PDA, a module as part of larger medical system, etc. Various embodiments of the system 100 can include one or more outputs such that information can be viewed and controlled either locally or remotely. In various embodiments, the system 100 can be a thin client application such that the system 100 will include the sensor, data acquisition, and communications, and demodulation, processing, and output systems would be in another device. For example, in some embodiments, the system 100 is provided to a network system where controls and processing are centralized for a network of sensors and the sensor and networking/communications part is onsite, near the subject. In some embodiments, the system 100 automates the initiation of measurements under certain predefined circumstances e.g., when person is detected in a room, at set time intervals, etc. In various embodiments, the system 100 can be used to perform non-contact measurement of depth of breath and relative tidal volume or absolute tidal volume. Various embodiments of the system 100 can be used as a cardiopulmonary and/or activity monitor.

In various embodiments, the system 100 can be integrated with other contact or non-contact medical monitoring devices, such as, for example, pulse oximeters, blood pressure cuffs, etc. In various embodiments, the system 100 can be integrated with an air flow sensor and a pulse oximeter to meet requirements of Type 3 Home Sleep Test. In various embodiments, sleep apnea detection can be performed, either with the system 100 alone or in combination with other devices. In some embodiments, the system 100 can be used to measure physiological response to particular stimuli e.g., questions, images, sounds, entertainment, activities, education. In various embodiments, the system 100 can be used by veterinarians as a non-contact cardiopulmonary monitor for animals. In various embodiments, the system 100 can be used by researchers as a non-contact cardiopulmonary monitor in animals, for example, to study vital signs during hibernation or for post surgery monitoring of animals. Some embodiments of the system 100 can be used in triage applications e.g., battlefield triage or disaster area triage. Various embodiments of the system 100 can be used to monitor cardiac, cardiopulmonary, and/or respiratory activity in infants and neonates.

Non-contact physiological motion sensors, according to various embodiments described herein can be used to obtain a measurement of respiratory motion, which can be used as a continuous respiratory monitor. This continuous respiratory monitor can be a stand-alone device, with its own display, buttons and/or external communications, or it can be a module integrated with other vital signs monitoring devices or other medical devices. This continuous respiratory monitor can provide respiratory waveforms. This continuous respiratory monitor can provide current values and historical plots for respiratory values including respiratory rate, tidal volume, inhale time, exhale time, inhale time ratio to exhale time ratio, depth of breath, abdominal excursion to chest excursion ratio, and/or airflow rate. This continuous respiratory monitor can provide information on the variability and historical variability, each in various frequency bands, of respiratory rate, tidal volume, inhale time, exhale time, inhale time ratio to exhale time ratio, depth of breath, abdominal excursion ratio, and/or airflow rate. This continuous respiratory monitor can provide indications and history of indications of the presence and degree of paradoxical breathing, the presence and degree of obstructed breathing, and/or the presence and degree of distressed breathing. This continuous respiratory monitor can provide information on the frequency, depth, and length of gasps and sighs. This continuous respiratory monitor can provide information on the frequency and duration of non-cardiopulmonary motion. This continuous respiratory monitor can provide information on changes in the shape of the breathing waveform, or changes in the harmonic content of the breathing waveform. Various embodiments of the continuous respiratory monitor system include an interface that provides alerts for high and low respiratory rates, rate history, tidal volume history, information related to inhalation/exhalation intervals, indication of paradoxical breathing, indication of obstructed breathing, subject position, activity level/monitoring, for distinguishing between motion and measured cardiopulmonary activity, health ranking (e.g., high, medium, and low) and signal quality ranking (e.g., alerts when signal is too low). Various embodiments of the system 100 can provide alerts for high respiratory rates, low respiratory rates, high variability of respiratory rates, low variability of respiratory rates, irregularity of breathing pattern, changes in breathing pattern, high inhale time to exhale time ratio, low inhale time to exhale time ratio, and changes in inhale time to exhale time ratio. Thresholds for these alerts can be values that are pre-set, values that can are set by the user, values that are calculated based on a patient's baseline respiratory rates, or values that are calculated based on a patient's baseline rates and historical variability of a patient's rates.

The system 100 can be used in systems that monitor sleep in subjects. For example, in some embodiments, the system 100 can provide a non-contact approach to replace piezoelectric or inductive chest straps for measuring respiratory effort and/or respiratory rates. In various embodiments, the system 100 can provide a non-contact approach to replace piezoelectric or inductive chest straps for measuring the difference in respiratory related motion for different parts of the body (e.g., as a paradoxical breathing indicator). In various embodiments, the physiological motion sensor can be used either alone or in combination with other devices to detect obstructive sleep apnea, central sleep apnea or other sleep disorders. In various embodiments, the system 100 can be used with an air flow sensor and/or a pulse oximeter for a Type 3 Home Sleep test. In various embodiments, the system 100 can be used with a wireless air flow sensor and/or a wireless pulse oximeter for a wireless Type 3 Home Sleep test with minimal patient contact. In various embodiments, the system 100 can be used alone as a Type 4 Home Sleep Test. In various embodiments, the system 100 can be used alone as a Type 4 Home Sleep Test that involves no contact with the subject and operates from a distance. In various embodiments, the system 100 can provide a non-contact way of measuring cardiopulmonary activity as well as limb and other body motion during sleep. Various embodiments of the system 100 can conform to Continua Health Alliance guidelines. In various embodiments, the system 100 can be used for sudden infant death syndrome (SIDS) monitoring or screening (e.g., in infants or neonates). Various embodiments of the system 100 can be used to monitor cardiopulmonary and/or cardiac activity in infants and newborns. Various embodiments of the system 100 can be used on neonates, infants, children, adults, and elderly subjects.

Various embodiments of the physiological motion sensors described herein can be used to obtain respiratory effort waveforms. As such, they can be used as part of a home sleep test as disclosed in U.S. Provisional App. No. 61/194,836 which is incorporated herein by reference in its entirety that includes pulse-oximetry and nasal airflow sensors to detect both central apnea and obstructive sleep apnea, and to differentiate between the two. Various embodiments of the respiratory effort sensor can also be used as part of a sleep assessment in a sleep laboratory or as part of a sleep apnea screening device used in the home. The respiratory effort information can also contain information about the degree of paradoxical breathing as disclosed in U.S. Provisional App. No. 61/200,761 which is incorporated herein by reference in its entirety. Various embodiments of the non-contact physiological motion sensors described herein can be used to obtain respiratory effort waveforms, respiratory rate, indication of paradoxical breathing, indication of activity, and heart rate. Various embodiments of the system 100 can be used as a home screening test for obstructive sleep apnea as disclosed in U.S. Provisional App. No. 61/194,836 which is incorporated herein by reference in its entirety and in U.S. Provisional App. No. 61/200,761 which is incorporated herein by reference in its entirety.

In various embodiments described herein, it can be possible to measure respiratory motion without any contact to the subject with a radar-based system specifically configured to measure physiological motion, and respiratory motion can be derived from the physiological motion signal. In addition to detecting respiratory rates from the motion, respiratory motion can also provide a measure of respiratory effort similar to that provided by piezoelectric or inductive chest belts designed to measure respiratory effort. In various embodiments, measurements of respiratory effort can be necessary to determine whether an event is a central apnea or an obstructive apnea. In various embodiments, respiratory motion can be measured with a radar-based system described herein overnight irrespective of the position of the subject in the bed.

In various embodiments, the physiological motion sensor can include a radar-based device that can be configured to detect paradoxical breathing (e.g., when the abdomen contracts as the rib cage expands or the rib cage contracts as the abdomen expands). In most cases, during obstructive apnea paradoxical breathing can be exhibited, although paradoxical breathing cannot indicate an airway obstruction. In various embodiments, an indication of paradoxical breathing and of the level of paradoxical breathing can be useful in detecting obstructive apnea.

Various embodiments of the radar-based physiological motion sensor can also measure non-cardiopulmonary motion (e.g., activity such as tossing and turning in bed, wakefulness, or involuntary movement during sleep). The level of activity can be used to estimate the quality of sleep, and it can be helpful in determining the sleep state of the subject. Various embodiments of the system 100 can also be used to determine when the person is in the bed or out of the bed, to track how often the subject is getting out of bed during the night, etc. Various embodiments of the system 100 can also measure the heart rate. During apneaic events, the heart rate can increase, and in some embodiments, the heart rate can be used to confirm an apnea that is indicated by other measurements.

Various embodiments of the system 100 can be used to estimate the tidal volume, or the amount of air inhaled and exhaled with each breath. When the tidal volume is accurately measured, it can be used to estimate the airflow. Various embodiments of the system 100 can include multiple-antenna hardware and software that is executable by a processor such that it can track the subject as he/she moves in bed during the night. This can provide information about how much the subject is moving within the bed, and it can improve the radar-based measurement of respiration and activity. The physiological motion sensor can be used in conjunction with other sensors to provide a more complete picture of respiration during sleep. Various embodiments of the system 100 can include additional sensors including, but not limited to, a nasal/oral airflow sensor and a pulse oximeter.

In various embodiments, the nasal/oral airflow sensor can provide either an indication of whether the patient is breathing, or with a more advanced sensor, an estimate of the velocity of the airflow. This can be used to accurately detect apnea, and with the more advanced sensors, it can also be used to detect hypopnea (reduction in airflow). An accurate measurement of airflow is critical to determine whether an event is a hypopnea or an apnea. The nasal/oral airflow sensor can include one or more thermistors, hot-wire anemometers, or pressure sensors. In some embodiments, a nasal/oral airflow sensor can be provided to measure the air flow through each nostril and the mouth independently. In most embodiments, an airflow sensor alone cannot determine whether an apnea is central or obstructive.

In various embodiments, the pulse oximeter can provide information on the effectiveness of respiration by arterial hemoglobin saturation or an estimate of blood oxygenation. Decreases in blood oxygenation can indicate the severity of an apneaic or hypopneaic event, and are important for clinical decisions. The pulse oximeter can also provide a heart rate. In various embodiments, pulse oximetry can be recorded on the finger or on the ear though in most embodiments, the finger measurements are generally considered more accurate.

In various embodiments, the pulse oximeter and oral/nasal airflow sensors can require contact with the patient. In various embodiments, the pulse oximeter and oral/nasal airflow sensors can be configured to transmit data wirelessly to the data recording device. In various embodiments, this recording device can be integrated with the radar-based physiological motion sensor device.

Various embodiments of the system 100 can include a wireless home sleep monitor, including a radar-based physiological motion sensor, a pulse oximeter with wireless communications, and a nasal/oral airflow sensor with wireless communications, operating without wires on the patient and with minimal contact to the patient. Various embodiments of the home sleep monitor can provide a complete picture of respiration during sleep (e.g., airflow, respiratory effort, and oxygenation). In various embodiments, the home sleep monitor system 100 can also provide a heart rate, variability in the heart rate, and information about motion during sleep. In various embodiments, the pulse oximeter and oral/nasal airflow sensor can be configured to independently send their data wirelessly to the hub, such that no wires would be required. This can provide an advantage over other commercially available home sleep monitors, which requires wires to the recording device or wires to a single body-worn device with then wirelessly, transmits data to the recording device.

Various embodiments of the physiological motion sensor system 100 can be used to obtain a spot check of vital signs, such as respiratory rate and heart rate, at a point in time or intermittently (e.g., at regular intervals, at specified times, on demand, etc.). In various embodiments, the system 100 can have different user-selectable time intervals over which the breathing rate can be measured (e.g., 15 seconds, 30 seconds, 60 seconds, etc.), a chosen number of breathing cycles (e.g., 2, 3, 5, etc.), or a more general indication of the measurement length (e.g., “quick,” “normal,” “extended”). In various embodiments, the system 100 can use signal quality, respiratory rate, respiratory rate variability, and respiratory waveform shape variability to automatically select a measurement interval. In various embodiments, the system 100 can recognize data with interference from non-cardiopulmonary motion, vibration, other radio-frequency signals, or circuit noise, and can not include it in rate calculation. This can improve the accuracy of rate readings. In various embodiments, the accuracy of rate readings can be further improved through rate estimation algorithms that include accuracy checks. Various embodiments of the system 100 can be configured to identify non-cardiopulmonary motion by the subject or other motion near the subject when extracting cardiopulmonary motion, which can result in greater accuracy of the readings and/or avoid displaying an error due to non-cardiopulmonary motion detection.

In various embodiments non-contact spot check of respiratory parameters can have a measurement mode in which the measurements are automatically started at regular intervals. Measurements at regular intervals can be used to provide a history of point-in-time measurements such that trends can viewed. In various embodiments, the measurements can be automatically started and/or made in the absence of a health care provider. In some embodiments, when the sensor has real-time signal-quality detection, portions of collected data with poor signal quality due to low signal power or subject motion are not used to estimate the respiratory parameters, and portions of the collected data with adequate signal quality are used to estimate the respiratory parameters. The device can perform each measurement for a fixed time period, or it can use an automatic mode such that the measurement length is chosen automatically based on signal quality and/or regularity of breathing. In some embodiments, the device can continue re-trying a measurement until enough signal of adequate quality is obtained to provide a respiratory spot check. In some embodiments, the operators of the interval respiratory measurement device can choose to operate the device in manual mode (for which the button can be pressed to initiate a measurement), or choose a time period for intermittent measurements. In various embodiments, the interval measurement device can offer a menu of intervals. For example, in some embodiments the menu can offer measurement intervals of 1 minute, 5 minute, 10 minute, 15 minute, 30 minute, 60 minute, 120 minute and 240 minute intervals. In some embodiments, the user can enter the interval length on a keypad, and be able to select any desired interval length. In some embodiments, the periodic measurements can continue until the stop button has been depressed, while in alternate embodiments, the user is able to program a time at which the periodic measurements can stop. Some embodiments of the interval respiratory measurement can display a history of the measurements and their associated time, alphanumerically and/or graphically.

In some embodiments, the respiratory rate interval measurement device can synchronize with other medical equipment. For example, a respiratory rate interval measurement device can be integrated with a patient-controlled analgesia pump, such that no additional doses of opioid drugs is given unless a respiratory rate is measured above a minimum programmed respiratory rate. In some embodiments, the respiratory rate interval measurement device can be integrated with another vital signs measurement device such that multiple vital signs are obtained at the same interval, such as blood pressure and respiratory rate.

Various embodiments of interval measurement of respiratory rate include, but are not limited to those where the measurement commences every N seconds after the start of the first measurement; those where the measurement commences N seconds after the start of the last measurement; those where the measurement commences N seconds after the end of the last measurement; those where the measurement commences after sensing signal quality such that intervals can be varied and only the number of measurements per N seconds is specified; those where the measurement is queued if the length overlaps with the next interval; and/or those where a measurement can be dropped if the length overlaps into the next interval. Various embodiments of the interval measurement can have an associated time-out, where the device provides an error code, message, or alert if it was not able to obtain the required length of good-quality data in that time. Alternatively, various embodiments of the interval measurement can run until a respiratory rate is obtained. In those embodiments where a time-out is implemented, the time-out can occur at a fixed time, a user-settable time, or it can be determined by other equipment. In embodiments in which the interval respiratory measurement is integrated with other vital signs measurements such as blood pressure or temperature, the time-out can be determined by other equipment; in some embodiments, the time-out can occur at the completion of these measurements. In some embodiments, the same button can be used to initiate measurement of all the vital signs. In some embodiments, if the time-out is reached, a measurement overlaps with the next interval, or a respiratory rate cannot be obtained for longer than a specified time period, an audible and/or a visual alert can be provided so the healthcare practitioner knows that a respiratory rate was not obtained at the specified interval.

Various implementations of interval measurement of respiratory rate can include real-time audio feedback for some or all types of poor signal quality. For example, in some embodiments, a ticking sound can indicate low received signal power, such that the user knows that he/she needs to reposition the sensor. Providing feedbacks regarding the signal quality can avoid delays in obtaining a measurement. Degradation of signal quality can result due to a variety of reasons including an improperly placed sensor. Various implementations of interval measurement of respiratory rate can use various communication methods including but not limited to, sending a page, sending an automated message, sending a SMS, sending an email or use other techniques to alert attending health care professionals if excessive errors or alerts are occurring so the healthcare practitioner is alerted and can reposition the sensor or provide the patient with the necessary medical attention. In some embodiments, audible or visual alerts can be used instead of or in addition to other alerting methods. Various implementations of interval measurement of respiratory rate can also include audio, visual, or remote alerts if an adverse trend in respiratory parameters is recognized. For example, in some embodiments, if a patient's respiratory rate is slowly decreasing, an alert will occur so that a health care professional knows that the patient needs care. In various embodiments, the alerts can be pre-programmed in the device or they can be user-settable. Various implementations of interval measurement of respiratory rate can also include audible, visual, or remote alarms if a respiratory parameter is measured outside of pre-defined parameters. The pre-defined parameters can be factory pre-sets; can be set by the user or health care provider; or can be based on the patient's baseline values.

In various embodiments, both time and frequency domain approaches can be used for assessment of validity of respiratory rate calculations. In various embodiments, the system 100 can provide a signal quality feedback system during and after the measurement. The signal quality feedback can indicate non-cardiopulmonary motion, signal interference, low signal power and/or clipping due to signal overload. In various embodiments, system self-test and environment-checks before measurement can be performed. In various embodiments, the system 100 can use a free-running signal source to reject RF interference, e.g., random frequency drifts can provide immunity against interference from sources operating in the same frequency band. In various embodiments, the system 100 can be integrated with other devices, approaches and peripherals used for chronic disease management in homes and other remote settings. For example, the system 100 can be used with blood pressure cuffs, thermometers in a home health management unit. Various embodiments of the system 100 can provide cardiopulmonary information as part of a health kiosk. Various embodiments of the system 100 can be used to measure the amount of air inhaled/exhaled with each breath (relative tidal volume) and the depth of breadth. Various embodiments of the system 100 can provide alerts of high or low heart or respiratory rates or irregular heart or respiratory rates. In various embodiments, the system 100 can be used to detect heart arrhythmia or respiratory sinus arrhythmia. Various embodiments of the system 100 can have an aiming or a focusing element to help the user aim the system properly for accurate measurements. In various embodiments, on-demand spot check measurements are provided. In various embodiments, the measurements can be initiated locally or remotely. Various embodiments of the system 100 can be integrated with audiovisual or other multimedia devices.

The system 100 can be used as a non-contact vital signs spot check to obtain respiratory rate and/or heart rate in one or more subjects. Embodiments of the vital signs spot check system 100 can be used in a hospital or skilled nursing facility for regular vital signs assessment of in-patients, or in any clinical setting for vital signs assessment of patients checking in for treatment of checkups. Embodiments of the vital signs spot check system 100 can be used in pediatric or neonatal wards for monitoring cardiopulmonary activity in infants and newborns. Various embodiments of the system 100 can include a local interface, including buttons and display, and can have electronic communications to a central site (such as a central nurse's station) or to a central database (such as an electronic medical record). In various embodiments, the system 100 can be a stand-alone device, or it can be a module providing one measurement (such as respiratory rate) or multiple measurements (such as either respiratory rate and tidal volume or respiratory rate and heart rate) integrated with another vital signs spot check device. In various embodiments, the vital signs spot check system 100 can display only a rate or rates that are measured. In some embodiments, the system 100 can be configured to display a snapshot of the heart and/or respiratory waveforms. In various embodiments, the non-contact vital signs spot check can be used for triage in an emergency room, a disaster area, or a battlefield as disclosed in U.S. Provisional App. No. 61/154,728 which is incorporated herein by reference in its entirety.

Various embodiments of the system 100 can include a sensor unit that is mounted in various positions in a room, including on the ceiling, on the wall, under the mattress, on the bed rail, at the head of the bed, at the foot of the bed, on a moveable cart or pole in a patient's room in a hospital, nursing home, or alternate care environment, etc. The sensor unit for the system 100 can communicate wirelessly or through installed infrastructure in the hospital, nursing home, or alternate care environment to a local patient monitor, a local vital signs spot check device, or a central unit in the hospital, nursing home, or alternate care environment. In the system 100, the sensor unit includes the antenna, transmitter, receiver, and analog-to-digital conversion of the radar-based sensor, and it also includes appropriate hardware and software (as required) for transmitting digital signals, either wirelessly or through wired infrastructure.

As shown in FIG. 6A, some embodiments of the system 100 can include a sensor unit 604 that is wirelessly linked with a patient monitor 605 in the patient's room. The system unit 604 can be configured to wirelessly transmit the digitized signals from the sensor unit 604 to the patient monitor 605 in the patient's room. The patient monitor 605 can include a processor 606 that can be configured to process the signals from the sensor unit 604. The processing can include, but is not limited to, DC compensation, filtering, demodulation, motion-detection, rate-finding, and possible calculation of other variables.

As illustrated in FIG. 6B, in various embodiments, the sensor unit 604 can include the processor 606 and associated digital components such that the sensor unit 604 is configured to process the digital signal, including perform DC compensation, filtering, demodulation, and motion detection, and transmit a processed signal to the patient monitor 605. In various embodiments, the processor 606 in the sensor unit 604 can be configured to perform rate estimation and/or calculation of other respiratory variables, or, alternatively, the patient monitor 605 can perform rate estimation and/or calculation of other respiratory variables from the processed signal. In those embodiments in which the patient monitor 605 performs rate estimation, the patient monitor 605 can use the same rate-estimation algorithm it uses for other respiratory waveforms it can input, including impedance pneumography

In various embodiments, the sensor unit 604 can include memory and/or other storage devices 607 that are configured to store measurement data (e.g. respiratory rate or other respiratory parameters) for an extended time period in addition to the processor 606 as illustrated in FIG. 6C. The memory and other storage devices 607 can be configured to store measurements obtained over a time period. In some embodiments the memory and/or storage devices 607 can be configured to store 24-96 hours of data. The sensor unit can be configured to synchronize the stored data with the patient monitor 605 in addition to transmitting the current data. Synchronizing the recorded data can enable a user or a health care provider to view the measurement history. The measurement history can include measurements that were obtained in the absence of the patient monitor 605 that were stored in the memory and/or storage devices 607. In various embodiments, the patient monitor 605 can display, transmit, and/or record one or more of the respiratory waveforms, the respiratory rate and other respiratory variables calculated from the signal, in addition to other physiological and vital signs information.

In various embodiments of the system 100 the patient monitor 605 can be permanently mounted in the patient's room or on a cart that is wheeled into the room or otherwise placed at the patient's bedside. In some embodiments, it can be important that the wireless link between the devices be correct. For example, the sensor unit that is measuring a particular patient is preferably linked to the patient monitor measuring that same patient. In those embodiments in which the patient monitor and the sensor unit are both permanently mounted in the patient's room, when the two devices are first installed, they can perform a synchronization process where they can exchange a pseudorandom sequence. In some embodiments, the pseudorandom sequence can be used to add pseudo-random noise (PN) to the data such that only a receiver with knowledge of the PN code will be able to communicate and decode the data. In those embodiments in which a patient monitor is brought into a patient's room for monitoring of specific patients and the sensor unit is permanently mounted in the room, a tethered bar-code reader or short-range RFID reader can be placed on the patient monitor, and a bar code or RFID tag can be placed on the sensor unit such that when the healthcare practitioner brings the device into the room, he/she brings the reader up to the sensor unit, and the reader reads the PN code embedded on the RFID, which is the same PN code used for communications. In those embodiments in which a patient monitor that is brought into a patient room for monitoring of specific patients and the sensor unit is permanently mounted in the room, the patient can wear a patient identification tag that is scanned by the healthcare practitioner before initiating the patient monitoring device, which can also be read by the sensor unit, which, in some embodiments, has an integrated tag reader. During streaming and/or synchronization, the sensor unit can include information about the identity of the patient being measured, and the patient monitoring device can ensure that the identity of the patient that it is monitoring is the same as that for which the respiratory data is provided for because both the patient monitor and sensor device will use the PN code provided by the RFID worn by the patient. In some embodiments where a patient monitor is brought into a patient's room for monitoring of specific patients and the sensor unit is permanently mounted in the room, the sensor unit is programmed with information about the location of the bed it is monitoring when it is installed, and an RFID tag or bar code is placed on the wall by the bed location, and the healthcare practitioner scans that when he/she brings the patient monitoring device into the room such that the patient monitoring unit receives the PN code read from the tag or bar code reader, and the code on the RFID tag or bar is programmed or read by the sensor unit when it is initially installed, and uses the PN code to encode the respiratory data. In various embodiments, the pseudorandom code could be replaced with another type of code.

In those embodiments in which the permanently mounted sensor unit wirelessly links with a vital signs spot check device that is brought from room to room to measure vital signs of different patients, the digitized signals from the sensor unit are wirelessly streamed to the vital signs spot check device in the patient's room, and the vital signs spot check device performs processing of the signals, including DC compensation, filtering, demodulation, motion-detection, rate-finding, and possibly calculation of other variables. In those embodiments in which the permanently mounted sensor unit wirelessly links with a vital signs spot check device that is brought from room to room to measure vital signs of different patients, the sensor unit contains a processor and associated digital components such that it processes the digitized signal, including DC compensation, filtering, demodulation, and motion detection, and streams a processed signal to a patient monitor; either the sensor unit also performs rate estimation and/or calculation of other respiratory variables, and streams these variables along with the respiratory waveform, or the vital signs spot check device performs rate estimation and/or calculation of other respiratory variables on the streamed waveform. In those embodiments in which the permanently mounted sensor unit wirelessly links with a vital signs spot check device that is brought from room to room to measure vital signs of different patients, the sensor unit contains a processor and associated digital components such that it processes the digital signal, including DC compensation, filtering, demodulation, and motion detection, and streams a processed signal to a patient monitor; the sensor unit contains hardware such that it can store the last calculated respiratory rate, other respiratory variables, and/or the waveform used to calculate the rate and/or other variables (“the last measured respiratory check”), and when a vital signs spot check device is brought into the room, it streams the last measured respiratory check to the vital signs spot check device.

In various embodiments, the vital signs spot check device can display, transmit, and/or record one or more of the following: the respiratory waveform used for the spot check, the respiratory rate, and/or other respiratory variables calculated from the signals, in addition to other physiological and vital signs information. In various embodiments, it is not required that the vital signs spot check device be kept in the same room as the sensor unit, instead the vital signs spot check device can be mobile and moved from room to room to measure vital signs of different patients. In various embodiments, it can be important that the wireless link between the devices be correct. For example, as discussed above the sensor unit that is measuring a particular patient is preferably linked to the vital signs spot check device measuring that same patient. Above described methods to synchronize the sensor with the continuous patient monitor can be used for the spot check device.

In various embodiments, a sensor unit configured to work with both patient monitors and vital signs spot check devices, with a PN code that can be synchronized for the purpose of coding messages. In various embodiments, after the devices have been paired with a PN code, they can wirelessly communicate their device type (either continuous monitor or spot check) and its desired information to the sensor unit, which then sends the appropriate information to the patient monitor or vital signs spot check devices. In various embodiments, the sensor unit can communicate data directly to a central server or station, either wirelessly or via wired infrastructure; this central server or station can be the sole location where the data is displayed and stored, or the central server can transmit the respiratory data to a patient monitor or vital signs check device.

Various embodiments of the vital signs spot check system described herein can be used in the home for management of chronic illnesses as disclosed in U.S. Provisional App. No. 61/196,762 which is incorporated herein by reference in its entirety, including COPD, diabetes, and congestive heart failure. As described above, in various embodiments, the system 100 can be connected to another device, including, but not limited to, a personal health system, another home healthcare device, a personal computer, a cellular phone, a set-top box, or a data aggregator. In various embodiments of the system, the device can connect via a wired or wireless connection to a central station that is remote (e.g., away from the home). In various embodiments, the system 100 can have a local display with some or all of the obtained data displayed on it. In some embodiments, the system 100 can communicate the information to another device via a wired or wireless connection to a central database that is remote (e.g., away from the home). In various embodiments, the device can operate with local control or can be controlled by another device via a wired or wireless connection. In various embodiments, the system 100 can operate automatically, or can be controlled by a central system that is remote (e.g., away from home). In various embodiments of the system, the vital signs spot check system 100 can be a module that is integrated into a personal health system or another home healthcare device, sharing its display and communications.

Various embodiments of the vital signs spot check system described herein can be used in the home to monitor the elderly, chronically ill, or others on a daily basis while they are sedentary and/or sleeping. The vital signs spot check system can be provided in homes, assisted living facilities, nursing homes, hospices, elder care facilities, etc. In some embodiments, this system can be wirelessly connected to a server that analyzes the data provided by the sensor to provide early indication and detection of acute illness, exacerbation of chronic illness, or other changes in health status. In various embodiments, this sensor can be mounted in many locations, including, but not limited to, the ceiling, the wall, on a table, under the mattress of the bed, on a bed rail, at the head of a bed, at the foot of a bed, or to a movable cart or post. In various embodiments, this Doppler radar-based sensor can provide one or more of the following variables: respiratory rate, respiratory waveform, depth of breath, pulse rate, activity/restlessness data, inhale time to exhale time ratio, and regularity or irregularity of respiration. Various embodiments of the system configured to be mounted on the ceiling or wall, a high-gain planar antenna can be used. In various embodiments, the antenna can be a three-by-three element array, a four-by-four element array, or an n-by-n element array. In some embodiments, the antenna can be a single aperture. In some embodiments, a direction-sensitive, multi-element antenna array can be used to monitor the position of the subject. In some embodiments, multiple persons can be detected and measured with a multiple-receiver system. In some embodiments, an RFID tag or other identification device can be worn by, clipped on to the garments of, or attached to the skin of the subject(s) under observation. In some embodiments, tags can allow a sensor to distinguish the subject under observation from other persons in the area. In some embodiments, a single system can be mounted facing towards the user's bed. In some embodiments, multiple systems can be mounted in the living area, including the user's bed and possibly the user's favorite chair or favorite spot on the couch to provide additional coverage. In some embodiments, the device can provide local alarms or alerts for potential indication of a disease state that requires immediate attention, including dangerous apnea, bradypnea, tachypnea, bradycardia, tachycardia, and periodic or Cheyne-Stokes breathing. In some embodiments, the device can wirelessly transmit to a server or to a health care professional the vital signs and activity parameters it collects, as well as flags or alerts for detected apnea, bradypnea, tachypnea, bradycardia, tachycardia, and irregular breathing. In various embodiments, the wireless link can be Zigbee, Bluetooth, Wireless X-10, or 802.11. In some embodiments, the device can also transmit information on the quality of the waveforms obtained and/or sent. In some embodiments, the device can be configured to transmit good-quality waveforms. In some embodiments, the device can send the waveforms obtained during non-cardiopulmonary motion, or other information calculated about motion. In some embodiments, the waveforms obtained during non-cardiopulmonary motion can be used to identify one or more of the following: minor movements, rolling over, restless leg syndrome, level of restlessness or entering or leaving bed. In various embodiments, the information transmitted by the device can be recorded and analyzed to identify early signs of illness. In various embodiments, the server or system that receives the information transmitted by the device can identify and summarize important events (e.g. apnea, shallow breathing, irregular breathing, bradycardia, tachycardia, restlessness, tachypnea, and bradypnea), provide daily summaries, provide long-term trends, and/or detect major changes in vital signs or health status. In some embodiments, periods of important events can be quantified by duration and severity. In some embodiments, daily summaries can include important events, clinically useful summaries of baseline values, and/or information on the overall quality of sleep. In some embodiments, quality of sleep can be derived from restlessness data based on the number, length, and position of motion-free periods and their interruptions while the subject is in bed. In some embodiments, trending and change-detection algorithms can be applied to these daily summaries to notify caregivers and/or health care professional of emerging changes in health status. In some embodiments, the device can form an ad-hoc network with other wireless monitoring devices; with each device providing a service to other devices to pull data from the device, and each device having the ability to poll data from the other devices, such that, through the cooperative use of the data, an event can be flagged with better accuracy. In some embodiments, web interfaces can be used to provide access to the obtained and/or the analyzed data to users, their caregivers, and their healthcare providers. In some embodiments, this system will be mounted in homes. In various embodiments, the systems and devices described herein can be configured to send automated alerts (911 call or code system) to health care providers or emergency personnel in the case of an acute or severe event. In some embodiments, for lower severity events and warnings, a more subtle message can be sent (e.g. Page, SMS, email, etc.).

In various embodiments, the vital signs spot check system 100 can be included in a health kiosk as disclosed in U.S. Provisional App. No. 61/128,743 which is incorporated herein in its entirety. Various embodiments of the kiosk vital signs spot check system 100 can be a standalone device that sends vital signs information to a kiosk computer. Various embodiments of the system 100 can require a local person to press the buttons on the device to initiate operation. In some embodiments, the system 100 can be controlled by a remote healthcare practitioner with a start signal sent to the device through the kiosk computer. In some embodiments, the system 100 can initiate the measurement automatically when the patient enters the kiosk area; the system 100 can sense the presence of the patient, or the system 100 can use data from another device that senses the presence of the patient. Various embodiments of the kiosk vital signs spot check system 100 can be a module that is integrated into the kiosk such that the patient is not aware of its presence. In such embodiments, the system 100 can be controlled by the kiosk computer, either with a remote healthcare practitioner initiating the measurement, or a measurement being initiated automatically, possibly a fixed time after the patient enters the kiosk or sits down. In various embodiments, the system 100 can measure respiratory rate only once, or it can continue to measure intermittently while the patient is at the kiosk, providing a rate history for the time the patient was in the kiosk to the remote healthcare provider.

In various embodiments, the cardiopulmonary information, activity and other physiological motion data collected by the system 100 can be used to assess and monitor psychological or psycho-physiological state or changes in psychological or psycho-physiological state. In various embodiments, the system 100 can monitor changes in psycho-physiological state induced by external stimuli (e.g., questions, sounds, images, etc.)

Various embodiments of the non-contact physiological sensor system 100 can be used to obtain respiratory rate, heart rate, and physiological waveforms that can be analyzed to help assess the psychological state of the measurement subject as disclosed in U.S. Provisional App. No. 61/141,213 which is incorporated herein by reference in its entirety. The psychological information can be used for many applications, including, but not limited to, various medical applications, security screening of subjects at airports, borders, and sporting events and other public areas, lie detection, and psychological or psychiatric evaluation. In various embodiments of the system 100 used in security screening applications information output from the system 100 can be used to help detect malintent.

Various embodiments of the physiological motion sensor system 100 can be used to provide physiological motion waveforms that can be used for synchronization of medical imaging with chest or organ motion as disclosed in U.S. Provisional App. No. 61/154,176 which is incorporated herein by reference in its entirety.

Various embodiments of the system described herein can be used to provide physiological motion waveforms that can be used for synchronization of mechanical ventilation, including non-invasive ventilation, with respiratory effort.

Various embodiments, the system 100 can be integrated with a pulse oximeter. The various embodiments described herein, the physiological motion sensor 100 can be used to sense respiratory information and can be operated in connection with a pulse oximeter that measures the patient's oxygen saturation. In various embodiments, the combination of the two sensor systems can provide information on ventilation and oxygenation, giving a more complete measurement of respiratory efficacy than either could alone as disclosed in U.S. Provisional App. No. 61/194,839 which is incorporated herein by reference in its entirety. These embodiments have applications in the monitoring of post-surgical patients, patients using opioid-based medications, patients at risk of respiratory depression, etc.

Various embodiments of the system 100 can be integrated with or connected to a patient-controlled analgesia system, and prevent additional doses of analgesia if the respiratory rate drops below a threshold, indicating the onset of opioid-induced respiratory depression. Various embodiments can also use additional respiratory variables in the calculation of when to prevent additional doses of analgesia, including tidal volume, inhale time to exhale time ratio, depth of breath, frequency of non-cardiopulmonary motion, duration of non-cardiopulmonary motion, length of pauses in breathing, frequency, depth, and length of gasps, frequency, depth, and length of signs, and/or shape of the breathing waveform. The thresholds in such embodiments can be at least one of pre-set in the factory, set by the healthcare professional, calculated based on patient baseline values. Various embodiments can also include alerts.

In various embodiments, the system 100 can be used to determine if a subject is breathing and/or if his/her heart is beating. In various embodiments, the system 100 can detect presence of and/or monitor cardiopulmonary information (respiratory and/or cardiac) from several meters away from a subject to the point of contact. In various embodiments, the system 100 can detect and monitor cardiopulmonary information (respiratory and cardiac) while in contact with the subject's body. In various embodiments, the system 100 can measure body surface motion associated with cardiopulmonary activity. In various embodiments, the system 100 can measure internal body motion associated with cardiopulmonary activity. In various embodiments, the system 100 can measure electromagnetically measureable internal and/or external body changes associated with cardiopulmonary activity, including but not limited to impedance changes. In various embodiments, the system 100 can perform the above described functions by itself or in combination with other monitoring devices.

In various embodiments, the physiological motion sensor described herein can be used to determine whether a subject requires cardiopulmonary resuscitation or use of a defibrillator (either an automated external defibrillator or a hospital defibrillator) by detecting whether the patient has a heartbeat as disclosed in U.S. Provisional App. No. 61/194,838 which is incorporated herein by reference in its entirety. In various embodiments, the system 100 can send a signal to an external medical device such that it can integrate information from the system with information from other sensors to determine whether resuscitation is required. This determination can be indicated to the user visually or audibly. In various embodiments, the system 100 can provide a signal to a defibrillator, such that if a heartbeat is detected, it is not possible to deliver an electrical shock to the patient. In various embodiments, the system 100 can send a signal to trigger external medical devices (e.g., defibrillator, ventilators, oxygen pumps, external respirators, etc.). The non-contact physiological motion sensor can be used after a defibrillator is used on a patient to determine if mechanical heart activity has resumed.

In various embodiments, the physiological motion sensor system 100 can be used to detect human motion at a distance and/or through radar-penetrable barriers. In various embodiments, this motion can include gross motion, such as walking, as well as small motion due to fidgeting or speech, and minute surface displacements resulting from cardiopulmonary activity. In various embodiments, the signals from the different sources can be separated by sophisticated signal processing and classified into biometric signatures unique for each individual as disclosed in U.S. Provisional App. No. 61/125,164, which is incorporated herein by reference in its entirety. In various embodiments, empirical mode decomposition as disclosed in U.S. Provisional App. No. 61/125,023, which is incorporated herein by reference in its entirety, can be used for identifying individual signatures of physiological motion, including heart and respiratory motion waveforms. In some embodiments, empirical mode decomposition as disclosed in U.S. Provisional App. No. 61/125,023, which is incorporated herein by reference in its entirety can be used for identifying patterns in the variability of the amplitude of physiological motion. In various embodiments, empirical mode decomposition as disclosed in U.S. Provisional App. No. 61/125,023, which is incorporated herein by reference in its entirety can be used for identifying patterns in the variability of rate of physiological processes, such as heart rate variability and respiratory rate variability. In various embodiments, empirical mode decomposition as disclosed in U.S. Provisional App. No. 61/125,023, which is incorporated herein by reference in its entirety, can be used for analyzing the interaction.

In various embodiments, many variables extracted from the cardiopulmonary motion signal can be used for biometric identification of individuals. In various embodiments, these variables include respiratory rate, inhale time, exhale time, inhale time to exhale time ratio, frequency of gasps, depth of gasps, length of gasps, frequency of signs, depth of signs, length of signs, depth of breath, presence of paradoxical breathing, degree of paradoxical breathing, tidal volume, ratio of abdominal excursion to chest excursion, harmonic content of breathing signal, ratio of the powers of different harmonics of the breathing signal, airflow rate, heart rate, and heart beat-to-beat interval. In various embodiments, the biometric identification would also include the variability of some or all of the above-mentioned variables in any number of frequency bands. In various embodiments, the biometric identification would also include the correlation between heart variables and respiratory variables. In various embodiments, the biometric identification would also include the frequency, duration, and amount of activity, and/or the frequency, duration, and amount of fidgeting.

Various embodiments of the system 100 can be used to determine the patient's tidal volume. Various embodiments of the system 100 can determine the relationship between displacement and tidal volume from medical record information, such that an accurately measured displacement can be converted to a tidal volume estimate as disclosed in U.S. Provisional App. No. 61/125,021, which is incorporated herein by reference in its entirety. In various embodiments, the system 100 can be used to determine the relationship between displacement and tidal volume based on patient maneuvers and medical record information, such that no contacting devices would be required to perform a calibration as disclosed in U.S. Provisional App. No. 61/125,018, which is incorporated herein by reference in its entirety. In some embodiments of the system, published formulae and the medical record can be used to predict the patient's vital capacity, such that if the patient performs a vital capacity maneuver by inhaling as deeply as possible and exhaling as fully as possible, the relationship between chest displacement and tidal volume can be calculated. In various embodiments, the system 100 can be calibrated before measurement, such that a tidal volume can be estimated. In various embodiments, the system 100 can be used to determine relationship between displacement and tidal volume via direct measurement: calibration with a spirometer or other device that accurately measures tidal volume as disclosed in U.S. Provisional App. No. 61/125,021, which is incorporated herein by reference in its entirety.

In various embodiments, relative tidal volume can be measured without calibration by providing information about whether the tidal volume is increasing or decreasing from a baseline value during continuous monitoring of a patient. In various embodiments of the relative tidal volume measurement, the relative tidal volume can be reset each time non-cardiopulmonary motion is detected, thereby avoiding errors in the relative tidal volume that result from changes in the relationship between chest displacement and tidal volume with the patient in different positions and with different spatial relationships between the sensor and the patient. Such an embodiment can be useful in non-ventilated or non-invasively ventilated critical care patients.

In various embodiments, data from the system 100 can be used to generate an activity index. In various embodiments, the system 100 can use the non-cardiopulmonary motion detection algorithm to determine the frequency and duration of subject activity or the percentage of time the subject is active. This information can be used to provide an activity index. In some embodiments, changes in the activity index can be used as indicators of a change in health state (e.g., if a patient's activity one day is significantly less than their baseline, it can indicate an illness). In various embodiments, the activity index can also be used during measurement of sleeping subjects to assess sleeping vs. waking states, insomnia, restless leg syndrome. In various embodiments, the activity index can be used to assess circadian rhythm disorders, alertness, metabolic activity, energy expenditure, and daytime sleepiness.

In various embodiments, digitized data from the Doppler radar-based sensor can be analyzed by algorithms that can differentiate cardiopulmonary motion (heart motion, pulse motion, respiratory motion, etc.) from non-cardiopulmonary motion. In some embodiments, the non-cardiopulmonary motion detection algorithm flags the data as non-cardiopulmonary motion if certain thresholds are reached; in various embodiments, this analysis can include comparing the power levels, eigenvalues, eigenvectors, best-fit line, RMS difference from a best-fit line, RMS-difference from a best-fit arc or circle, origin of a best-fit circle, radius of a best-fit circle, or any combination thereof, of current data frames with the previous data frame or frames. In various embodiments, these frame(s) can be weighted equally or the weighting can carry some modeled decay factor. In various embodiments, frames that exceed the thresholds can be flagged as non-cardiopulmonary motion events. In some embodiments, the frames can be compared against a power threshold and frames that fall below this power threshold are flagged as low-power signal events. In various embodiments, the power threshold can be close to the noise floor. In some embodiments, a frame is flagged as an activity event if it receives a non-cardiopulmonary signal flag but not a low-power flag. In some embodiments, frames flagged as activity are counted and stored. In some embodiments, the number of frames flagged as activity events are divided by the total number of frames from which the activity count is derived. In some embodiments, the output of this system is the activity index. In various embodiments, the number of frames used to derive the activity index can be varied. In some embodiments, the activity index can represent the entire history since the system was been switched on. In other embodiments it can only represent the most recent history, or the history over a recent time period (e.g. over the past 5 minutes, the past 10 minutes, the past 15 minutes, past 30 minutes, the past 1 hour, the past 2 hours, etc.). FIG. 6D illustrates a block diagram of an embodiment of a system configured as an activity index indicator. The embodiment of the system illustrated in FIG. 6D comprises a motion detector 608, which can be similar to the sensor unit described above, a power thresholder 609, and an activity detector 610. In various embodiments the system can further comprise counters, dividers, etc. which can be used to derive the activity count. In some embodiments, the system can be configured to display and/or record a history of activity. In various embodiments, the activity history can be used to assess changes in the degree of activity over time. In some embodiments, the activity index can be assessed each day for the previous 24 hours, such that day-to-day changes in activity can be deduced and used for diagnostic purposes. In some embodiments, the activity index for periods less than a day can be compared with that same period in previous days, such that daily patterns of activity can be assessed, and changes in those patterns can be detected and investigated. In some embodiments, activity data and/or the activity index can be used in conjunction with vital signs measured with the radar sensor to determine quality of sleep as well as sleep state. In some embodiments, the system can be used to provide a non-contact sleep state monitor and/or sleep quality monitor. In some embodiments, the degree of activity at different times of the day can be assessed to determine diurnal activity patterns. In some embodiments, the activity index can be used to determine the degree of convalescence and/or to quantify convalescence. In some embodiments, the system can be used by autonomous vehicles in battlefield triage to help identify if fallen troops may still exhibit signs of activity. In another embodiment, the radar sensor can simply be used to monitor an area for signs of activity above that of the ambient noise floor. In some embodiments, the system can be network-enabled such that the activity data and/or the activity index can be viewed at a remote station and/or be stored in an Electronic Health Record or other database.

In some embodiments, the activity index can be used as part of a continuous Doppler radar respiration monitor, and can be displayed on the screen of such a monitor. The display of one such embodiment is shown in FIG. 6E: the top trace 614 shows an instantaneous respiratory waveform after filtering and demodulation, and the bottom trace 616 shows both the subject's respiration rate history and the places where the subject exhibited activity. In various embodiments, an activity index 618 can also be displayed. In this example embodiment, the activity indicator is able to distinguish between motions from the subject breathing versus motions from the subject conducting other extraneous motion such as rolling, talking or coughing. In some embodiments, the system can be network-enabled such that the data displayed in FIG. 6E can also be viewed by a remote station and/or be stored in an Electronic Health Record or other database.

Various embodiments of the system 100 can be used to detect apnea, or the cessation of respiratory activity. For example, in some embodiments, if the physiological motion sensor detects no local maximum above a specified threshold, the system 100 can detect cessation of breathing as disclosed in U.S. Provisional Application No. 61/072,982 which is incorporated herein by reference in its entirety.

In various embodiments, the device can use an algorithm to determine whether there are no local maxima above specified threshold because breathing has ceased or because the subject is no longer present as disclosed in U.S. Provisional App. No. 61/072,983 which is incorporated herein by reference in its entirety and in U.S. Provisional App. No. 61/123,135, which is incorporated herein by reference in its entirety. In some embodiments, this algorithm can include analyzing two frequency bands: a high-frequency band and a low-frequency band, which are separated by software filters that is executable by a processor. If a breathing subject exists, the device can tell presence of a subject from the breathing signal which is mostly located in the low frequency band (below approximately 0.8 Hz). However, if the subject is not breathing, the device can still detect other motion including heart or other involuntary motion containing higher frequency components. Consequently, the device can determine presence of a non-breathing subject or the absence of a subject by comparing average power of different frequency bands with a threshold power level.

Various embodiments of the device can differentiate between the presence or absence of a subject based on frequency analysis and thresholds of the cardiopulmonary and non-cardiopulmonary signals obtained by the motion sensor. In various embodiments, the non-contact physiological motion sensor could be used to determine whether a subject is present as disclosed in U.S. Provisional App. No. 61/123,135, which is incorporated herein by reference in its entirety and in U.S. Provisional App. No. 61/001,996 which is incorporated herein by reference in its entirety and in U.S. Provisional App. No. 61/154,732 which is incorporated herein by reference in its entirety. For example, in a home monitoring scenario, the system 100 can be used to track how long the patient is in a specific position or a specific room. For example, in a kiosk scenario, the system could determine when a subject is present in the kiosk.

In various embodiments, the non-contact physiological motion sensor can also be used in security applications in a through-the-wall mode to determine whether there are people present in a container, or in a room. Because the sensor can be used to detect heart rate, it can be used to detect people who are hiding and/or holding their breath.

In various embodiments, the device can detect the presence or absence of a subject based on an algorithm as disclosed in U.S. Provisional App. No. 61/072,983, which is incorporated herein by reference in its entirety and in U.S. Provisional App. No. 61/123,135, which is incorporated herein by reference in its entirety. In some embodiments, this algorithm can include analyzing two frequency bands: a high-frequency band and a low-frequency band, which are separated by software filters that are executable by a processor. If a breathing subject exists, the device can tell presence of a subject from the breathing signal which is mostly located in the low frequency band (below approximately 0.8 Hz). However, if the subject is not breathing, the device can still detect other motion including heart or other involuntary motion containing higher frequency components. Consequently, the device can determine presence or absence of a subject by comparing average power of different frequency bands from threshold power level. In some embodiments, when the device is directed towards a specific bed or chair, subject presence can be detected by whether or not the physiological motion activity is above a threshold, wherein the threshold is set based on baseline measurements. In some embodiments, respiration processing can be switched off if no subject is present.

Various embodiments of the system 100 described herein include a radar-based physiological motion sensor. Various embodiments of the system 100 can include a source of radiation, one or more receivers to receive radiation scattered by the subject, a system (e.g., an analog to digital converter) to digitize the received signal. Various embodiments of the system 100 can also include a processor, a computer or a microprocessor to process the digital signal and extract information related to the physiological motion. In various embodiments, the processor can be controlled by a controller. The information related to the physiological motion can be communicated to a user in various ways (e.g., displayed visually or graphically, transmitted electronically over a wired or a wireless communications link or network, communicated audibly through an internal voice or an alarm, etc.).

Various embodiments of the system 100 described herein can operate with no contact and work at a distance from a subject. Various embodiments of the system 100 can operate on subjects that are in any position, including lying down, reclined, sitting, or standing. Various embodiments of the system 100 can work at various distances from the subject, from, for example, 0.1 to 4.0 meters. In some embodiments, the system 100 can be positioned in various locations relative to the subject, including, but not limited to, in front of the subject, behind the subject, above the subject, below the subject, to the side of the subject, or at various angles to the subject. In some embodiments, the system 100 can operate while being positioned on the subject's (e.g., patient's) chest. In these embodiments, the system 100 can be laid on the subject's chest, held to the subject's chest by a user, or worn on the subject's chest with a strap, necklace, or harness.

Various embodiments of the system 100 can use multiple receiver channels in combination with specialized algorithms to determine the direction of the target, to isolate physiological motion from spatially separated non-physiological motion, to simultaneously detect physiological motion from different subjects, to track the angle of a single subject, or to isolate the physiological motion from a first subject when one or more other subjects are within the field of view

In various embodiments, multiple receive antennas and receive channels can be added to provide multi-channel outputs. These additional receive channels can be used to determine the direction of the target, to isolate physiological motion from spatially separated non-physiological motion, to simultaneously detect physiological motion from different subjects, or to isolate the physiological motion from a first subject when a second subject is within the field of view. Algorithms used to provide this information from multiple antennas include, but are not limited to, direction-of-arrival, independent component analysis, and blind source separation as disclosed in U.S. Provisional App. No. 61/141,213 which is incorporated herein by reference in its entirety and in U.S. Provisional App. No. 61/204,881 which is incorporated herein by reference in its entirety as disclosed in U.S. Provisional App. No. 61/137,519 which is incorporated herein by reference in its entirety.

In various embodiments, the physiological motion sensor system 100 can be a stand-alone device, with its own display, user interface, clock, recording hardware and software, signal processing hardware and software, and/or communications hardware and software; this can all be integrated in one unit, or can include multiple units, connected by a cable, such as USB. Alternatively, the physiological sensor can be integrated as part of a system that can include additional monitoring devices (physiological and/or non-physiological), and use that system's display, user interface, clock, recording hardware and software, signal processing hardware and software, and/or communications hardware. In various embodiments, the sensor can receive an analog or digital synchronization signal from the system, such that data from the sensor can be synchronized with signals from other sensors and events, or it can transmit an analog or digital synchronization signal to the system, or it can have an internal clock that is synchronized with the system clock and use time stamps on the data for synchronization. In some embodiments, the sensor can be a device with its own signal processing hardware and software, with two way communication to the system which includes display, recording, and/or communications beyond the system, and possibly additional signal processing of the waveforms from the device and, if included, waveforms from other sensors. In this case, the device would receive commands from the system for starting measurements, stopping measurements, and other hardware control signals. In some embodiments, the device can perform the initial signal processing and provide a waveform that is analyzed by the system. The data can be analyzed in real time or through post-processing as disclosed in U.S. Provisional App. No. 61/204,880 which is incorporated herein by reference in its entirety.

In various embodiments, the sensor system 100 can be provided with alarms which can issue alerts if irregularities or abnormalities in the patient's breathing are detected. In some embodiments, the system 100 can also activate alarms (e.g., when the subject is not breathing for more than 10 seconds or is breathing faster than approximately 20 breaths/minute for more than 10 seconds).

In various embodiments, physiological waveforms related to respiratory effort, chest wall movement due to the underlying heart motion, and peripheral pulse movement, etc., can be obtained by the physiological motion sensor as disclosed in U.S. Provisional App. No. 61/141,213 which is incorporated herein by reference in its entirety. Information derived from these waveforms can include, but is not limited to, respiratory rate, inhale time as disclosed in U.S. Provisional App. No. 61/141,213 which is incorporated herein by reference in its entirety, exhale time as disclosed in U.S. Provisional App. No. 61/141,213 which is incorporated herein by reference in its entirety, inhale time to exhale time ratio as disclosed in U.S. Provisional App. No. 61/141,213 which is incorporated herein by reference in its entirety, frequency, depth, and length of gasps as disclosed in U.S. Provisional App. No. 61/141,213 which is incorporated herein by reference in its entirety, frequency, depth, and length of sighs as disclosed in U.S. Provisional App. No. 61/141,213 which is incorporated herein by reference in its entirety, depth of breath as disclosed in U.S. Provisional App. No. 61/072,983, which is incorporated herein by reference in its entirety, presence of and degree of paradoxical breathing as disclosed in U.S. Provisional App. No. 61/194,836 which is incorporated herein by reference in its entirety and in U.S. Provisional App. No. 61/194,848 which is incorporated herein by reference in its entirety and in U.S. Provisional App. No. 61/200,761 which is incorporated herein by reference in its entirety, tidal volume as disclosed in U.S. Provisional App. No. 61/125,021 which is incorporated herein by reference in its entirety and in U.S. Provisional App. No. 61/125,018, which is incorporated herein by reference in its entirety, abdominal excursion to chest excursion ratio as disclosed in U.S. Provisional App. No. 61/141,213 which is incorporated herein by reference in its entirety, harmonic content of breathing signal as disclosed in U.S. Provisional App. No. 61/141,213 which is incorporated herein by reference in its entirety, shape of the breathing waveform as disclosed in U.S. Provisional App. No. 61/141,213 which is incorporated herein by reference in its entirety, airflow rate as disclosed in U.S. Provisional App. No. 61/072,983, which is incorporated herein by reference in its entirety and in U.S. Provisional App. No. 61/125,021 which is hereby incorporated by reference in its entirety, distressed breathing indication as disclosed in U.S. Provisional App. No. 61/072,983, which is incorporated herein by reference in its entirety, unforced vital capacity as disclosed in U.S. Provisional App. No. 61/125,021, which is incorporated herein by reference in its entirety, heart and pulse rate, average heart, pulse and breath rate, beat-to-beat interval, heart rate variability, blood pressure, pulse transit time, cardiac output, other respiratory signals, correlation between heart and respiratory rates or waveforms, frequency, duration, and amount of activity as disclosed in U.S. Provisional App. No. 61/125,019, which is incorporated herein by reference in its entirety, frequency, duration, and amount of fidgeting and lung fluid content

The variability of these variables in various frequency bands is also subject to analysis, including heart rate variability and respiratory rate variability, but also variability of changes of the shape of the heart or respiratory waveform, changes in the depth of breathing, and changes in the degree of paradoxical breathing. These can be measured as a spot check, monitored continuously while a patient is at rest, monitored at specific times related to questions being asked, statements being made, or specific tasks being performed, or they can be monitored in subjects going about their normal activities.

The information derived from these waveforms can be displayed on a display unit. In various embodiments, information provided on screen can include, but is not limited to, respiratory rate, inhale time, exhale time, inhale time to exhale time ratio, depth of breath, presence of and degree of paradoxical breathing, tidal volume, abdominal excursion to chest excursion ratio, heart or pulse rate, average heart rate, average pulse rate and average breath rate, beat-to-beat interval. In various embodiments, information provided in waveforms can include, but is not limited to, respiratory waveform, heart waveform obtained non-contact, heart waveform obtained with the device contacting the chest, and pulse waveform. In various embodiments, the analysis provided on-screen can include respiratory rate history, heart rate history, activity index (the percentage of time the subject is physically active) as disclosed in U.S. Provisional App. No. 61/125,019, which is incorporated herein by reference in its entirety, tidal volume vs. time as disclosed in U.S. Provisional App. No. 61/125,021, which is incorporated herein by reference in its entirety, air flow rate vs. lung volume as disclosed in U.S. Provisional App. No. 61/125,021, which is incorporated herein by reference in its entirety.

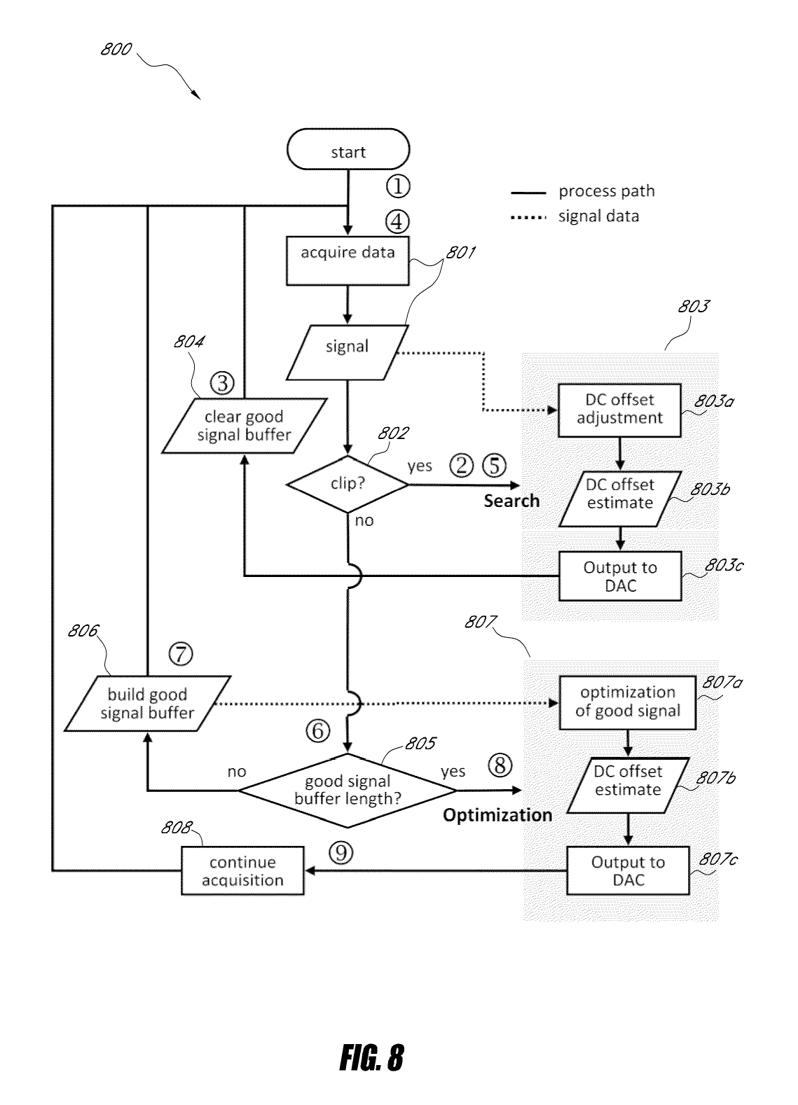

As described above, in various embodiments, the physiological motion sensor 700 can be implemented as a continuous wave radar transceiver. In various embodiments, the transceiver can be a single transmitter with a single quadrature receive channel as disclosed in U.S. Provisional App. No. 61/072,983, which is incorporated herein by reference in its entirety as shown in FIG. 7. In some embodiments, the sensor 700 can include a single transmitter 701 with multiple receiver channels or antennas 702, 703, 704 (e.g., a SIMO system) as disclosed in U.S. Provisional App. No. 61/072,983, which is incorporated herein by reference in its entirety and in U.S. Provisional App. No. 61/125,027, which is incorporated herein by reference in its entirety. In some embodiments, the sensor 700 can include multiple transmitters, each at a different frequency, and multiple receiver channels, or antennas each which can receive each frequency as disclosed in U.S. Provisional App. No. 61/125,027, which is incorporated herein by reference in its entirety and in U.S. Provisional App. No. 61/137,519 which is incorporated herein by reference in its entirety.

In various embodiments, the transceiver includes a transmitter and a receiver. In a continuous wave implementation, a transceiver can generate a single-frequency signal which is fed to the antenna. The transceiver can operate at any frequency from 100 MHz to 100 GHZ, including, but not limited to, frequencies in the 902-928 MHz ISM band, the 2.400-2.500 GHz ISM band, the 5.725-5.875 GHz ISM band, the 10.475-10.575 GHz motion detection band, and the 24.00-24.25 GHz ISM band. This signal can be generated internally with a voltage controlled oscillator (VCO) 705, which can either be phase-locked or to optionally not phase-locked a crystal or external clock. In some embodiments, if the device is integrated in an external system, the signal can be supplied by the external system. In various embodiments, the signal source can be generated internally and synchronized with an external signal, or it can be generated in an external system. In various embodiments, the board can include an RF switch, which can change the amount of RF power transmitted by approximately 10 dB or more.

In various embodiments, the receiver can be homodyne (also known as direct-conversion) with complex mixers 706, 707, 708 that can generate quadrature outputs (also known as quadrature demodulation) as disclosed in U.S. Provisional App. No. 61/072,983, which is incorporated herein by reference in its entirety as disclosed in U.S. Provisional App. No. 61/128,743 which is incorporated herein in by reference in its entirety and in U.S. Provisional App. No. 61/137,519 which is incorporated herein by reference in its entirety. In various embodiments, the receiver can also be a low-IF receiver as disclosed in U.S. Provisional App. No. 61/128,743 which is incorporated herein by reference in its entirety, which includes a heterodyne receiver in which the intermediate frequency (IF) can be directly digitized. In various embodiments, the intermediate frequency can be in the range from approximately a few Hz to approximately 200 kHz. In some embodiments, the intermediate frequency can be greater than 200 kHz. In various embodiments, the transceiver can also use a heterodyne or super-heterodyne receiver as disclosed in U.S. Provisional App. No. 61/128,743 which is incorporated herein by reference in its entirety. In various embodiments, the transmitter and receiver can include a single antenna or an array of antennas acting as a single antenna. The quadrature outputs from the receivers can be processed by an analog signal processor 709 before being digitized by an analog to digital converter 710.

In various embodiments, the DC offset can be eliminated through AC coupling or other DC-cancellation methods. In some embodiments, the DC-cancellation method can utilize a digitally controlled signal source to act as a non-time-varying (DC) reference that the original signal is compared against. In some embodiments, the digitally controlled signal source is a voltage divider with a digitally controlled potentiometer. When the comparison is performed with a difference function, this approach can remove the DC offset while preserving the time-varying signal. In some embodiments, DC cancellation is initiated with a search function, which iteratively searches for the correct DC-offset value, at the start of the DC-cancellation cycle. In some embodiments, DC cancellation is initiated by using an additional acquisition device to instantly provide the rough initial estimate of the DC-offset by acquiring the full signal before amplification and compensation. Once the initial DC-offset value is found and subtracted from the signal, the digitally-controlled reference can be fine-tuned by analyzing the newly compensated and amplified signal and then optimizing to find a better DC-offset value. The new DC-offset value can be found utilizing several methods including, but not limited to: the first read value, the median over a respiration cycle, the mean over a respiration cycle, or the center point find of a respiration arc in a complex constellation (found by calculating the mean of the in-phase signal and the mean of the quadrature signal, and setting the DC-offset values for the I and Q channels respectively). Using the above described method, the DC-offset-cancelling reference signal can be dynamically adjusted in response to large or subtle changes in the radar view to ensure minimal signal loss or distortion while maintaining proper resolution of the acquisition device. In various embodiments, DC-cancellation can include modulation of the transmitted or received RF signal. Utilizing a phase-sensitive synchronized demodulator, amplifier and low-pass filtering, signals can be extracted from high-noise, large DC-offset environments. In some embodiments, this can be similar to signal chopping with a lock-in amplifier. Modulation can be achieved in several ways, including but not limited to: physical means such as vibration or electrical means such as modulating phase, amplitude or frequency of the transmitted or received signal.

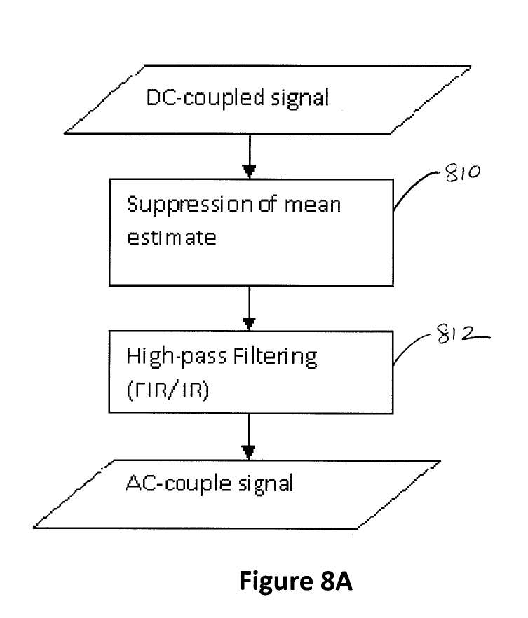

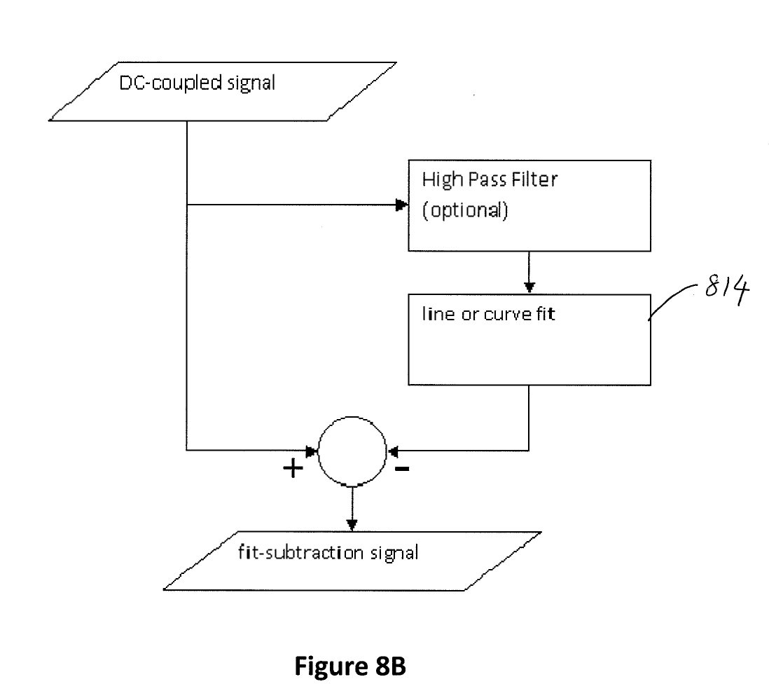

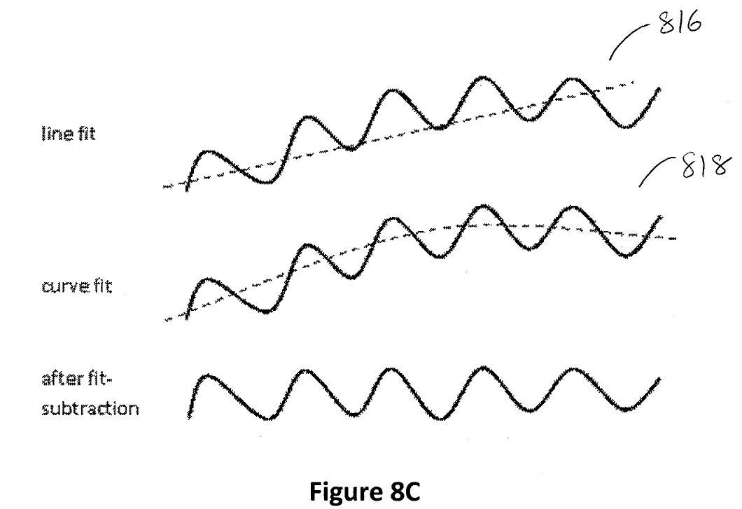

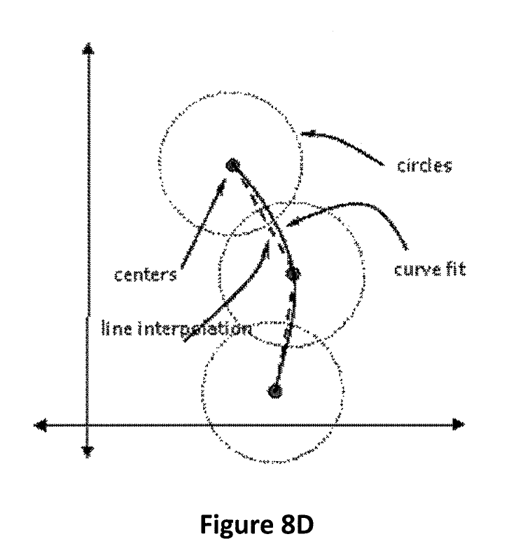

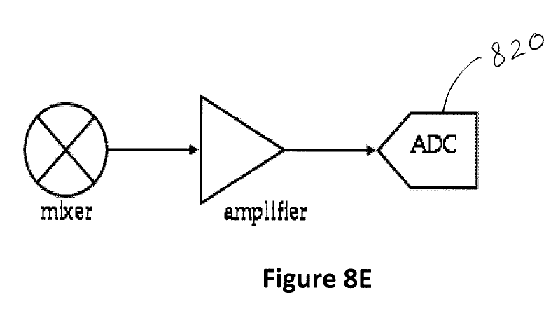

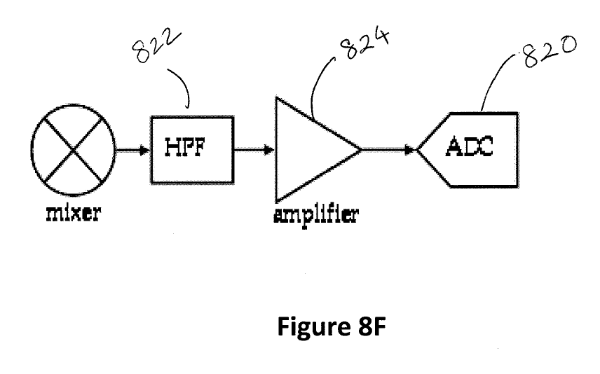

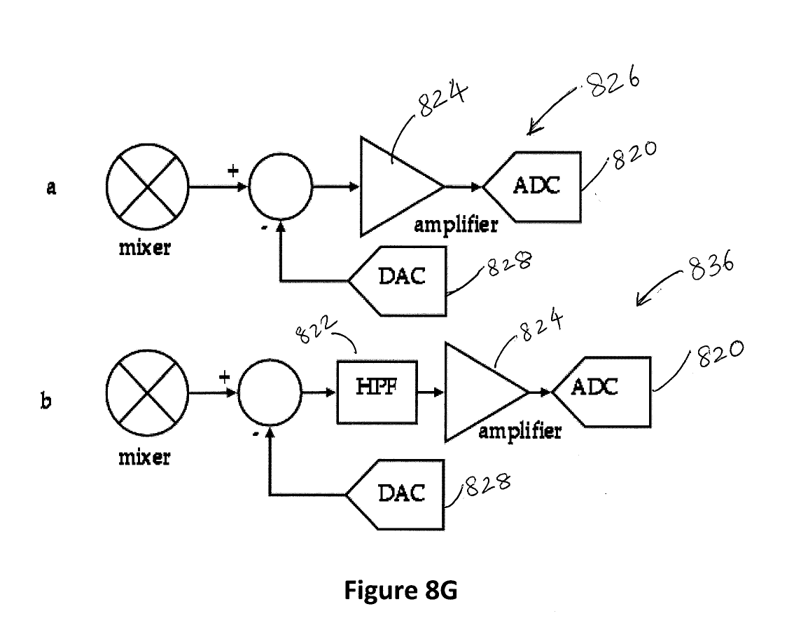

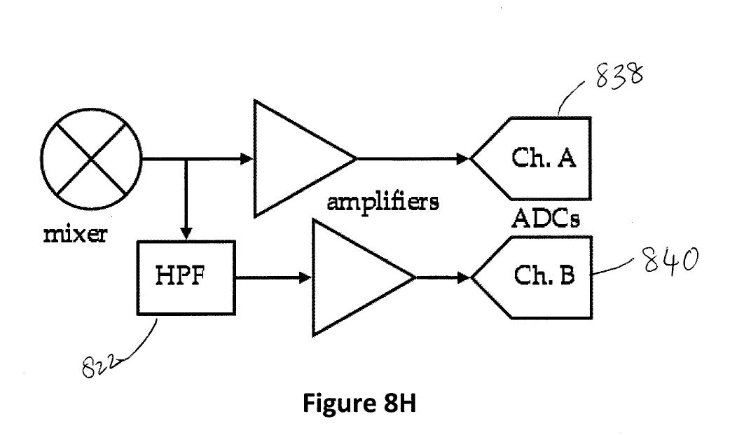

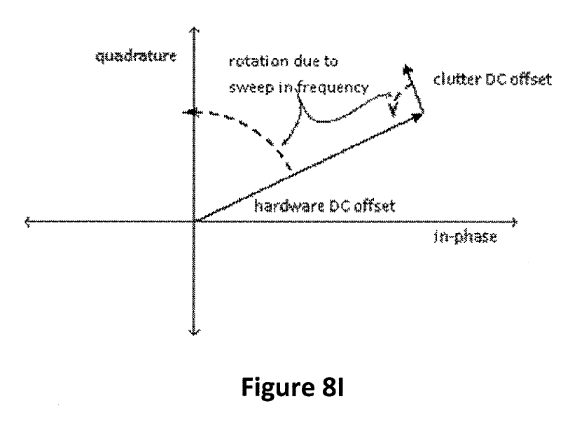

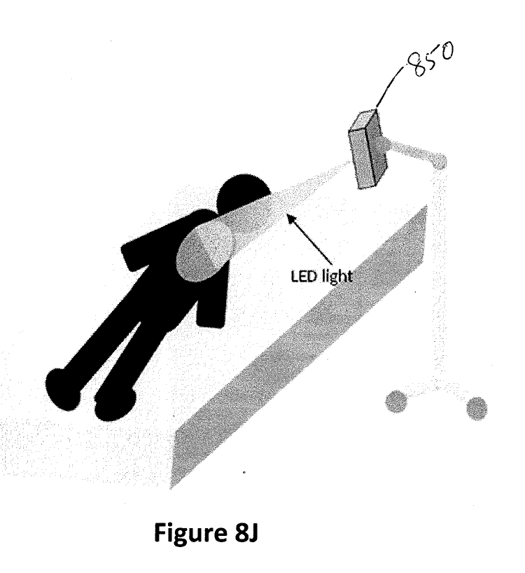

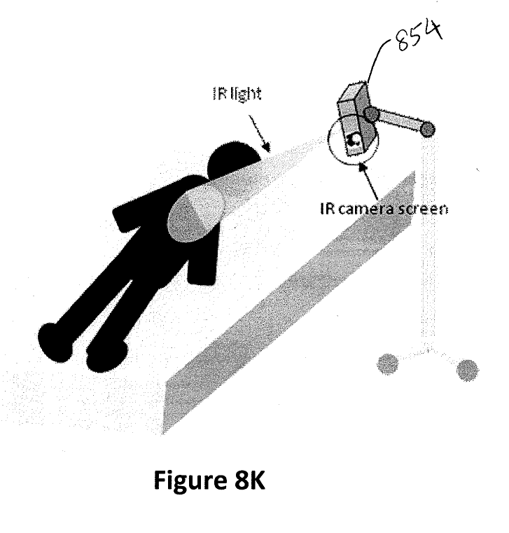

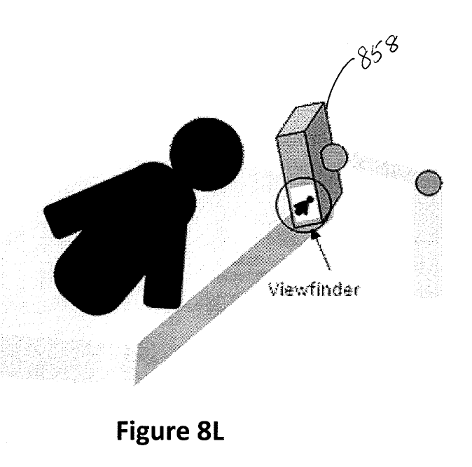

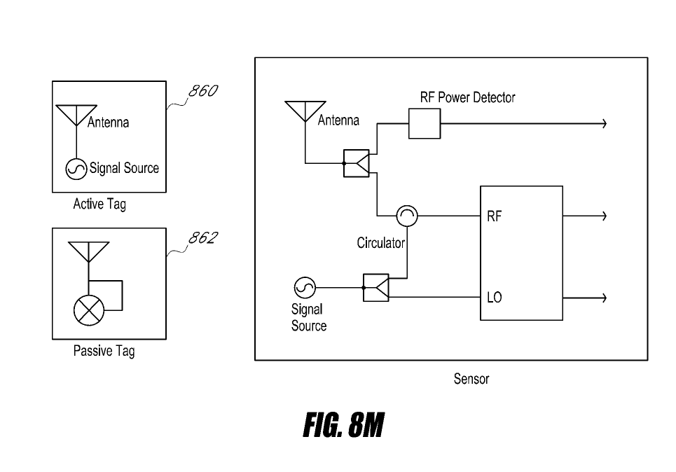

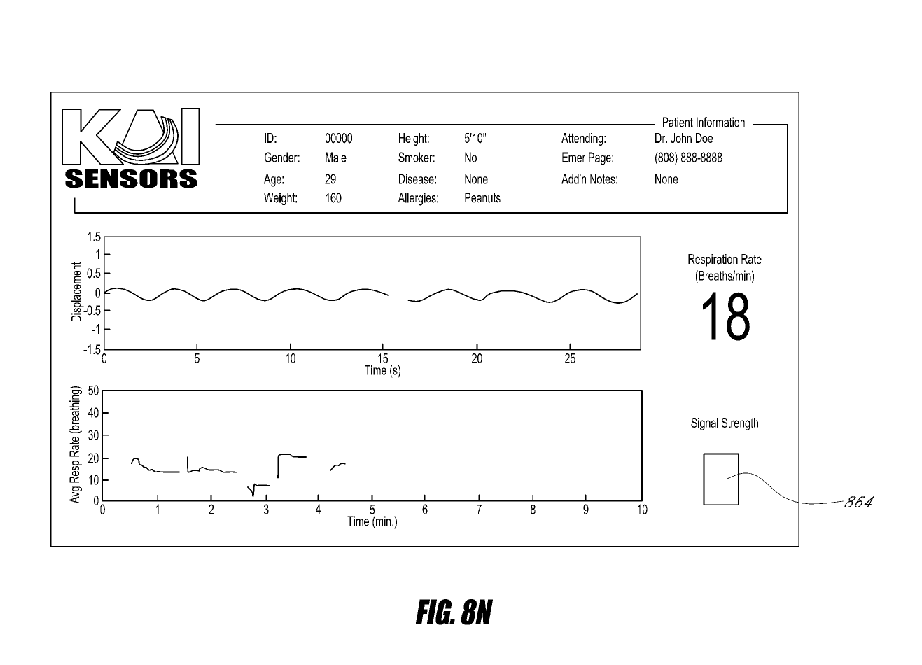

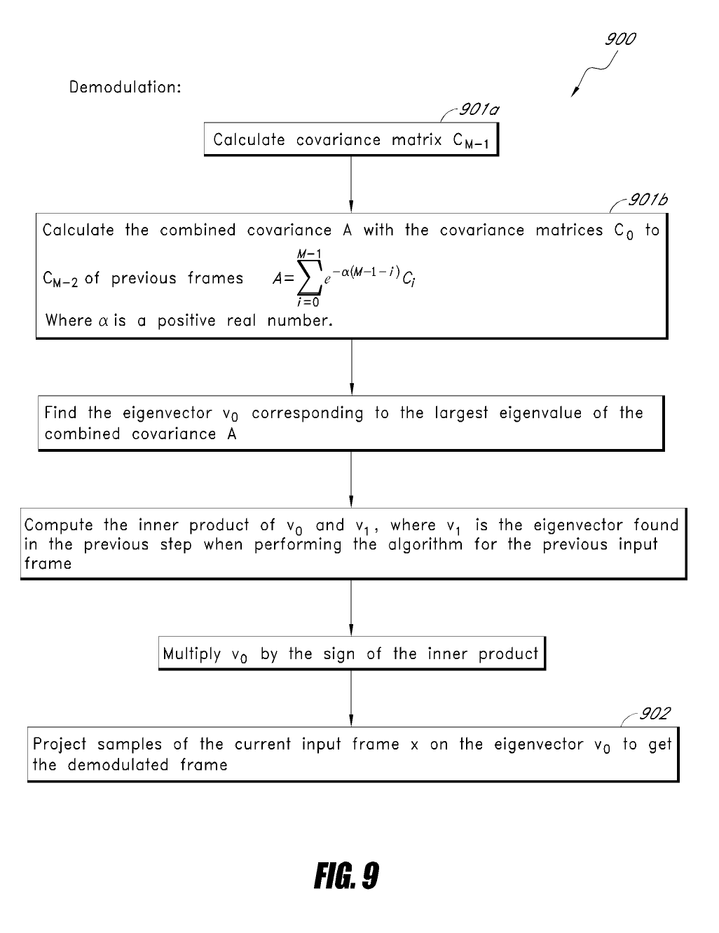

FIG. 8 illustrates a flowchart of an embodiment of a method configured to perform DC cancellation 800. At the beginning, an analog-to-digital converter (ADC) acquires the motion signal obtained by transforming the Doppler shifted received signal as shown in block 801. If in block 802, it is determined that the signal is being clipped, then the method proceeds to block 803. In block 803, the estimated DC offset is adjusted depending on at least one of the following factors gain of the system, input range of the ADC and various other factors as shown in blocks 803a and 803b. The estimated DC offset value is output to a digital-to-analog converter (DAC) as shown in block 803c. A good signal buffer configured to store continuously acquired signal that has no clipping is cleared as shown in block 804, the method returns to block 801 and the signal is re-acquired.

If in block 802, it is determined that the signal is not being clipped, then the method proceeds to step 805 wherein the good signal buffer length is checked against a threshold length. In various embodiments, the threshold length can be set by a user or a system designer. In various embodiments, the threshold length can be at least the number of samples in a full respiration cycle which can be greater than approximately 6s. If the good signal buffer length is less than the threshold length then method proceeds to block 806 wherein the good signal buffer is built by acquiring more signal. However, if the good signal buffer length is greater than the threshold length then the method proceeds to block 807 wherein the estimated DC offset value is optimized as shown in blocks 807a and 807b. During optimization, the good signal buffer is analyzed in several ways, for example by calculating the average, median or midrange voltage value. For quadrature systems, the arc-center point can be optimized. After optimization, the DC offset value is output to the DAC as shown in block 807c and the method proceeds to block 808 to continue signal acquisition.

In various embodiments of the system 100, the signal conditioning does not include high-pass filtering, DC-blocking or DC-cancellation hardware, and the DC offsets are acquired along with the signal, and removed in software. In some embodiments, a two-step method is used to suppress the DC component in a signal, in which the first step concerns the removal of the static DC offset due to the circuit, while the second step addresses the suppression of the time-varying DC offset due to the clutter, temperature and other factors. In some embodiments, in the first step, an estimate of the DC offset is determined by various methods including, but not limited to, using the value of the first sample acquired, the mean of the first few samples, or the mean of the first frame. In other embodiments, the DC offset can be measured during calibration at the factory, and this factory value can be subtracted from each frame. In some embodiments, the estimated DC offset is subtracted from the signal prior to demodulation. In some embodiments utilizing quadrature receivers, different values are calculated and subtracted for each quadrature channel. In some embodiments, the same DC offset is subtracted from every sample and/or every frame of the signal. In some embodiments utilizing frame-based processing, the second step can deduce and suppress a DC estimate from every demodulated frame by using the value of the first sample in the frame or the mean of the samples in the frame and suppressing the DC offset by subtracting this value from that frame before further processing. In some embodiments, a band-limited signal can be reconstructed from the zero-mean frames by compensating for the discontinuity across consecutive frames. In some embodiments, the discontinuity compensation uses the last sample of the previous frame and the first sample from the current frame, and then adds a constant value to the samples in the current frame such that the difference between the values of the samples specified earlier is close to zero. In some embodiments, the second step is applying a high-pass filter to the signal after it has been conditioned with the coarse estimate of the DC offset subtraction in the first step. In some embodiments, the high pass filter is applied to the signal prior to demodulation; in other embodiments, the high-pass filter is applied to the signal after demodulation. In various embodiments, the cut-off frequency of the high-pass filter can be adjusted to meet signal requirements. In some embodiments, this cut off frequency can be between 0.01 Hz and 0.1 Hz. In some embodiments, the high-pass filter cutoff can be determined adaptively, such that it is as high as suitable for a given respiratory rate. In various embodiments, the high pass filter can be implemented either as a finite impulse response filter (FIR) or an infinite impulse response filter (IIR).

An embodiment of a method for DC compensation is shown in FIG. 8A. As illustrated in FIG. 8A, the DC-coupled signal has the mean suppressed as shown in step 810, and then high-pass filtered as shown in step 812 to generate an AC-coupled signal.

In some embodiments, high-pass filtering the signal can be optional and instead of high-pass filtering the signal fitted line or curve can be subtracted. FIG. 8B illustrates a flow chart of an embodiment of a method for DC compensation in which high-pass filtering is optional. In the method illustrated by FIG. 8A, a curve-fitting or line-fitting and subtraction algorithm can be used with a preset amount of recorded data. In various embodiments, the duration of the recorded data can be 15 seconds, 30 seconds, 60 seconds or some other duration. The method comprises fitting the raw signal, or the signal after the rough DC estimate is removed, or the signal after high-pass filtering to a line or curve as shown in step 814. The fitted line is subtracted from the signal, removing the slowly-varying DC offset to obtain a fit-subtraction signal. In various embodiments, this fit-subtraction can be obtained before demodulation, and can be applied to the I & Q signals individually. In some other embodiments, this fit-subtraction can be obtained after demodulation. In some embodiments, the signal can be fit to a line as shown by trace 816 of FIG. 8C. In some embodiments, the signal can be fit to a quadratic polynomial or parametric curve, as shown by trace 818 of FIG. 8C.

In some embodiments, demodulation can involve an arctangent-based demodulation algorithm utilizing a circle-find or arc-find function, which provides a center and/or a radius as shown in FIG. 8D. In some embodiments utilizing arctangent-based demodulation, the center is used as the reference point and used to find the phase change generated as an object moves back and forth in space. In some embodiments, the movement of the arc-center is tracked over time. In some embodiments, the tracked center over time is fit to a curve which is subtracted in 2 dimensions. In some embodiments, the path is interpolated between time tracked center key points. In some embodiments, the change in the radius is tracked over time. In some embodiments, DC offset compensation such as, but not limited to, AC coupling, first sample subtraction or mean value subtraction can be utilized after arc-tangent demodulation. In some embodiments, the tracking circle-find algorithm is used instead of another DC offset compensation method. In various embodiments, center-tracking can replace the first step, the second step or the first and second steps of the previously described two-step DC-offset compensation algorithm.

In various embodiments of the system 100, the signal transmitted by the one or more transmitters described above is scattered by the subject and the surrounding and subsequently received by said one or more receivers described above as a radar-based cardiopulmonary motion sensor. In various embodiments, the Doppler-shifted signal can be transformed to a to an analog motion signal with a homodyne receiver or a heterodyne receiver. Alternatively, the Doppler-shifted signal can be down converted to an intermediate frequency which can be directly digitized, and the motion signal can be generated digitally. In various embodiments, the analog motion signal requires signal and the low-intermediate frequency conditioning before it is digitized. In various embodiments, the signal conditioning system 100 can include one or more baseband amplifiers. In various embodiments, the signal conditioning system 100 can include one or more analog anti-aliasing filters. In various embodiments the signal conditioning system 100 can include a method to remove DC offset, including, but not limited to, high-pass filtering, AC-coupling, or DC-offset removal as described in this document. In various embodiments, one or more of the baseband amplifiers are fixed amplifiers. In various embodiments, one of more of the baseband amplifiers is variable gain amplifiers (VGA). In various embodiments, the VGA can have two or more stages. In various embodiments, the VGA can have continuously tunable gain. A VGA is controlled by digital control signals. In various embodiments, the gain levels of the VGA can be determined by the user or dynamically by the processor through signal analysis as disclosed in U.S. Provisional App. No. 61/141,213 which is incorporated herein by reference in its entirety.

In some embodiments, the receiver can have one quadrature output per antenna or an array of antennae. In some embodiments, the receiver can have multiple outputs with different analog filtering and/or amplification, to isolate different information before digitization and digital signal processing. This can be advantageous in improving the dynamic range for each physiological motion signal. For example, each baseband signal would be split to have different gain and filtering for the heart signal than for the respiration signal as disclosed in U.S. Provisional App. No. 61/141,213 which is incorporated herein by reference in its entirety. In various embodiments, the system 100 can include digital signaling or a digital-to-analog converter (DAC) and hardware such that the hardware is controllable by software. In various embodiments, the hardware can be controlled in several ways, which can include but are not limited to: turning sections or components of the transceiver and the signal conditioning system on and off, which can be used in various embodiments to conserve power, for a controlled power-up, or for self-tests; turning the received and/or transmitted RF signal on and off, which can be used in various embodiments to decrease exposure to radio signals or for self-tests; setting the receiver gain, which can be used to increase the dynamic range of the system; compensation for DC offsets in the signal conditioning; controlling amount of gain in signal conditioning before acquisition; modifying the range of the data acquisition, which can be used to increase the dynamic range of the system; modifying the antenna pattern of the system, which can change the area covered by the antenna beam; and changing the frequency of the transmitted signal. In various embodiments, the hardware settings can be selected automatically by the software, manually by the user, or a combination of automatically and manually for different settings as disclosed in U.S. Provisional App. No. 61/141,213 which is incorporated herein by reference in its entirety.

Some embodiments of the system 100 utilize direct-conversion receivers that produce DC offsets that are much larger than the time-varying cardiopulmonary signal after down-conversion to baseband. In such embodiments, the DC offset is produced by hardware reflections in the transceiver system and by static objects or “clutter” in the radar environment. If these large DC offsets are not removed, they limit the amount of gain that can be used in signal conditioning, and therefore they reduce the effective dynamic range of the receiver system. In some embodiments utilizing direct-conversion receivers, DC offsets are removed through high-pass filters that AC-couple the signal, removing the DC offset before amplification and acquisition. In some embodiments utilizing direct-conversion systems to measure physiological motion at relatively low frequencies, this high pass filtering can distort the physiological signal, reducing the accuracy of assessments of vital signs and other parameters from the physiological signal. In some embodiments that do not utilize AC coupling, high-resolution ADCs are used to provide sufficient adequate dynamic range to compensate for the reduced amplification. In those embodiments of the system that are used to obtain heart and/or pulse parameters in addition to respiratory parameters, the dynamic range of the system can be required to be optimized to measure heart, pulse and/or respiratory parameters. In some embodiments, respiration signals can be in the 100 uV range with heart signals in the 1 uV range, and with DC offsets as high as 500 mV, and commercially available ADCs can be inadequate to acquire the DC offsets and the heart signals with adequate resolution.

FIG. 8E shows an example of a DC-coupled data acquisition system in which the analog-to-digital converters (ADC) 820 include anti-aliasing filters. In some embodiments, heart and respiration are acquired with the DC offsets by utilizing a high-resolution ADC to provide a dynamic range of greater than 120 dB, which requires an ADC with an effective resolution 20 bits or higher. In various embodiments, the factors that affect the dynamic range of the system include, but are not limited to the following: the intrinsic noise of the RF system, the intrinsic noise of the RF environment, the intrinsic noise of the baseband signal conditioning, the converter noise of the ADC, the input range of the ADC, the quantization noise of the ADC, the gain in the baseband portion of the circuit, and the power received by the RF port of mixer. In various embodiments, the quantization noise can be a function of the resolution and range of the ADC. In various embodiments, the quantization level can be given by the following equation:

Quantization Level=(Full range)/((2̂n−1)); where n=bit depth

In some embodiments, the desired signal to be acquired should be at least 2 times greater than the quantization level (which is also referred to as quantization noise). In some embodiments, the desired signal to be acquired should be at least 10 times greater than the quantization level (which is also referred to as quantization noise) to provide adequate resolution for rate-finding. In some embodiments, the maximum gain in the baseband portion of the circuit is related to the maximum DC offset expected in all potential uses of the device. In various embodiments, the maximum gain can be determined analytically or through observation. In various embodiments, the amplification is selected to be as high as possible while avoiding causing the DC offsets to place the signal outside of the input range of the ADC. In some embodiments, the gain range can be from 5 V/V to 100 V/V. In various embodiments, increasing the power of the VCO does not necessarily improve the dynamic range as this can also increase the DC offset proportionally. In some embodiments, the power received by the RF port of the mixer can be improved through increasing antenna sensitivity and reducing connector and component losses. In various embodiments, the signal power at the baseband is also related to the efficiency of the of the IQ demodulator which can be improved by reducing conversion loss.

In some embodiments, the whole signal, including the time-varying physiological motion signal and the DC offset, is acquired and the DC offsets are removed digitally. In some embodiments, the gain is relatively low (in some embodiments, the gain is in the 5-50 V/V range) with an ADC with a 24-bits or higher resolution. In some embodiments, a fully differential signal path between the mixer and the ADC is utilized to improve noise rejection in the common mode. In some embodiments, the ADC over-samples the signal and utilizes interpolation, decimation and filtering, to extract an extra bit of data for every 4× the signal is oversampled.

In some embodiments, to increase the dynamic range, the DC offset can be compensated for in the hardware. FIG. 8F shows an embodiment with a high-pass filter (HPF) 822 before an amplifier 824 to provide AC coupling. When the DC offset is removed with a high-pass filter 822, the gain of the amplifier 824 can be increased as long as the maximum expected signal amplitude is not larger than the input range of the ADC 820. In this embodiment, the gain can be in the 1,000 V/V range to avoid wasting ADC resolution with acquiring the DC offset in a DC-coupled system. In various embodiments, the high-pass filter can be designed to reduce phase and amplitude distortion of the physiological signal. High-pass filters can also introduce transient affects, especially with low frequency physiological signals where the time constant of the HPF must be relatively high because the filter must have a low cutoff frequency.

FIG. 8G shows two systems 826 and 836 that can be used for DC offset compensation. System 826 of FIG. 8G shows a method of DC offset compensation introduced at or before the amplifier 824 which subtracts a DC voltage from the signal before amplification without a high-pass filter. In some embodiments, this DC offset compensation can be provided by a digital to analog converter (DAC) 828 and a comparator to reduce most of the DC offsets. In such embodiments, the comparator checks for clipping of the signal, either with analog comparators near the input range of the ADC 820 or digitally after acquisition. In such embodiments, this method can not remove all of the DC offset, but it can allow the gain to be increased before acquisition. In some embodiments, this DC-offset-subtraction method can allow the gain to be increased enough for the remaining DC offset, respiratory signal, and heart signal to be acquired by a high-resolution ADC. In some embodiments, when the DC offsets are understood to be at particular levels without significant change over time, the compensating value can be fixed without adjustment. In some embodiments, this compensating value can be set during a factory calibration. In some embodiments, the DC value used for compensation can be adjusted in real-time based on the DC offset of the acquired signal. In some embodiments, an extra step of AC coupling, as illustrated in the system 836, can be added after the DC offset compensation, such that the transient effects of the time constant of the AC-coupling filter are reduced because the DC offset value is decreased, and enabling less stringent requirements of the high-pass AC-coupling, reducing the distortions introduced by the filter. AC coupling can be added by including a High pass filter 822 before the amplifier as illustrated by the system 836.

FIG. 8H shows a parallel acquisition technique that is utilized in some embodiments to optimize the signal conditioning for both respiratory activity and cardiac activity. In some embodiments, for optimal acquisition of a respiration signal, distortion due to an AC coupling filter should be reduced. Accordingly, a DC-coupled acquisition for respiratory activity is performed by channel A 838 as shown in FIG. 8H. In some embodiments, this is achieved by a high-resolution ADC device, without any DC offset compensation. In some embodiments, DC offset compensation can be utilized for channel A 838. In FIG. 8H, channel B 840 acquires the heart signal, and includes an AC coupling HPF 822 in order to accommodate the higher gain in channel B′s amplifier, which is required for acquisition of the much smaller heart signal. In this embodiment, gain for the heart signal in the B channel can be in the 10,000 V/V range or higher, depending on the implementation of the HPF and the performance of the amplifier. In some embodiments, DC offset compensation can be performed before the high-pass filter 822 in channel B 840, as illustrated in FIG. 8H. In some embodiments, the HPF 822 can be tailored the attenuate the respiration signal as well as the DC offset.

In some embodiments, frequency agility of the voltage controlled oscillator (VCO) can be achieved with the assistance of a phase-locked loop (PLL). In some embodiments, with digital control of the PLL, the radar sensor carrier frequency can be tuned to specific frequencies in the ISM band. Because the DC offset of the system is complex (different on the I and Q channels), sweeping the frequency of the local oscillator causes the DC offset vector to rotate in the I/Q plane as illustrated in FIG. 8I. In some embodiments, the frequency of the VCO can be selected to remove either the in-phase, I, or the quadrature, Q, component of the DC offset vector. In some embodiments, high-gain amplifiers without AC coupling can be used to improve the signal-to-noise ratio at acquisition. In some embodiments in which the frequency is selected such that one component is removed, only one channel is needed to acquire a signal. In some embodiments, the frequency can be quickly alternated between two chosen frequencies selected such that one frequency can provide DC offset compensation for one channel (Q) with the other frequency can provide DC offset compensation for the other channel (I). In some embodiments, acquisition can be timed to only acquire the channel that has its DC offset compensated for, and acquisition can alternate as the frequency alternates between the two frequencies. In some embodiments, multiple frequencies can be acquired that provide DC offset compensation for either the I or the Q channel, and the values can be compared as the frequency changes. In some embodiments, adjustments to the frequency can be made to compensate for changes in the hardware to match with preset DC-offset compensation in baseband.

In some embodiments, signal chopping is used to avoid or remove DC offsets. In some embodiments, a PLL-controlled VCO chops a signal to provide DC offset compensation. Chopping is a modulation of the source of a signal at a certain frequency. This modulation frequency is used as a reference when the signal returns, allowing static elements, such as DC offsets, to be removed. In some embodiments, the VCO can be switched on and off at the chopping frequency. In some embodiments, the PLL can control the phase of the VCO and modulate that the phase at the chopping frequency. In some embodiments, the frequency of the VCO is modulated at the chopping frequency by the PLL. In some embodiments the received signal is acquired above the Nyquist frequency of the chopping modulation and the signal is demodulated digitally. In some embodiments, a lock-in amplifier is synced to the chopping frequency and used to remove the DC offsets of both I & Q separately. In some embodiments, an RF switch or phase shifter in the VCO-to-antenna path is utilized and turned off and on at the chopping frequency. In some embodiments, one or more of the above embodiments are utilized together.

In some embodiments, a variable phase shifter and a variable attenuator in the LO path of the mixer are used to cancel the hardware reflections of the transceiver system. The phase is set to be in anti-pole to the cumulative hardware reflections and the attenuator is set to match the magnitude of the cumulative hardware reflections. In some embodiments, the phase shifter and attenuator are set once at assembly to match the system. In some embodiments, the phase shifter and attenuator are directly digitally controlled or controlled through digital-to-analog converters by a processor to cancel hardware and clutter reflections.

Various embodiments of the system including the radar-based physiological motion sensor can include wired or wireless communication systems. The various embodiments can use standard or proprietary communication protocols, or combinations thereof. Such protocols can include technologies from all layers of the TCP/IP networking model, including, but not limited to, serial, USB, Bluetooth, Zigbee, Wi-Fi, Cellular, TCP/IP, Ethernet, SOAP, etc. For example, Ethernet can be used as the link layer protocol while TCP/IP is used for routing, and SOAP is used as an Application layer protocol. On the other hand, only TCP/IP over Ethernet can be used, without additional packaging at the Application level. In the later case, data collected from the radar system 100 can be formatted and directly packaged as TCP payload. In some embodiments, this can include a timestamp for when the data was collected, the data, and an indicator for the quality of the data. This data is attached with a TCP header and then becomes the IP payload. The IP header (addresses) is attached to the payload and then is encapsulated by Link layer headers and footers. Finally, physical layer header and footers are added and the packet is sent via the Ethernet connection. To access data from the connection, a user or a client should have a program to listen to a specified port on their Ethernet connection where the packets are being sent.

In some embodiments of the system 100 that utilizes a directional antenna, the accuracy and reliability of the transmitted radiation (or aiming vector) is well aligned with the target's torso. The aiming vector is defined as the normal vector from the center of a planar antenna. In some embodiments, an aiming aid can provide guidance by indicating what is in the radar's field of view. In some embodiments, an aiming aid can provide an indication of how well the sensor has been aimed. In some embodiments, an aiming aid can be placed on the radar sensor or integrated into the radar sensor. As illustrated in FIG. 8J, some embodiments can include placing a directional LED 850 (light emitting diode) or some other type of directional light source on the center of the radar front panel. In some other embodiments, an infra-red light and/or an infrared detector can be installed on the front panel to make an infrared viewfinder 854 as shown in FIG. 8K. In some other embodiments, an optical viewfinder 858 can be installed as shown in FIG. 8L. In some embodiments, the directional light can illuminate the area within the radar sensor's field of view, such that the illuminated area is in the path and the path is effectively visible. In other embodiments, the directional light can provide a point of light at the center of the aiming vector, such that the user is aware of the direction in which the system 100 is aimed. In some embodiments, a directional infrared light can be used in conjunction with an infrared imaging device and a display such that the user can see the field of view on the display. In some embodiments, a display on the device can show the area within the radar sensor's field of view, so the user can see where the radar sensor is aimed by looking at the device, without knowledge of the target. In some embodiments, this can be implemented as a viewfinder. In some embodiments, this can be implemented as a digital camera and a digital display.

In some embodiments, an aiming aid can be realized through an additional device placed on the patient, subject, or target. In some embodiments, the device placed on the subject can be a tag that emits a radio-frequency signal. In some embodiments, this tag can be placed directly on the subject's chest area, or on the subject's clothing in the chest area. In some embodiments, this device can be affixed with an adhesive, worn on a lanyard, or clipped to clothing. In some embodiments, this tag can be disposable. In some embodiments, an aiming aid can be achieved by measuring the power from the radio-frequency signal of the tag that is received by a power detector that uses the same directional antenna as the sensor. In other embodiments, the power detector can use its own directional antenna. The detected power from the tag is greatest when the received radio-frequency power at the tag's frequency is at its maximum. In some embodiments, the power can be indicated with a bar graph. In some embodiments, the radio-frequency tag is an active beacon tag 860 that emits a radio-frequency signal at a slightly different frequency from the radar sensors' transmitted and received signal. In other embodiments, the radio-frequency tag can be a passive tag 862 that reflects a harmonic of the radar sensor's transmitted signal, or that emits another modification of the radar sensor's transmitted signal. In other embodiments, a radio-frequency identification tag (RFID tag) can be used to provide additional information including, but not limited to, the patient's identification number. A schematic for radio-frequency tags and a sensor set is shown in FIG. 8M. In some continuous monitoring embodiments, the tag can be used to indicate the presence or absence of the desired target and to ensure that the desired target is being measured.

In some embodiments, the radar sensor can include multiple antennas, each with a receiver, such that it can determine the direction of a signal source. In some embodiments, this can be used to determine the direction of the target and to provide feedback to the user on how to better aim the device toward the target. In some embodiments, this multiple-receiver sensor can be used in conjunction with a radio-frequency tag, such that the sensor can determine the direction of the tag and provide feedback to the user on how to better aim the device toward the tag. In some embodiments, a multiple antenna sensor used in conjunction with a radio frequency tag can differentiate or separate the desired target's signal from interference with a software defined smart antenna technique.

In some embodiments, the power of the physiological signal can be used to provide an indication of whether or not the device is properly aimed. In some embodiments, if the physiological signal power is high enough, the device can be considered to be aimed well enough.

FIG. 8N shows a screen shot of an embodiment of the display associated with a continuous vital signs monitor with a radio-frequency tag-based power indicator. As the direction from the antenna to the tag changes, the radio-frequency tag power indicator 864 shows strength of the received power from the radio-frequency tag. This power indicator can be greatest when the sensor is best aligned with the tag. The user can change the sensor position until this value is at its maximum.

In various embodiments, the digitized quadrature signals can be processed using various algorithms to provide respiratory and pulse waveforms.

In the system 100, deviation of the phase is proportional to the chest motion divided by the wavelength of the carrier signal, and the amplitude of the signal is not significantly affected by chest motion, such that when the phase is plotted in the I/Q plane, the I/Q constellation is distributed along an arc of a circle or a full circle. In embodiments in which the chest motion is small compared to the signal's wavelength, the arc sweeps a small portion of the circle, such that it can be approximated by a line, and the phase can be demodulated through linear methods. Alternatively, if the chest motion is large compared with the carrier signal's wavelength, the I/Q constellation samples are distributed on a larger arc that cannot be approximated by a line. In some embodiments in which the transceiver operates at approximately 5.8 GHz, when the chest motion due to the respiration is 0.5 cm, the phase deviation due to the chest motion is 70°; a 70° arc cannot be approximated as a line in the complex constellation. In these embodiments, non-linear demodulation based on arctangent function can extract phase information directly from arc-distributed samples.

In various embodiments, one of three basic types of demodulation can be used to convert quadrature signals to a motion waveform: linear demodulation, non-linear demodulation, and heuristic methods; any of these methods can use any of the raw (unfiltered) signal, the filtered signal, or a segmented signal for demodulation.

In various embodiments, the quadrature signals can be demodulated using any of several algorithms, including but not limited to linear demodulation, arc-based demodulation algorithm (e.g., arc-tangent demodulation with center tracking) or non-linear demodulation algorithm. Demodulation algorithms can include any of the following methods, but not limited to, projecting the signal in the complex plane on a best-fit line, projecting the signal in the complex plane on the principal eigenvector, or aligning the signal arc to a best-fit circle and using the circle parameters to extract angular information from the signal arc. Linear demodulation can use any of many algorithms, including projecting the signal in the complex plane on the principal eigenvector, or projecting the signal on the best-fit line. Arctangent demodulation can extract phase information which is corresponding to the chest motion associated with cardiopulmonary activity as explained herein. In quadrature systems, data collected by two orthogonal channels (e.g., In-phase (I) and quadrature phase (Q)) lie on a circle centered at a DC vector of the channels. After tracking center vector of the corresponding circle and subtracting it from the data samples, phase information of received signal can be extracted through an arctangent function.

In some embodiments, linear demodulation is the projection of the signal on a linear vector. In some embodiments, the signal is rotated until a maximal projection on the x or y plane is achieved. In some embodiments, a best fit line is estimated, and the data is projected on the best-fit line. In some embodiments, specific key points, such as the end points of an arc, are connected to form a line, and the signal is projected on this line. In some embodiments, the signal is projected on the line that provides the most variance in the signal.

In some embodiments, the hardware can be used in conjunction with the software to enable types of linear demodulation. In some embodiments, the carrier radio frequency can be adjusted with a phase-locked-loop or other method to put one of the channels in the null, such that most of the signal is on the other channel; the signal in the non-null channel is used. In some embodiments, a phase-shifter in the RF circuit can be tuned to a point where one channel is in the null, and the signal on the other channel can be used.

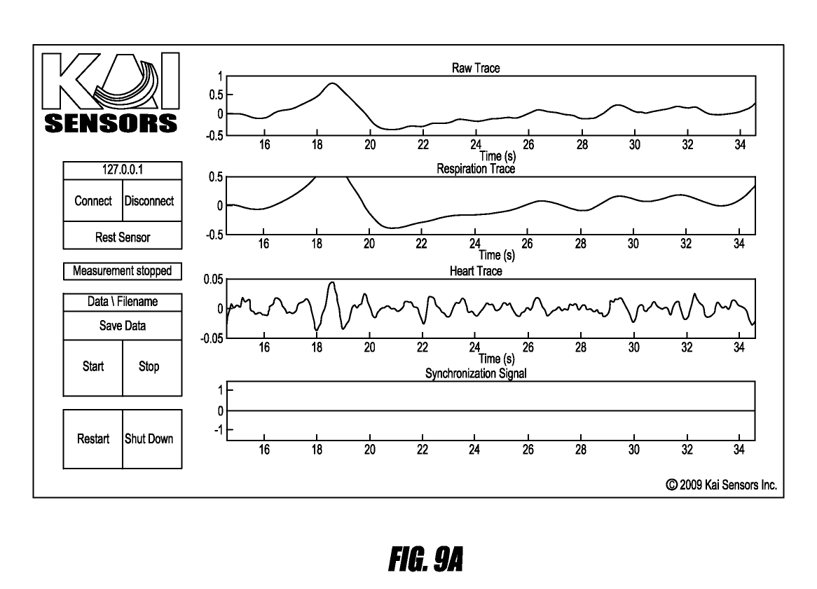

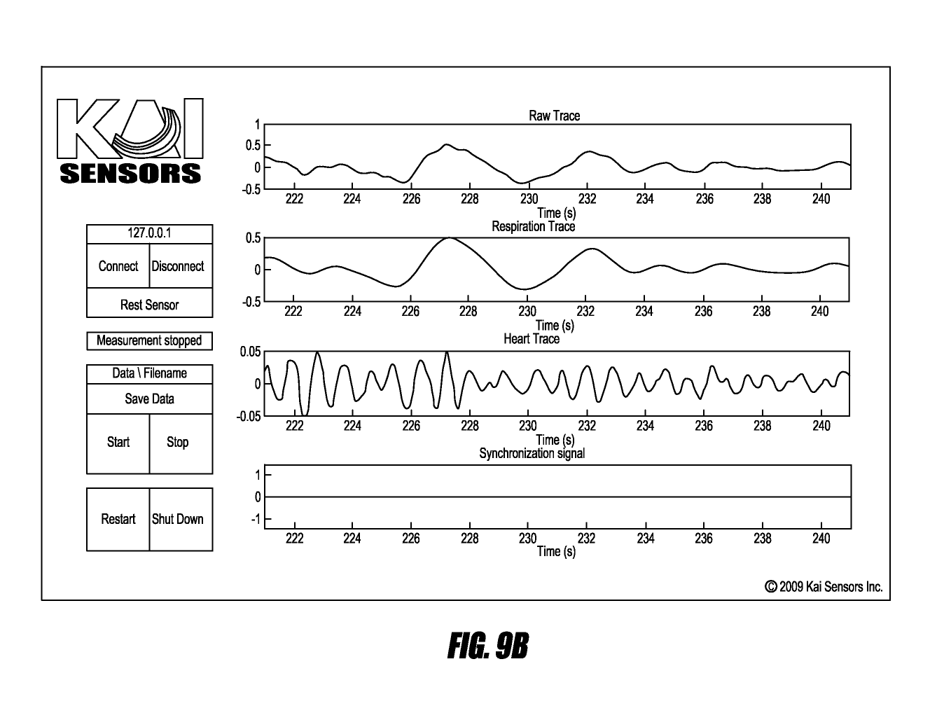

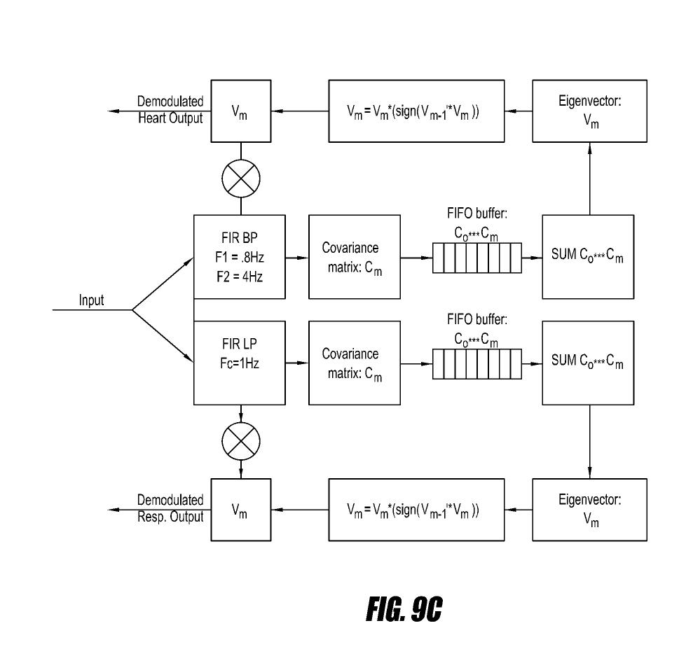

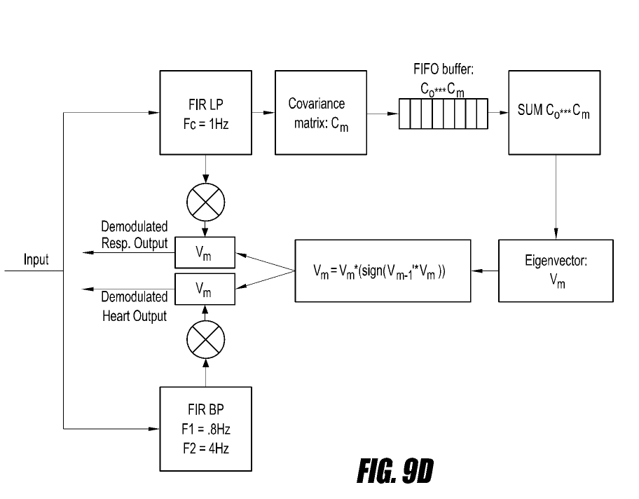

An embodiment of a linear demodulation algorithm is further described below and illustrated in FIG. 9. In one embodiment, the algorithm comprises computing covariance matrices for a subset of input frames as shown in block 901a including the most recent frame and projecting the data on a primary vector or an eigenvector of said covariance matrix as shown in block 902. If it is determined that the current eigenvector is in a reverse direction as compared to a previously determined eigenvector then the algorithm is configured to rotate the current eigenvector by 180 degrees.

In various embodiments, the linear demodulation algorithm comprises the following steps:
1. Compute covariance matrix C_{M-1} of the current input frame x as shown in block 901a.
2. Using C_{M-1} and covariance matrices C₀ to C_{M-2} of previous frames, compute an A-matrix as shown in block 901b given by the equation:

$$A = {\sum }_{i = 0}^{M - 1} {e}^{- α \cdot ( M - 1 - i )} {C}_{i}$$

where α corresponds to a damping factor and can be a positive real number. In various embodiments, the value of α can range from approximately 0.1 to approximately 0.5. In one embodiment, α can be 0.2. M corresponds to the number of frames in the buffer and can range from 2 to 15. In one embodiment, M can be 10.
3. Find the primary vector or eigenvector v₀ corresponding to the largest primary value or eigenvalue of A as shown in block 901c.
4. Compute the inner product of v₀ and v₁, where v₁ is the eigenvector found in step 3 when performing the algorithm for the previous input frame as shown in block 901d.
5. Multiply v₀ by the sign of the inner product found in step 4 as shown in block 901e.
6. Project samples of the current input frame x on the eigenvector v₀ calculated in step 5 to get the demodulated frame as shown in block 902.

If a target's periodic physiological motion variation is given by x(t), and the wavelength of the radar signal is λ, the quadrature baseband output, assuming balanced channels, can be expressed as:

$$B ( t ) = {A}_{r} exp ( i * ( θ + \frac{4 πΔ x ( t )}{λ} ) ) + DC$$

where DC is a complex number representing the non-time-varying voltage values of the I and Q channels, θ is the constant phase shift due to the transceiver architecture and target range, and Ar is the amplitude of the baseband signal. From (1), it is obvious that if DC, which comes from clutter, intra-circuit reflection, and self-mixing is estimated and removed, the angle deviation, which is linearly proportional to actual physical motion of a target x(t), can be extracted simply by the arctangent function. However, if the low-frequency or direct-current component of the phase shift caused by x(t) is removed, or if DC is not removed, arctangent demodulation is not straightforward.

In some embodiments, after the signal is digitized, a representation of the signal on the I/Q plot is utilized. In some embodiments, a DC-coupled acquisition system is used and the constellation due to respiration in the I/Q plane can be a straight line, an arc, an ellipse, a figure-8-like shape, a crescent shape, an egg-like shape, a circle, or a combination of the above. In some embodiments, digital signal processing can remove the DC offsets and/or slow changes in the DC offset over time. In some embodiments, the signal acquired by the ADC or the raw signal can be low-pass filtered before demodulation, but in other embodiments, it can not be low-pass filtered before demodulation. In some embodiments, the raw or filtered signal can be segmented through time decimation, quantization in IQ space, or through the estimation of key data points of the signal shape. In embodiments in which key data points are estimated, the signal processing algorithm can use a method to reduce the signal to a few points for representation, including, but not limited to identifying the following points: end points such as the extrema of an arc, points of minimum or maximum velocity; points of minimum or maximum acceleration; centers of clusters of point density, points of largest change in direction, or points of largest change in segment length; self intersection points; points of intersection of a fitted shape or a fitted shape's axis; or mid-point between other key points. In some embodiments, the above methods for estimating key points can also be used on a 2D gradient of the points or of the path.

In some embodiments, the signal in the I/Q plane is segmented before further processing. Various embodiments of methods to create a segmented representation of the signal can use a weight all samples in the frame equally, or can weight samples differently. Various embodiments of methods to create a segmented representation of the signal can use a predefined number of samples, a number of samples limited by the time of one cycle, a number of samples based on a multiple of the cycle time, a number of samples based on the time of many cycles, an adaptively set number of samples. In some embodiments, the samples used with the above methods to create a segmented representation of the signal can be defined spatially, as a present path length in the I/Q plane, a path length based on the length of the full cycle path, or a path length based on the shape of the I/Q sample constellation.

In various embodiments, once either the DC value in (1) or the center of a circle corresponding to the quadrature sample distribution is estimated, the output samples can be relocated with respect to the DC vector or the center of arc can be relocated to the origin of the complex axis. In some embodiments, the angle of the relocated arc is then linearly proportional to the physical motion of a target.

In embodiments utilizing non-linear demodulation, the movement around the center of a circle describes the movement of objects in relation to the sensor, and a center-find algorithm can be implemented. In some embodiments, the center is found by identifying the best-fit circle through least-squares methods or maximum likelihood estimator which can define a circle with based with geometric or algebraic methods, based on non-linear or linear least-squares fitting to samples distributed along an arc. In some embodiments, the signal can be rotated before the center-find algorithm is implemented. In some embodiments, the signal can be fitted with circles, ellipses, shapes in parametric paths, or a variety of shapes in a look-up library of shapes and key points. In some embodiments, the methods above can use the raw signal, a filtered signal, a segmented signal or a set of key points. In some embodiments, all permutations of 3 points can be used to calculate a set of estimated center points by finding the point that is equidistant from all three, and then the center can be calculated from the set of estimated center points using a geometric center, center of mass, radian, mean, or other method. In some embodiments, any subset of all permutations of 3 points can be used rather than all permutations.

In some embodiments, the arc is segmented (divided into sections), and the intersection of the perpendicular vectors of the sections is used to give an estimate of the center using a least mean square error, maximum likelihood estimation, or other method. In some embodiments, the end points of an arc define a chord of a circle, and the normal vector at the midpoint of the chord is defined as the perpendicular axis of the arc; segments along the arc each have a normal vector, which intersects the arc's perpendicular axis at the center point. In some embodiments, the mean, midpoint or median of the intersect points along the perpendicular axis can be defined as the center of the arc. In some embodiments, intersection outliers along the axis are removed before the center-estimation algorithm is applied. In some embodiments, a line fit is performed to find the perpendicular axis of the arc, which intersects the midpoint between the end points.

In some embodiments where the carrier wavelength is shorter than the displacement of the chest, such that a complete circle is formed in the I/Q plane, the center can be found by a best fit circle, center of mass, geometrical center, 2D low-pass filter with peak-finding, or look-up table fitting the data to a variety of circles.

In some embodiments, over a period of time, slight movement of the subject, temperature change, or other sources can cause a variation of the DC value. In some embodiments, the error between the fitted circle and the data is monitored and can trigger a new fitting or center-find when the error is above a set threshold. In some embodiments, after the initial circle is estimated, a second circle is estimated at each frame and used to track estimation error. In some embodiments, this error can be defined as the distance between the tracked center and the estimated center, the difference between the tracked radius and the estimated radius, the mean square error between the signal points and the fitted circle, or a combination of above. For example, in some embodiments, if the error between the estimated and the tracked center exceeds a threshold, the tracked center becomes the new estimated center. In some embodiments, these thresholds can be set in the code, by the user, or proportionally adjusted according to respiratory rate, circle radius, and/or phase displacement. For example, in some embodiments, the center error threshold can be half the radius of the estimated circle. In some embodiments, a re-estimation of the circle center can be periodic over time, occurring at pre-defined intervals, after a number of respiration cycles or repetition of respiratory patterns. In some embodiments, re-estimation of the circle center can be triggered by non-respiratory motion, such that after non-respiratory motion is detected, the algorithm searches for a new circle center. In some embodiments, the data that the tracked circle fits to can be all the data from the time the circle was estimated circle to the most recent data, 2D low-pass filtered data, time-weighted 2D filtered, only the current respiration cycle, or other subsets and/or altered versions of the data.

In some embodiments, demodulation is performed in real-time as the center is estimated. In some embodiments, demodulation is performed retrospectively for an optimal center from a built up buffer in memory. In some embodiments, the center is tracked periodically over time and fit to a line, quadratic curve, geometric shape or polynomial interpolation and used as moving center during demodulation.

In some embodiments, before center-finding or circle-estimation, the arc is smoothed, identified, or defined via one of several methods. In some embodiments, a 2D gradient is applied to the complex samples, and the arc's trajectory is defined by the gradient peak values. In some embodiments, the principal vector for small segments can be estimated and those principle vectors can be used to provide the trace of the arc. In some embodiments, the endpoints of the arc are estimated from the density of the samples, as high density points have high probability to be an endpoint of the arc. In some embodiments, the endpoints of the arc are estimated from a 2D gradient, to identify the points of direction change and zero-velocity. In some embodiments, an arc trajectory is adjusted such that arc has the endpoints identified by one of the end-point-finding methods. In some embodiments, all samples are adjusted such that they are along the arc trajectory defined by one or more of the above methods, at the nearest point to the sample.

In some embodiments, the radius of the circle is analyzed. The radius of the circle has a correlation to the distance between the radar and the subject as well as the radar cross section. The radar cross section is related to the area and the reflectivity of the radar target. For vital signs monitoring, the radar target can be the moving parts of the subjects body during respiration, such as the chest and abdomen. In some embodiments, the radius of the circle, and/or changes in the radius of the circle, can be used to determine the position of the subject relative to the radar and/or changes of the position of the subject relative to the radar. In some embodiments, the radius of the circle and/or changes in the radius of the circle can be used to calibrate the depth of breathing calculation, and adjust the calibration for changes in position. In some embodiments, the radius and chest movement information can be used to determined relative tidal volume respiration. In some embodiments, the radius can be used to calibrate the relative tidal volume estimates. In some embodiments, changes in the radius and/or center point can be used to detect non-physiological motion. In some embodiments, changes in the fit of the samples in the I/Q constellation to a best-fit circle calculated using historical data can be used to detect non-physiological motion.

In some embodiments, a best-fit line is repeatedly computed for small and consecutive subsets of the samples. In some embodiments, the changes in direction of the best-fit lines are used to infer the cardiopulmonary motion. In some embodiments, these changes are accumulated to produce a demodulated signal. In some embodiments, the velocity is deduced from the number of points in a given spatial window of the signal. In some embodiments, the velocities can be processed by various ways including summation to produce the demodulated cardiopulmonary motion.

In some embodiments, demodulation is performed based on selection of a key point in the complex plot, based on the gradients, velocities or point densities. In some embodiments, the resulting key point is an end point in the trajectory (e.g. an extrema). In some embodiments, the demodulated signal is calculated as the distance of each successive sample to the key point.

In some embodiments, the I/Q data points can be translated to a polar coordinate plane. When the DC component is removed, the origin becomes the center of the arc. The movement of the object is described by the change in phase of the data over time.

In some embodiments, a rate can be found without demodulation by finding points of direction change, and using them to estimate a respiratory rate.

In some embodiments of the system 100 in which both respiratory and heart and/or pulse motion are being acquired, the same best-fit line or circle is used for demodulation of all physiological motion. In other embodiments of the system 100 in which both respiratory and heart and/or pulse motion are being acquired, the different best-fit lines or circles are used for demodulation of respiration and heart signals. In some embodiments, when linear demodulation is achieved by projecting the signal on the principal eigenvector, the heart signal can be estimated by independently calculating and optimizing the eigenvectors used to demodulate the heart signal and those used to demodulate the respiration signal. FIGS. 9A and 9B contrast the heart trace obtained with a vector locked to the respiration vector with the heart trace obtained with independent eigenvectors used for heart and respiration demodulation. FIG. 9A, with locked vectors, has a noisier heart trace. FIG. 9B, with independent vectors, has a less noisy heart trace.

In some embodiments, independent demodulation of the heart and respiration signals can be achieved by filtering the I and Q signals to isolate the heart signal and the respiratory signal, before using a demodulation method to combine the heart I and Q signals and the respiration I and Q signals. In various embodiments, any of various linear, nonlinear, and heuristic demodulation methods can be used. In some embodiments, the filtering is performed with bandpass filters. In some embodiments, the filtering is performed with adaptive filters. In some embodiments, the filters are IIR filters. In some embodiments, the filters are FIR filters. In some embodiments, the signals are processed frame by frame. In some embodiments, the sample rate is 100 Hz with a frame rate of 96 samples/frame. In other embodiments, the sample rate is 1000 Hz and the data is down sampled to 100 Hz with a frame rate of 96 samples/frame. In some embodiments, over each frame, raw data is filtered using a FIR band-pass filter with cutoffs at 0.8 Hz and 4 Hz. In some embodiments, the filter is designed with a Kaiser window with beta of 6. In some embodiments, the filter has 420 taps.

In some embodiments, a covariance matrix is calculated from the filtered data and stored in a FIFO buffer of size M. Next, the eigenvector of the sum of the covariance matrices in the FIFO buffered are found. Then, any sign changes are corrected for. Finally, the input frame is projected onto the sign corrected eigenvector, resulting in the demodulated frame. FIG. 9C depicts the demodulation process as described above. FIG. 9D depicts the demodulation process of systems with respiration based heart processing.

In various embodiments, many different algorithms can be used alone or in combination to isolate different physiological motion signals from the combined physiological motion signal and surrounding noise. These include, but are not limited to fixed filters as disclosed in U.S. Provisional App. No. 61/141,213 which is incorporated herein by reference in its entirety, adaptive filters as disclosed in U.S. Provisional App. No. 61/141,213 which is incorporated herein by reference in its entirety, matched filter, wavelet, empirical mode decomposition as disclosed in U.S. Provisional App. No. 61/125,023, which is incorporated herein by reference in its entirety, blind source separation as disclosed in U.S. Provisional App. No. 61/141,213 which is incorporated herein by reference in its entirety, Direction of Arrival (DOA) information as disclosed in U.S. Provisional App. No. 61/125,020, which is incorporated herein by reference in its entirety and in as disclosed in U.S. Provisional App. No. 61/141,213 which is incorporated herein by reference in its entirety, independent component analysis as disclosed in U.S. Provisional App. No. 61/141,213 which is incorporated herein by reference in its entirety, smart antennas as disclosed in U.S. Provisional App. No. 61/141,213 which is incorporated herein by reference in its entirety, and empirical mode decomposition as disclosed in U.S. Provisional App. No. 61/125,023, which is incorporated herein by reference in its entirety as disclosed in U.S. Provisional App. No. 61/141,213 which is incorporated herein by reference in its entirety. One embodiment used to isolate the heart signal from the combined signal is first extracting the respiratory signal, then subtracting this from the combined signal, and then filtering (either fixed or adaptive filtering) the remainder signal to obtain the relatively smaller heart signal. Another embodiment used to isolate the heart signal is cancelling harmonics of respiration signal combined with minimum mean squared error estimation.

For some applications, it is important to determine the beginning and end of breaths or beats, or to determine the peak of each breath or beat, such that breath-to-breath or beat-to-beat intervals can be calculated. Peak detection involves finding local maxima and minima that meet various defined properties in a signal. There are many variations of peak detection that can be used in various embodiments of this device, including, but not limited to maxima above a threshold preceded and followed by minima below a threshold (in various embodiments, the threshold can be fixed or can be based on previous peaks and valleys); perform a least-squares quadratic fit between peaks, valleys, and/or zero-crossings and determine the peak of this function (this method provides interpolation). In some embodiments, the above algorithms can be performed after removing the baseline variation of the signal. In some embodiments, the peak detection algorithm can include finding zero-crossings of the derivative of the signal. In some embodiments, it is also possible to use zero-crossings to estimate the interval of each breathing cycle, by selecting either the positive or negative zero-crossings. In some embodiments, valley detection can replace peak detection.

For some applications, it is desirable to estimate the rate of the cardiopulmonary signals. In some embodiments, the rate of the signals can be estimated in the time domain, using peak detection as disclosed in U.S. Provisional App. No. 61/128,743 which is incorporated herein by reference in its entirety as described above or zero-crossing detection as disclosed in U.S. Provisional App. No. 61/141,213 which is incorporated herein by reference in its entirety, and calculating either the time required for a specific number of peaks, by calculating the average peak-to-peak interval, or by determining the number of peaks in a specified time period. The rate can also be estimated in the frequency domain. This can be calculated as the Short Time Fourier Transform, using a window that can be of predetermined length or a variable length depending on the signal. The respiration rate can also be calculated in the frequency domain using the instantaneous frequency as calculated with the Hilbert-Huang Transform after applying empirical mode decomposition as disclosed in U.S. Provisional App. No. 61/125,023, which is incorporated herein by reference in its entirety.

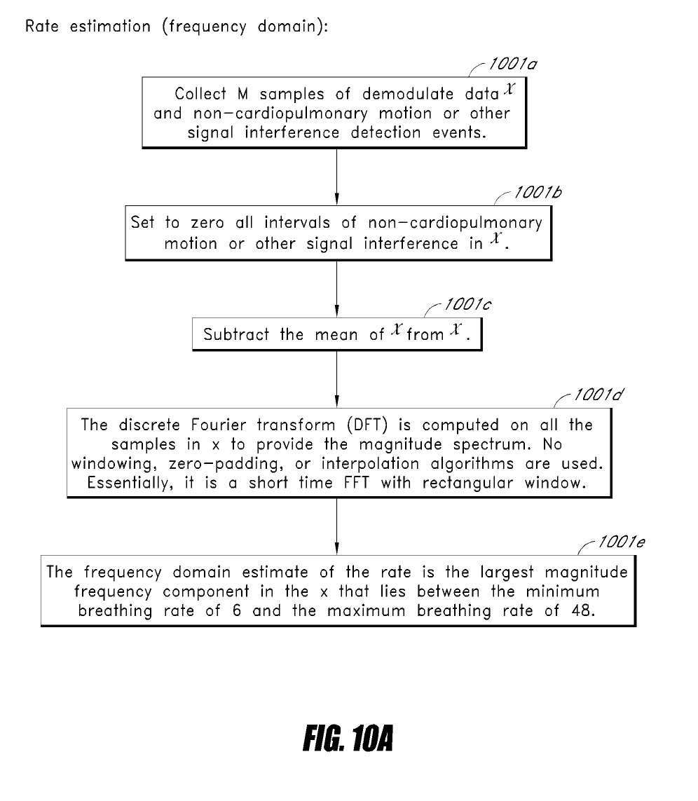

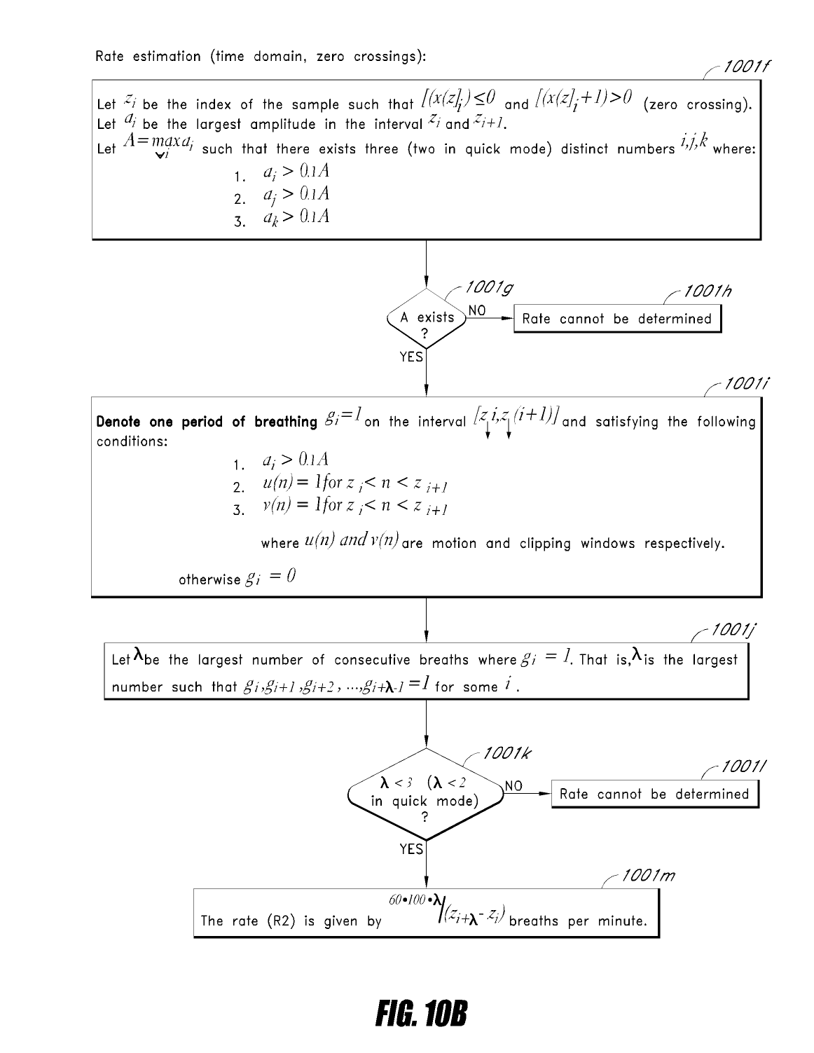

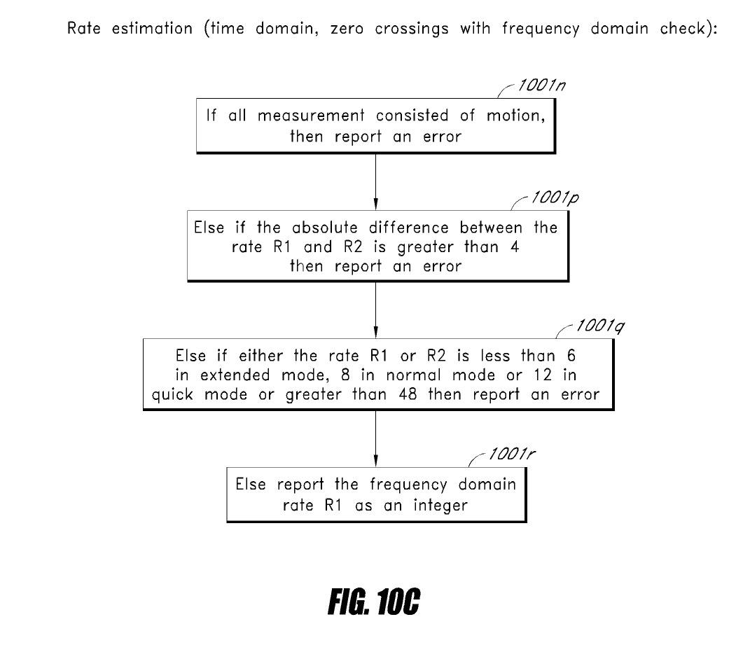

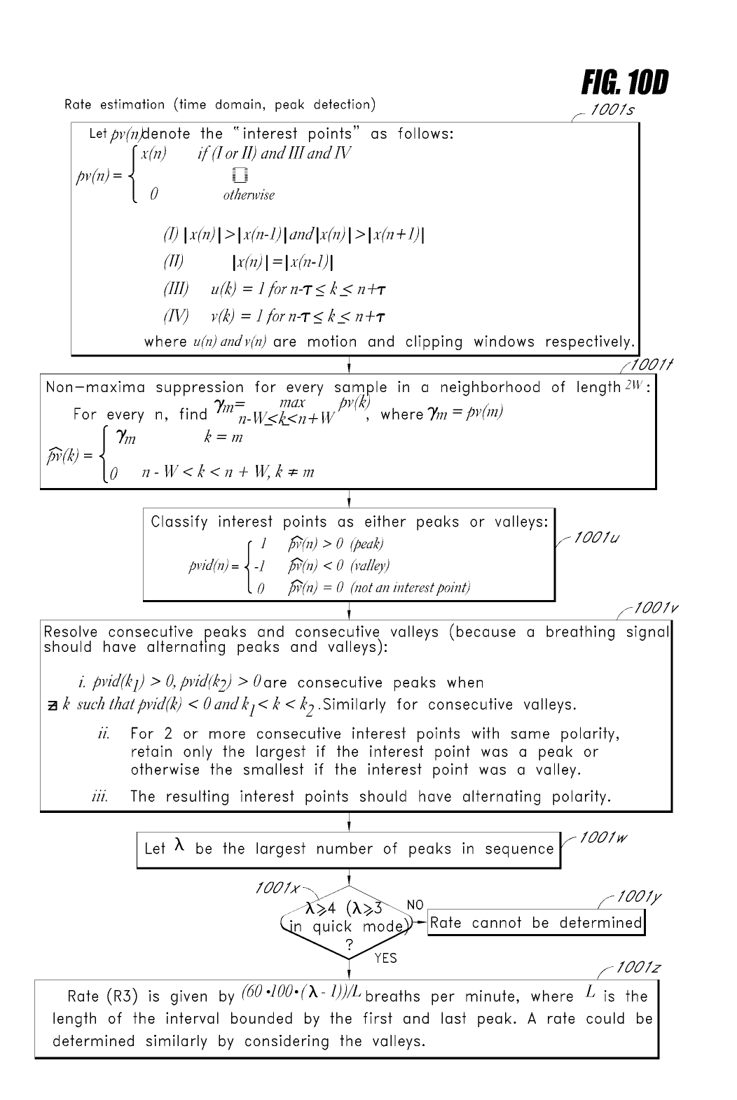

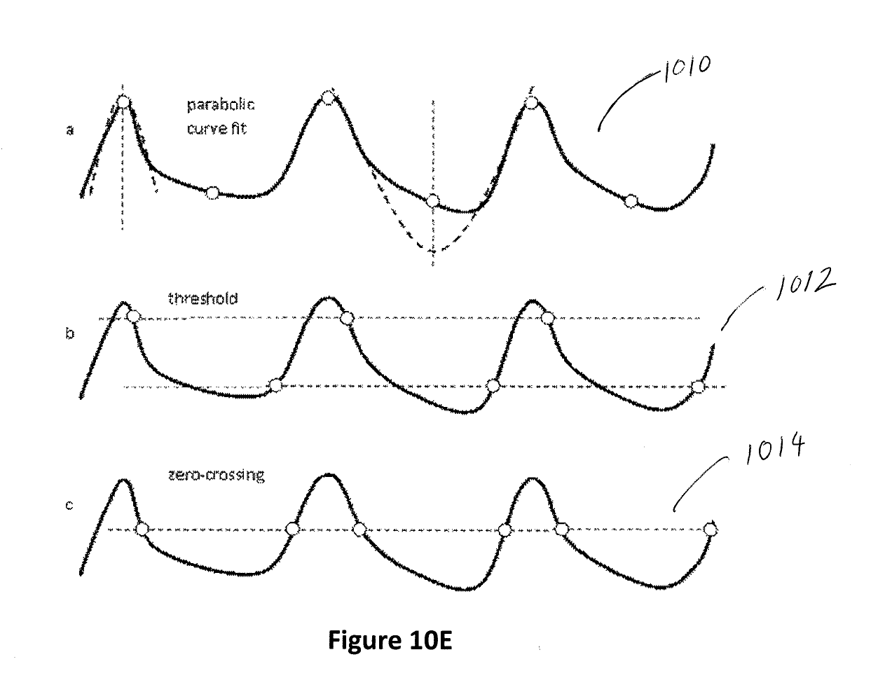

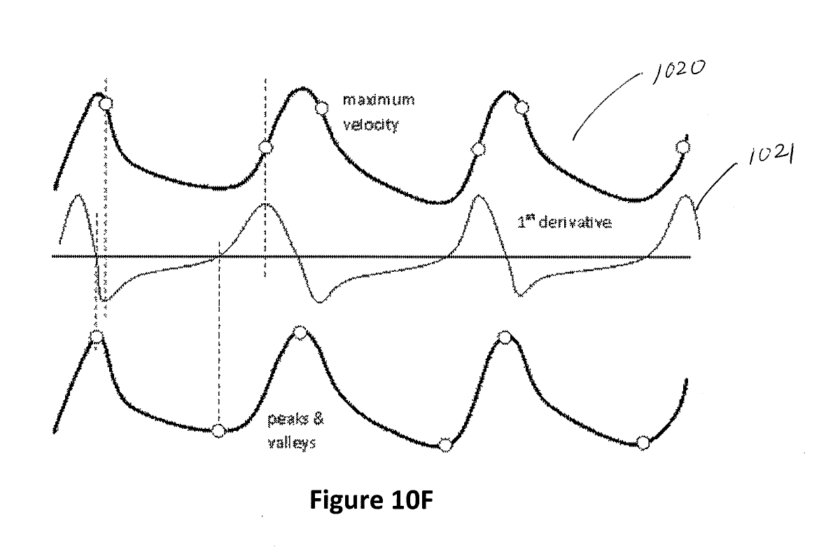

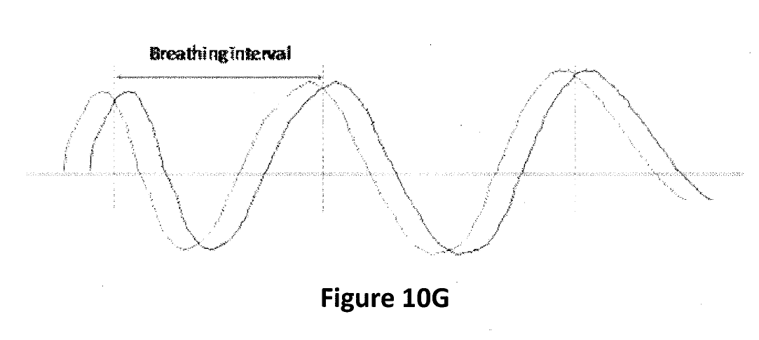

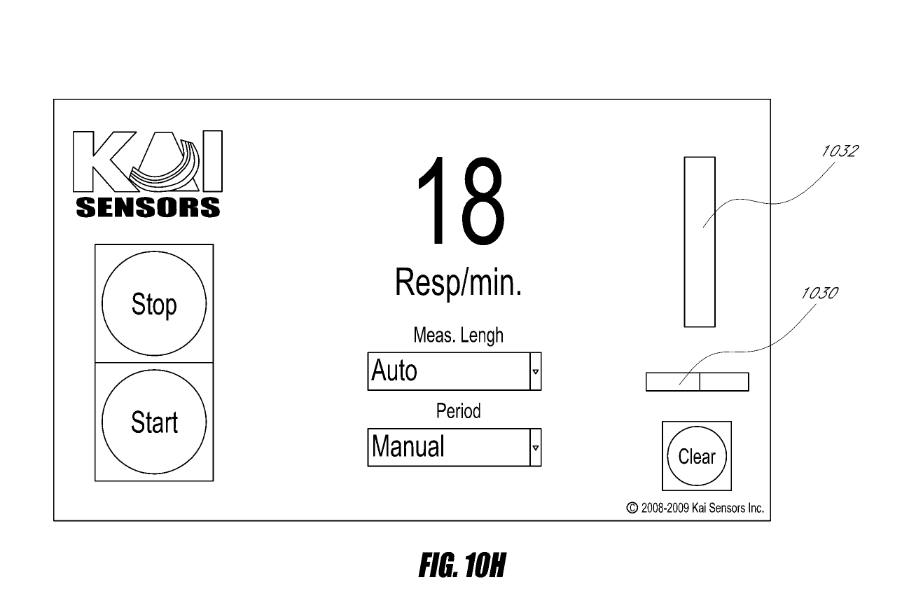

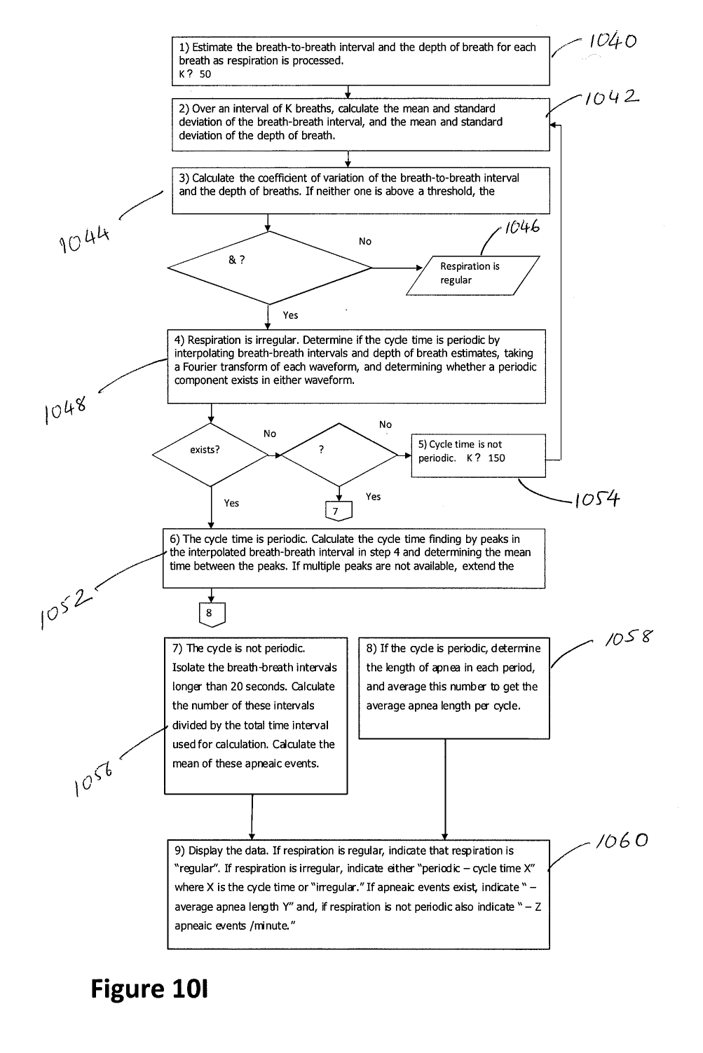

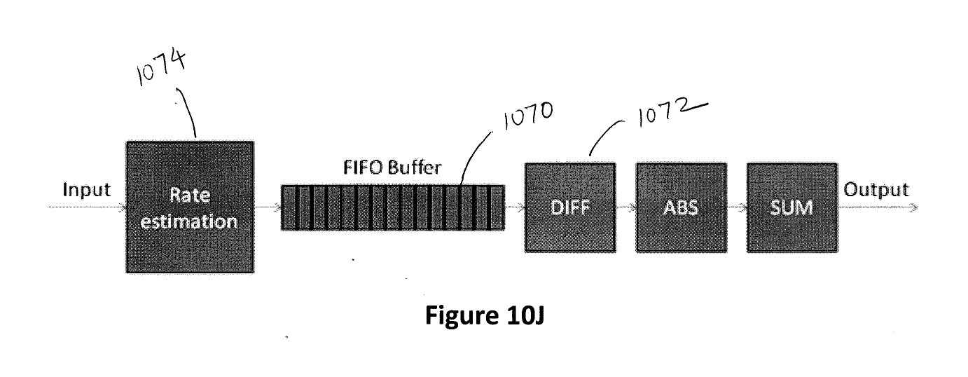

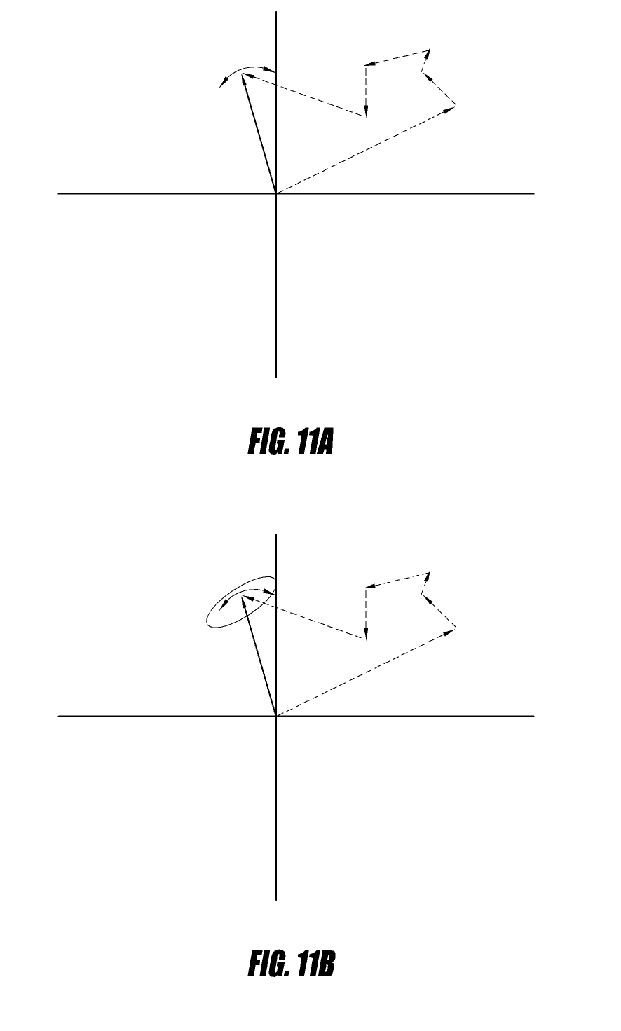

An embodiment of a frequency domain rate estimation algorithm is further described below and illustrated in FIG. 10A. The frequency domain rate estimation comprises the following steps:
1. Collect M samples of demodulated data x and non-cardiopulmonary motion or other signal interference detection events as shown in block 1001a, where M is the number of samples for rate estimation and in various embodiments can be 1440, 2880, 4320 or some other number.
2. Set to zero all intervals of non-cardiopulmonary motion or other signal interference in x as shown in block 1001b.
3. Subtract the mean of x from x as shown in block 1001c.
4. Determine the rate using frequency domain information as follows:
   i. A Fourier transform (e.g., discrete Fourier transform) is computed for all the samples in x to provide the magnitude spectrum as shown in block 1001d. No windowing, zero-padding, or interpolation algorithms are used. In some embodiments, the Fourier transform can include a short time fast Fourier transform with rectangular window.
   ii. The frequency domain estimate of the rate is the largest magnitude frequency component in x as shown in block 1001e. In various embodiments, the frequency domain estimate of the rate can be the largest magnitude frequency component that lies between a breathing rate of 6 and a breathing rate of 48.

An embodiment of a time domain rate estimation algorithm is further described below and illustrated in FIG. 10B. The time domain rate estimation comprises the following steps:
1. Collect M samples of demodulated data x and non-cardiopulmonary motion or other signal interference detection events as shown in block 1001a of FIG. 10A, where M is the number of samples for rate estimation and in various embodiments can be 1440, 2880, 4320 or some other number.
2. Set to zero all intervals of non-cardiopulmonary motion or other signal interference in x as shown in block 1001b of FIG. 10A.
3. Subtract the mean of x from x as shown in block 1001c of FIG. 10A.
4. Determine the rate using time domain information as follows:
   a. Let zi be the index of the sample such that x(zᵢ)≦0 and x(zᵢ₊ᵢ)>0 thereby identifying positive zero crossings in the input frame as shown in block 1001f. In various embodiments, negative zero crossings can also be identified.
   b. Let aᵢ be the largest amplitude in the interval zᵢ and zᵢ₊₁.
   c. Let A=max aᵢ for all i, such that there exists three (two in quick mode) distinct numbers i, j, k where:
      i. aᵢ>0.1A
      ii. aⱼ>0.1A
      iii. aₖ>0.1A
   d. If in block 1001g it is determined that there exists no such A, then the rate cannot be determined as shown in block 1001h.
   e. Otherwise denote one period of breathing gᵢ=1 on the interval [zᵢ, zᵢ₊₁] and satisfying the following conditions as shown in block 1001i:
      i. aᵢ>0.1 A
      ii. u(n)=1 for zᵢ<n<zᵢ₊₁
      iii. v(n)=1 for zᵢ<n<zᵢ₊₁
   where u(n) and v(n) are motion and clipping windows respectively.
   f. Otherwise gᵢ=0.
   g. Let λ be the largest number of consecutive breaths where gᵢ=1. That is λ is the largest number such that gᵢ, gᵢ₊ᵢ, gᵢ₊₂, gᵢ₊₃, . . . , g_{i+λ-1}=1 for some i, as shown in block 1001j.
   h. If in block 1001k, it is determined that λ<3 (λ<2 in quick mode), then the rate cannot be determined, otherwise the rate is given by (60×100×λ)/(z_{i+λ}−zᵢ) breaths per minute as shown in block 1001m.

In various embodiments, the rate estimation algorithm can use both the frequency domain estimate and the time domain estimate to determine the respiration rate as illustrated in FIG. 10C. An advantage of employing the two methods simultaneously is two-fold. First, comparing the result of these two approaches will help determine if breathing is regular. Secondly, the redundancy introduced by employing two algorithms can help in mitigating risk of inaccuracies in determining the respiratory rates. For example, with reference to the embodiments of the time domain rate estimation algorithm and the frequency domain rate estimation algorithm described above, if the algorithms determined that all measurements consisted of non-cardiopulmonary motion as shown in block 1001n or other signal interference then an error message is reported. In some embodiments, if the difference between the rates estimated by the two algorithms is greater than 4 as shown in block 1001p then an error is reported. In some embodiments, if the rate estimated by either the frequency domain rate algorithm or the time domain rate algorithm is less than 6, then an error is reported as shown in block 1001q. In some embodiments, if the rate estimated by either the frequency domain rate algorithm or the time domain rate algorithm is less than 8 or 12, then an error is reported as shown in block 1001q. In some embodiments, if the rate estimated by either the frequency domain rate algorithm or the time domain rate algorithm is greater than 48, then an error is reported. In various embodiments if the rate estimated by the either the frequency domain rate algorithm or the time domain rate algorithm is between the range of 12 and 48, then the frequency domain rate is reported. In some embodiments, the rate estimated by the either the frequency domain rate algorithm or the time domain rate algorithm can be between the range of 8 and 48 or 6 and 48 to be considered as accurate.

An embodiment of a peak detection algorithm to estimate a rate is further described below and illustrated in FIG. 10D.
1. Collect M samples of demodulated data x and motion detection events as shown in block 1001a of FIG. 10A, where M is the number of samples for rate estimation and in various embodiments can be 1440, 2880, 4320 or some other number.
2. Set to zero all intervals of non-cardiopulmonary motion or other signal interference in x as shown in block 1001b of FIG. 10B.
3. Subtract the mean of x from x, as shown in block 1001c of FIG. 10C.
4. The time domain estimate of the rate is found as follows:
   a. Let pv(n) denote the interest points as follows:

$$pv ( n ) = \{ \begin{matrix}x ( n ) & if ( I or II ) and III and IV \\ & □ \\ 0 & otherwise\end{matrix} \\ ( I ) \vert x ( n ) \vert > \vert x ( n - 1 ) \vert and \vert x ( n ) \vert > \vert x ( n + 1 ) \vert \\ ( II ) \vert x ( n ) \vert = \vert x ( n - 1 ) \vert \\ ( III ) u ( k ) = 1 for n - τ \leq k \leq n + τ \\ ( IV ) v ( k ) = 1 for n - τ \leq k \leq n + τ$$

where u(k) and v(k) are motion and clipping windows respectively, as shown in block 1001s.
b. Non-maxima suppression for every sample in a neighborhood of length 2W is performed, as shown in block 1001t by the following method:

$$For every n , find {γ}_{m} = {max}_{n - W \leq k \leq n + W} pv ( k ) , where {γ}_{m} = pv ( m )$$

$${pv}^{⋒} ( k ) = \{ \begin{matrix}{γ}_{m} & k = m \\ 0 & n - W \leq k \leq n + W \cdot k \neq m\end{matrix}$$

c. Classify interest points as either peaks or valleys, as shown in block 1001u, by using the following equation:

$$pvid ( n ) = \{ \begin{matrix}1 & pv ( n ) > 0 & ( peak ) \\ - 1 & pv ( n ) < 0 & ( valley ) \\ 0 & pv ( n ) = 0 & ( not an interest point )\end{matrix}$$

d. Resolve consecutive peaks and consecutive valleys, as shown in block 1001v, since a breathing signal should have alternating peaks and valleys. In various embodiments, the resolution can be done as follows:
   i. pvid(k₁)>0, pvid(k₂)>0 are consecutive peaks when [symbol] k such that pvid(k)<0 and k₁<k<k₂. A similar method can be followed to identify consecutive peaks.
   ii. For 2 or more consecutive interest points with same polarity, retain only the largest if the interest point was a peak or otherwise the smallest if the interest point was a valley.
   iii. The resulting interest points should have alternating polarity.
e. Let λ be the largest number of peaks in sequence. If λ<4 (λ<3 in quick mode), then the rate cannot be determined, otherwise the rate is given by 60×100×λ/L breaths per minute, where L is the length of the interval bounded by the first and last peak. A rate could be determined similarly by considering the valleys.

In some embodiments, the respiratory rate is calculated from the sinusoid calculation by fitting a sinusoidal equation to each respiratory cycle, multiple cycles, or cycles over a period of time at least as long as the longest expected respiration period, using least mean square methods or maximum likelihood estimator methods.

In some embodiments, a rate is estimated by counting repeating key points. Key points are points in a respiration cycle that are identifiable using specific algorithms. In some embodiments, key points can be, but are not limited to: peaks, valleys, zero crossings, points of fastest change, points of no change and points where there is the greatest change in direction.

In some embodiments, each peak is found by using a parabolic curve fit as shown by trace 1010 of FIG. 10E, and identifying the peak as the maxima of the parabolic curve, or the center of the parabolic curve. In some embodiments, a peak is found using a high threshold value, and finding the highest point above that threshold. In some embodiments, where there are multiple peaks in a cycle, the peak can be the highest, first, middle or last in the cluster of peaks. This cluster can include, but is not limited to, one or more of the following scenarios: peaks that happen within a period of time shorter than the respiration cycle, peaks that are clustered in a time period determined in the frequency domain, peaks between which the signal does not cross zero, peaks between which the signal does not cross a threshold, peaks with an amplitude much less than the respiration signal amplitude, and/or peaks following a known clustering pattern. Valley key points can be found the same way as peaks by inverting the polarity of the signal, or by identifying minima and low thresholds rather than maxima and high thresholds. In some embodiments, a first derivative with peak finding methods above can be used to identify the points of peak velocity as illustrated by trace 1020 and trace 1021 of FIG. 10F. In some embodiments, a first derivative and zero crossing can be used to identify find peaks and valleys. These zero crossings of the derivative happen twice a cycle—once for maximum inhale and once for maximum exhale. In some embodiments, after peak-detection, the respiratory rate is estimated from the time between peaks. In some embodiments, the time between key points of a respiration cycle is the respiration period, with rate being the inverse of period. In some embodiments, zero crossings are expected to occur twice a cycle, and every-other crossing is ignored in rate-finding. In some embodiments, zero crossings for an expected cycle duration are ignored. In some embodiments, the rate at which zero crossings occur is calculated, and this value is divided by two to determine the respiratory rate. In some embodiments, only the negative-to-positive zero crossings are considered. In some embodiments, only the positive-to-negative zero crossings are considered. In some embodiments, the rate is calculated from negative-to-positive zero crossings and from positive-to-negative zero crossings, and the two rates are averaged. In some embodiments, a rate is calculated from every other zero crossing, then calculated from the alternate zero crossings, and then the two rates are averaged.

In some embodiments, peak-finding algorithms are used. There are many variations on peak-finding algorithms, including, but not limited to:
Perform a least-squares parabolic curve fit to the data between two peaks, two valleys, or two zero-crossings and determine the peak or valley of this function. See e.g. trace 1010 of FIG. 10E.
Find a maxima above a threshold followed by a minima below a threshold and define the maxima as a peak (in various embodiments, these thresholds can be fixed or can be based on previous peaks and valleys); inversely, find a minima with absolute value above a threshold followed by a maxima above a threshold and define the minima as a valley as illustrated by the trace 1012 of FIG. 10E.
Find a maxima above a threshold between two minima, each below a threshold to determine a peak (in various embodiments, these thresholds can be fixed or can be based on previous peaks and valleys) and determine that is the peak; inversely, find a minima with absolute value above a threshold between two maximum, each above a threshold to determine a valley
Find the zero-crossings of the zero-mean signal and label the largest absolute values between every two zero-crossings as peaks or valleys as illustrated by trace 1014 of FIG. 10E.
Find the zero-crossings of the derivative of the function, and determine whether they are peaks or valleys as illustrated in FIG. 10F.
Find maxima above a threshold in amplitude and separated by a time greater than a threshold such that if two maxima are above the amplitude threshold but closer in time than the second threshold, they can not be counted as 2 peaks. The same process can be performed for minima.

In some embodiments, the respiratory rate can be determined in the I/Q plane, without demodulating the signal. In some embodiments, specific parts of the respiration cycle in the I/Q plane can be marked with key points. In some embodiments, the key points are selected by points in the signal path that have the greatest change in direction, speed (length), or both. In some embodiments, the key points are selected by a series of points on a path that have zero or small values for speed (length). If a significant number of key points occur in an area, an event area is formed. In some embodiments, detection occurs when the signal moves into, leaves or stays in the event area for a certain period of time.

A wavelet transform provides a frequency and transient analysis of a signal. In some embodiments, the demodulated signal uses a wavelet transform to analyze the rate information. In some embodiments, event warnings (such as non-respiratory motion detection and low signal power) can be used to mark sections of the transform to be ignored during analysis. In some embodiments, the wavelet basis function can be tailored to match certain respiration wave shapes. In some embodiments, rate can be the result of the strongest or longest frequency. In some embodiments, the rate can be the average of the most prominent frequencies with or without weighting due to relative length and/or strength. In some embodiments, rate can be provided as two rates, highest and lowest, if the transform shows a range of rates for irregular breathing.

In some embodiments, a signal that indicates irregular breathing can be separated into sections in which breaths are similar, and these rates for each section can be estimated separately. In some embodiments, the wavelet power spectrum can separate sections in time by frequency and power. When time sections are separated by frequency and power, one can derive rate and amplitude irregularities in breathing. In some embodiments, the sections can be separated by empirical mode decomposition, which provides instantaneous frequency data. In some embodiments, amplitude thresholds can be used to mark when there is a change in amplitude. In some embodiments, the separate sections are analyzed separately for rate, and the result could include both numbers, an average of the numbers, or a weighted average of the numbers, depending on length of time.

In some embodiments, the irregular breathing can have some periodic pattern that can be found and displayed using wavelets or analysis of the repetition of rate and amplitude of a sequence of sections. In some embodiments, common patterns can be recognizable and comparable to certain pulmonary conditions. In some embodiments, the wavelet power spectrum of certain pulmonary conditions can produce a pattern of frequency and power over time. This pattern can be cross correlated to a wavelet power spectrum of a patient with irregular breathing. In some embodiments, the patient's pattern can be matched with a pattern from a library of patterns, indicating particular pulmonary conditions to indicate the presence of that particular pulmonary condition. In some embodiments, the patterns in the library can be time stretched, time shifted, frequency stretched or frequency shifted to find a match. In some embodiments, an index can be made for a particular condition depending on the correlation to the library pattern due to matching of power measured over one or more respiration cycles.

In some embodiments, the rate of the respiratory signal can be estimated in the time domain by tracking the points where a signal crosses a time-delayed version of itself as shown in FIG. 10G. In some embodiments, the time delay can be adaptively set, possibly by means of spectrum analysis or pre-learned patient-data, to ensure that the delay is long enough to suppress small variations or noise while short enough delay can compare the correct cycles and account for irregularity in the breathing period.

In some embodiments, the time-domain signal can be pre-conditioned before rate estimation. In some embodiments, when peak to peak intervals are used to estimate rate, the envelope of the signal can be normalized to improve the rate-estimation algorithm based on peak-finding. In some embodiments, if the signal is clipping, then the clipping period can not be used for estimating rate. In some embodiments, when the signal is clipping, the transmitting power can be adjusted so that receiving power is within the proper range.

In some embodiments, each breath can be identified, and then the time between the onset of breaths can be used to estimate the respiratory rate.

In some embodiments, a breath can be inferred by the ratio of the duration of an inhale to the duration of an exhale. In some embodiments, a segment of a signal is determined as a candidate breath by detection of a peak and a valley, calculating the ratio of the duration of the inhale to the duration of exhale, and determining whether the ratio lies within a certain interval, in which case the segment is declared a breath. In various embodiments, the interval is determined by various methods including, but not limited to, a fixed interval determined from a base population, an interval based on the patient's height, weight, and other information, or an adaptive interval based on prior observations for a given patient.

In some embodiments, features that highlight the core aspects of a breathing signal are extracted from a database of breaths. In some embodiments, these features include the inhale time to exhale time ratio, the length of pauses in breathing, the ratio of the length of a pause in breathing to the breathing period, the depth of breath, the inflection points of the breath, and/or the mean, variance and kurtosis of the breath. In some embodiments, these features include particular coefficients in the wavelet decomposition of the signal or particular coefficients of the Fourier transform of the signal. In various embodiments, the same features extracted from the database of breathing signals are again extracted from the new signal being considered. In some embodiments, the new signal features are compared to the database of features, and if a match is found, then the signal is labeled as a breath. In some embodiments, the peak of the breath is identified based on information in the database.

In some embodiments, the signal is correlated with a database of breathing signals. If the correlation coefficient is above a certain threshold for a breath in the database, the signal is identified as a breath. In some embodiments, an autocorrelation is performed to determine the existence of a pattern in the signal, and this identified pattern is extracted and correlated with new samples to locate the new breaths.

In some embodiments in which the carrier frequency is high enough that a respiration traces at least a full circle in the I/Q plane, a constant modulus-detection algorithm can be used for a breath detection. In some embodiments, a constant modulus signal can indicate a breath. In some embodiments, the constant-modulus is determined by the distribution of the modulus of the samples in the I/Q plane from a center or the origin when the signal is zero mean. In some embodiments, the signal is found to be constant-modulus when the distribution has small variance.

In some embodiments, a patient-specific library of breath shapes can be obtained during a supervised initial period of use, and then these shapes can be matched to breaths with a pattern recognition algorithm. In some embodiments, the distance between breaths can be used to estimate the respiratory rate. In some embodiments, during this initial period of use, one or more of the patient's breath shapes can be identified and recorded, and after the initial training sequence, data can be buffered and cross correlated with the training breath or breaths. In such embodiments, the subset of points with the highest correlation represents a breath.

In some embodiments, the breath to breath rate variability can be identified by measuring the time taken per breath, and calculating the variability in this variable. In some embodiments, an average rate can be identified by measuring the time taken over N breaths where N is greater than 1.

In some embodiments, the autocorrelation of a subset of data can be used to determine the respiratory rate. In various embodiments, the segment of data can be a fixed length, or set adaptively based on the respiratory rate. In some embodiments, the respiratory rate is estimated from the autocorrelation by taking the first large peak from zero.

In embodiments where breath-breath intervals are obtained, the rate can be computed as any of the following: the most recent breath-breath interval, the mean breath-breath interval over a specified time interval, the median of breath-breath intervals over a specified time interval, a weighted average of breath-breath intervals over a specified time interval, the mean breath-breath interval over a specified number of breaths, the median of breath-breath intervals over a specified number of breaths, or a weighted average of breath-breath intervals over a specified number of breaths. The number of breaths or the time interval over which breaths are averaged can be fixed or it can vary adaptively based on the breath-breath interval or the regularity of respiration.

In various embodiments, signal processing can determine both the points of inhalation and exhalation and count them over time. For every block of data, a respiration rate can be calculated and buffered based on detected inhalation or exhalation events. The rates can be stored until a designated number of consecutive inhalation events or exhalation events are detected (e.g., 3, 5, 10, 15, 20). In some embodiments, 3 can be set as the default rate. In some embodiments, the device can be configured to return or display the median value of the inhalation and exhalation events found. In various embodiments, if an interruption (e.g., non-physiological motion or other interfering signal) is detected during the reading, any respiration rate values stored in the buffer will be cleared and no values will be buffered until the interruption has ceased as disclosed in U.S. Provisional App. No. 61/128,743 which is incorporated herein by reference in its entirety.

In various embodiments, instead of calculating the respiration based on blocks of data, it is also possible to calculate the respiration based on each inspiration peak to inspiration peak interval as disclosed in U.S. Provisional App. No. 61/128,743 which is incorporated herein by reference in its entirety. In some embodiments the system (e.g., a spot-check monitor) could measure a specified number of peaks before displaying a respiration rate, or it could measure for a specified time interval. In various embodiments, the time interval or the number of peaks could be automatically extended if the measured respiration rate is varying more than a few breaths per minute to ensure an accurate reading of in irregular rate as disclosed in U.S. Provisional App. No. 61/204,880 which is incorporated herein by reference in its entirety.

A non-contact spot check of respiratory parameters can have a measurement mode in which the time interval over which respiration is measured is automatically selected. In some embodiments, the measurement length is calculated based on signal quality and the respiratory waveform, such that the respiratory waveform is only used to estimate the rate when a signal with adequate signal quality is used. In this case, the device can extend the measurement signal until a long enough respiration signal with adequate signal quality can be obtained. The automatic selection of measurement mode can also be used in conjunction with respiratory pattern irregularity detection, such that intervals are extended if the subject is breathing irregularly, so an accurate estimation of the subject's respiratory rate can be provided. Embodiments of automated selection of the measurement interval include the following and various combinations of the following: the measurement continues until it obtains N seconds of good-quality data; the measurement continues until it obtains N continuous seconds of good-quality data; the measurement continues until it obtains M complete breath-to-breath intervals of good-quality data; the measurement continues until it obtains M consecutive, complete breath-to-breath intervals of good-quality data; the measurement continues until it obtains N consecutive seconds of good-quality data and at least M consecutive, complete breath-to-breath intervals; the measurement continues until it obtains N consecutive seconds of good-quality data or M consecutive, complete breath-to-breath intervals, whichever comes first; if breathing obtained in N consecutive seconds of good-quality data indicates irregular breathing, extend the measurement until a) breathing appears to be regular (the irregular was a false alarm), b) a periodic pattern repeats, or c) T seconds have passed and breathing is still irregular and non-periodic; and if breathing obtained in M consecutive breath-to-breath intervals of good-quality data indicates irregular breathing, extend the measurement until a) breathing appears to be regular (the irregular was a false alarm), b) a periodic pattern repeats, or c) T seconds have passed and breathing is still irregular and non-periodic. Various embodiments of automated measurement length selection can or can not have an associated time-out, where the device provides an error code or error message if it was not able to obtain the required length of good-quality data in that time. In various embodiments N can have values between approximately 10 seconds and approximately 150 seconds. For example, in various embodiments N can be 15 seconds, 30 seconds, 60 seconds or 120 seconds. In various embodiments, M can have values between 2 to 10 breaths. For example, in various embodiments, M can be 3 or 4. In various embodiments T can have values between approximately 30 seconds to approximately 10 minutes. For example, in some embodiments, T can be approximately 3 minutes. Various embodiments of automated measurement length selection can run until an answer is obtained. In some embodiments, a time-out can be implemented to limit the length of time for which the automated measurement lasts. The time-out can be a fixed time, a user-settable time, or it can be determined by other equipments. The time-out can be determined by other equipment if the respiratory spot check is integrated with other vital signs spot checks such as blood pressure or temperature; the time-out can come at the completion of these measurements. In one embodiment, the same button is used to initiate measurement of all the vital signs. In some embodiments, the respiration spot check device can determine the rate with as much data of usable quality as is obtained during the other vital signs measurements.

In some embodiments, the measurement interval can be increased if the respiration is irregular, and decreased if the respiratory rate is very regular.

Any implementations can include real-time audio feedback for some or all types of poor signal quality. For example, a ticking sound can indicate low received signal power, such that the user knows he/she needs to reposition the sensor, and does not wait indefinitely for a reading when the sensor is improperly placed.

Any implementation can make use of a page, automated message, SMS, email or otherwise to alert attending health care professionals if excessive alerts are occurring so the sensor can be repositioned properly or the patient can receive the necessary medical attention that is causing the alert triggers.

In some embodiments, the respiration spot check device can automatically choose the interval of measurement, given heuristics on the minimum quality of the data acquired. For example, after the respiration spot check device has acquired a minimum number of breathing intervals (in some embodiments, described as inspiration peak to inspiration peak) or has acquired enough data of usable quality for a specified length of time, it can automatically halt the measurement and return a rate. FIG. 10H illustrates a screen shot of an embodiment of a display associated with a radar based sensor device that is configured to operate in the Auto Mode. The screen shot of the display associated with this method can include a graphic 1030 (e.g. a horizontal bar) that fills to indicate the time elapsed and the time remaining and a graphic 1032 (e.g. a vertical bar) that fills to indicate the number of breathing intervals that have been obtained with sufficiently high quality. The rate returned can be calculated using methods in the time domain, frequency domain, or any combination thereof.

In some embodiments, a spot-check monitor including the radar-based physiological motion sensor could measure a specified number of peaks before displaying a rate as disclosed in U.S. Provisional App. No. 61/128,743 which is incorporated herein by reference in its entirety and in U.S. Provisional App. No. 61/137,532 which is incorporated herein by reference in its entirety. The spot-check monitor could measure a user-selectable number of peaks (e.g., 3, 5, 10, 15) for a certain time interval (e.g., 10 seconds, 15 seconds, 20 seconds, 30 seconds, 45 seconds, 60 seconds, or other time interval) as disclosed in U.S. Provisional App. No. 61/128,743 which is incorporated herein by reference in its entirety and in U.S. Provisional App. No. 61/137,532 which is incorporated herein by reference in its entirety.

In various embodiments of the system, the software that is executable by a processor can automatically extend the time interval or number of peaks included for a rate estimate if respiration is irregular or varying more than a few breaths per minute as disclosed in U.S. Provisional App. No. 61/128,743 which is incorporated herein by reference in its entirety. In some embodiments, the software that is executable by a processor can only provide a respiratory rate if variability in rates is low over the measurement interval as disclosed in U.S. Provisional App. No. 61/128,743 which is incorporated herein by reference in its entirety. In some embodiments, the software that is executable by a processor can provide an indication of the level of variability as disclosed in U.S. Provisional App. No. 61/128,743 which is incorporated herein by reference in its entirety.

Information regarding the regularity or irregularity of a respiratory waveform can be calculated and displayed or communicated. Irregularity estimation can utilize any respiratory waveform, including, but not limited to, those obtained by the system 100, Doppler radar, ultra wide-band radar, impedance pneumography, chest straps, airflow measurements, and load cells. When nurses perform the observational portion of a respiratory assessment, they assess the rate, regularity, and depth of respiration. Many currently available technologies provide a respiratory rate, and some provide information on depth of breath. However, none provide information on the regularity or irregularity of respiration.

Various embodiments of the assessment of the regularity of respiration are intended to identify periods of apnea, periodic breathing, or Cheyne-Stokes respiration. In some conditions, periods of apnea do not occur in regular cycles, but periodic breathing and Cheyne-Stokes respirations typically have a regular cycle length. According to the literature, the cycle length of periodic breathing varies from less than 10 seconds for infants with high respiratory rates, to 150 seconds for patients with severe congestive heart failure (CHF). Other conditions that can cause periodic breathing include, but are not limited to, opioid overdose, altitude acclimation, sleep, and pulmonary hypertension. Irregularity in breathing can occur in the amplitude, or depth, of breaths and in the duration of breaths.

In some embodiments, the respiratory waveform can be analyzed by performing an auto-correlation function. Auto-correlation is a method to find repeating patterns within a signal by comparing a signal with itself over time. In some embodiments, the sampled section is much longer than the expected period respiration repetition. In some embodiments, the sample section is close to the length of the respiration repetition. In some embodiments, the autocorrelation function can be used to determine regularity or irregularity of the signal, and to find the periods of irregular breathing is irregular by finding peaks of the autocorrelation function. If breathing is irregular with no periodicity, there can be no major peaks in the autocorrelation function. If the breathing rate is regular, but the amplitude is modulated or there are periodic apneas, the first major peak in the autocorrelation function can be the respiratory period, and the second major peak that is not a multiple of the respiratory period can be the period of the periodic breathing. Thus, in some embodiments, the autocorrelation function can be used to determine the regularity of breathing, the respiratory rate, and period of periodic breathing.

In some embodiments, a wavelet transform function is utilized to create an index of repeating patterns in the respiration signal. A wavelet transform localizes a signal in both frequency and time by looking through a window that is both translated in time and frequency. In some embodiments, the transform reveals the longer repetition pattern of respiration. In some embodiments, the transform reveals the periodicity of the irregular breathing pattern, if it is periodic. In some embodiments, the transform reveals the power spectrum of the sections to compare the amplitude of the sections. In some embodiments, both frequency and amplitude of the different sections are analyzed. In some embodiments, the patterns of the transform can be compared to known patterns created by particular respiratory conditions to provide an initial diagnosis of a patient depending on the correlation with the known conditions.

Irregularities in respiration can be in the depth of respiration and in the length of the breath-to-breath interval. Therefore, the irregularity index can encompass one or more of assessment of the regularity of the breath-to-breath interval (or respiratory rate) and assessment of the amplitude of the respiratory signal (or the tidal volume or depth of breath). Various embodiments can present this information in one of the following ways: an indication of regularity or irregularity of respiration (a binary state); an integrated “regularity index” that compiles a variety of information about the regularity of respiration into a single number or a single bar graph; separate indications of the regularity of the breath-to-breath interval and of the depth of breath; and/or individual indications of several measures of irregularity. In some embodiments, the user can be able to select a method to display the information on regularity from options including some or all of the above methods.

Various embodiments of the respiratory regularity assessment algorithm can also assess the cycle length of periodic or Cheyne-Stokes respiration, either for an individual cycle or as an average over several cycles, and provide information on this. Various embodiments of the respiratory regularity assessment algorithm can also assess the length of apnea in each cycle or the average length of apnea over several cycles, and provide information on this. In some embodiments, the display can include information on the cycle length of periodic breathing, and the history of the cycle length of periodic breathing. In some embodiments, the display can include information about the length of apnea in each cycle, and the history of the length of apnea in each cycle.

In some embodiments, the irregularity can be assessed over several intervals, which can be described in time (seconds or minutes) or in number of breaths. For example, in some embodiments, the intervals can include 10 breaths, 30 breaths, and 60 breaths. In other embodiments, for example, the intervals can include 20 seconds, 60 seconds, and 150 seconds. In other embodiments, a single interval can be selected for estimation of irregularity, based on the longest time period or greatest number of breaths that would be relevant. In some embodiments, this value can be a default value (in some embodiments, 150 seconds) that can be changed by the user. In other embodiments, the value can be fixed.

Some embodiments can use breath-breath intervals in the calculations. In various embodiments, breath-to-breath intervals can be defined as the time between maximum inhalation points, the time between maximum exhalation points, the time between consecutive positive zero crossings, the time between consecutive negative zero crossings.

Irregularity in breath duration can be calculated from one or more of the following: standard deviation of breath-to-breath interval (or respiratory rate); frequency of apneaic events (absence of breaths longer than a threshold); or coefficient of variation of breath-to-breath interval (or respiratory rate).

Irregularity in breath depths can be calculated from one or more of the following: standard deviation of breath depths (or signal amplitude or tidal volume) or coefficient of variation of breath depths (or signal amplitude or tidal volume).

In embodiments which use respiratory rate for determination of irregularity, the respiratory rate is preferably calculated in a relatively short interval such that the rate does not average so many breaths that irregularity is not detected.

Various embodiments of the respiratory regularity assessment algorithm can determine whether irregular breathing is periodic. In various embodiments, one or more of the following methods can be used to determine whether irregular breathing is periodic:
Interpolate between the breath-breath interval calculations (with the data set encompassing the length of the interval vs. time, with the time point at the end of the breath for which the interval in which it was calculated) and perform the Fourier transform or calculate the power spectral density of the resulting waveform. Determine if it has a significant periodic component.
Interpolate between the breath-breath interval calculations (with the data set encompassing the length of the interval vs. time, with the time point at the end of the breath for which the interval in which it was calculated) and perform an autocorrelation. Determine if it has a significant periodic component.
Interpolate between the breath-breath interval calculations (with the data set encompassing the length of the interval vs. time, with the time point at the end of the breath for which the interval in which it was calculated) and determine peaks of the resulting waveform. Determine if the difference between the peaks is consistent by calculating the coefficient of variation of the difference between the peaks and determining whether it is low enough to indicate periodic breathing.
Identify the cessation of apneaic events, and determine the cessation-of-apnea to cessation-of-apnea intervals. Determine whether the difference between the cessation of apneas is consistent by calculating the coefficient of variation of the difference between the events and determining whether it is low enough to indicate periodic breathing by comparing to a threshold.

In some embodiments the methods described above (Fourier transform or PSD, autocorrelation, coefficient of variation) can be applied on the envelope of the respiration signal to determine the periodicity in the amplitude irregularities. In various embodiments, the envelope is determined by a variety of methods including, but not limited to, interpolating the peak amplitudes or squaring the signal and applying a low pass filter.

Various embodiments require multiple periods of irregular breathing to determine whether irregular breathing is periodic. In some embodiments, the interval used to calculate whether breathing is regular can be of adequate length for this determination. In other embodiments, that interval can be extended to ensure multiple periods are included. In some embodiments, the interval use to calculate irregularity can be doubled or tripled for this step.

In some embodiments, the algorithm can calculate the cycle time of periodic breathing or Cheyne Stokes respirations. One or more of the following methods can be used to calculate the cycle time.
Interpolate between the breath-breath interval calculations (with the data set encompassing the length of the interval vs. time, with the time point at the end of the breath for which the interval in which it was calculated) and perform the Fourier transform or calculate the power spectral density of the resulting waveform. Determine the frequency of the maximum power frequency component, and invert this to calculate the cycle length of periodic breathing
Interpolate between the breath-breath interval calculations (with the data set encompassing the length of the interval vs. time, with the time point at the end of the breath for which the interval in which it was calculated) and determine peaks of the resulting waveform. Calculate the average time difference between the peaks as the cycle length of periodic breathing.
Identify the cessation of apneaic events, and determine the cessation-of-apnea to cessation-of-apnea intervals. Calculate the average time difference between the cessation of apneas as the cycle length of periodic breathing.

In some embodiments, the length of apneaic events can be calculated. Algorithms to estimate this value include: isolate the breath-breath intervals longer than a threshold (in some embodiments, 20 seconds, or a user-settable value) and identify these as apneaic events with a length equal to the breath-breath interval; or identify the plateaus between the cessation of exhalation and the beginning of inhalation (or vice versa) that are longer than a threshold (in some embodiments, 20 seconds, or a user-settable value) and identify these as apneaic events with a length equal to the duration of the plateau, or pause in breathing.

In some embodiments, the frequency of non-periodic apneaic events can be calculated and displayed. In some embodiments, this value is estimated by isolating the breath-breath intervals longer than a threshold (in some embodiments, 20 seconds, or a user-settable value) and identifying these as apneaic events. In some embodiments, the total number of apneaic events in the measurement interval are counted, and divided by the length of the measurement interval to determine the frequency of apneaic events. In some embodiments, the average time between apneaic events is calculated, and inverted to determine the frequency of apneaic events.

In some embodiments, an integrated irregularity index can be calculated. Possible implementations of an integrated irregularity index include:
A value, that is 0 for regular respiration, and can vary up to 6, with 1 point added for each of the following: irregular breath-breath interval; irregular breath depths; periodic breath-breath interval; periodic breath depth; periodic breath depth period>threshold (in some embodiments, 60 seconds); periodic breath-breath interval period>threshold (in some embodiments, 60 seconds); periodic breathing includes apnea>threshold (in some embodiments, 20 seconds); non-periodic irregular breathing includes apnea>threshold (in some embodiments, 20 seconds) more frequently than threshold (in some embodiments, once per 10 minutes)
A value that is 0 for regular respiration, that increases by one point for each N % in the coefficient of variation of the breath-to-breath interval and by one point for each N % in the coefficient of variation in the depth of breath. (In some embodiments, N can be 20%)

The information about the regularity or irregularity of respiration can be displayed in a variety of ways. Some, but not all, embodiments of the display include the following:
If respiration is regular, indicate that respiration is “regular”. If respiration is irregular, indicate either “periodic—cycle time X” where X is the cycle time or “irregular.” If apneaic events exist, indicate “—average apnea length Y” where Y is the average apnea length and, if respiration is not periodic also indicate “—Z apneaic events/minute,” where Z is the frequency of apneaic events.
Display the integrated irregularity index as a number.
Display the integrated irregularity index as a bar graph, which is green for very regular breathing, yellow for somewhat irregular breathing, and red for very irregular breathing.
Display an alert on the screen, with an accompanying audio alert, if respiration is irregular.

In some embodiments, the user can select the display method from a variety of choices, including, but not limited to, some or all of those listed above.

FIG. 10I illustrates a flow chart of a method that is used to assess the regularity of respiration. The method comprises the following steps:
1. Estimate the breath-to-breath interval and the depth of breath for each breath as respiration is processed as shown in block 1040.
2. Over an interval of 50 breaths, calculate the mean and standard deviation of the breath-breath interval, and the mean and standard deviation of the depth of breath as shown in block 1042.
3. Calculate the coefficient of variation of the breath-to-breath interval and the depth of breath as shown in block 1044. If neither one is above a threshold, the respiration is considered regular as shown in block 1046. If the coefficient of variation of either the breath-breath interval or the depth of breath is above a threshold, the respiration is considered irregular as shown in block 1048, and additional processing is performed. In some embodiments, the threshold can be 25%.
4. If the respiration is irregular, determine whether the cycle time is periodic by interpolating between breath-breath intervals and depth of breath estimates, taking a Fourier transform of each waveform, and determining whether a periodic component exists in either waveform as shown in block 1048. If a periodic component exists in at least one of the waveforms, the cycle time is periodic as shown in block 1052. If a periodic component does not exist in either waveform, the cycle time is not periodic as shown in block 1054.
5. If the cycle time is not periodic, repeat step 2 with a longer interval of breaths (150 breaths). If the cycle time is still not periodic, skip to step 7.
6. If the cycle time is periodic, calculate the cycle time finding by peaks in the interpolated breath-breath interval in step 4 and determining the mean time between the peaks as shown in block 1052. If multiple peaks are not available, extend the interval used for this step.
7. If the cycle is not periodic, isolate the breath-breath intervals longer than 20 seconds as shown in block 1056. Calculate the number of these intervals divided by the total time interval used for calculation. Calculate the mean of these apneaic events.
8. If the cycle is periodic, determine the length of apnea in each period, and average this number to get the average apnea length per cycle as shown in block 1058.
9. Display the data as shown in block 1060. If respiration is regular, indicate that respiration is “regular”. If respiration is irregular, indicate either “periodic-cycle time X” where X is the cycle time or “irregular.” If apneaic events exist, indicate “—average apnea length Y” and, if respiration is not periodic also indicate “—Z apneaic events/minute.”

In some embodiments, the following algorithm is used to provide indication of irregularity. Rates calculated by the rate estimator 1074 are stored in a FIFO buffer 1070 of length N where N is an integer. N represents the amount of data used to calculate the irregular breathing index. The sum of the absolute value of the differences of the rate values stored in the FIFO buffer 1070 is then taken, as shown in FIG. 10J. For elements 1 to N of buffer x, the block DIFF 1072 will return [x2−x1 x3−x2 . . . xn−xn−1]. The output of this calculation is the irregular breathing index. This index can then be compared with a predetermined threshold such that if the irregular breathing index is greater than the threshold, a subject's respiratory pattern is considered irregular.

A non-contact physiological measurement system can provide many respiratory variables, including respiratory rate, depth of breath, irregularity of pattern, inhale time to exhale time ratio, duration of apnea, frequency of apnea, and cycle length of periodic breathing, as well as the history and changes from baseline for all of these variables. However, this can be too much information for a nurse or nurse aide to process and track adequately, especially in situations where time only permits a quick glance at the monitor. In some embodiments of the system, in addition to, or instead of, displaying some or all of the respiratory variables, an “integrated respiratory status” value could be displayed, which combines all the respiration-related variables obtained into a single number that indicates the patient's overall respiratory well-being. In various embodiments, the integrated respiratory status index can be displayed as a number, as a bar graph, and can additionally be distilled to a green-yellow-red system, such that the color displayed indicates good-uncertain-poor respiratory status. In some embodiments, the integrated respiratory status can be calculated as a spot check variable. In some embodiments, the integrated respiratory status can be calculated continuously.

In some embodiments, the integrated respiratory status can take information from multiple sources. In some embodiments, this can be a pulse oximeter and a measurement of respiratory effort.

In some embodiments, the integrated respiratory status would use thresholds to assign points based on each parameter in real time, with the thresholds factory programmed. Each parameter can have one threshold, or can have several thresholds indicating the degree of severity. The sum of the points for each threshold would be the integrated respiratory status. In some embodiments, the integrated respiratory status would use thresholds to assign points based on each parameter in real time, with the thresholds factory programmed, and would also use historic information on each parameter to detect changes in each variable and assign additional negative or positive points based on changes in a good or bad direction. In various embodiments, each parameter can have one threshold, or can have several thresholds indicating the degree of severity. In some embodiments, the sum of the points for each threshold would be the integrated respiratory status.

In some embodiments, the integrated respiratory status would be a linear combination of all the real-time variables. In some embodiments, the integrated respiratory status would be a non-linear combination of all real-time variables. In some embodiments, the integrated respiratory status would be a linear combination of all real-time variables and of the derivative of each variable such that changes in the variable would be included. In some embodiments, the integrated respiratory status would be a non-linear combination of all real-time variables and of the derivative of each variable such that changes in the variable would be included.

In some embodiments, the integrated respiratory status would be computed based on a subset of parameters determined by a nurse or nurse aide. In this case, the parameters chosen would be the most appropriate for the monitored patient.

In some embodiments, the software that is executable by a processor can make an assessment of signal quality to prevent the display of incorrect rates. In various embodiments, the assessment can include four steps. In various embodiments, the first step can employ the non-respiratory signal detection algorithm to suppress any portions of the signal with motion other than respiration. In the second step, the software that is executable by a processor can compute the respiration rate using a time domain approach and a frequency domain approach, described above, separately, thereby producing two respiration rates for the same signal. The third step includes comparing the two rates resulting from the time and frequency domain approaches and determining if they are close to a certain number of breaths. In various embodiments, a smaller difference between the two rates can imply regular breathing intervals and regular breathing depths. In various embodiments, the software that is executable by a processor can regard regular breathing intervals and regular breathing depths as the two signal quality measures upon which it can confidently provide an accurate rate. In various embodiments, the fourth step includes checking if either one of the rates lies outside of a pre-determined interval for respiration rates in which case the software that is executable by a processor cannot provide a rate. Otherwise, the respiration rate can then be computed in various embodiments as the average of the two rates or by simply choosing either one of the rates.

In various embodiments described herein, a Doppler radar system with complex signal processing can monitor paradoxical breathing based on the complex constellation of the received motion signal based on target motion, including both chest and abdomen motion. The complex constellation is the plot of the quadrature signal vs. the in-phase signal. In various embodiments, paradoxical breathing can be an important sign of obstructed breathing, respiratory muscle weakness, or respiratory failure. Paradoxical breathing can also occur with some types of paralysis. With paradoxical breathing, the abdomen and rib cage move in opposite directions rather than in unison, example when the rib cage expands, the abdomen contracts, and when the abdomen expands, the rib cage contracts.

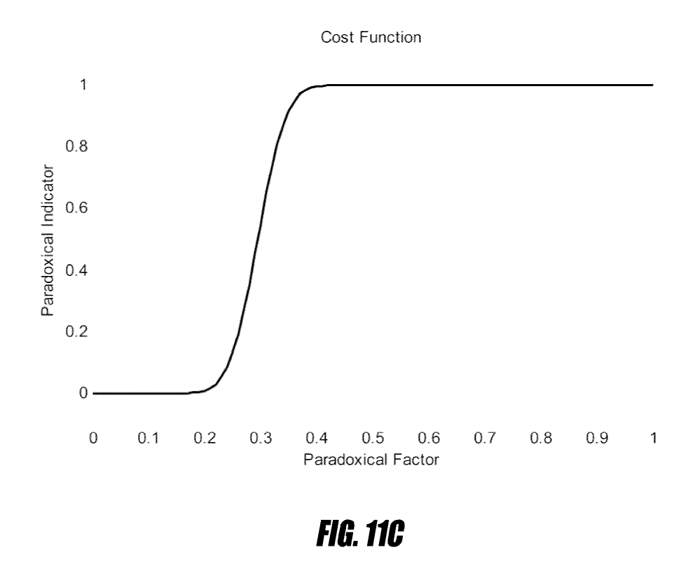

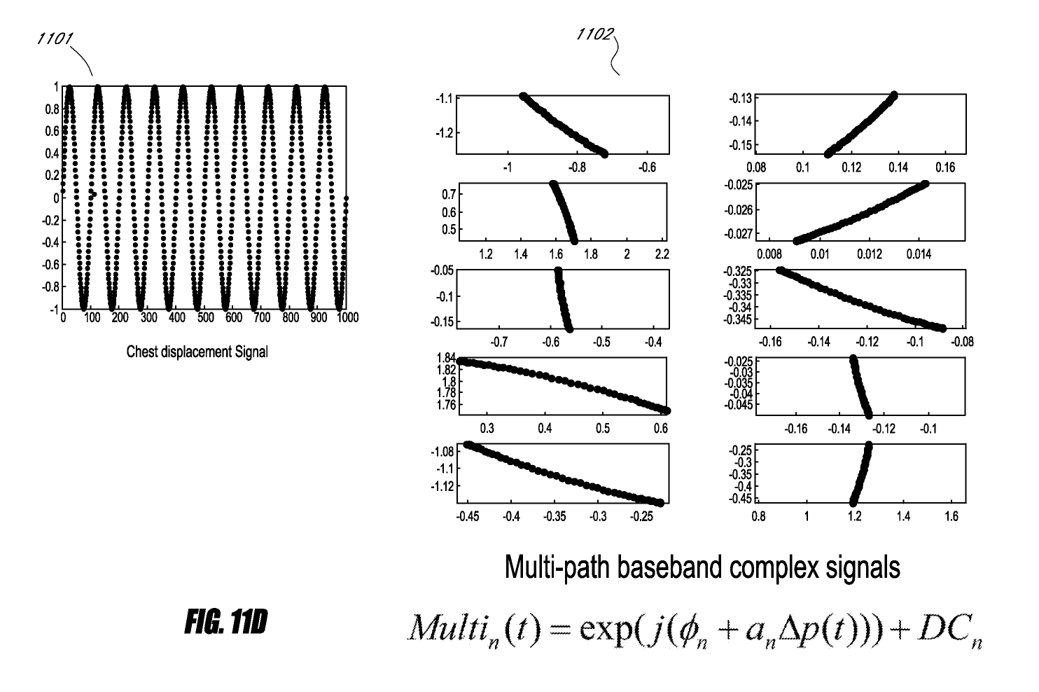

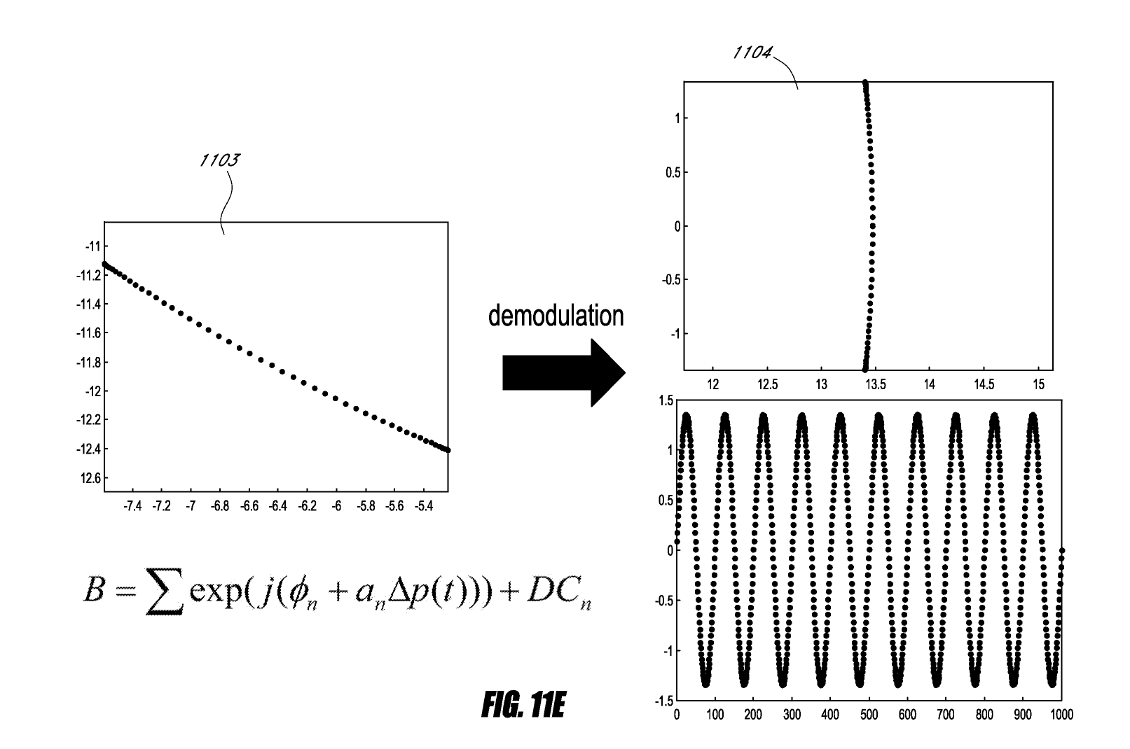

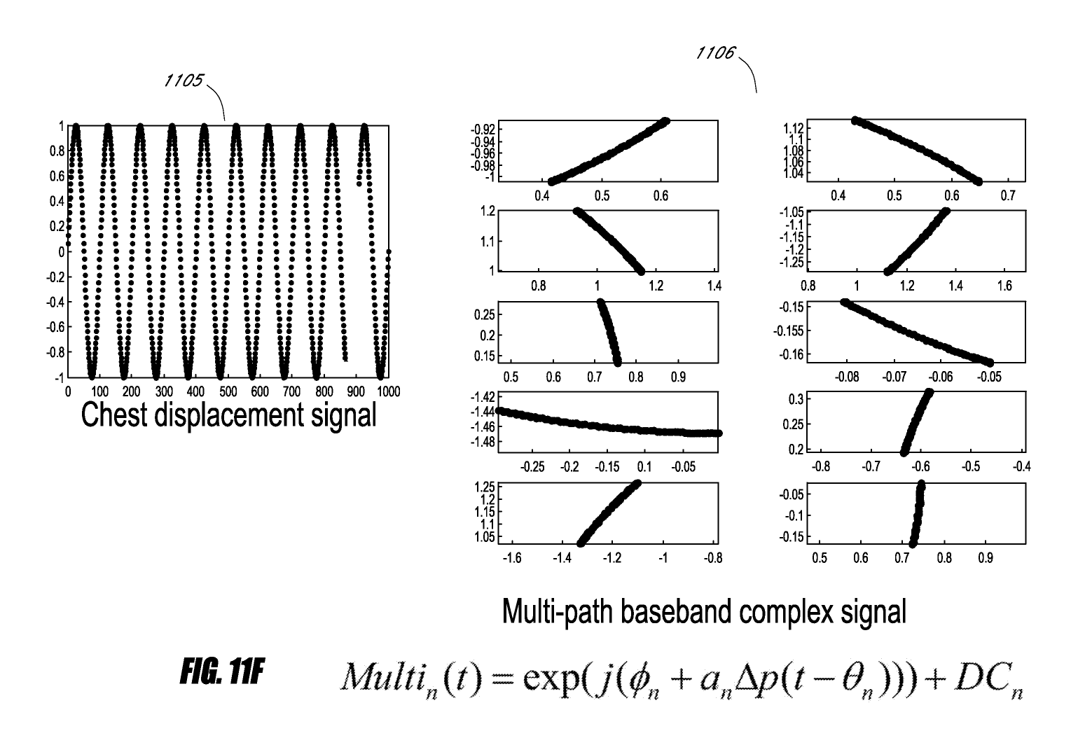

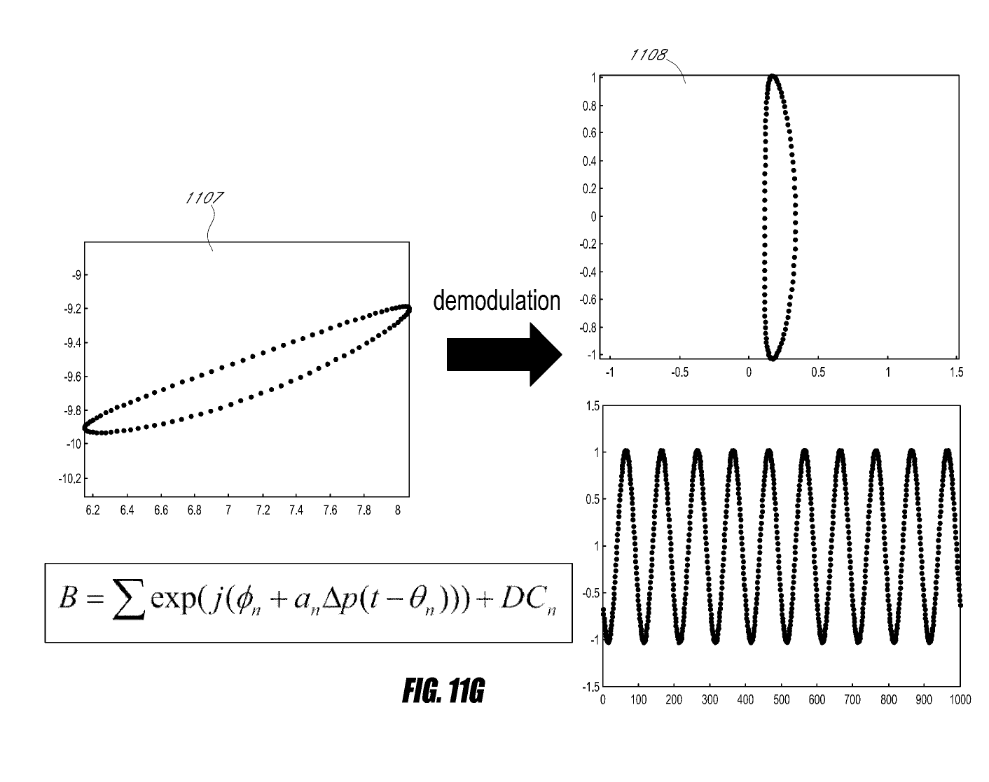

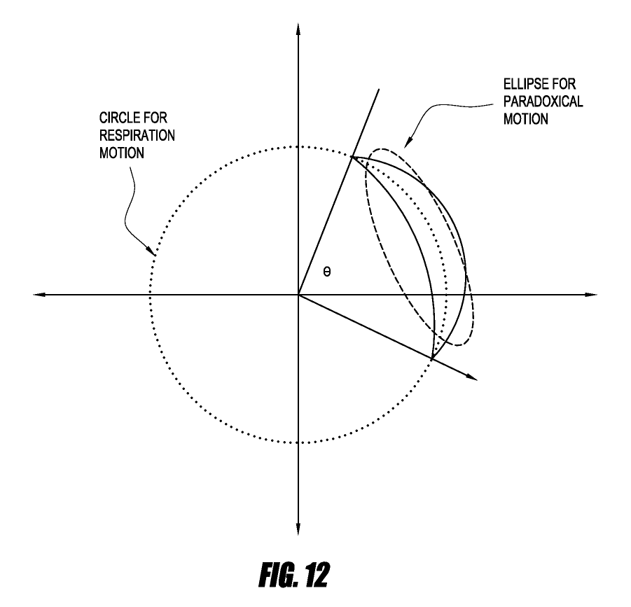

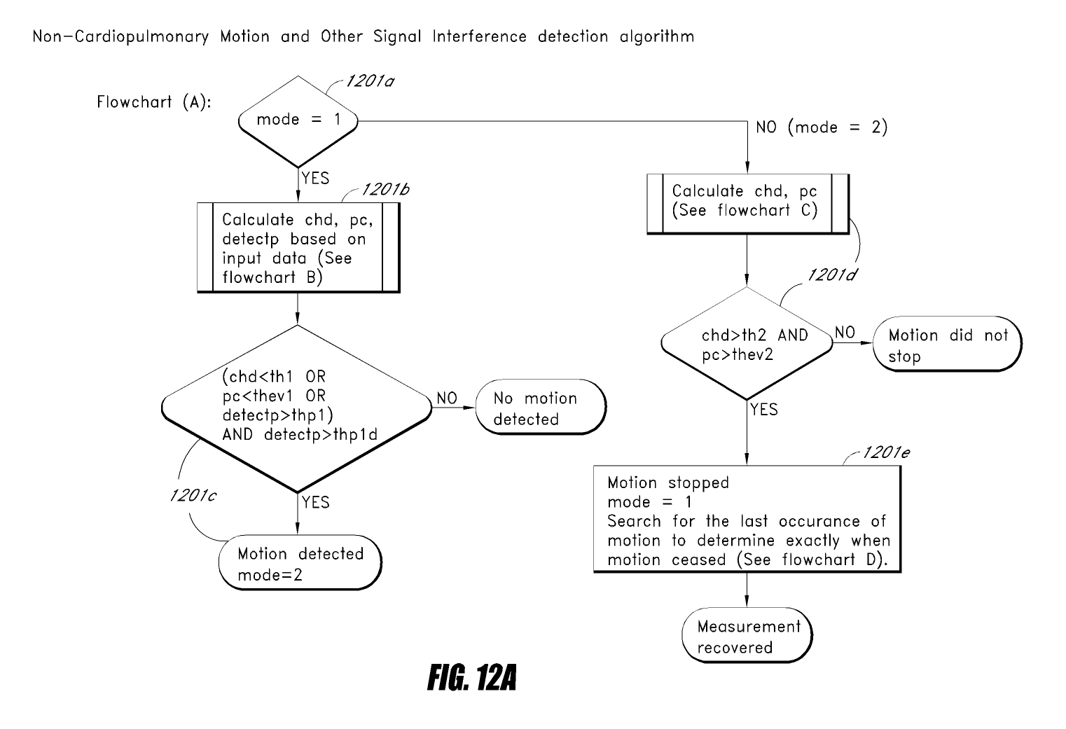

Obstructive apnea is commonly defined as an 80-100% reduction in airflow signal amplitude for a minimum of 10 seconds with continued respiratory effort. The rib cage and abdomen can move out of phase as the patient tries to breathe, but the airway is blocked. A quadrature Doppler radar system, such as the one described above, can monitor this paradoxical breathing based on the complex constellation due to the target's chest and abdomen motion. Since a human's physiological signal such as breathing is a very narrow band signal (˜less than 1 KHz) compared to the radar carrier signal, all the reflected signals will be phase modulated on a coherent carrier signal. Therefore, if human body parts, for example the chest and abdomen, are expanding or contracting simultaneously, the received reflecting signals from different paths (reflecting from different body parts) will only shift the phasor of the carrier signal but not the phase modulated narrow band carrier signals. Shift of the phasor of phase modulated narrow band carrier signals can also occur when different body parts are moving at the same frequency but with different amplitude or phase delay, as is the case in paradoxical breathing. Consequently, in the former case, the shape of the complex plot at the baseband due to the respiration will not change and will form a fraction of a circle (an arc) which is similar to the one from the a single source, while in the latter case the phasor of the baseband signal changes during the periodic motion (such as breathing), resulting in distortion of the complex constellation. This fact can be used to detect paradoxical breathing. Simplified phasor diagrams of those the two cases in the previous paragraph are described in FIGS. 11A and 11B as disclosed in U.S. Provisional App. No. 61/194,836 which is incorporated herein by reference in its entirety and in U.S. Provisional App. No. 61/194,848 which is incorporated herein by reference in its entirety and in U.S. Provisional App. No. 61/200,761 which is incorporated herein by reference in its entirety.

FIG. 11A illustrates the phasor diagrams for normal breathing and FIG. 11B illustrates the phasor diagrams for paradoxical breathing. During the normal breathing, only the phasor of carrier signal is shifted as different phase delayed carrier signals represented by the dashed vector are superimposed, while during the paradoxical breathing, not only the phasor of carrier signal but also that of baseband signal are shifted thus resulting in different complex constellation shape from FIG. 11A.

In various embodiments, comprising measurement of a motion causing a Doppler shift that is narrowband compared to the carrier signal (<<1%), multiple reflections from synchronized sources do not distort the shape of the complex motion signal, but reflections can change the signal power due to destructive or constructive interference of reflected carrier signals with different time delays. In various embodiments, comprising measurement of a motion signal causing a Doppler shift that is narrowband compared to the carrier signal (<<1%), multiple reflections from synchronized sources do not result in distortion of the complex motion signal unless the multi-path occurs over a range that is comparable (>1%) to the electrical wavelength (>300 km) corresponding to the frequency of the cardiopulmonary signal (<1 kHz), which is the frequency of the phase modulation on the carrier signal. In various embodiments, the signals reflected from different body parts can be handled as multi-path signals causing Doppler shifts on the carrier signal with a very narrow signal band and with time delays much less than those corresponding to the wavelength of the phase modulation frequency (>300 km), and consequently there is no shape change of the complex signal as long as all the body parts expand or contract simultaneously. However, if there is time delay (or phase shift) between the expanding or contracting motion of different body parts, such as in paradoxical breathing, the complex constellation is distorted and becomes an elliptic or ribbon shape rather than a small arc or line shape. Paradoxical breathing can be detected by comparing the ratio of two primary vectors (e.g., eigenvectors) and amplitudes of the signals projected on each primary vector. A dedicated cost function given by the equation can identify paradoxical breathing events from the processed outputs and provide indication of paradoxical breathing.

The paradoxical factor can be calculated as the ratio of the largest eigenvalue to the second largest eigenvalue multiplied by the ratio of the maximum amplitude of the signal projected on the principal vector to the maximum amplitude of the signal projected onto the vector orthogonal to the principal eigenvector. A cost function can convert the paradoxical factor to a paradox indicator, which can be used to indicate paradoxical breathing.

The input to the cost function will be the paradoxical factor and the cost function will transform it to a value which is between 0 and 1. In some embodiments, the cost function can be given by the following equation

$$Cost ( input ) = \frac{1}{v \times \sqrt{2 π}} {\int }_{x 1}^{x 2} exp ( \frac{- {( input - m )}^{2}}{2 \times {v}^{2}} ) d x ,$$

where x1, x2 are range of paradoxical factor which can be 0 and 1, while m and v are boundary input values between paradoxical and non-paradoxical and v is emphasizing factor of paradoxical factor. For example, if m is close to x1 then paradoxical indicator threshold is set to lower paradoxical factor. On the other hand, as v increases paradoxical indicator changes more dramatically as paradoxical factor changes. If the paradoxical indicator is near one, it is likely that there is paradoxical breathing; if the paradoxical indicator is near zero, it is unlikely that there is paradoxical breathing. A threshold can be set on the paradoxical indicator to provide a yes/no output, or two thresholds can be applied to achieve a green-yellow-red output corresponding to likely paradoxical breathing, uncertain output, and unlikely paradoxical breathing.

In one embodiment, of this invention, m is set to 0.3 and v is set 0.04. The cost function with these values of m and v is shown in FIG. 11C.

FIGS. 11D and 11E illustrate the baseband outputs with multi-path delayed signals when the body parts exhibit simultaneous body expansion and contraction motion while FIGS. 11F and 11G illustrate the baseband outputs with multi path delayed signals when the body parts expand or contract with different phase delays. Referring to FIGS. 11D and 11E, reference numeral 1101 of FIG. 11D illustrates a motion signal (e.g., chest displacement signal). The multi-path based complex signals are shown in plots identified by 1102. The summed multi-path signal is shown in plot 1103 of FIG. 11E. Plot 1104 shows the demodulation signal which is approximately linear indicating absence of abnormal breathing (e.g., paradoxical breathing).

Referring to FIGS. 11F and 11G, reference numeral 1105 of FIG. 11F illustrates a motion signal (e.g., chest displacement signal). The multi-path based complex signals are shown in plots identified by 1106. The summed multi-path signal is shown in plot 1107 of FIG. 11G. Plot 1108 shows the demodulation signal which is approximately linear indicating absence of abnormal breathing (e.g., paradoxical breathing).

Various embodiments, representing alternate methods for distinguishing paradoxical breathing from non-paradoxical breathing are proposed; these methods include methods for distinguishing an ellipse, circle, moon-shape, or other shapes from an arc or line. The quadrature data from a Doppler radar sensor used to measure respiratory motion is visualized with a plot of the in-phase and quadrature data on the abscissa and ordinate axis respectively, hereby referred to as an I/Q plot. On an I/Q plot, a full respiration cycle, of non-paradoxical motion can produce an arc-like shape. If there is one object oscillating towards and away from the radar, such as a chest during respiration, there can be an arc. If respiration involves more than one signal source such as the abdomen moving out of phase with the chest, an elliptical shape or other shape can form. In this case, there can still be an underlying arc path but a distinguishable separation of the inhale and exhale paths in the I/Q plane, creating an ellipse or a curved ellipse shape similar to a kidney bean shape. Due to path-length differences causing a phase difference between the signal sources, the signal shape on the I/Q plane can also look like a crescent moon, a figure-8 or ribbon shape, an egg shape, a circle, or a combination of above.

In some embodiments, a process to determine whether the shape is an arc or another shape is executed on one or more successive frames of the data. In some embodiments, the frame length is determined based on the algorithm's ability to determine a line fit to the data in the corresponding frame length. In some embodiments, the frame length is fixed and short to allow a line fit on most of the expected signals. In some embodiments, the frame length changes adaptively. In some embodiments, the frame length is changed adaptively based on the respiratory rate of the subject. In some embodiments, the frame length is changed adaptively based on the error between the data and the best-fit line.

In some embodiments, a step in the process of determining the shape consists of determining the best-fit line to segments of the data. The best-fit line can be found using various methods including, but not limited to, the eigen-decomposition of the covariance matrix formed by the in-phase and quadrature data from the time frame, or using a least-squares estimation to find a and b in the equation y=ax+b. In some embodiments, an orientation vector pointing to the direction of movement in the I/Q plot is then deduced for every time frame. The orientation vectors computed in that process serve to identify the type of trajectory in the I/Q plot. The ellipse and arc/line are differentiated by the change in phase between consecutive orientation vectors: in an ellipse, the sign of the phase change is constant, while in an arc/line the sign of the phase change flips at the endpoints. In some embodiments, an arc/line is concluded when the positive and negative phase signs are equally present, and therefore, normal breathing is concluded. In some embodiments, an ellipse, and therefore, paradoxical breathing, is concluded when one phase sign is dominant. In a line or an arc, there is a 180 degree phase shift at the end of the arc/line, while the phase change is less in any of the other shapes that can indicate paradoxical breathing. In some embodiments, the total phase change between successive orientation vectors or a small set of orientation vectors (in some embodiments 3-4 vectors) is assessed, and if it is greater than a threshold (in some embodiments, 170 degrees), this indicates non-paradoxical breathing, and if there is never a phase change greater than the threshold, paradoxical breathing is indicated.

In some embodiments, a model of the signal generated by two or more sources can be created. In some embodiments, this model can include such factors as carrier frequency, relative signal reflection, relative angle of arrival, relative path distance difference, source objects' movement in terms of displacement, phase difference, frequency of respirations, and respiratory pattern. In some embodiments, the model can be able to distinguish different patterns such as a sinusoidal pattern, a square-wave like pattern, a pulse train pattern, a triangle-wave like pattern, a saw-tooth like wave pattern, or a rectified sinusoidal wave pattern. In some embodiments, a representative equation of the model is compared and fitted against the signal in the I/Q plot and used to describe the movement of the objects. In some embodiments, if the signal matches a model of non-paradoxical breathing most closely, non-paradoxical breathing is indicated, and if the signal matches a model of paradoxical breathing most closely, paradoxical breathing is indicated.

In some embodiments, a circle-fitting algorithm is used that can estimate the center of the circle on which the data lies, identifying a best-fit arc for the data. In some embodiments, a carrier frequency is selected to produce an arc shape that is easier for the arc detection algorithm to detect. Higher carrier frequencies produce higher phase shifts for the same amount of displacement, and therefore the arc subtends a larger central angle for the same amount of displacement with a higher carrier frequency.

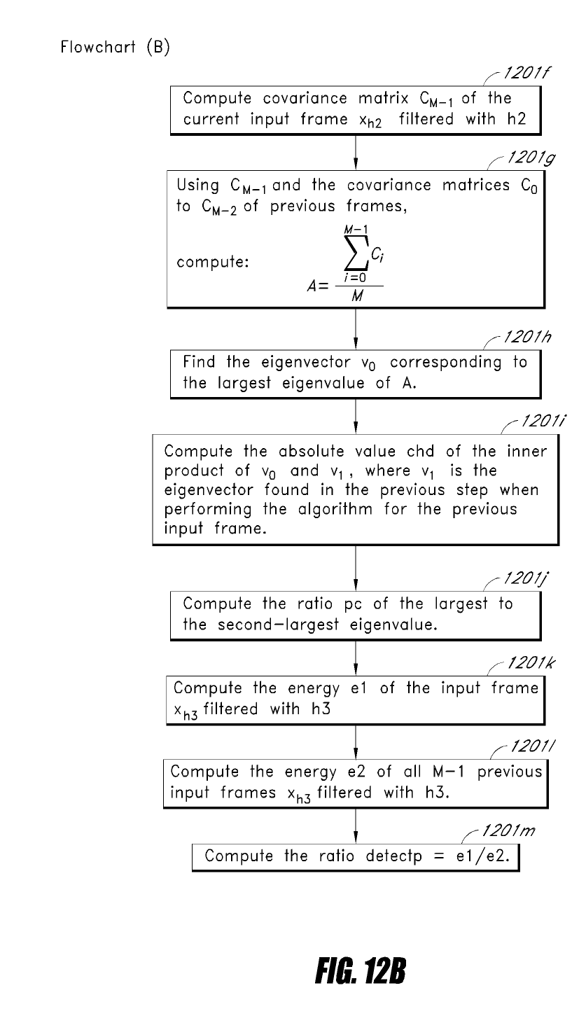

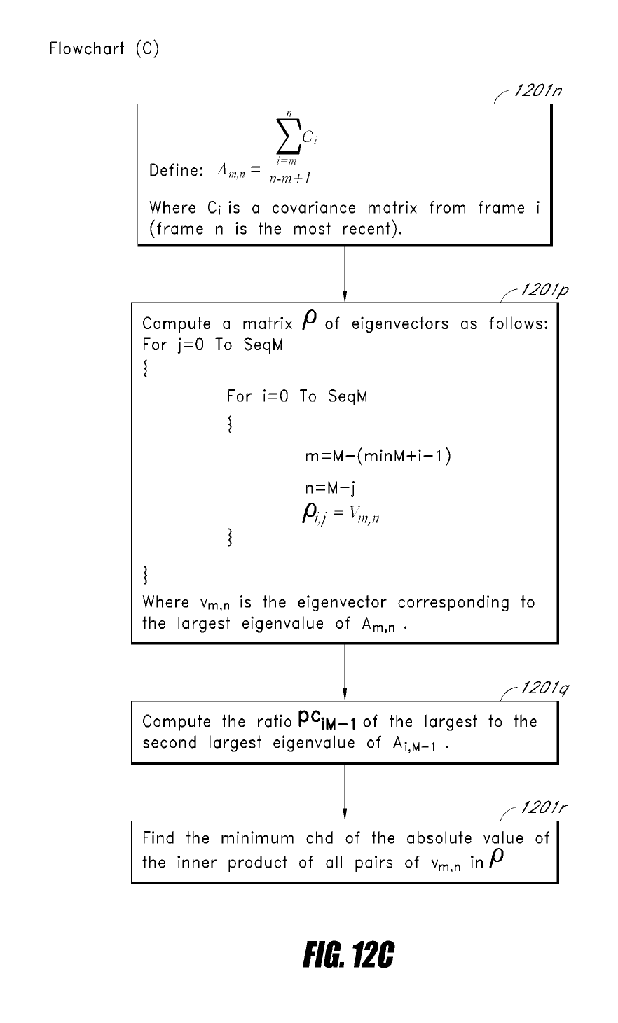

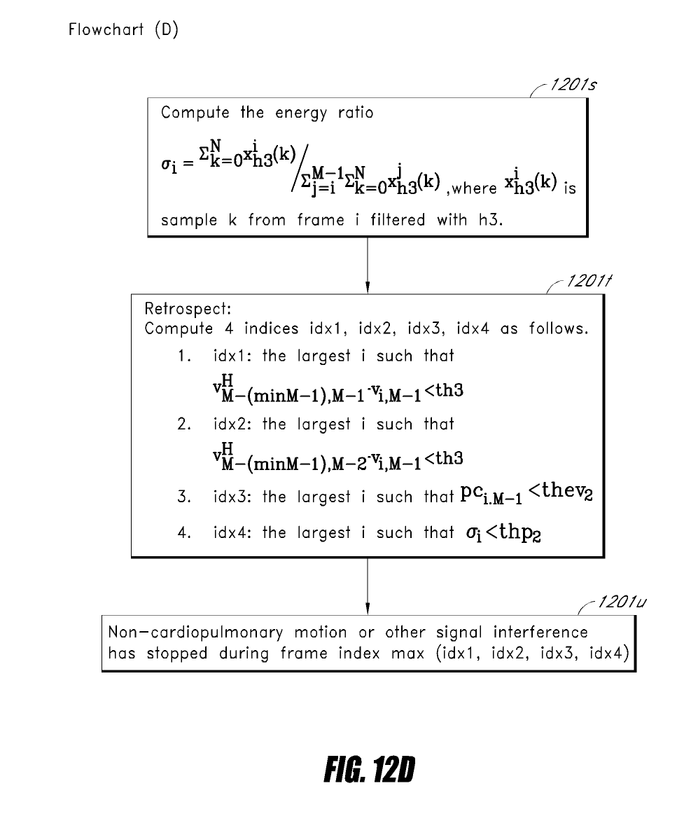

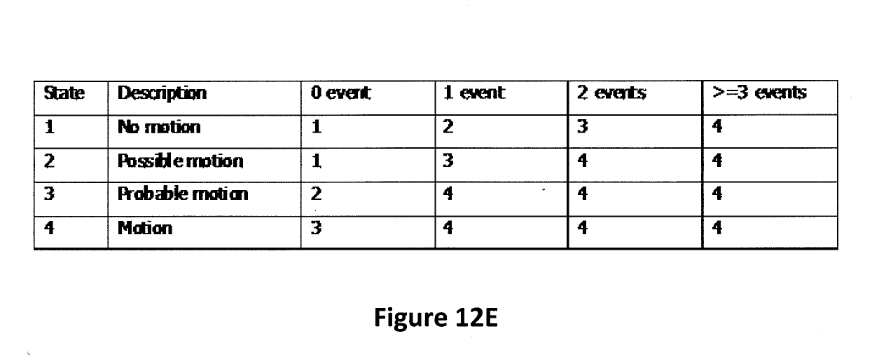

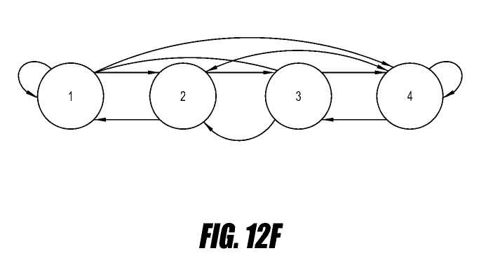

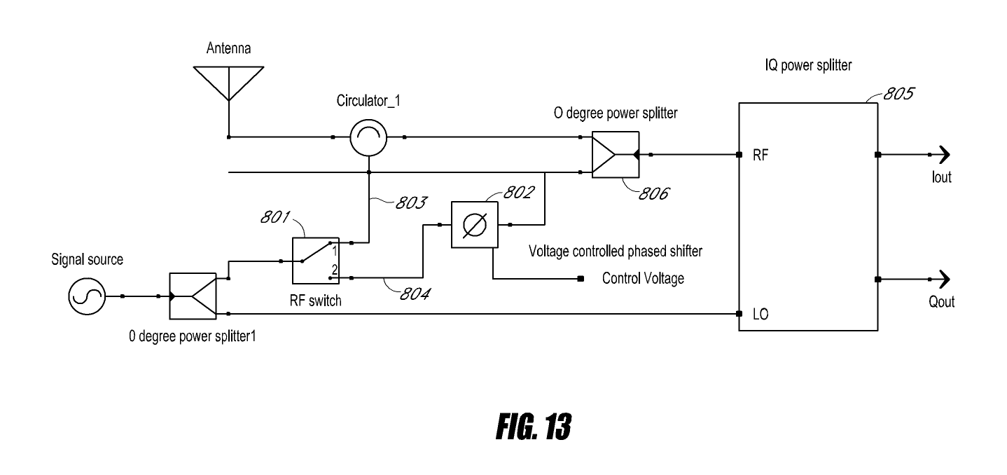

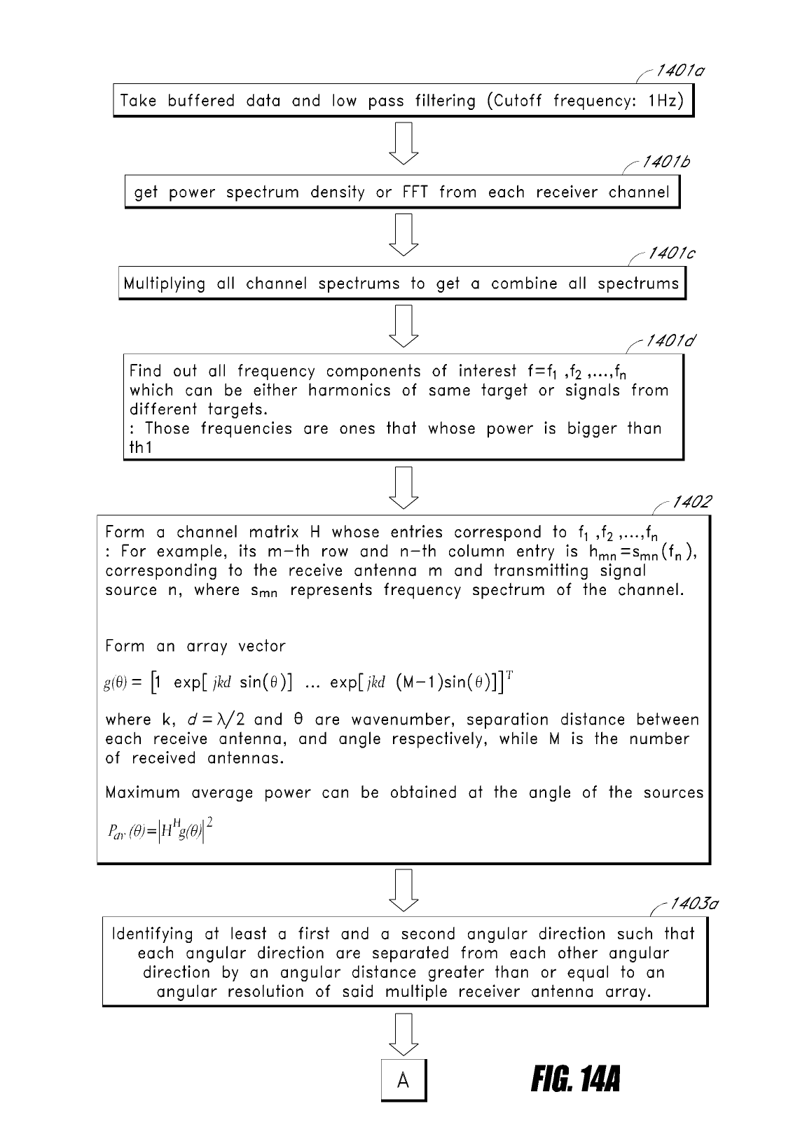

In some embodiments, the data samples in the I/Q plane are fitted to an arc. In some embodiments, there can be an expected angle subtended by an arc for the respiratory movement, which can be bounded on the upper end. In some embodiments, this upper boundary of the phase change, can be related to the carrier frequency and the maximum expected displacement caused by respiration for all body types. In some embodiments, this upper boundary for the angle subtended by respiration can be specific to the patient and the carrier frequency, utilizing patient information and/or historically measured breathing data. In some embodiments, during paradoxical breathing, an arc can be fitted to data that has an elliptical shape, as shown in FIG. 12. In some embodiments, the ellipse fitting algorithm can be limited to finding ellipses whose major radius is less than a constant multiplied by the circle's radius, depending on the carrier frequency. In some embodiments, the dimensions of the ellipse are compared to the dimensions of the circle as an indicator of paradoxical breathing. In some embodiments, an ellipse can be fitted to the data samples in the I/Q plane. In some embodiments, the signal to be fitted to an ellipse shape is defined by a full respiration cycle, by many respiration cycles, or over a period of time longer than a respiration cycle. In some embodiments, the eccentricity of the ellipse is an indicator of paradoxical breathing.

In some embodiments, the beginning and ending of inhalation and exhalation of the respiratory cycle are marked and used to separate the data into two sections: inhalation and exhalation. In some embodiments, each section is analyzed separately and compared with one or more methods to determine whether paradoxical breathing is present. In some embodiments, a circle is fit to each section and the centers, radii, or both are compared, and if they are significantly different, that indicates paradoxical breathing. In some embodiments, the centers for exhale and inhale are compared to the center of the best fit circle for the whole respiratory cycle, and if they are significantly different, that indicates paradoxical breathing. In the case of the crescent-moon shape in the I/Q plane, shown in FIG. 12, the inhale trace and exhale trace would indicate different circle centers. In some embodiments, if the center of the circle flips to the opposite side of the signal, then there is a change from a concave shape to a convex shape, and this indicates paradoxical breathing. In some embodiments, a linear fit is used and the position, angle, least mean square error, and/or combination of these are compared, and if the compared values are significantly different, this indicates paradoxical breathing.

In some embodiments, the area bounded by the signal can be used to indicate paradoxical breathing. This area can be bounded by a single respiration cycle's path; by a representation of the signal by parametric path, connected arcs of two best fit circles (see above), or by a polygon created by signal key points; or by multiple cycles and bounded by the outermost data points. In some embodiments, if the area bounded by the signal is greater than a threshold, paradoxical breathing is indicated, and if the area bounded by the signal is less than a threshold, normal breathing is indicated. In various embodiments, the threshold or thresholds can be set permanently, or they can be based on the carrier frequency.

In some embodiments, a best-fit circle can be found for the whole breathing cycle, such that the center of this signal can be determined, and the variance in the distance between the data points and the center can be tracked. In some embodiments, if the variance in the distance between the data points and the center is below a threshold, normal breathing is indicated, and if the variance in the distance between the data points and the center is above a threshold, paradoxical breathing is indicated. In some embodiments, the average distance and variance of the distance between data points and the center are tracked during inhalation and exhalation separately, and if the distance is significantly different between inhale and exhale, paradoxical breathing is indicated.

In some embodiments, the shape in the I/Q plane can be compared with a library of shapes including shapes indicative of paradoxical breathing and/or shapes indicative of non-paradoxical breathing shapes, such that the shape in the I/Q plane can be matched to a shape in the library and the categorization of the shape from the library can be used to indicate paradoxical breathing or non-paradoxical breathing. In some embodiments, the library of shapes includes individual images, and each of these images can be cross-correlated with a normalized image of the I/Q plane. In some embodiments, the image that results in the highest correlation represents the shape most similar to the one on the I/Q plane, indicating a match between the data and the shape. In some embodiments, each shape in the library can be associated with information, including, but not limited to, whether or not it indicates paradoxical breathing and/or the degree of paradoxicity indicated by the shape. In some embodiments, the average cross-correlation factor between the shape and all paradoxical images in the exhaustive search is compared with average cross-correlation factor between the shape and all non-paradoxical images in the exhaustive search, and the group (paradoxical or non-paradoxical) with higher correlation indicates the classification of the data as paradoxical or non-paradoxical. In some embodiments, the library of images form sub-images to a large image encompassing the entire library, while the I/Q plane image is used as the mask for the cross-correlation. The result of the cross correlation can be analyzed individually for each sub-image or as a group for paradoxical vs. non-paradoxical shapes. In some embodiments, the sub-images can be strategically placed to form a gradient, in the x or y direction, of paradoxicity levels from least to most paradoxical or vice versa. In some embodiments, the algorithm to match shapes can be based on the image processing technique of locating key points in the complex constellation. In such embodiments, the key points are selected such that they can be detected consistently under various distortions of the complex constellation including homographic transformations. In some embodiments, every shape in the library has an associated set of key points, and the algorithm matches the key points found in the unknown shape to the key points for every shape in the library. In some embodiments, the matching process assumes that the shape in the library undergoes affine transformations to result in the unknown shape. In such embodiments, the parameters of the transformation can be deduced from the input and output points of the system. In some embodiments, the RANSAC algorithm is used to optimally select the set of points that can lead to determining the parameters of the transformation. The unknown shape can then be matched to a library shape by finding the library shape with the largest number of key points matched with the key points in the unknown shape (by an affine transformation).

In some embodiments, paradoxical breathing can be indicated by looking at the variance between the samples and the principal vector in the I/Q plane. In some embodiments, paradoxical breathing can be detected by analyzing the variance between the samples and the best-fit arc or circle. In some embodiments, in each frame, the variance of samples and the principal vector, best-fit arc, or circle is computed. Non-paradoxical breathing should have a smaller variance than paradoxical breathing. In some embodiments, a threshold can be used to determine if data is paradoxical or not. In some embodiments, if the variance is greater than a set threshold, then the data is said to indicate paradoxical breathing. In some embodiments, the number of samples used to compute the variance can contain the current frame or the current frame plus a history of frames. In some embodiments, the said threshold can be computed through any combination of the sample size, theoretical data, and/or simulated data.

In some embodiments in which the carrier frequency is high enough that a respiration cycle traces at least a full circle in the I/Q plane, a constant modulus-detection algorithm can be used for paradox detection. In some embodiments, a constant modulus signal can indicate non-paradoxical breathing, and a non-constant-modulus signal can indicate paradoxical breathing. In some embodiments, the constant-modulus is determined by the distribution of the modulus of the samples in the I/Q plane from a center or the origin when the signal is zero mean. In some embodiments, the signal is found to be constant-modulus when the distribution has small variance.

In some embodiments, direction of arrival algorithms can be used to identify two or more points that are moving with respiratory activity. In some embodiments, paradoxical breathing is indicated by a negative correlation between motion at one point and motion at another point. In some embodiments, a high-resolution sensor can be used in conjunction with DOA algorithms to map the chest motion and identify the direction of motion at different points. Points moving out of phase would indicate paradoxical breathing.

In various embodiments, the radar-based physiological motion sensor can detect non-cardiopulmonary signals or motion events as described herein. In various embodiments, a signal with a single stable source can be considered as a cardiopulmonary signal and a signal that is unstable or has multiple sources can be considered a non-cardiopulmonary signal as disclosed in U.S. Provisional App. No. 61/123,017 which is incorporated herein by reference in its entirety and in U.S. Provisional App. No. 61/125,019, which is incorporated herein by reference in its entirety. In various embodiments, a signal with a single stable periodic scatterer can be considered a cardiopulmonary signal, and a signal that is unstable or has multiple scatterers can be considered to include non-cardiopulmonary motion or other signal interference.

In various embodiments, the physiological signals can be analyzed to determine the quality of the signal, including, but not limited to, detection of non-cardiopulmonary motion, detection of high signal-to-noise ratio, detection of low signal power, detection of RF interference, and detection of signal clipping. Additionally, signal quality can be measured by analyzing the signal in the complex plane to determine how much the scattered data samples are smeared with respect to an arc or a principle vector. The samples of a high-quality signal should lie very close to an arc or a principle vector, and significant deviation from that arc or vector can indicate a lower-quality signal. In some embodiments, the low-signal cutoff can be calculated based on a threshold, either in the spectral domain or the time domain. In some embodiments, the low signal power threshold can be calculated from the effective number of bits provided by the analog-to-digital converter and the full-scale voltage of the baseband circuit. In some embodiments, the clipping indicator can be triggered when the digitized voltage exceeds a maximum value as disclosed in U.S. Provisional App. No. 61/141,213 which is incorporated herein by reference in its entirety.

In various embodiments, non-cardiopulmonary motion (e.g., motion of objects in the vicinity of the subject or physical movement by the subject) can be detected in a variety of ways. For example, in some embodiments an excursion larger than the subject's maximum chest excursion due to cardiopulmonary motion (or breath) can be an indication of non-cardiopulmonary motion. Similarly, a significant increase in signal power can indicate motion.

In those systems where linear demodulation is suitable, significant changes to the best-fit vector, primary vector or eigenvector of the covariance matrices can indicate non-cardiopulmonary motion. The best-fit vector, primary vector or eigenvector is the vector on which the signals are projected. Significant changes to the best-fit vector, primary vector or eigenvector can also indicate a new relationship between the antenna and the subject and further indicates non-cardiopulmonary motion. Changes to the best-fit vector, the eigenvector or the primary vector can be detected by calculating the inner product of the normalized current vector and the normalized previous vector. If the inner product is below a threshold, then it is possible that non-cardiopulmonary motion is present. When linear demodulation is used, a significant change in the ratio of the eigenvalues, or of the RMS error of the data to the best-fit line, or of the RMS difference between the complex constellation of the signal and the best-fit vector, indicates that the detected motion does not fit the line well which can indicate presence of non-cardiopulmonary motion or signal interference as disclosed in U.S. Provisional App. No. 61/141,213 which is incorporated herein by reference in its entirety.

When arc-based demodulation is used, significant changes in the location of the origin, changes in the radius of the circle the arc is on, or changes in the position of the arc on the circle can indicate a change in the relationship between the antenna and subject, which can in turn indicate presence of non-cardiopulmonary motion. In those systems where arc-based demodulation is used, a change in the RMS error of the data to the best-fit arc or RMS difference between the complex constellation of the signal and the best-fit circle is an indication of a non-cardiopulmonary motion signal or other signal interference as disclosed in U.S. Provisional App. No. 61/141,213 which is incorporated herein by reference in its entirety.

In various embodiments, noise that affects the I and Q channels equally, including thermal noise and some types of noise from radio interference, can be estimated by the excursion of the signal from a line or arc in the complex plane, and the signal power can be calculated by the length of the line or arc. Thus, a signal-to-noise ratio can be estimated, and can be used as an indicator of the quality of the signal as disclosed in U.S. Provisional App. No. 61/141,213 which is incorporated herein by reference in its entirety.

In various embodiments, when motion or another non-respiratory signal is detected, the device can not display a respiratory rate as disclosed in U.S. Provisional App. No. 61/123,017 which is incorporated herein by reference in its entirety. The non-cardiopulmonary motion detection algorithm can be used to enable some embodiments to operate as an activity monitor.

An example of a non-cardiopulmonary motion detection algorithm is further described below and illustrated in FIGS. 12A-12D. The algorithm can be executed by a processor and is configured to detect non-cardiopulmonary motion or other signal interference by looking at the change in direction of the eigenvectors, the ratio of the eigenvalues and the change of energy in the signal, as shown in block 1201b. The algorithm starts in mode 1, as shown in block 1201a, by assuming that no non-cardiopulmonary motion or other signal interference is present and switches to mode 2 as shown in block 1201c as soon as any non-cardiopulmonary motion or other signal interference is detected. When in mode 2, the algorithm similarly checks the change in direction of the eigenvectors and the ratio of eigenvalues, as shown in block 1201a to determine if the non-cardiopulmonary motion or other signal interference has ceased. If motion ceases, then the algorithm will find the earliest time (the retrospect) with no motion, as shown in block 1201e. The algorithm comprises the following steps:

1. Mode=1
a. Compute covariance matrix C_{M-1} of the current input frame xₕ₂ filtered with a first filter having a filter function h2, as shown in block 1201f of FIG. 12B. In some embodiments, the first filter can be a low-pass filter.
b. Using C_{M-1} and the covariance matrices C₀ to C_{M-2} of previous frames, compute an A-matrix

$$A = \frac{{\sum }_{i = 0}^{M - 1} {C}_{i}}{M} ,$$

as shown in block 1201g of FIG. 12B, where M is the number of preceding frames to consider and in some embodiments can be 32. In various embodiments M can be larger or smaller than 32.
c. Find the eigenvector v₀ corresponding to the largest eigenvalue of A, as shown in block 1201h of FIG. 12B.
d. Compute the absolute value chd of the inner product of v₀ and v₁, where v₁ is the eigenvector found in step c when performing the algorithm for the previous input frame, as shown in block 1201i of FIG. 12B.
e. Compute the ratio pc of the largest to the second-largest eigenvalue, as shown in block 1201j of FIG. 12B.
f. Compute the energy eᵢ of the input frame x₃ filtered with a second filter having a filter function h3. In various embodiments, the second filter can be a high-pass filter, as shown in block 1201k of FIG. 12B.
g. Compute the average energy per frame e₂ of all M−1 previous input frames x₃ filtered with h3, as shown in block 12011 of FIG. 12B.
h. Compute the ratio detectp=eᵢ/e₂, as shown in block 1201m of FIG. 12B.
i. If (chd<th1 OR pc<thev1 OR detectp>thp1) AND detectp>thp1d), as shown in block 1201b and 1201c then non-cardiopulmonary motion or other signal interference is detected, switch to Mode=2. In various embodiments th1 can have a value between approximately 0.6 and approximately 1. In various embodiments, thev1 can have a value in the range 4 and 12. In various embodiments, thp1 can have a value in the range 4 and 20. In various embodiments, thp1d can have a value between approximately 0.1 and approximately 0.8.

2. Mode=2
a. Calculate an A′-matrix given by the equation

$${A}_{m , n} = \frac{{\sum }_{i = m}^{n} {C}_{i}}{n - m + 1} ,$$

where Cᵢ is a covariance matrix from frame i (frame n being the most recent), as shown in block 1201n of FIG. 12C.
b. Compute a matrix ρ of eigenvectors as follows, as shown in block 1201p of FIG. 12C:

For j = 0 To SeqM
{

For i = 0 To SeqM
{

i. m = M − (minM + i − 1)
ii. n = M − j
iii. ρ_{i,j} = v_{m,n}

}

}
$ρ = [ \begin{matrix}{v}_{M - ( minM - 1 ) , M - 1} & … & {v}_{M - ( minM - 1 ) , M - SeqM} \\ ⋮ & ⋱ & ⋮ \\ {v}_{M - ( minM - SeqM - 1 ) , M - 1} & … & {v}_{M - ( minM - SeqM - 1 ) , M - SeqM}\end{matrix} ] ,$

where SeqM is about 5 in some embodiments and corresponds to the number of preceding frames to consider, where minM is the number of frames prior to current frame to consider and is about 8 in some embodiments, where v_{m,n} is the eigenvector corresponding to the largest eigenvalue of A_{m,n}.
c. Compute the ratio pc_{i,M-1} of the largest to the second largest eigenvalue of the matrix A_{i,M-1}, as shown in block 1201q of FIG. 12C.
d. Find the minimum chd of the absolute value of the inner product of all pairs of v_{m,n} in ρ, as shown in block 1201r of FIG. 12C.
e. Compute the energy ratio

$${σ}_{i} = {\sum }_{k = 0}^{N} {x}_{h 3}^{i} ( k ) / {\sum }_{j = i}^{M - 1} {\sum }_{k = 0}^{N} {x}_{h 3}^{j} ( k ) ,$$

where xₕ₃ⁱ(k) is sample k from frame i filtered with h3, as shown in block 1201s of FIG. 12D.
If (chd>th2 AND pc_{M-(minM-1),M-1}>thev2) then non-cardiopulmonary motion or other signal interference has stopped, switch to Mode=1, as shown in blocks 1201d and 1201e of FIG. 12A. In various embodiments, th2 can have a value between approximately 0.6 and approximately 1. In various embodiments, thev2 can have a value between approximately 4 and approximately 12.
g. Retrospect: Compute 4 indices idx1, idx2, idx3, idx4 as follows, as shown in block 1201t.
   idx1: the largest i such that v_{M}^{H}−(minM−1),M−1·v_{i,M-1}<th3.
   idx2: the largest i such that v_{M}^{H}−(minM−1),M−2·v_{i,M-1}<th3.
   idx3: the largest i such that pc_{i,M-1}<thev2.
   idx4: the largest i such that σᵢ<thp2.
In various embodiments, th3 can have a value between approximately 0.6 and approximately 1. In various embodiments, thp2 can have a value between approximately 4 and 12. In one embodiment, thp2 can be 5. In one embodiment, th3 can be approximately 0.97.
h. Then, non-cardiopulmonary motion or other signal interference has stopped during frame index max(idx1, idx2, idx3, idx4), as shown in block 1201u.

An oscillating object with a relative displacement greater than half the wavelength of the carrier wavelength in Doppler radar can produce a constellation on the I/Q plot in the shape of a circle. Movement less than half the wavelength can produce an arc, or a portion of a circle. The center of the circle and arc is the combined DC offsets from hardware and from clutter reflections, which produce self-mixing in a direct-conversion system. In some embodiments, a center-find algorithm is used to calculate the center of the circle on which the data points lie. In some embodiments, the movement of the center of the circle or arc more than a threshold value is an indicator of non-cardiopulmonary movement. In some embodiments the center of the mass, or geometric center, of points in the signal is an estimate of the DC offsets, and movement of the center of mass more than a threshold value is an indicator of non-respiratory movement. In some embodiments, a change in the excursion of the envelope in the constellation of data points more than a threshold value can be an indication of non-respiratory motion. In some embodiments, a change in the distance from the center to the points greater than a threshold value is an indicator of non-respiratory movement.

In some embodiments, the onset of motion can be determined by comparing the frequency content of the signal in consecutive frames. In general, the cardiopulmonary signals tend to have fairly localized frequency content with very little change of frequency content in time, and the non-cardiopulmonary signals are more spread out in the frequency spectrum. For this reason, in some embodiments, the onset of motion can be determined by comparing the frequency content of consecutive frames. In some embodiments, when the difference in frequency content of consecutive frames exceeds a certain threshold, the onset of non-cardiopulmonary is identified in the current frame. In some embodiments, spectral subtraction is used to determine the similarity between the frequency content of consecutive frames: the magnitude spectrum of the current frame is subtracted from a weighted sum of the magnitude spectrum of previous frames. In some embodiments, the weights correspond to a decaying exponential. In some embodiments, if the residual energy from spectral subtraction is above a threshold, non-cardiopulmonary motion is identified in the current frame, and, conversely, absence of non-cardiopulmonary motion is re-established whenever the residual from spectral subtraction is below a threshold. In some embodiments, the method is applied separately on the signals pre-demodulation and post-demodulation. In some embodiments, a Cepstrum-based method can be used as an alternative to the spectrum-based method described above. In some embodiments, the frequency properties are assessed after the signal is demodulated. In other embodiments, the frequency properties are assessed before the signal is demodulated.

In some embodiments, the onset of motion can be determined from the wavelet decompositions. In some embodiments, the coefficients of the wavelet decomposition provide the necessary information to identify the type of motion observed: in the case of non-cardiopulmonary motion, the coefficients of interest are those that correspond to the small scales of the wavelet decomposition, and a large magnitude for these coefficients is indicative of the onset of non-cardiopulmonary motion.

In some embodiments, a constant modulus detection scheme can be used to differentiate a cardiopulmonary signal from a cardiopulmonary signal plus non-cardiopulmonary motion. A signal that is constant modulus has a constant complex magnitude. Although cardiopulmonary signals do not necessarily have a constant phase, a DC-coupled cardiopulmonary signal can have a constant modulus if there is no non-cardiopulmonary motion present.

In some embodiments, the signal is compared to a cardiopulmonary motion signal. In some embodiments, no direct attempt is made to identify a non-cardiopulmonary motion signal, but it is inferred as such when the signal does not fit one of the possible cardiopulmonary motion signals. In some embodiments, features are extracted from a database of cardiopulmonary motion signals. In some embodiments, these features highlight the core aspects of a cardiopulmonary signal. In some embodiments, these features include the inhale to exhale ratio, the depth of breath and the inflection points. In other embodiments, these features include the mean, variance and kurtosis of the breath. The same features extracted from the database of cardiopulmonary signals are again extracted from the new signal being considered. The new signal features are compared to the database of features. In some embodiments, if a match is found, then the signal is labeled as a cardiopulmonary motion, and otherwise, a non-cardiopulmonary motion signal is inferred. In some embodiments, the features are selected from the wavelet decomposition of the cardiopulmonary signal. In some embodiments, a mother wavelet is chosen appropriately for this decomposition, and the wavelet coefficients from different scales are chosen to exemplify a specific cardiopulmonary signal.

In various embodiments, a tag attached on a patient's torso can modulate the reflected signal by phase shift keying, frequency shift keying, or another modulation method, to provide a unique identity code of a patient. In some embodiments, the code on this modulated signal can be a patient ID code, which can be synchronized with hospital databases. When the tag is on the patient's torso, the encoded signal is also phase-modulated with the Doppler effect associated with a target's cardiopulmonary motion. In some embodiments, the Doppler-shifted signal from the tag can be compared with the Doppler-shifted signal from non-tag reflections in a correlator or by calculating the correlation coefficient between the two signals. In some embodiments, when the correlation is high between reflected signals encoded with the identification code and the reflected carrier signal, it indicates the absence of non-cardiopulmonary motion. In some embodiments, this correlation can be used to determine whether the received signals are contaminated by other interfering Doppler signals due to non-cardiopulmonary motion, such as the motion of the subject's other body parts, or motion of objects other than the desired subject. In some embodiments if there is a strong correlation between the reflected carrier signal and the tag-encoded signal, the system can not indicate non-cardiopulmonary motion, and if the correlation is weak, the system can indicate cardiopulmonary motion. In some embodiments, the indication of non-cardiopulmonary motion can cease when the correlation between the reflected carrier signal and the tag-encoded signal is high.

In some embodiments, the tag can include an accelerometer to provide similar information as that from the correlator with encoded signal. In some embodiments, acceleration information can be included in the information encoded on the reflected signal, such that the receiver can determine the amount of acceleration on the tag. In some embodiments, acceleration of the tag greater than a threshold can be used to indicate non-cardiopulmonary motion.

In some embodiments, it is possible to detect the number of signal sources, or the number of moving items in the field of view, and/or the location of these signal sources using one or more of several methods, including, but not limited to: identifying patterns in the I/Q plot that are associated with a specific number of sources; utilizing empirical mode decomposition to determine the number of modes, and deriving the number of sources from the number of modes; utilizing independent components analysis, with a number of independent receivers, to identify the number of independent sources; utilizing blind source separation with a number of independent receivers; and utilizing a direction of arrival algorithms, with an array of receivers at known spacing, to determine the number of sources.

In various embodiments, once the number of sources is identified, a threshold can be set on the number of sources, such that when the number of sources exceeds that threshold, non-cardiopulmonary motion is indicated. In various embodiments, once the directions of the sources are identified, if the directions of the sources change more than a threshold value, non-cardiopulmonary motion is indicated. In embodiments utilizing methods such as ICA and BSS, in which the direction of sources, as such, is not identified, the linear combination of input signals from different sources can be used as an analog for direction, such that changes in this linear combination greater than a threshold are identified as non-cardiopulmonary motion.

In some embodiments, a video camera with motion detection signal processing can be used to identify non-cardiopulmonary motion. In some embodiments, infrared detectors or cameras can be used as temperature sensors to monitor for non-cardiopulmonary movement. In some embodiments, pressure sensors in the bed, chair or floor can be used to detect non-cardiopulmonary motion. In some embodiments, laser scanners and range finders can be used to monitor change in distance and/or position, indicating non-cardiopulmonary motion. In some embodiments, passive acoustic scanners can listen for movement and/or breathing, and movement above a threshold can indicate non-cardiopulmonary motion. In some embodiments, active ultrasound scanners and range finders can be used to detect non-cardiopulmonary motion.

As the carrier frequency is increasing or the wavelength is decreasing, there is a greater phase modulation due to the same physical target motion. In some embodiments, when samples lie along an arc with a larger central angle, the center of the circle can be more accurately determined with a LMSE algorithm. Thus, in some embodiments, if the frequency of a carrier signal increases, more accurate circle parameters can be estimated. Some embodiments can use a 24-GHz system, with a wavelength of 1.25 cm, which results in more than 360 degrees of phase modulation with a 1-cm target motion. In some embodiments, non-cardiopulmonary motion can be indicated by changes in the center point or the radius of the circle or arc where the data samples lie; non-cardiopulmonary motion can be indicated when the center point deviation or the radius change is greater than a corresponding threshold value. In some embodiments, non-cardiopulmonary motion can be indicated when the constant modulus condition is violated for the arc or circle. In some embodiments, a weighted combination of these indicators can be used to provide indication of non-cardiopulmonary motion.

In various embodiments, several of these methods and other methods can be combined in a variety of ways. There are different methods for weighing different data that can be used. For example, in some embodiments, if a system uses the change in power and change in eigenvalue to detect motion, rather than independently identifying motion with these parameters, their changing values can be jointly analyzed. Let P be the normalized change in power and E be the normalized change between eigenvalue from each frame. Let TH be an acceptable threshold to indicate motion. In some embodiments, the joint detection method can be characterized by P*½+E*½>TH. In these embodiments, the weight factors are for both the eigenvalue and then power is set to ½. In other embodiments they may not be weighed equally. In other embodiments, power can be separated into separate bands and weighted. As such, in some embodiments, the joint detection can be characterized by P1*W1+P2*W2+Pi*Wi+E*We>TH, where Pi is the normalized change in power in a particular band and Wi is weight factor for the power band. In various embodiments, the weights can be equal or certain bands, such as respiration band, can be weighed more heavily.

In some embodiments, a state machine model can be developed to model motion detection. In some embodiments, instead of motion or no motion, more states can be added to better model the real world system. In some embodiments, 4 states can represent no motion, possible motion, probable motion, and motion. In some embodiments, states can change or remain the same depending on the number of trigger events that have occurred. In various embodiments, trigger events can include, but are not limited to, changes in power levels, changes in eigenvectors, and changes in eigenvalues. In some embodiments, trigger events can be replaced by a point system where the event and the severity of the event can be accounted for. In some embodiments, the transition table can be as shown in FIG. 12E, and the state diagram can be as shown in FIG. 12F. In some embodiments, the state machine can be a Markov chain with transition probabilities as follows:

$$P = ‖ \begin{matrix}{a}_{0} & {a}_{1} & {a}_{2} & 1 - {a}_{0} - {a}_{1} - {a}_{2} \\ {a}_{0} & 0 & {a}_{1} & 1 - {a}_{0} - {a}_{1} \\ 0 & {a}_{0} & 0 & 1 - {a}_{0} \\ 0 & 0 & {a}_{0} & 1 - {a}_{0}\end{matrix} ‖$$

where ai is the probability i events occur. In some embodiments, aᵢ can be characterized by a Poisson random variable with mean λ.

In some embodiments, additional states can be added to provide more quantization levels for describing motion.

In various embodiments, three signal quality measures are computed before applying the rate estimation algorithm on the demodulated signal. First, an algorithm is used to highlight subset of samples of the demodulated signal with non-respiratory signal or interference. Secondly, an algorithm is used to highlight subsets of samples of the demodulated signal that have low power compared to a threshold. Thirdly, an algorithm is used to highlight subsets of samples with clipping. In various embodiments, the rate estimation algorithm also takes into account the low quality samples as determined by the three algorithms and flags them such that they would not affect the accuracy of the rate result. In various embodiments, the rate estimation algorithm uses only the samples that passed these quality checks and attempts to produce a rate based on these. In various embodiments, the rate estimation algorithm can set the flagged samples to zero. If too many of the samples are flagged, the system will not detect a sufficient number of breaths in the interval to for the time-domain rate estimation, and it will report an error. In various embodiments, the rate estimation further uses its own quality check measure. In various embodiments, the rate estimation algorithm is a cross-check of the rate results of a time domain approach and a frequency domain approach for rate estimation. In various embodiments, if the rate determined by the time domain approach differs from the rate determined by the frequency domain method by more than a threshold, the cross-check quality check fails. In various embodiments, if the cross-check quality check fails, the rate estimation communicates the possible reason for this failure. It will attribute the failure to one of these conditions when met in this order: low signal power, signal clipping, non-respiratory signal or interference. If none of these conditions are met, the rate estimation fails with a generic error.

In those embodiments of the system when the center of the circle is estimated from the arc, it is possible to distinguish between inhalation and exhalation by whether the phase of the signal viewed in the complex plane is moving clockwise or counter-clockwise (whether the phase is decreasing or increasing). Differentiation between inhale and exhale is important for some embodiments of triggering applications, some embodiments of synchronization applications, and for embodiments that require calculation of inhale time, exhale time, or the inhale time to exhale time ratio. Some examples of applications that would benefit from differentiation between inhale and exhale for inhale time/exhale time ratio include but are not limited to monitoring of chronic illness, biofeedback for management of chronic illness, and biofeedback for stress.

In some embodiments, information such as differentiation between inhalation and exhalation can be found using non-linear demodulation. With linear demodulation, the direction of movement is ambiguous; however, direction of motion is directly related to the direction of phase change. In some embodiments, the time of exhalation and the time of inhalation can be compared. In some embodiments, even if linear demodulation is used, the side of the line on which the center is can be estimated, such that inhalation can be differentiated from exhalation.

Signals from the system 100 can be used to calculate inhalation time, exhalation time, the length of pauses in breathing, and the ratio of inhalation time to exhalation time. To determine the inhale time—exhale time ratio, the peak inhalation and exhalation points can be determined. This requires that the radar preserve the phase information, such that the direction of phase change can be determined. In a continuous-wave, direct-conversion Doppler radar, this requires that the signal be downconverted with a quadrature mixer, also known as an I/Q demodulator. The quadrature downconversion preserves all the phase information of the signal. After quadrature downconversion, the signal can be plotted in the I/Q plane, and if the target is moving, it can trace out an arc or a circle in the I/Q plane. Depending on how the in-phase (I) and quadrature (Q) downconversion is implemented, either clockwise motion or counterclockwise motion in the I/Q can indicate motion towards the sensor, and motion in the other direction can indicate motion away from the sensor. This depends on the design of the sensor, and can be consistent for all measurements with that sensor design. The maximum inhalation point and maximum exhalation point can be determined in the I/Q plane or after demodulation. To determine maximum inhalation points and maximum exhalation points, it is preferable to determine whether the motion is clockwise or counterclockwise around the origin of the I/Q plane. The center of the I/Q plane can be challenging to determine in some cases because of DC offsets introduced to the I and Q channels are not related to the phase of the signal. While the center of the circle is obvious when a full circle or most of a circle is traced in the I/Q plane, it may not be obvious if the arc in the I/Q plane is very small, and it can be approximated by a line, especially when there is noise on the signal. The phase resolution and signal-to-noise ratio is preferably adequate to determine whether the arc is concave or convex, or which side of the line the center of the arc is on, so it can be determined whether the phasor is moving clockwise or counterclockwise. While the center of the circle is calculated to use an arc-based demodulation algorithm, for determining the inhale-exhale ratio, in some embodiments, it is only necessary to determine which side of the arc the center is on. In some embodiments, this could be used in combination with a linear demodulation method.

In various embodiments, algorithms that can be used to determine which side of the arc the origin is on include, but are not limited to: determining the best-fit circle to the arc with a method such as least squares or maximum likelihood estimator; drawing a line between the ends of the arc and determining which side most of the points are on; fitting the shape with a library of shapes, for which the location of the center is known; and using several permutations of key points, and identifying points that are equidistant from these points, and determining which side of the data most of these points are on. In some embodiments, if the side of the arc the center is on cannot be determined with adequate certainty, the device can provide an error message rather than an inhale/exhale ratio.

In some embodiments, the demodulated data can use center-finding and non-linear demodulation to determine whether the phase is changing in the clockwise and counter-clockwise direction. If the clockwise direction relates to inhalation (depending on hardware implementation), then after demodulation, peaks are maximum inhalation points and valleys as maximum exhalation points. In various embodiments, any peak-finding methods, including, but not limited to, those disclosed elsewhere in this document can be used for finding the peaks, and it can be used in the inverse to find valleys.

After exhalation, there can be a pause before inhalation begins. In some embodiments, the “maximum exhalation point” could be estimated at the point where inhalation begins rather than when exhalation stops or at the minimum valley point. In some embodiments, the length of this pause can be assessed separately from inhalation time and exhalation time. In some embodiments, the first derivative of demodulated data can be used to estimate the exhalation stop points as shown in FIG. 10F. The output of the first derivative function can provide a significantly different value at the point where inhalation starts relative to the values during exhalation through to the maximum exhalation point. Moreover, the sign of the function output during inhalation can be opposite to those of exhalation. It can be achieved by tracking the difference of the signal samples adjacent to each other for the fixed samples for example 500 samples which can be about 0.5 second at 1-kHz sampling rate followed by averaging 499 outputs. Assuming that noise is coming from additive white Gaussian noise, by averaging differences noise can be significantly reduced. In some embodiments, the algorithm defines the maximum exhalation point as the last point in a plateau before a decrease (or increase) greater than a threshold; the plateau continues as long as the threshold is not crossed. In some embodiments, when the absolute value of the first derivative of the demodulated data is below an amplitude threshold for a period longer than a time threshold, that period is considered a pause. In some embodiments, the pause is added to the previous segment (either inhalation or exhalation). In some embodiments, the pause is analyzed separately, and not included in the inhale time exhale time ratio calculation.

In some embodiments, the beginning of inhalation is determined by computing the power of the signal in consecutive intervals beginning from the peak of exhalation of the previous breath and continuing to the peak of the inhalation of the following breath. In some embodiments, the consecutive intervals are of length 100 milliseconds. Inhalation starts at the beginning of the longest sequence of monotonic power levels. In some embodiments, the inhalation period is the time above the zero line and the exhalation is the time below the zero line as shown by trace 1014 of FIG. 10E.

In some embodiments, peaks and valleys can be found after removing a DC offset and/or baseline variation of the signal. In various embodiments, the baseline of the signal can be removed by any method, including but not limited to: high-pass filtering; empirical mode decomposition; line-fitting and subtraction; and/or mean-finding and subtraction.

In some embodiments, maximum inhalation and exhalation points are determined before demodulation. The data constellation on the I/Q plot can mark certain points that have significance after demodulation. In some embodiments, the points where the gradient of the I/Q signal becomes zero are either maximal inhale or maximal exhale points. In some embodiments, their position relative to the other points and the center of the arc or circle can be used to determine whether they are maximum inhalation points or maximum exhalation points.

In some embodiments, a combination of the different peak-finding and valley-finding approaches can be used to ensure that an inhalation or exhalation has not been missed.

In some embodiments, the inhale-exhale ratio can not be calculated if the total inhale-inhale or exhale-exhale time is greater than a threshold which is based on the previous breath or several previous breaths, so that if a maximum inhale point or a maximum exhale point is missed by the algorithm, the inaccurate data can not be used to calculate an inhale-exhale ratio. In some embodiments, this can be an indication of irregular breathing. In some embodiments, non-cardiopulmonary motion detection can be implemented before calculation of the inhale-exhale ratio. In some embodiments, breaths in which non-cardiopulmonary motion is detected can not be used for calculation of the inhale-exhale ratio. In some embodiments, samples in which non-cardiopulmonary motion is detected can be removed before the signal is demodulated and/or the maximum inhale-exhale points are removed, and if adequate data remains, the maximum inhale and maximum exhale points can be calculated from the remaining data.

Once the maximum inhalation and maximum exhalation points are determined, the inhale time and exhale time for each breath can be calculated. In some embodiments, the inhale time is calculated as the time between a maximum exhalation and the following maximum inhalation. In some embodiments, the exhale time is calculated as the time between the maximum inhalation and the following maximum exhalation. In some embodiments, the inhale time to exhale time ratio is typically calculated using an inhale time and the following exhale time, but it could be calculated using an exhale time and the following inhale time. In some embodiments, the ratio is calculated by dividing the inhale time by the exhale time for each breath.

In some embodiments, the value of the ratio can be updated with each breath. In various embodiments, the value for each breath can be displayed, or an weighted average of previous values can be used. In some embodiments, the weighted average can have an exponential weight. In various embodiments, a history for the inhale-time to exhale time ratio can be displayed in addition to the current value.

In various embodiments, of the system 100, the deviation of the phase is proportional to the chest motion divided by the wavelength of the carrier signal, such that the phase deviation can be assessed in signal demodulation, and the depth of breathing can be obtained by multiplying a conversion factor to the phase deviation.

Assuming that target's periodic physiological motion variation is given by x(t), the quadrature baseband output assuming balances channels can be expressed as:

$$B ( t ) = {A}_{r} exp ( i * ( θ + \frac{4 πΔ x ( t )}{λ} ) ) + DC$$

where DC is complex number representing each channel's static voltage value.

Non-linear demodulation extracts the phase information, θ+4πΔx(t)/λ. The static value, θ, caused by the nominal distance of the target, can be removed easily by subtracting the mean value of the output, assuming x(t) is zero mean periodic motion. The direction of the phase trajectory can be used to differentiate between inhalation and exhalation. For example, in some embodiments, if the direction is counter clockwise, the target is inhaling and when the direction is clockwise, the target is exhaling. After non-linear demodulation, the output is directly proportional to the phase deviation caused by the physical chest motion, 4π/λ×Δx(t) [rad]. The absolute motion in the direction of the antenna can be calculated by multiplying λ/4π[cm/rad−1] to the demodulated output.

Depth of breathing can be defined as absolute displacement of the chest or lungs from the maximum inspiration point to the maximum expiration point. In some embodiments, this parameter can be estimated as the absolute distance of the minimum to the maximum. In some embodiments, this parameter can be estimated as the absolute distance from the maximum expiration position to maximum inspiration. In some embodiments, this can be calculated by calculating the angle subtended by the arc at the center in each breath. In other embodiments, the average over several breaths can be used.

In some embodiments, the end-points of the arc can be estimated using various algorithms, including, but not limited to: points of minimal velocity, the center of clusters of point density, or points of largest change in direction. In various embodiments, these end-points can be used in conjunction with a center-finding algorithm that identifies the circle center to identify the angle subtended by the arc.

In some embodiments in which a high frequency carrier signal can be used where the expected chest displacement of a human subject is many times the carrier wavelength, the depth of breath is estimated by counting the rotations of the signal around the center. In some embodiments, direction of rotation between clockwise and counter-clockwise can indicate inhale or exhale.

In some embodiments, movement from respiration upon the chest and abdomen can be differentiated through direction of arrival techniques. In some embodiments, two signals, one from the chest and one from the abdomen, combine in the complex I/Q plane and can provide information about their movement, such as displacement. In some embodiments, these signals from different points on the chest can be combined to provide an overall estimate of depth of breath.

In some embodiments, the depth of breath can be calculated along with other respiratory parameters, including, but not limited to: respiratory rate, inhale time to exhale time ratio, and irregularity of respiration. In some embodiments, thresholds can be set, and when the depth of breath crosses those thresholds, an alarm can be sounded. For example, in one embodiment, a post-operative patient can have a threshold set for the minimum acceptable depth of breath. If the depth of breath drops below this threshold for more than 3 consecutive breaths, a visual, audio, and/or remote alarm can be initiated. In some embodiments, the depth of breath can be used to trigger other medical devices. For example, on a patient receiving patient-controlled analgesia, the PCA pump may not allow additional opioid doses to be initiated if the depth of breath is below a threshold. In various embodiments, the threshold can be set to a factory default value, can be settable by the user, or can be automatically set based on a patient's baseline values or other information from the patient's medical record.

In various embodiments, the system 100 can perform a self-check to check for improper operation and/or environmental interference. In some embodiments, the self-check can be performed automatically. In various embodiments of the system, a self-test can be performed periodically to determine if portions of the hardware are malfunctioning. In various embodiments, the self-test can be performed by digitally controlling the activation of various components of the system and analyzing characteristics such as, but not limited to, channel noise level, channel imbalance and DC offset values. Although the self-test can be integrated as part of the system's start-up procedures, in various embodiments, the system 100 can require commands from the central controller to initiate the various self-test checks. In addition to hardware status, RF interference tests can be performed by comparing the normal transmitted RF power and reduced transmitted RF power. This can ensure that the received signal is not a result of an extra-sensor device producing cardio-pulmonary like signals.

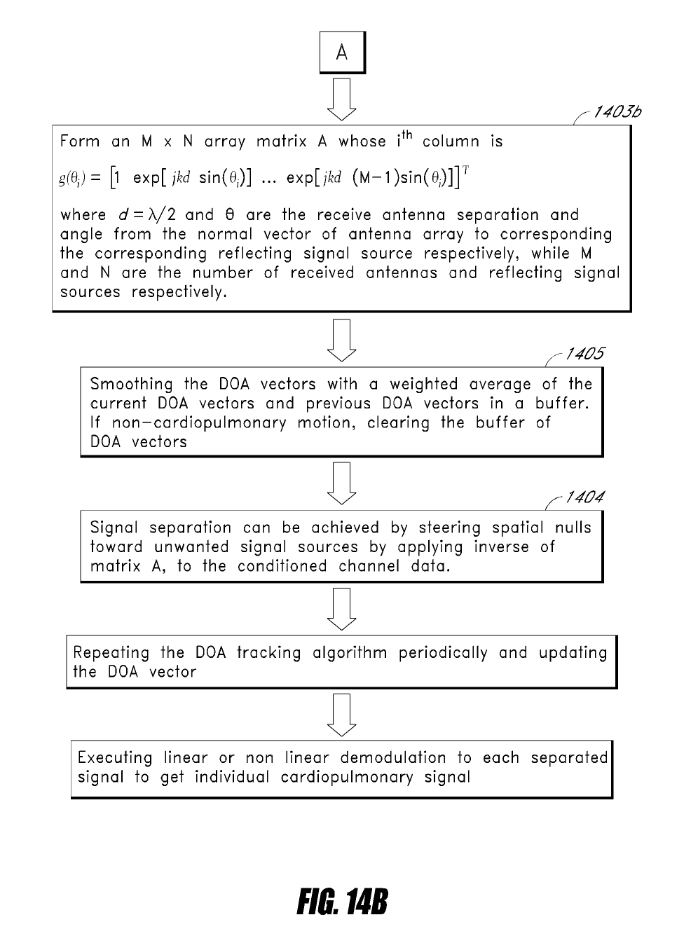

FIG. 13 illustrates a block diagram of a self testing circuit 1300. In various embodiments, the self testing circuit includes an absorptive SPDT switch, 1301 and voltage controlled phase shifter 1302. The SPDT switch 1301 can be used for selecting either transmitting path 1303 or self testing path 1304. A voltage controlled phase shifter implemented on self testing path generates an artificial signal which is inputted in to RF input port of IQ demodulator 1305 through 0 degree power splitter 1306. The signal makes either full circle or partial of arc depending on the control voltage on complex constellation plot. The plot can be used to test the signal source, IQ imbalance, external interference, baseband signal conditioning, and data acquisition.

In various embodiments, a processor configured to execute a direction of arrival algorithm can be used to isolate cardiopulmonary motion from spatially separated non-cardiopulmonary motion based on their differing angles from the antenna as disclosed in U.S. Provisional App. No. 61/125,027, which is incorporated herein by reference in its entirety and in U.S. Provisional App. No. 61/125,020, which is incorporated herein by reference in its entirety. In various embodiments, a processor configured to execute a direction of arrival algorithm can be used to isolate separate two spatially separated cardiopulmonary motion signals based on their differing angles from the antenna. In various embodiments, a processor configured to execute a direction of arrival algorithm can be used to track the angle to a subject. To use direction of arrival, the radar-based physiological motion sensor includes at least two antennas in each plane in which it is desired to assess the direction of the source, and/or to separate spatially separated motion for subject separation and for non-cardiopulmonary motion cancellation.

In various embodiments, it is often desirable to have a wide antenna beam width, to ensure that the beam covers the subject in all probable positions. However, this wide beam width means that motion away from the subject can still be in the antenna's mean, and therefore can still affect the measurement. In various embodiments, direction of arrival (DOA) processing from multiple receive antennas can provide a wide angle of scanning to detect the subject, and then a narrower angle for measurement of a subject's physiological motion, avoiding interference from motion away from the subject. In some embodiments, the signals from the antennas can be processed as an antenna array, which has a narrower beam width than any of the individual antennas. Through processing, the beam of this array can be effectively steered towards the desired source, so the antenna beam is focused on the source and any motion outside the beam will be attenuated according to the antenna pattern in that direction. Additionally in various embodiments, the angle to the target subject can be detected and presented in the interface, either as the angle or as a more general indication of the direction (i.e., straight, left, or right), effectively providing tracking of the subject.

In various embodiments, the signals from the different antennas can be used to detect and track the angle of an interfering source, and the signals from the antennas can be combined such that there is a null in the antenna pattern in the direction of the interfering motion, enabling continued detection of respiratory waveform in the presence of spatially separated motion. Any of several DOA algorithms can be used for this technique. These approaches can be used in a SIMO system including one transmitter and multiple receiver antennas. The DOA algorithms can be implemented in a MIMO system including multiple transmitters, each transmitting at a different frequency, and multiple receivers. Other advanced DOA algorithms including but not limited to MUSIC or ESPRIT could also be used to separate sources at different angles from the antenna.

In various embodiments, DOA processing can be used to isolate rib cage and abdominal breathing as disclosed in U.S. Provisional App. No. 61/125,020, which is incorporated herein by reference in its entirety. In various embodiments, DOA processing can be used to isolate leg motion from cardiopulmonary motion, enabling detection of restless leg syndrome during sleep. In various embodiments, multiple subjects can be monitored with one device using DOA processing as disclosed in U.S. Provisional App. No. 61/194,880 which is incorporated herein by reference in its entirety. As described above, in various embodiments, a Doppler radar system 100 can monitor a human's physiological signals such as respiration or heart waveforms, and respiratory and heart rates can be extracted. By employing multiple antennas in the system, direction of arrival (DOA) processing can be achieved, enabling detection of the angular direction of targets. In various embodiments, multiple targets' physiological signals can be separated based on DOA processing obtained by an arrayed Doppler radar. In various embodiments, separating these physiological signals can enable the waveforms of each target to be separated for the display or communication of waveforms and for the extraction of rates. If multiple people are within the antennas' field of view, each person's respiratory rates can be obtained with this signal processing scheme, as long as their angular separation is greater than the resolution of the array and there are no more people within the field of view than antennas and receivers in the plane the people and the antenna share is less than the number of antennas and receivers. In some embodiments, the multiple antennas can be separated by a distance λ/2. In various embodiments employing three antennas, two subjects who are separated by approximately 15 to 20 degrees can be simultaneously tracked and monitored. By increasing the number of antennas the angular separation between the two subjects can be further reduced.

One embodiment of a method for separating multiple cardiopulmonary signals is illustrated in FIG. 14 and includes:
1. As illustrated in blocks 1401a-1401d, the method includes determining the frequency components f=f₁, f₂, . . . , fₙ of the buffered data that are most likely to contain the cardiopulmonary signals. In some embodiments, these frequency components can be determined by measuring the power spectral density of the combination of the channels, and applying a cost function to the output. In some embodiments, the power spectrum density of the combination of channels can be determined by obtaining the power spectral density from each receiver and multiplying them to get a combined spectrum. In some embodiments, a low-pass filter is applied before obtaining the power spectral density from each receiver. In some embodiments, the cutoff frequency of said low-pass filter is 1 Hz.
2. As shown in block 1402, the method further includes identifying the angular direction of each frequency component. In some embodiments, the angular frequency components are identified by forming a channel matrix H whose entries correspond to the frequency components most likely to contain the cardiopulmonary signals found in Step 1, using this channel matrix and an array vector corresponding to each angle from the target to calculate the maximum average power at each angle. In some embodiments, the m^{th} row and n^{th} column of the channel matrix entry can be hₘₙ=sₘₙ(fₙ), corresponding to the receiver antenna m and moving scatterer, where sₘₙ represents frequency spectrum of the channel. In some embodiments, an array vector corresponding to each angle from the target is formed. In some embodiments, the array vector is given by equation (1):

g(θ)=[1exp[jkd sin(θ)] . . . exp[jkd(M−1)sin(θ)]]^{T} (1)
where k is the wavenumber, d=λ/2 is the separation distance between each receiver antenna and θ is the angle from the antenna normal vector to the target, while M is the number of received antennas. In some embodiments, the maximum average power that can be obtained at each the angle of the scatterers is given by equation (2):

Pₐᵥ(θ)=|H^{H}g(θ)|² (2)
3. As illustrated in blocks 1403a and 1403b, the method further includes eliminating angles that are separated from each other by an angular distance less than the angular resolution of the multiple receiver antenna array, and identifying at least a first and second angular direction such that each angular direction is separated from each other angular source by an angular distance greater than or equal to an angular resolution of said multiple receiver antenna array.4. Generating a DOA vector with unity magnitude for each target in the said angular direction. In various embodiments, an M×N array matrix A is formed, whose ith column is given by the equation (3)

g(θᵢ)=[1exp[jkd sin(θᵢ)] . . . exp[jkd(M−1)sin(θᵢ)]]^{T} (3)
where d=λ/2 and θ are the receive antenna separation and angle respectively, while M is the number of received antennas. In those embodiments where there are other moving objects in the vicinity of the subject which can scatter the radar signal and are separated by an angular distance greater than the angular resolution of the multiple receiver antenna array, N denotes the number of moving scatterers.
4. In various embodiments, smoothing the DOA vectors with a weighted average of the current DOA vectors and previous DOA vectors in a buffer, as shown in block 1405.
5. Separating the signal from each angular direction by steering spatial nulls towards the other angular directions, as shown in block 1404. In various embodiments, the signal separation can be achieved by steering spatial nulls toward unwanted signal sources by applying inverse of matrix A, estimated in step 4, to the conditioned channel data.

S=A⁻¹Rₓ (4)
6. In various embodiments, applying the non-cardiopulmonary motion detector to each separated output, and if non-cardiopulmonary motion is detected, clearing the buffer of DOA vectors
7. In various embodiments, demodulating each of the separated signals individually, and processing each signal to obtain information corresponding to cardiopulmonary motion.
8. Outputting information on at least one of the angle to each target, cardiopulmonary motion related to the target.

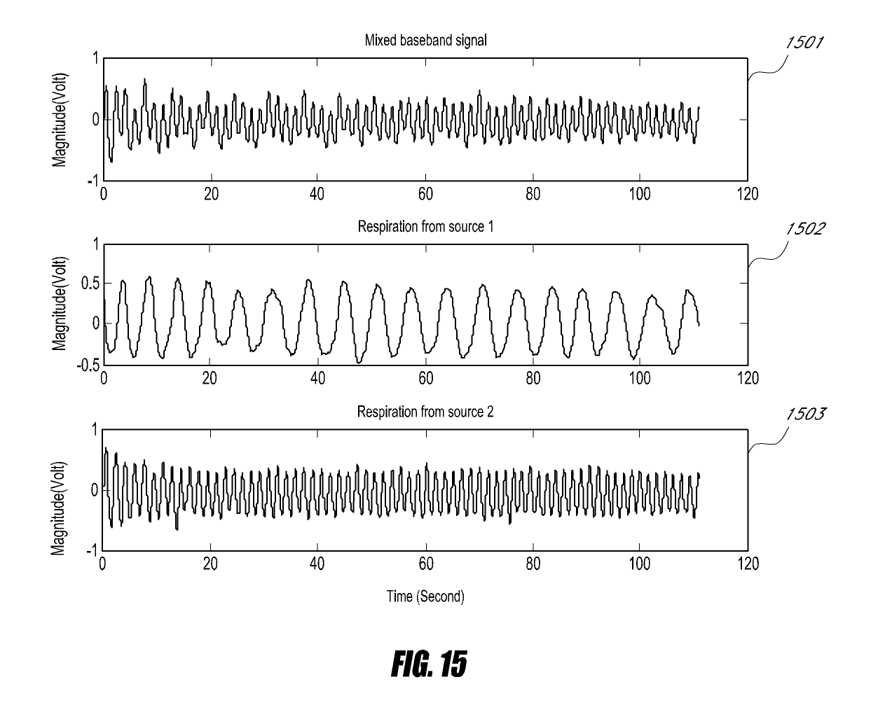

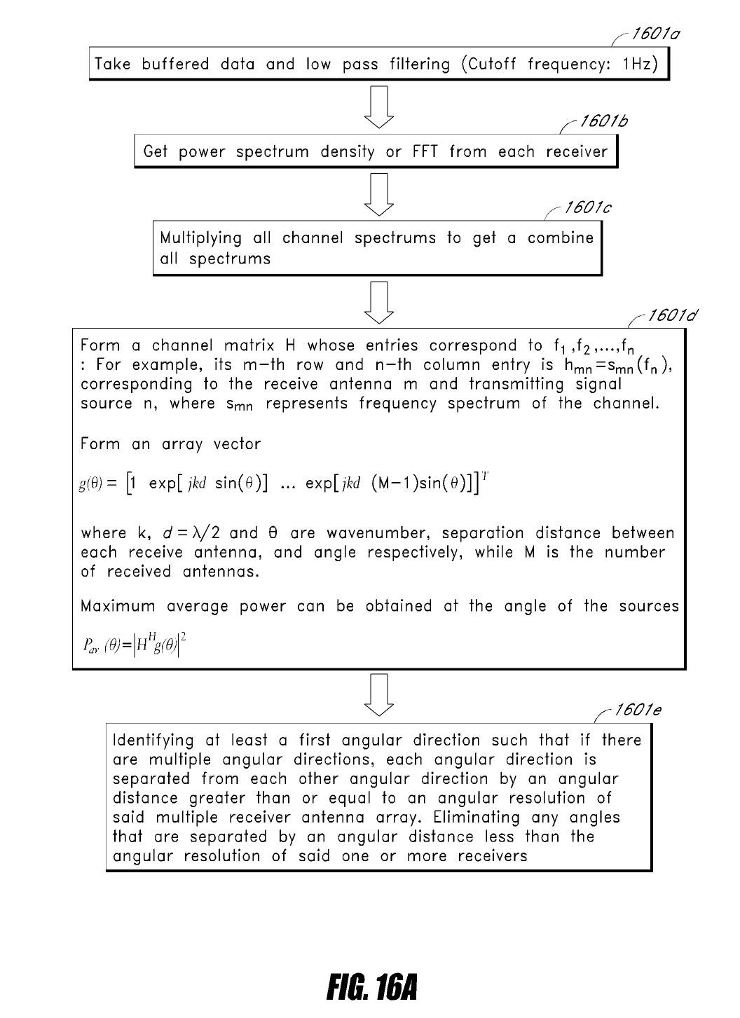

FIG. 15 illustrates the separation of respiratory signals from two targets. Plot 1501 illustrates a mixed baseband signal which is separated using DOA processing. Plot 1502 illustrates the respiratory signal from a first subject or source and plot 1503 illustrates the respiratory signal from a second source or subject. In various embodiments, a body-worn identification tag including a system configured to perform DOA processing can be used to help identify and enhance measurement of a targeted subject as disclosed in U.S. Provisional App. No. 61/200,876 which is incorporated herein by reference in its entirety.

Alternatively to separating and analyzing two distinct signals, in various embodiments of the device, the system 100 can use the DOA algorithm to track a single, desired, cardiopulmonary signal, while nulling one or more undesired cardiopulmonary or non-cardiopulmonary signals. In some embodiments, the desired subject can be tracked with an RFID tag. In some embodiments, the desired subject can be tracked with biometrics. In some embodiments, the desired subject can be tracked based on a known initial position. In this case, only the desired signal will be demodulated and only the angle information and/or cardiopulmonary information related to the desired target will be outputted. The various embodiments of the system 100 can include DOA processing algorithms to track a subject or patient as disclosed in U.S. Provisional App. No. 61/125,020, which is incorporated herein by reference in its entirety and in U.S. Provisional App. No. 61/194,836 which is incorporated herein by reference in its entirety. For example, in some embodiments, DOA processing can be used to track a sleeping subject throughout the night as the subject tosses and turns while sleeping.

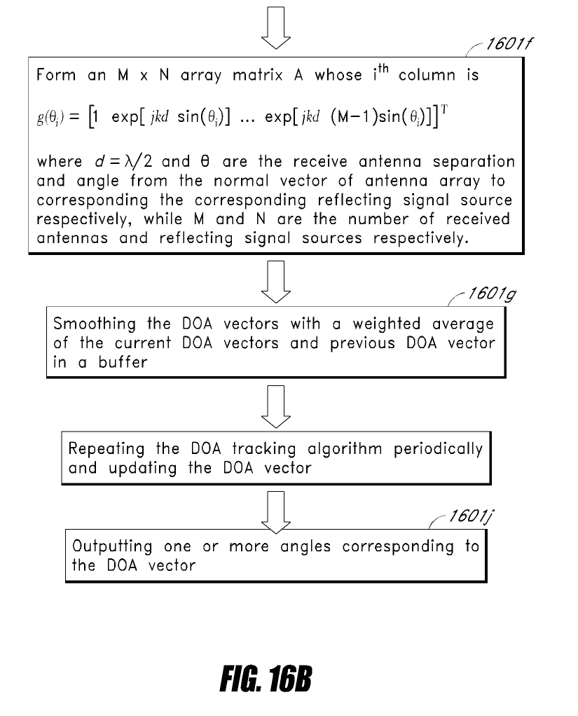

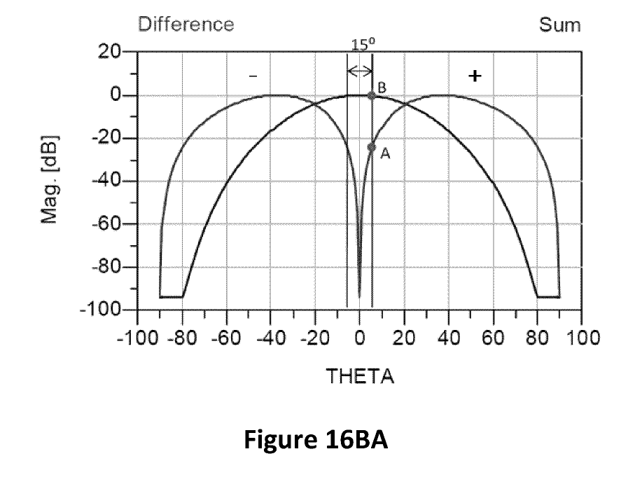

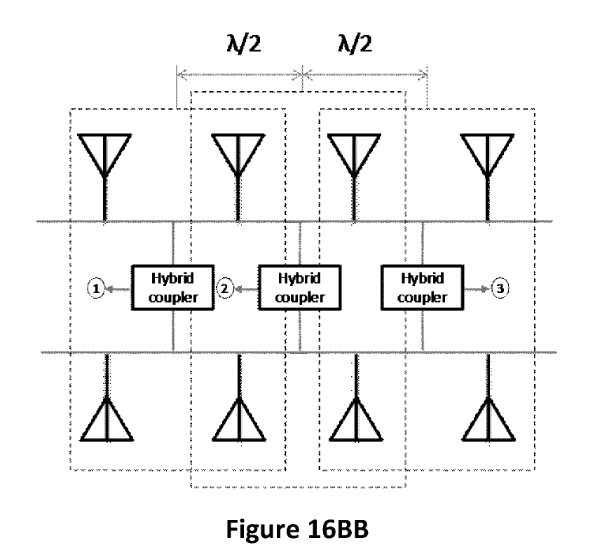

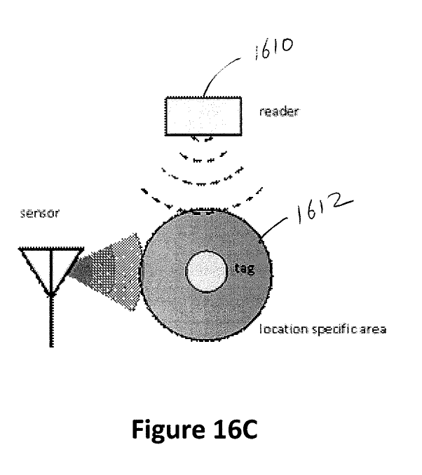

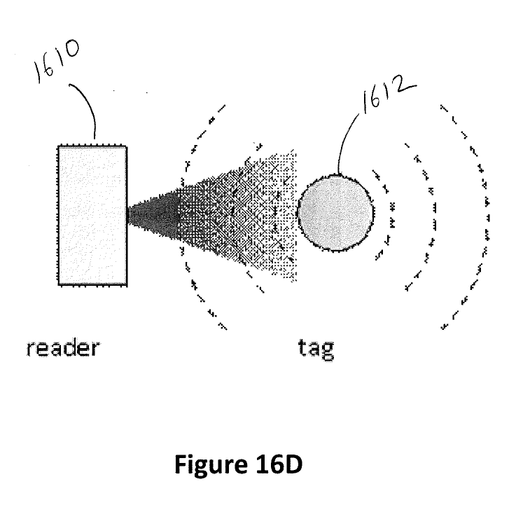

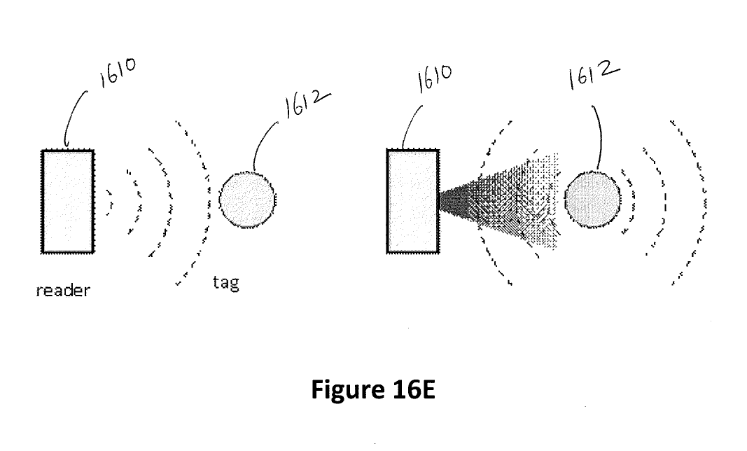

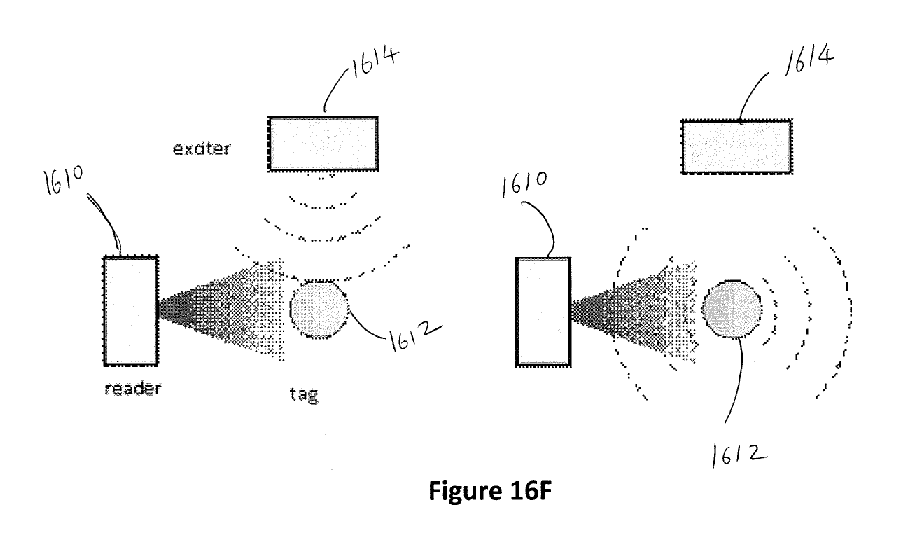

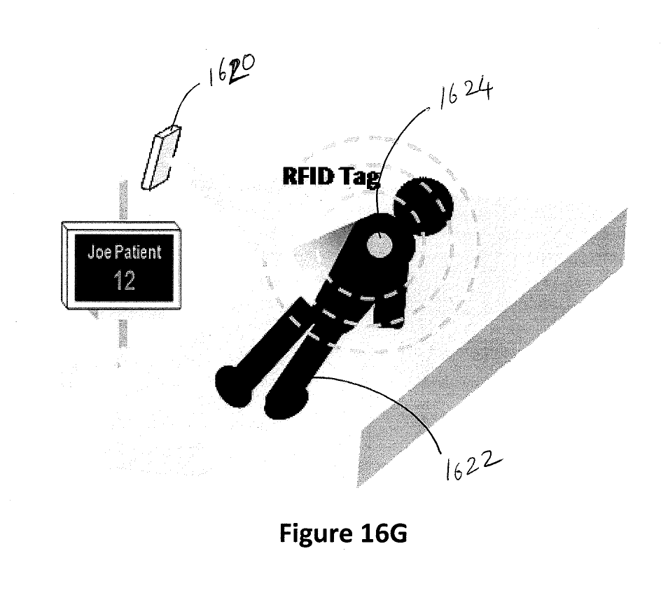

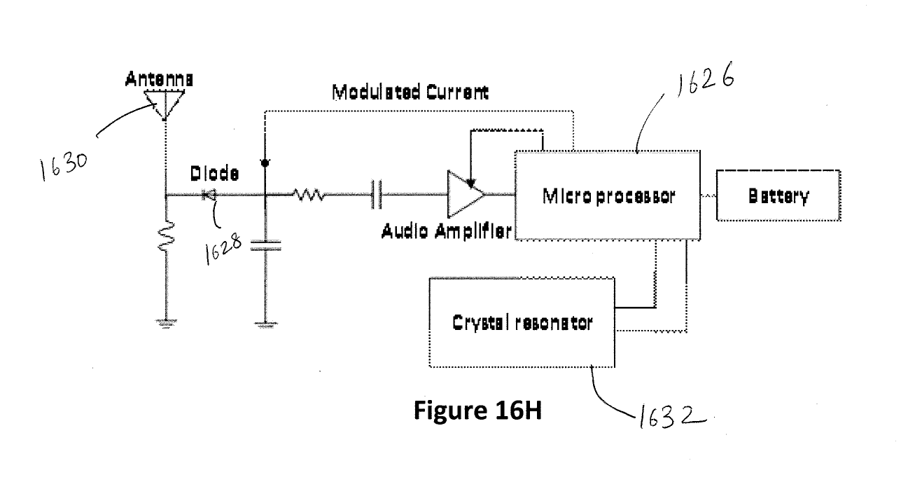

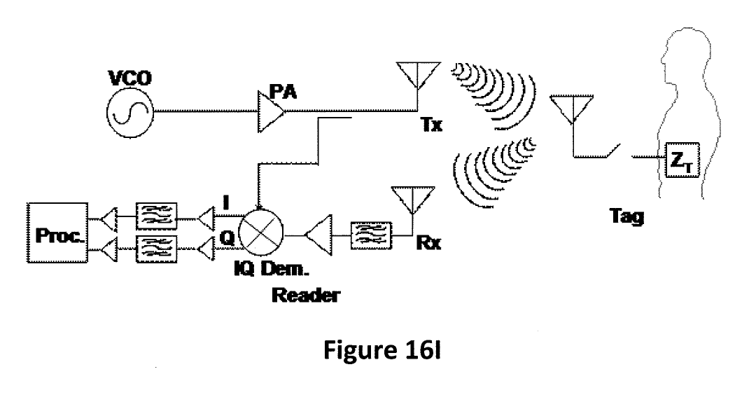

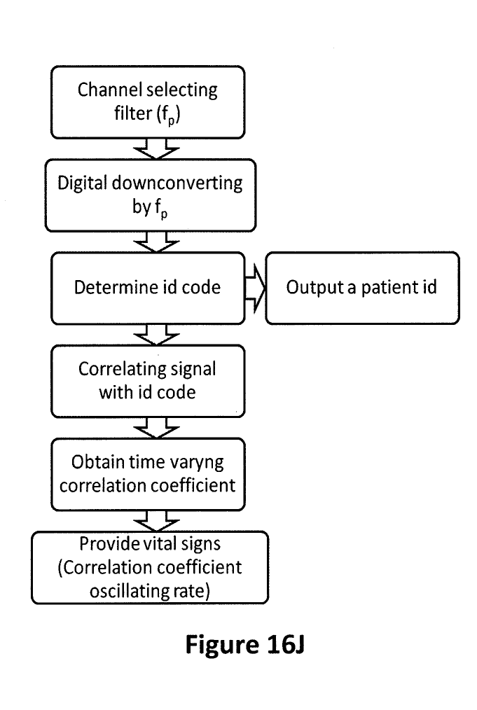

One embodiment algorithm for tracking the direction of one or more cardiopulmonary signals is described below as illustrated in FIG. 16 and includes:
1. As illustrated in blocks 1601a-1601c, the method includes determining the frequency components f=f₁, f₂, . . . , fₙ of the buffered data that are most likely to contain the cardiopulmonary signals. In some embodiments, these frequency components can be determined by measuring the power spectral density of the combination of the channels, and applying a cost function to the output. In some embodiments, the power spectrum density of the combination of channels can be determined by obtaining the power spectral density from each receiver and multiplying them to get a combined spectrum. In some embodiments, a low-pass filter is applied before obtaining the power spectral density from each receiver. In some embodiments, the cutoff frequency of said low-pass filter is 1 Hz.
2. As illustrated in step 1601d, the method further includes identifying the angular direction of each frequency component. In some embodiments, the angular frequency components are identified by forming a channel matrix H whose entries correspond to the frequency components most likely to contain the cardiopulmonary signals found in Step 1, using this channel matrix and an array vector corresponding to each angle from the target to calculate the maximum average power at each angle. In some embodiments, the m^{th} row and n^{th} column of the channel matrix entry can be hₘₙ=sₘₙ(fₙ), corresponding to the receiver antenna m and moving scatterer, where sₘₙ represents frequency spectrum of the channel. In some embodiments, an array vector corresponding to each angle from the target is formed. In some embodiments, the array vector is given by equation (1):

g(θ)=[1exp[jkd sin(θ)] . . . exp[jkd(M−1)sin(θ)]]^{T} (1)
where k is the wavenumber, d=λ/2 is the separation distance between each receiver antenna and θ is the angle from the antenna normal vector to the target, while M is the number of received antennas. In some embodiments, the maximum average power that can be obtained at each the angle of the scatterers is given by equation (2):

Pₐᵥ(θ)=|H^{H}g(θ)|² (2)
3. As illustrated in block 1604e, the method further includes eliminating angles that are separated from each other by an angular distance less than the angular resolution of the multiple receiver antenna array, and identifying at least a first and second angular direction such that each angular direction is separated from each other angular source by an angular distance greater than or equal to an angular resolution of said multiple receiver antenna array.
4. Generating a DOA vector with unity magnitude for each target in the said angular direction. In various embodiments, an M×N array matrix A is formed, as shown in block 1601f, whose ith column is given by the equation (3)

g(θᵢ)=[1exp[jkd sin(θ)] . . . exp[jkd(M−1)sin(θᵢ)]]^{T} (3)
where d=λ/2 and θ are the receive antenna separation and angle respectively, while M is the number of received antennas. In those embodiments where there are other moving objects in the vicinity of the subject which can scatter the radar signal and are separated by an angular distance greater than the angular resolution of the multiple receiver antenna array, N denotes the number of moving scatterers.
5. In various embodiments, smoothing the DOA vectors with a weighted average of the current DOA vectors and previous DOA vectors in a buffer, as shown in block 1601g.
6. Separating the signal from each angular direction by steering spatial nulls towards the other angular directions. In various embodiments, the signal separation can be achieved by steering spatial nulls toward unwanted signal sources by applying inverse of matrix A, estimated in step 4, to the conditioned channel data.

S=A⁻¹Rₓ (4)
7. [In various embodiments, applying the non-cardiopulmonary motion detector to each separated output, and if non-cardiopulmonary motion is detected, clearing the buffer of DOA vectors.
8. In various embodiments, demodulating each of the separated signals individually, and processing each signal to obtain information corresponding to cardiopulmonary motion.
9. Outputting information on at least one of the angle to each target, cardiopulmonary motion related to the target as shown in block 1601j.

In various embodiments, empirical mode decomposition (EMD) algorithms can be used to isolate the signal from motion as disclosed in U.S. Provisional App. No. 61/125,023, which is incorporated herein by reference in its entirety including motion due to but not limited to non-cardiopulmonary motion by the subject, cardiopulmonary motion of one or more people other than the intended subject, non-cardiopulmonary motion of another person or other people, motion of other objects in the environment, motion of the radar system.

Various embodiments of the system 100 can include a combination of Empirical Mode Decomposition and Direction of Arrival processing as disclosed in U.S. Provisional App. No. 61/125,027, which is incorporated herein by reference in its entirety. In some embodiments, the DOA processing can be used to separate motion signals that occur at different angles. Subsequently EMD processing can be used to extract the desired physiological motion signal from non-physiological motion and other signal interference that remains after DOA processing. Various embodiments can include a processor configured to execute a motion compensation algorithm. Motion compensation can suppress interference with cardiopulmonary signals caused by movement of other body parts or movement by another person in the antenna's field of view. The cardiopulmonary signal can be in a low frequency range e.g., from a few Hz to a few kHz even including harmonics, while other non-cardiopulmonary motion can be wideband because it moves more quickly; for example, an impulse response can include all frequency components. In some embodiments, the motion compensation algorithm can separate low pass filtered and high pass filtered versions of the data or signal and find at least two primary vectors (e.g., principle eigenvectors) for the high pass filtered data or signal. The low pass filtered data or signals which include the cardiopulmonary signal, can be projected on the orthogonal subspace spanned by these primary vectors of the high pass filtered signal. This subspace can contain reduced or minimal motion interference. This approach can provide information related to the respiratory signal with greater accuracy when used with multiple spatially separated antennas.

In Doppler radar-based monitoring of physiological motion, different sources of motion can be differentiated using various methods, including the following:

Direction of Arrival algorithms with multiple receivers

Blind Source Separation with multiple receivers

Empirical Mode Decomposition with a single receiver

Independent Components Analysis with multiple receivers

A signal receiver with a mechanically steered antenna

An active array, with an electrically steered antenna beam

In various embodiments, facial recognition software can be used to identify the number of people in the antennas' field of view, and can be used in conjunction with DOA algorithms or other source separation algorithms to focus on the desired subject.

In various embodiments, the desired target can wear a tag that can be used for aiming and/or identification of the desired target. In some embodiments, the signal strength from the tag can be used to aid with aiming. In some embodiments, a tag can be used in conjunction with DOA processing to determine the direction of the tag and to focus the receive beam of a multiple-receiver system in this direction. In some embodiments, the tag can provide a harmonic of the transmitted signal or a modulated version of the transmitted signal. In some of these embodiments, the signal can be obtained from the tag signal rather than the overall Doppler signal, to ensure that the signal comes from the desired source. In some embodiments, a retro-directive antenna sends the signal back in the same direction using a phased array or corner antennas.

In some embodiments, monopulse tracking techniques can be applied to track the direction of the source with higher resolution in connection with the DOA process illustrated in FIG. 16BA. Rather than finding the maximum power coming from the direction of the summation of two squinted beams, this method tracks the minimum power coming from the direction of the subtraction of the two beams. An error signal voltage can be calculated by multiplying the difference between the two beams with the sum of the two beams. A bigger absolute value of the error signal voltage implies the more offset of the source direction from the estimated direction. The polarity of the error signal voltage provides the direction of the offset. For example, negative means a source is located on the left from the estimated direction, while positive means the right side.

In various embodiments utilizing DOA algorithms, the DOA algorithms can include second lobe cancellation, MUSIC (eigenvector decomposition) and/or Esprit. Various DOA algorithms can include steps of finding the angle of the desired source and of undesired source, and of maximizing the desired source power to undesired source power ratio.

In various embodiments which require multiple receivers, the following arrangements of antenna arrays can be used: linear, circular, random, rectangular 2D array, antennas or placed in the room corners. In some embodiments 2D array is composed of planar antennas that are distributed on the plane whose vector is directing to targets. In some embodiments 2D array is composed of omnidirectional antennas that are distributed on the plane whose vector is parallel to targets. In some embodiments where antennas are placed in the room corners, at least 3 antennas can be used to determine a point at which the motion occurs.

In some embodiments, array size can be reduced by sharing antennas as shown, for example, in FIG. 16BB. In the figure, four antennas comprise one single cell that has 6 dB higher gain than a single antenna. Furthermore, column antennas in each cell are shared for its adjacent cells, resulting in a compact array feature.

In some embodiments, a bistatic radar can be used, where the receiver is spatially separated from the transmitter.

Noise reduction can be obtained through filtering, wherein the filter passes signals in the physiological band and attenuates signals outside of that band.

Since the cardiopulmonary signal has low frequency components, an oversampling and averaging method can be applied to reduce noise with inexpensive data acquisition devices. By oversampling, the uncorrelated noise power (such as AWGN) on baseband signals can be reduced by a factor of 1/N by averaging N samples, while keeping the same signal power, resulting in a SNR that is N times greater with oversampling and averaging than with Nyquist sampling.

Noise reduction can be obtained through performing empirical mode decomposition and selecting the one or more modes that contain the physiological signal(s) and using only those to reconstitute the signal. The empirical mode decomposition algorithm adaptively separates the signal into intrinsic mode functions (IMFs) which are adaptively created based on the highest-energy intrinsic time scales in the data, and thus capture the most important information in the signal. IMFs have well-defined Hilbert transforms. This empirical mode decomposition algorithm can be used to process the digitized output of a radar designed to measure cardiopulmonary motion of a subject. The quadrature outputs of the radar signal can be processed with an EMD algorithm including at least one of bivariate EMD, complex EMD, or rotation-variant EMD. The IMFs of the I and Q channels can be combined with a linear or nonlinear demodulation algorithm. Then a motion signal can be constructed from the IMFs containing the signal, without the IMFs that contain only noise, resulting in significant noise reduction as disclosed in U.S. Provisional App. No. 61/125,023, which is incorporated herein by reference in its entirety.

In various embodiments, an identification (ID) system is used to provide positive patient identification in conjunction with remote vital signal sensing as illustrated in FIG. 16C. Various embodiments of an ID system have two basic components: a reader 1610 and a tag 1612. The tag 1612 is a device placed on or near the patient that emits or re-emits a signal. This emitted or re-emitted signal is modulated in such a way that it is encoded with unique identification that marks that signal as being from a specific tag. In some embodiments, this unique identification indicates a patient identification number that is used in hospital records. The reader 1610 is a device that takes the modulated signal from the tag 1612 and identifies the coded information. In some embodiments, the reader 1610 can also provide the source signal that the tag 1612 modulates and re-emits. In order for an identification system to link the vital-sign assessment to a particular patient, it is sufficient to ensure that the patient is within the area in which the direction-sensitive and range-sensitive sensor can measure. In some embodiments, direction sensitivity in a remote-sensing radar is achieved through use of a directional antenna that is insensitive to signals outside of a limited angle range in two dimensions. In various embodiments, range sensitivity is limited either through power sensitivity or range-gating of pulse signals. A location-specific ID system should be have an active area within of this three dimensional space of sensor sensitivity.

In some embodiments, the tags can be encoded with a patient identification number. In some embodiments, the vital signs monitor could access patient information (name, etc.) with information obtained from this tag and display patient information for the patient being measured on the display. In some embodiments, the vital signs monitor could transmit vital signs information with the patient identification number such that in a central nursing station, the vital signs would be displayed with the patient identification number, or such that the vital signs would be stored with the patient's electronic medical record.

In some embodiments, at the initiation of a continuous measurement, the nurse would synchronize the vital signs monitor with the tag worn by the patient, such that it can only monitor, display, transmit, and/or record vital signs when that tag is in the field of view, until a new measurement is initiated, with a new tag.

FIG. 16D shows an embodiment of an active tag 1612 emitting a signal modulated with a unique ID signature that is received by the reader device 1610. In this embodiment, the reader 1610 has a directional antenna that detects the tag's 1612 signal from a specific angle range. In various embodiments, the power of the tag 1612 can be adjusted to limit the range in which the tag can be sensed such that the ID area is the same area sensed by the vital-sign monitor.

FIG. 16E shows a tag 1612 receiving a signal and either re-emitting the signal modulated with unique ID information (passive) or emitting a new signal (active). In various embodiments, in order for the ID to be location specific, the transmit and/or the receive apparatus should be directional. In various embodiments, the tag 1612 can either emit or re-emit in an omni-directional fashion or utilizing some sort or retro-directive method such as a corner reflector or a phased array.

In some embodiments, a signal is sent by an exciter, received by the tag, re-emitted in an omni-directional direction, with the signal modulated by the tag in such a way that there is identifiable information in the signal, and then detected by a receiver. In some embodiments, the tag reflects the signal back to the source using, for example, a retro-directive array or a corner reflector. In some embodiments, the exciter can be co-located with the receiver. In some embodiments, the exciter and receiver are together in a transceiver architecture. In some embodiments, modulation can be amplitude modulation, phase modulation, or frequency modulation of the carrier signal. In some embodiments, the tag can return a signal that has orthogonal polarization for linear polarization or counter rotation, for circular polarization. In some embodiments, the tag can return a signal that is a harmonic of the carrier signal. In some embodiments, digital information is modulated by methods including, but not limited to: pulse width, pulse delay, pulse amplitude, and pulse density.

FIG. 16F is similar to FIG. 16E in which the tag receives a signal and emits or re-emits a modulated signal with a unique ID. However, this is a more general form in which the exciter 1614 and the reader 1610 are separate and not necessarily co-located. In this case both the exciter 1614 and the reader 1610 can be directional in order to make the affective area specific to the area sensed by the vital-sign monitor. In some embodiments, the exciter and the reader may not be co-located.

In some embodiments of an active tag, a battery-operated RFID tag is sensed by a reader with a directional antenna co-located with vital-sign sensor.

In some embodiments, an infra-red LED tag pulses a unique ID, which is read by an IR-sensitive camera. This camera data is analyzed to restrict vital-sign sensing to periods when the LED is in a specific area in the camera's view. In various embodiments, the camera is either ceiling mounted or co-located with the sensor.

In some embodiments, an ultra-sonic tag is utilized which has a modulated sonic signal at a frequency above that which humans can hear. In some embodiments, ultrasonic microphones can be placed for triangulation to position of tag, and the tag position can be analyzed to indicate whether it is within the range and angle from which the radar-based vital signs sensor can operate.

In some embodiments, the reader is located with the patient and identifies coded information in the vital-sign sensor's RF signal. The reader responds with an omni-directional signal indicating proper ID acquisition. In various embodiments, this response signal can include, but is not limited to: IEEE 802.11 (wifi), Bluetooth, zig-bee, ultra-sonic, infra-red and/or ISM band RF radiation.

In some embodiments, a tag re-emits RF radiation from vital-sign sensor's transmitter modulated with its unique ID. In various embodiments, the reader, with a directional antenna, can be ceiling-mounted, floor mounted, or co-located with the vital-sign sensor. In some embodiments, the reader can have a directional antenna. In some embodiments, the tag re-emits an omni-directional signal.

In some embodiments, a camera is mounted on the ceiling or co-located with the sensor, and uses facial recognition algorithms to indicate whether the patient is in specific areas of a hospital room before recording vital-signs. In some embodiments, when the healthcare practitioner initiates the measurements, he or she synchronizes the sensor with the face of the patient.

In some embodiments, a camera is mounted on the ceiling or co-located with the sensor, and the patient's tag or hospital gown has a unique pattern that can be deduced by the image-processing algorithms.

Some embodiments of the system can use a Doppler radar-based identification system that can provide positive patient identification while acquiring vital sign signals. In some embodiments, the identification system can provide alternative means of acquiring physiological signals. FIG. 16G illustrates the basic concept of enabling positive identification (ID) using a tag attached on the patient. The tag reader, or reader unit 1620, transmits a continuous wave (CW) signal towards the subject 1622 using a somewhat directive antenna beam illuminating the subject 1622. As the signal is reflected from the subject's thorax, its phase is modulated proportionally to the thorax's cardiac and respiratory motion. When this signal is received and downconverted, there is a baseband Doppler signal at the cardiopulmonary signal frequency. In various embodiments, the ID tag 1624 can be attached to the patient's upper body, either attached to the clothing or adhered to the skin of the patient with an adhesive. In some embodiments, the tag 1624 can be battery operated; however, it can be passive in the sense that it can not generate transmit signals on its own, but when the signal transmitted by the reader unit 1620 illuminates the tag 1624, the tag 1624 can modulate the backscatter by changing the reflection coefficient from the antenna at a programmed frequency. In some embodiments, the reflection coefficient from the antenna can be changed by periodically connecting the antenna to a load by controlling the bias current of a diode connecting the antenna and a load, resulting in generation of sidebands that carry ID information. In some embodiments, the periodic connection of the antenna to a load requires a local battery on the tag.

One embodiment of the passive transponder RFID technology is shown in FIG. 16H. The illustrated embodiment is a crystal 1632 based two-way radio powered by a watch battery. This tag is passive in the sense that it does not generate a signal by itself, however it requires a battery to power a microprocessor 1626 and provide a modulating current to the diode. The backscatter from the tag is modulated by the bias current to the diode 1628, which changes the impedance “seen” by the tag antenna 1630, and thus the power reflected from the antenna. The modulating current is produced by a microprocessor 1626 driven by a low frequency clock, (in some embodiments, the clock is in the 10 kHz range). Thus, the modulated backscatter appears at the sideband frequency (in some embodiments, in the 10 kHz range), and can be easily separated from the baseband Doppler signal through filtering in the digital domain. The data acquisition sampling rate is preferably greater than twice the sideband frequency range (in some embodiments, 20 kHz) to avoid aliasing. In some embodiments in which a low-IF architecture is used, the sampling rate is selected considering that the sampling rate is preferably at least double the low IF frequency+double the sideband frequency. In some embodiments, the tag antenna 1630 is omni-directional to ensure that the backscatter can be detected by the reader if the subject changes position. In some embodiments, multiple tags can be used to provide signal diversity, for example on the front and back of the subject, but in other embodiments, only one tag is needed. In some embodiments, the tag can convey a unique patient's code on carrier signal or reflected signal by one of several methods, including but not limited to: frequency modulation, frequency shift keying (FSK), pulse width modulation, and phase shift keying (PSK). In some embodiments, these modulated reflected signals are then demodulated and converted to binary identification numbers.

In some embodiments, a patient's ID number is encoded on the reflected carrier signal by using conventional modulation methods including but not limited to PSK or FSK modulation. In some embodiments, codes can be set by several bits including pilot bits for both cases. In some embodiments, pilot bits can let the system know the first bit of the patients' ID number and can be consecutive three bits with value one or high. In case of PSK, a fixed offset frequency of more than one cycle can comprise one bit of code bit. In some embodiments, each bit's value can be assigned by shifting the phase of modulated signal from 0 to 180 degree. In some embodiments using the system illustrated in FIG. 16H, PSK can be achieved by switching the load attached to the antenna via the diode to provide the phase shift. In some embodiments, the bit values change whenever the current bit phase is 180 degrees different from the previous bit. In some embodiments utilizing FSK, two different frequencies are used for modulating the reflected signal, one of which represents zero while the other does one. In some embodiment using the system illustrated in FIG. 16H, this could be achieved by switching the diode at the crystal frequency and half the crystal frequency for a fixed period. In other embodiments using the system illustrated in FIG. 16H, four frequencies can be used to provide 2-bit data. In other embodiments using the system illustrated in FIG. 16H, more than 4 frequencies can be used.

In some embodiments, the same radar front-end can be used to detect both the ID information appearing in the sidebands, and the Doppler shift generated by the subject's physiological motion, from the portion of the signal reflected by the thorax and not the tag as shown in FIG. 16I. The most important difference between the ID information and the Doppler shift generated by physiological is the bandwidth, which affects the required sampling rate. The sampling rate for the combination radar sensor-ID reader is preferably adequate for detection of the sidebands generated by the tag and for the baseband Doppler shift generated by the subject's physiological motion. After complex down-conversion, the sidebands can appear at a low IF frequency (in some embodiments, this would be in the 10-kHz range—the same frequency as the crystal) that can be digitized and further demodulated in digital domain. The baseband Doppler shift can be near DC, at frequencies below 10-Hz. The baseband signal conditioning is essentially the same for both the tag reader and the direct-conversion Doppler radar sensor of physiological motion, but in the tag reader system, it needs to accept signals that are sufficiently wideband to include both the baseband Doppler signal and the sidebands generated by the tag. In some embodiments, the signal generated by the tag can have a much lower power than that reflected from the torso, in which case the dynamic range of the receiver is preferably adequate to detect both signals. In various embodiments, this can require one or more of the following methods: AC-coupling the signal to remove DC offsets before amplification and using a high-resolution analog-to-digital converter; applying a method of DC cancellation or DC compensation in analog processing before a high-gain stage and using a high-resolution analog-to-digital converter; separately processing the sideband and the baseband Doppler signal such that each has appropriate gain and filtering; and/or using a high resolution analog-to-digital converter.

In some embodiments, in addition to the identification signals provided by the tag, it is also possible to obtain signals about physiological motion from the Doppler shift of the sideband signals generated by the tag, referred to here as the sideband Doppler signal. Once the signal is digitized, the sideband signals (those generated by the motion of the tag) can be separated from the baseband Doppler signals (those reflected by the thorax without the tag). In some embodiments, the sideband Doppler signal can be digitally downconverted to baseband, and processed the same way that the baseband Doppler signal is processed. Since the ID tag itself is attached to the moving surface, signals reflected from the tag antenna can contain a similar Doppler shift as that produced by the moving chest. If there were no modulation on the tag, these two signals would add and it would be challenging to separate them. However, since the tag backscatter is shifted in frequency by modulating diode bias current, the Doppler shift, as well as the ID information, can appear on these sidebands. Since the modulated backscatter from the tag (sideband Doppler shift) is originating only from the chest region physically attached to the tag, and the carrier Doppler shift results from the illumination of a larger area that can include the hands, arms, shoulders, and legs, it is expected that two signals can exhibit subtle differences. In some cases, the modulated backscatter can be more immune to fidgeting motion, since there are fewer potential sources of non-cardiopulmonary motion attached to the tag. In some embodiments, the Doppler-shift signal obtained from the tag can be compared with the Doppler shift signal obtained from the non-tag reflections. In some embodiments, significant differences in the two signals can indicate non-cardiopulmonary motion in the signal obtained with the non-tag reflections. In some embodiments, the two signals can be compared with a cross correlation function, and the degree of correlation between the signals can be used to determine whether or not to indicate non-cardiopulmonary motion. In some embodiments, the Doppler-shift signal obtained from the tag reflection can be used for physiological processing. An additional advantage of the sideband signals is that they can not suffer from distortion due to ac coupling, in embodiments where an ac-coupled receiver is used, and they can also be less affected by 1/f noise.

In some embodiments, a desired or designated subject in a home environment could be continuously monitored, provided there is adequate coverage of all rooms with one or more reader and the subject is wearing a tag.

FIG. 16J is a flow chart illustrating an embodiment of the identification-reading and vital signs signals processing of the sideband signals. In this embodiment, the ID code is encoded on the signal by the RFID tag, using fixed-length PSK codes at a fixed offset frequency. In this embodiment, the encoded signal is modulated on the signal reflected by the RF tag's microprocessor, resulting in a sideband signal offset from the carried frequency by the frequency of the PSK modulation. Since the amplitude of the correlation coefficient is proportional to the position or delay of the reflected encoded signal, the amplitude variation of the correlation coefficient can be used to provide vital signs which can be used for information diversity or confirmation when obtaining vital signs from the baseband Doppler signal

The system 100 including the radar-based physiological sensor can be configured in variety of ways as described below.

An example system configuration can include a Spot Check monitor configured as a single piece or a two piece system and adapted to operate at 2.4 GHz. The system 100 can further include a single antenna, direct conversion or a homodyne receiver and a high-pass filter. The system 100 can further include a processor configured to process signals using the linear demodulation algorithm described above. In various embodiments, the processor can also be configured to estimate the rate (e.g., respiratory rate, heart rate, etc.) using one or more rate finding algorithms.

As described above, in various embodiments, the monitor can include a homodyne receiver. In various embodiments, the homodyne receiver is used for its simplicity and for its phase noise cancellation property. In various embodiments, to eliminate mirror imaging at baseband after down converting the RF signal, the system includes complex demodulation, which provides quadrature analog outputs. In various embodiments, to get a focused beam, a 2 by 2 arrayed patch antennas are used. In various other embodiments, smaller or larger array patch antennas or a single (non-array) patch antenna can be used. For example, to get a more focused beam, more antennas can be used in the array. In various other embodiments, other (non-patch) antenna configurations can be used. In various embodiments, the quadrature outputs can be anti-alias filtered and the DC signal can be removed with a high-pass filter. The filtered signal can be sampled with an analog to digital converter (ADC) and the digitized data is subsequently processed in the processor. In some embodiments, the physiological motion signal is analyzed to determine whether the signal has low quality due to noise, interference, and/or non-physiological motion. In some embodiments, the physiological motion signal is separated from noise, interference and/or non-physiological motion. Then the physiological motion signal is processed to determine respiratory waveform, and the respiratory rate. In some embodiments, the respiratory rate is extracted from the respiratory rate waveform.

FIG. 17 illustrates an embodiment of the system 100 configured as respiratory rate spot check measurement device. The device illustrated in FIG. 17 includes a source of electromagnetic radiation 1701 (e.g., a voltage controlled oscillator) and a transceiver 1702. In some embodiments, the transceiver 1702 can include a single antenna to transmit and receive the signals. The signal received from said one or more objects that scatter radiation and have motion is directed to at least one mixer 1704 through a power splitter 1703. In some embodiments, the power splitter can be a 2-way 0 degree power splitter. In various embodiments, the signal from the source 1701 can be mixed with the received signal at the mixer. In various embodiments the system 100 can include two mixers (e.g., 1704 and 1705) that can output an in-phase and a quadrature-phase component. The signals output from the mixer can be conditioned and sampled by a data acquisition system (DAQ or DAS) 1706. In various embodiments, the signal can be conditioned to remove aliasing, for example by low-pass filtering. In various embodiments, the signal can be conditioned, for example, by high-pass filtering, low-pass filtering, DC-cancellation, amplifying, etc. The digital acquisition system 1706 can include multiplexers, analog-to-digital converter (ADC), digital-to-analog converter (DAC), timers, buffers, etc. The output of the digital acquisition system 1706 can be communicated to a computer or a processor for further signal processing. In some embodiments the computer or the processor can be in electronic communication with an output unit that is configured to perform an output action based on the information obtained after signal processing. For example, in some embodiments, the output unit can include a display unit configured to display. In some embodiments, the output unit can include a printer configured to print or an audible system configured to sound an alarm or and audible system configured to speak the respiratory read or a medical device (e.g., a defibrillator) configured to use the information or a home healthcare device configured to collect information from various medical devices and transmit the information to a central database or a health kiosk computer configured to transmit the information to a remote healthcare practitioner. In some embodiments, the computer or processor can be in electronic communication with an input unit that is configured to control system. In some embodiments, the input unit can be a start button or a health kiosk computer configured to allow a remote healthcare practitioner to initiate the measurement or a home healthcare device configured to initiate the measurement.

In various embodiments, the cardiopulmonary related motion of the body surface can be measured either from a distance or by contacting the body surface. In those embodiments, wherein the antenna is in contact with the body methods to isolate body surface reflections from internal reflections are used to enable measurement of the internal body motion. Various internal cardiopulmonary related changes can also be electromagnetically measured for surface and internal body parts and tissues, including impedance changes associated with heart beat.

One embodiment of a respiration rate spot checker is illustrated in FIG. 18. The system includes a radar-based physiological sensor 1801 similar to the various embodiments described above, a computational unit, and a display unit. In various embodiments, the computational unit and the display unit can be housed together in single housing 1802 (e.g., a laptop, a handheld computer, a PDA, etc.). The sensor 1801 can communicate with the computation unit and/or the display unit wirelessly or over a wired connection using the various communication protocols discussed above. In various embodiments, the sensor 1801, the computation unit and the display unit can be housed together in a single housing. In certain embodiments, the sensor 1801 and the computational unit can be housed together in single unit and the display unit can be separate.

In various embodiments, the spot check monitor can be configured to operate when a start button is actuated. In various embodiments, the monitor can start measuring the physiological motion signal in the operational mode. In various embodiments, a user can select one of three modes: quick mode, extended mode, or continuous mode. Each of the three modes can require a different number of consecutive breaths without motion before providing a rate. For example, in the quick mode, approximately 2 consecutive breaths without motion can be required to calculate the rate, in the extended mode, approximately 6 consecutive breaths without motion can be required to calculate the rate while in the normal mode, approximately 3 consecutive breaths can be required to calculate the rate.

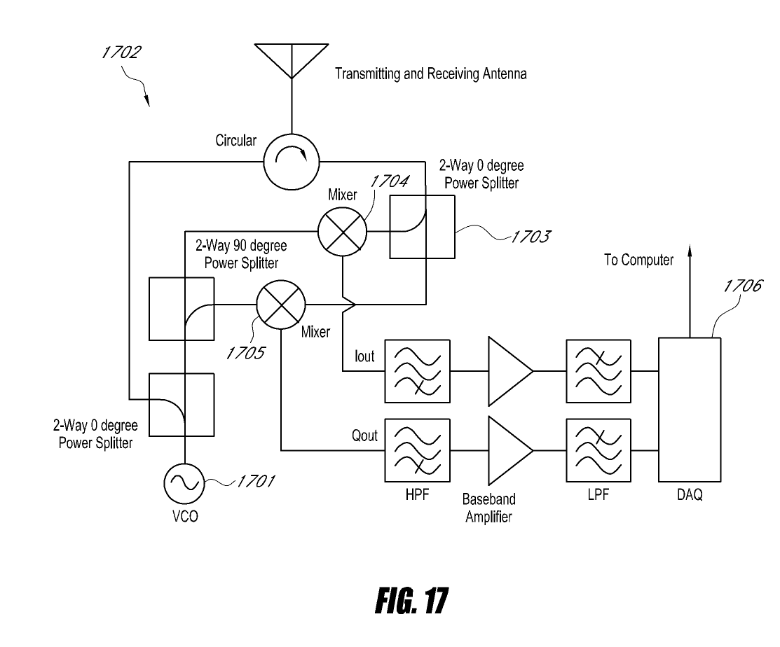

FIG. 19 illustrates an embodiment of an interface (e.g., a display screen) configured to output cardiopulmonary or cardiovascular related information (e.g., respiration rate, respiratory waveform, heart rate, pulse rate, etc.). The embodiment illustrated in FIG. 19 is a screen shot of a display displaying the measured respiratory rate. In various embodiments, a signal processing unit (e.g., the computation unit of FIG. 18) can determine the peak inhalation points of the subject and count them over time using one or more algorithms. In various embodiments, the system 100 can buffer a respiration rate for every block of data. In various embodiments, if an interruption (e.g., interruption created due to non-cardiopulmonary motion or other signal interference) is detected during the reading, any respiration rate values stored in the buffer will be cleared and no values will be buffered until the interruption has ceased. Once the approximate required number of breaths is read consecutively, the device returns the median value recorded, to ensure that the reading is as accurate as possible. In some embodiments, the required number of breaths can be 3. In various embodiments, the required number of breaths can be 5, 10, 15, 20 or some other value in the range from 3-30. In various embodiments, the interface can have a status indicator 1901 configured to show a status. For example, the status indicator 1901 can be a bar which will grow as each consecutive breath is read. As soon as the required number of breaths is read, the status indicator can stop growing. The measured respiratory rate can be indicated in area 1902 of the display. In various embodiments, controls can be provided on the interface configured to control the system. For example, a start and a stop button 1903 and 1904 can be provided on the display interface illustrated in FIG. 19. In various embodiments, the measurement can be interrupted if the stop button is actuated, in which case no values can be returned.

In various embodiments of the system, the respiration rate can be determined by using a rate estimation algorithm which uses two processes, e.g., a time domain approach and/or a frequency domain approach to determine the respiration rate: a frequency domain estimate and a time domain estimate. A first advantage of employing two methods is that comparing the result of the two approaches can help to determine if breathing is regular. A second advantage is that the redundancy introduced by employing two algorithms can help in risk mitigation for inaccurate respiratory rates. In various embodiments, the time domain rate estimation uses the zero crossings with positive or negative slope in the signal to recognize a breath. The peak of the signal between two consecutive positive zero crossings or two consecutive zero crossings is compared against a threshold to determine if the two consecutive zero crossings actually include a breath. In some embodiments, the positive zero crossings will be used, and if there are not enough breaths for a rate to be calculated, the negative zero crossings will be used. Additionally, a Fourier transform is computed on all the samples to provide the signal spectrum. In various embodiments, the frequency domain estimate of the rate can be the largest magnitude frequency component in the signal. The time domain and the frequency domain rate estimates can be compared. In various embodiments, the difference between the two results can indicate the degree to which the signal does not fit the assumptions of either the time or frequency domain approaches. For example, a difference of 0 can indicate a perfect match between the time domain and the frequency domain approach. In various embodiments, the frequency domain calculation can serve as a cross check to the measurement obtained from the time domain approach or vice versa. In various embodiments, the two rates can serve as a cross check for accuracy. A mismatch between the frequency domain and time domain calculations can also indicate possible irregular breathing. Various embodiments of the device can require a low variability in the respiratory rate to provide a measurement or a reading to ensure that measurement or readings provided are accurate. In some embodiments, the system could display or otherwise communicate an indication of level of variability of the measured rate, i.e., how much the rate varied during the measurement interval. The variation in the measured rate can be used in medical analysis by the health care professional.

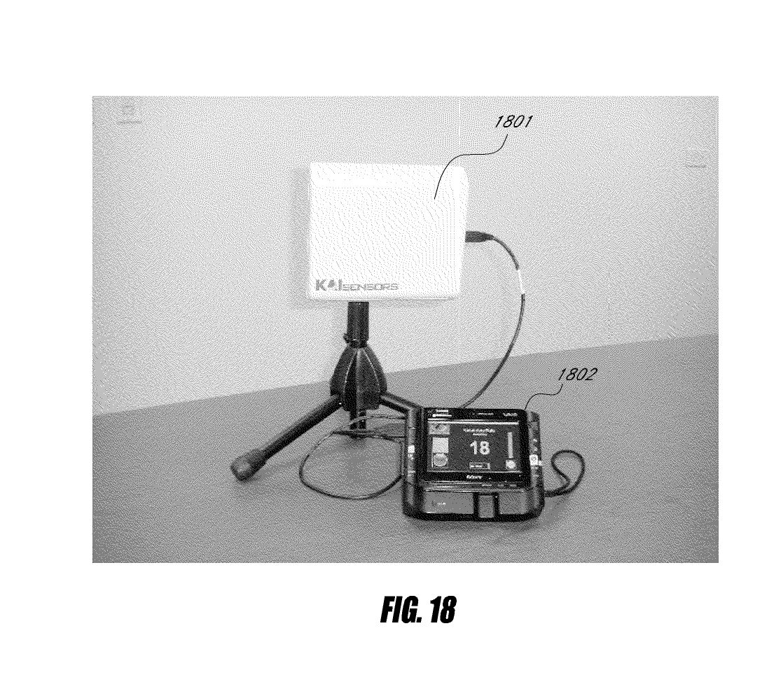

FIG. 20 illustrates a screen shot of a display device. The display device is in communication with a system 100 that uses both time domain approach and frequency domain approach to calculate the respiration rate as discussed above. The system 100 can be configured to perform the measurement over a fixed period in a range between approximately 15 seconds to approximately 1 minute. For example, in some embodiments, in the quick mode the system 100 can perform a measurement over a 15 second time interval, in the normal mode, the system 100 can perform a measurement over a 30 second time interval and in the extended mode, the system 100 can perform a measurement over a 60 second time interval. These time intervals correspond to intervals commonly used by healthcare practitioners when counting respiratory excursions to estimate respiratory rate. In other embodiments, the time intervals for the three modes can be different. A status indicator 2001 can indicate the time that has passed during the measurement and the time that remains for the measurement. In some embodiments, the display can also have a control button 2002 that can allow a user to choose a mode of operation (e.g., quick, normal or extended). Other controls such as a start button 2003 and a stop button 2004 can also be provided on the display to control the system. In some embodiments, the display can also provide a status indication of the system. For example, in FIG. 20, the display indicates the status of the power source and the battery power for the computation unit. In some embodiments, the previously measured rate can also be displayed. In some embodiments a clear button 2005 can also be include to remove the displayed respiratory rates from the screen. In various embodiments errors in estimating a respiration rate for example due to the presence of non-cardiopulmonary motion or other signal interference can also be displayed on the display device.

FIG. 21 illustrates another embodiment of a system 100 including a sensor 2101, a computational unit and a display unit housed in a single housing 2102.

In various embodiments, the rate-estimation algorithm, described above, operates on all the data obtained during the measurement interval. In various embodiments, the rate-estimation algorithm can detect a non-respiratory signal (e.g., non-cardiopulmonary signal or other signal interference) and use this information to identify the signal quality. Samples of data having low signal quality can be rejected. For example, samples having an excursion larger than the subject's maximum breath can result from non-cardiopulmonary motion or other signal interference and thus can be rejected. In some embodiments, samples exhibiting a significant increase in signal power can also result from non-cardiopulmonary motion and thus can be rejected. In some embodiments, the non-cardiopulmonary motion detection algorithm described above can be used to detect non-respiratory signals or other signal interference. In various embodiments, additional inputs to signal quality indication can include low signal power, signal clipping due to high signal power, and low estimated signal to noise ratio. In various embodiments, the values that are rejected due to low signal quality can be set to zero before proceeding with rate estimation.

As discussed above, in various embodiments, the time domain rate estimation uses the zero crossings with positive or negative slope in the signal to recognize a breath. The peak of the signal between two consecutive positive zero crossings or two consecutive zero crossings is compared against a threshold to determine if the two consecutive zero crossings actually include a breath. In some embodiments, the positive zero crossings will be used, and if there are not enough breaths for a rate to be calculated, the negative zero crossings will be used. Additionally, a Fourier transform is computed on all the samples to provide the signal spectrum. In various embodiments, the frequency domain estimate of the rate can be the largest magnitude frequency component in the signal. The time domain and the frequency domain rate estimates can be compared and the accuracy of the estimated rate can be determined.

In various embodiments of the system (e.g., a system using a 2.4-GHz ISM band) using linear demodulation algorithm to demodulate the sample, significant changes to the best-fit vector or eigenvector on which the signals are projected can indicate a new relationship between the antenna and the subject, which can indicate the presence of non-cardiopulmonary motion or signal interference. When linear demodulation is used, a change in the ratio of the eigenvalues, or of the RMS error of the fit to the best-fit line, can also indicate that the detected motion does not fit the line well consequently indicating non-cardiopulmonary motion or other signal interference.

The various embodiments of the respiratory rate spot check measurement device described above can be adapted to be used in a health kiosk. The spot check measurement device described with reference to FIGS. 17-21 can be in communication with one or more master control systems such that the spot check monitor can be controlled by one or more master control systems. Various embodiments of the system initiate a measurement by at least one of a local operator by pressing a button on the device, remote activation by a healthcare practitioner, automatic initiation when the presence of the patient in the kiosk is sensed. Various embodiments of the device can sense the presence of a patient in the kiosk and communicate that information to the kiosk computer. Various embodiments of the device can take an input from another sensor, communicated through the kiosk computer that indicates the presence of the patient in the kiosk. Various embodiments of the system 100 can communicate with the one or more master control systems using any standard or proprietary communication protocol, or any combination thereof. Such protocols can include any communication technology, which can or cannot be included in TCP/IP or OSI network layers, including, but not limited to, serial, USB, Bluetooth, Zigbee, Wi-Fi, Cellular, WiMAX, Ethernet, and SOAP. For example, Ethernet can be used as the link layer protocol while TCP/IP is used for routing, and SOAP is used as an Application layer protocol. On the other hand, only TCP/IP over Ethernet can be used, without additional packaging at the Application level. In the later case, data collected from the radar system 100 can be formatted and directly packaged as TCP payload. This can include timestamp for when the data was collected, the data, and an indicator for the quality of the data. This data is attached with a TCP header and then becomes the IP payload. The IP header (addresses) is attached to the payload and then is encapsulated by Link layer headers and footers. Finally, physical layer header and footers are added and the packet is sent via the Ethernet connection. To access data from the connection, the client should have a program to listen to a specified port on their Ethernet connection where the packets are being sent. Various embodiments of the system 100 can comply with the Continua Health Alliance medical device communications guidelines, including control and communication via USB or Bluetooth.

FIG. 21A illustrates an embodiment of the radar-based cardiopulmonary monitoring system configured as a non-contact respiratory rate spot check measurement device. The transceiver 2110 illustrated in FIG. 21A includes a source of electromagnetic radiation (e.g., a voltage controlled oscillator).

In embodiments using a direct conversion radar system operating at a radio frequency of approximately 2.4 GHz to measure respiratory motion, the phase deviation due to cardiopulmonary activity can result in a complex constellation in the I/Q plane that with points with a distribution that is linear, arc-shaped, FIG. 8, elliptical, egg shaped, or a combination of above. In some embodiments, a phase-lock loop (PLL) circuit is employed to control the frequency of the RF oscillator. Frequency selectivity within the ISM band is possible in embodiments with a broadband antenna that matches over the ISM band and with a radiation source that has frequency agility over the same ISM band provided by a tunable frequency synthesizer.

In some embodiments, antenna elements utilizing an air dielectric are used to provide directional radiation with low loss and broad-band matching. In some embodiments, spread spectrum techniques are used to introduce a pseudo-random phase noise to a frequency synthesizer that utilizes a phase-locked oscillator, and therefore would otherwise have low phase noise. In some embodiments, pseudo-random phase noise with range-correlation in direct-conversion systems can be used to mitigate RF interference, because other transceivers can not have the same pseudo-random phase modulation. In some embodiments, the spread spectrum is optimized for physiological monitoring through manipulation of noise bandwidth and amplitude. In some embodiments, the pseudo-random phase modulation is provided with a programmable logic device (PLD).

In some embodiments, the complex constellation has an arc that can use non-linear arc-based demodulation. In some embodiments, linear demodulation can be used to provide an estimation of relative movement.

In some embodiments, the transceiver 2110 includes an active IQ demodulator that provides differential quadrature baseband (or intermediate frequency) signals. In some embodiments, the baseband signals from a differential active quadrature demodulator are filtered and amplified in a fully differential baseband signal conditioning stage, and then digitized with a differential input analog-to-digital converter (ADC). In some embodiments, DC cancellation, rather than an AC coupling filter is used to reduce signal distortion. In some embodiments, the high dynamic range of a high resolution ADC allows for the extraction of a relatively small time varying signal from a relatively large DC offset of a direct conversion system with DC coupling. In some embodiments, a 24-bit ADC is used. In some embodiments, the signal is oversampled and then decimated and interpolated to improve the resolution of the system.

Some embodiments use arc-based demodulation to extract phase information from the baseband signal, such that the demodulated signal is linearly proportional to the actual chest motion and it is possible to estimate depth of breath. In various embodiments, as the length of the arc increases, the ambiguity in the signal polarity can be reduced, which enables differentiation between inhalation and exhalation, such that it is possible to estimate the duration of inhalation and the duration of exhalation, as well as estimation of the ratio between inhale time and exhale time.

The system illustrated in FIG. 21A shows a system powered through 5V USB bus power, with a processor 2112, memory and/or storage 2114, an aiming light 2116, and a touch screen OLED display 2118 integrated in the sensor unit. This example system also has a transceiver 2110, or radio section, that includes a broadband directional antenna 2120, a PLL-controlled oscillator with frequency agility with pseudo-random phase noise, and an active direct-conversion quadrature demodulator with differential IF ports. Fully differential DC-coupled baseband signal-conditioning leads into a high resolution ADC for acquisition.

In some embodiments, the processor 2112 can be integrated into the same housing as the sensor radio and can process the radar signals to provide vital sign information on the integrated display in a single standalone unit. In some embodiments, the integrated processor runs the core algorithms and provides rate and other information to a separate host computer. In some embodiments, the integrated processor can run a real-time open source operating system with memory and file management to run under the core algorithms. In some embodiments, the integrated user interface (in the example, the OLED touch-screen display 2118) is used to initiate a respiratory measurement. In some embodiments, the host computer provides a command over a communications interface (in the example, USB) to initiate measurements.

In some embodiments, proper aiming of the device can be aided through an integrated light source. In some embodiments, a high-intensity directional LED can be used to visually illuminate the areas that are included in the antenna field of view. In some embodiments, the sensor contains a button that can be used to turn the integrated light source on and off; in this example, this button is on the integrated OLED touch screen.

In some embodiments, the sensor's integrated display provides instant feedback, including, but not limited to progress, error messages, retry messages, low-signal information, results, and other information. In some embodiments, the integrated screen is touch-sensitive allowing for context specific use of buttons and an easy-to-use user interface. In some embodiments, an organic light emitting diode (OLED) display is used for its increased color gamut, viewing angles, brightness, contrast, and power usage due to the lack of need for a backlight as with a liquid crystal display (LCD).

One possible embodiment of a respiration spot check device can be similar to the system illustrated in FIG. 21 above which comprises a sensor 2101 and a computational unit 2102 that is integrated with a display. In the illustrated embodiment, the computational unit and display are housed together in the laptop. However, in some embodiments, all three parts can be housed inside one single unit, individually, or any combination thereof (i.e., computational unit and sensor in one housing with display as a separate unit). FIG. 21B illustrates a screen shot of an embodiment of a display device. The device can start measuring the subject when the start button 2128 is depressed. The user can select one of three modes: quick, extended, or normal, which requires a different number of consecutive breaths without motion before providing a rate. In some embodiments, the signal processing can determine the peak inhalation points of the subject and count them over time. For every block of data, the device can buffer a respiration rate. If an interruption is detected during the reading, any respiration rate values stored in the buffer can be cleared and no values can be buffered until the interruption has ceased. (Interruptions can be caused by non-respiratory motion or other interference.) In some embodiments, once the designated number of breaths is read consecutively (3 is set as the default value), the device can show a rate calculated from the median breath-to-breath interval. As each of these consecutive breaths are read, the vertical bar 2130 illustrated in FIG. 21B can fill higher, until the it has reached the designated number. When the bar is filled, a respiratory rate 2132 can be displayed. The reading can also be ceased if the stop button is depressed, in which case no values can be returned. If the maximum time interval for the measurement mode expires before the minimum number of breaths are measured, the device can display an error message. In some embodiments, rather than calculating the respiration based on blocks of data, it is also possible to calculate the respiration based on each inspiration peak to inspiration peak interval. In some embodiments, the spot-check monitor could measure a specified number of peaks before displaying a respiration rate, or it could measure for a specified time interval. In some embodiments, the time interval or the number of peaks could be extended if the measured respiration rate is varying more than a few breaths per minute, to ensure an accurate reading of in irregular rate. In some embodiments, the respiration spot check can be network-enabled such that settings can be set and taken remotely, and results of measurements can be stored in an Electronic Health Record.

In some embodiments, the spot check hardware described above can be configured such that respiratory measurements can be programmed to occur intermittently, periodically, or at pre-defined intervals. In some embodiments, an external computer, including, but not limited to, a tablet, a desktop, a laptop, a PDA, or a smartphone, can be used to control the spot-check device in interval mode. In some embodiments, the external computer can communicate control commands (start, stop, reset, etc) and capture data from the spot-check device using either a custom or standard communications protocol. In some embodiments, software on the external computer can provide statistical analysis of rate history and can communicate with an electronic health record (EHR) to store data or cross reference with other data in the EHR to improve the identification of statistical trends and anomalies. In some embodiments, the display of the external computer can be used to display the historical data, and to provide other information on the patient being measured.

In some embodiments, the respiratory rate interval measurement device can operate as a stand-alone device. The standalone device would include timing of the interval measurements, display of the history of measurements, and all alerts and alarms required.

The interval respiratory measurement device has real-time signal-quality detection, such that portions of collected data with poor signal quality due to low signal power or subject motion are not used to estimate the respiratory parameters, and portions of the collected data with adequate signal quality are used to estimate the respiratory parameters. The device uses an automatic mode such that the measurement length is chosen automatically based on signal quality and/or regularity of breathing. In some embodiments, the device can continue re-trying a measurement until enough signal of adequate quality is obtained to provide a respiratory spot check.

Communications can be used to link the interval respiratory measurement with a central monitoring station, such as a nurses station or a remote care center, or it can transmit data to a central storage area, such as an electronic medical record or a non-hospital clinical information database.

The interval respiratory measurement device can be configured to display any parameter that can be measured by a Doppler radar sensor, including but not limited to respiration rate. The interval respiratory measurement can operate from a variety of angles and distances so long as the device is aimed on the subject.

In one possible configuration, a homodyne receiver is used for its simplicity and phase noise cancellation property. To eliminate mirror imaging at baseband after down converting the RF signal, the system has complex demodulation, which provides quadrature outputs. A single high gain antenna array can be used for transmit and receive, providing a focused beam width, which can mitigate possible interference sources in the surrounding environment. The sensor can be mounted on the bed rail during interval measurements. The quadrature outputs are anti-alias filtered and sampled by an analog to digital converter (ADC) followed by signal processing, which isolates the physiological motion signal from noise, interference, and non-physiological motion. In some embodiments, the signal is DC-coupled and digitized with a 24-bit ADC, and the DC offset is removed in software. Then the physiological motion signal is processed to determine the parameter(s) of interest.

One possible embodiment of a interval respiratory measurement device is as follows. The system can comprise a sensor unit that measures the respiratory rate, and a controlling PC that sends messages to the sensor unit to start measurements at pre-defined intervals and provides the interval user interface. In some embodiments, a single sensor unit can include the display, user interface, and timing for interval measurements, such that a controlling PC is not required. In some embodiments, another medical device can control the sensor unit. In some embodiments, a portion of the sensor unit can be placed in a controlling medical device, and in other embodiments, the controlling medical device can only communicate with the sensor unit. The controlling PC can send a message to the sensor unit to start a measurement when the start button is depressed, and at pre-defined intervals following the measurement.

In some embodiments, from the main menu of the interval respiratory rate measurement software on the controlling PC, operators can choose to operate the device in manual mode (for which the button can be pressed to initiate a measurement), or choose a time period for intermittent, or interval, measurements. One embodiment of a user interface for an intermittent spot check is shown in FIG. 21C. For example, if a user chooses 5 from the menu, then a measurement can begin every 5 minutes starting when the start button is depressed. In some embodiments, intermittent measurements can only stop repeating when the stop button 2134 is depressed. In some embodiments of the intermittent mode, a history of the measurements and their associated time can be displayed as in FIG. 21D. In some embodiments, a message bar can also be available to provide further information such as the current mode and period of measurements as illustrated in FIG. 21E or instructions to mitigate a potential error during a measurement. In some embodiments, this message bar can also be used to provide pertinent information during manual measurements as shown in FIG. 21F. In some embodiments, the interval respiratory measurement device can be network-enabled such that settings can be set and taken remotely, and results of measurements can be stored in an Electronic Health Record.

An example configuration of system 100 can include spot check monitor configured in various embodiments as a single piece or a two piece system and adapted to operate at a radio frequency of approximately 5.8 GHz. Various embodiments of the system 100 can include DC-cancellation circuit to reduce the delay between the motion signal and the electronic indication of the motion. In various embodiments, DC-cancellation can enable faster synchronization between the motion sensor and the output device (e.g., a display or an imaging system). DC cancellation or low-IF at 5.8 GHz can make arc demodulation relatively more accurate. DC cancellation typically improves the synchronization time, which can be important for integration with an imaging system or a ventilator.

In embodiments using radio frequency in the 5.8 GHz range, the phase deviation due to the chest motion associated with cardiopulmonary activity can increase by more than two times when compared to embodiments using radio frequency in the 2.4 GHz range. In various embodiments, this phenomenon can result in non-linear baseband output such that the complex constellation more closely approximates an arc rather than a line. In these embodiments, arc-based demodulation algorithms can be preferred over other demodulation algorithms. In various embodiments, arc-based demodulation algorithms can provide results having greater accuracy by appropriately resolving this non-linear effect. In various embodiments, DC cancellation can be preferred over an AC coupled filter as DC cancellation can reduce signal distortion. In embodiments without DC cancellation, the origin of the circle where signal samples are scattered cannot be determined with sufficient accuracy.

When arctangent demodulation is used, significant changes in the location of the origin, or changes in the radius of the circle of the arc is on, or changes in the position of the arc on the circle can indicate a change in the relationship between the antenna and subject, which can indicate the presence of non-cardiopulmonary motion or other signal interference. In some embodiments, a change in the relationship between the subject and the antenna can be detected if the calculated inner product of the normalized current vector and the normalized previous vector is below a threshold. In a system where arctangent demodulation is used, a change in the RMS error of the fit to the best-fit arc can also indicate non-cardiopulmonary motion or other signal interference.

An example configuration of system 100 can include a continuous physiological monitor configured to operate in the frequency range of approximately 2.4 GHz and further configured as a two piece system. The continuous physiological monitor is configured to provide vital signs information and/or physiological waveforms over extended periods of time and not just periodic snapshots. Various embodiments of the continuous vital signs monitor can be configurable to operate in a spot check or a continuous mode. Various embodiments of the monitor can be configured to monitor at least one of the heart waveforms and variables and respiratory waveforms and variables. Various embodiments of the monitor can include a single antenna or an antenna array combined to operate as a single antenna, a direct-conversion or homodyne receiver and a high-pass filter. In various embodiments, multiple antennas can be used. Various embodiments of the monitor can include other electronic components such as filters, amplifiers, multiplexers, etc. In various embodiments, the system 100 can include a processor configured to execute the eigenvector-based linear demodulation algorithm or an arc-based demodulation algorithm other algorithm described above. In some embodiments, the system 100 can be configured to determine the heart rate and/or the respiratory rate.

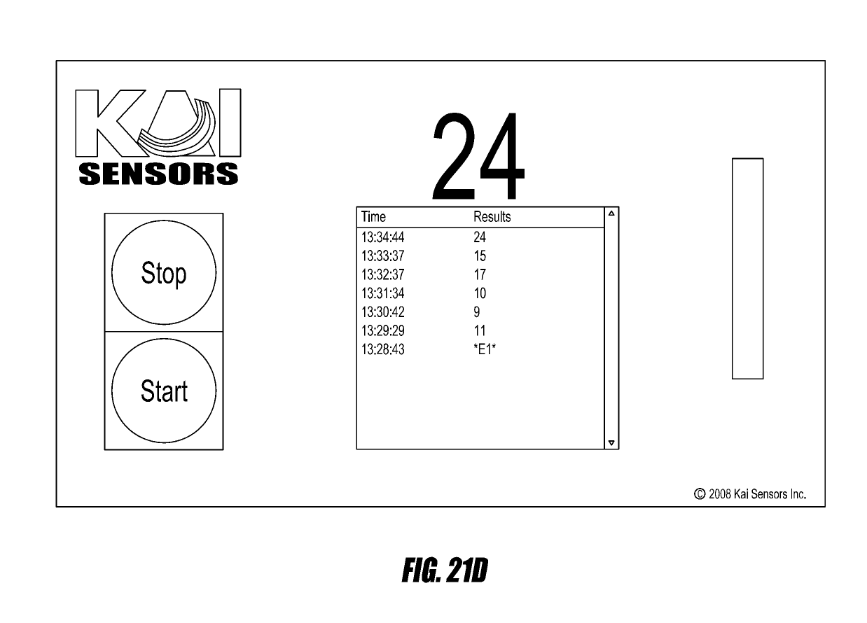

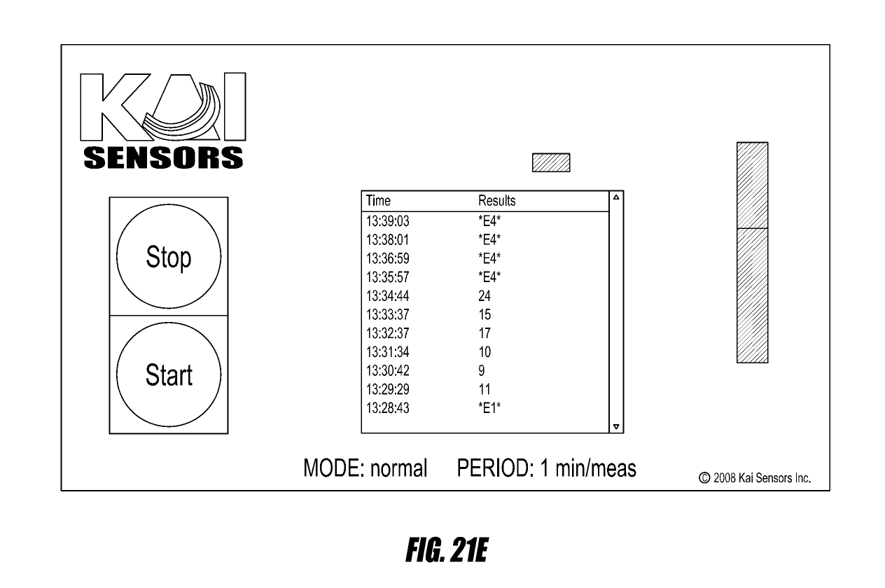

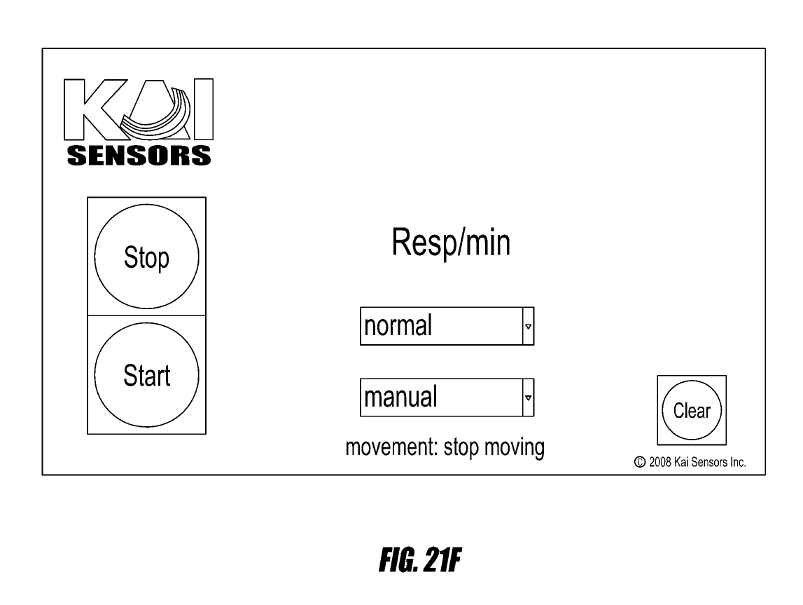

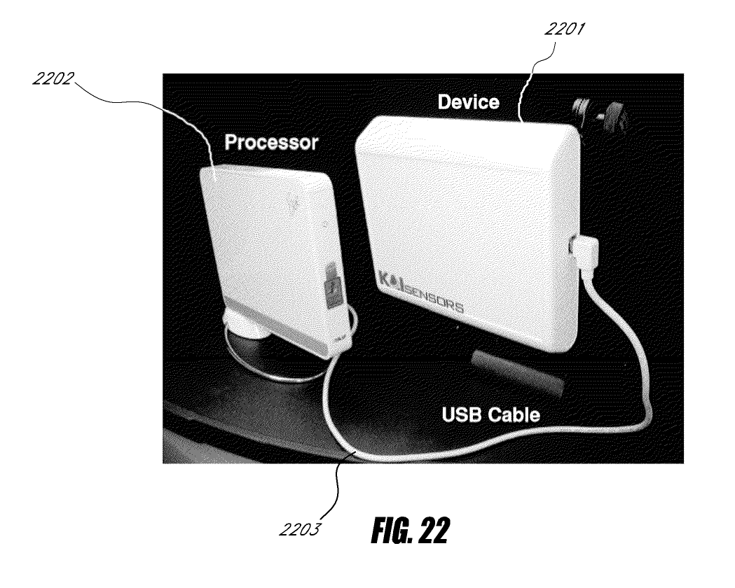

The system illustrated in FIG. 17 can be adapted to operate as a continuous vital signs monitor. The system illustrated in FIG. 17 is a continuous-wave radar transceiver with a homodyne receiver. One advantage of this configuration is the simplicity of the system. Another advantage of the system is its ability to cancel or reduce phase noise. In various embodiments, the transceiver 1702 can operate in the 2.4 GHz-2.5 GHz or the 5.8 GHz ISM band. In various embodiments, the transceiver can operate in a frequency range outside this band. In various embodiments, the source 1701 can be configured to generate both the transmitted signal and the local oscillator signal for the receiver. Such a configuration can be referred to as an internal voltage-controlled oscillator. In various embodiments, the oscillator can be free-running, phase-locked to a crystal, or phase-locked to an external reference. In other embodiments, the local oscillator can be generated externally to the rest of the circuit. In various embodiments, complex demodulation can be used to generate quadrature outputs. An advantage of this technique can be the elimination of mirror imaging at baseband after down converting the RF signal. In various embodiments, another advantage of this technique is the ability to use linear or nonlinear complex demodulation algorithms to avoid phase demodulation nulls that can plague single-mixer receivers used for this application. In some embodiments, the quadrature outputs can be amplified and anti-alias filtered before analog-to-digital conversion. To improve the dynamic range, in various embodiments, the DC offset can be removed with a high-pass filter, and variable gain amplifiers (VGAs) can be provided to ensure that the full input range of the ADC is utilized. In various embodiments, the VGAs can be controlled by digital control signals. In various embodiments, the gain levels of the VGA can be determined either by the user or dynamically by the processor through signal analysis. In various embodiments, DC-cancellation can be used instead of a high-pass filter. In various embodiments, after the signal is sampled by the analog to digital converter (ADC), it can transmitted over a wired or wireless communication link (e.g., Bluetooth, USB, etc.) to a processor that performs signal processing. In various embodiments, the processor can include a digital signal processor, a microprocessor or a computer. In various embodiments, the processor can be on the same board as the ADC, on a separate board, or in a separate unit. In various embodiments, the processor can use a linear demodulation algorithm to generate the combined physiological motion waveform. In various embodiments, the processor can use digital filters to further isolate respiration and heart signals from the combined physiological motion signal. In various embodiments, the respiration and heart signal can be isolated using with fixed digital filters. The signal processing algorithm can also determine a signal-quality parameter, including whether the signal has very low power (below 0.0001-0.0004 W) or very high power (above 5 to 10 W). In various embodiments, the algorithm can also determine if there is non-physiological motion. In various embodiments, the processor can stream data on a frame-by-frame basis over Ethernet using TCP/IP. In other embodiments, the processor can stream data with a protocol compliant with the Continua Health Alliance guidelines. In other embodiments the processor can stream data with a proprietary protocol. In various embodiments, each packet will contain a time stamp of when the data was taken, and at least one of the combined physiological waveform (heart and respiration before they are separated), respiration waveform, and heart waveform, respiration rate, heart rate, and signal-quality parameter. FIG. 22 illustrates an embodiment of a continuous wave monitor 2201 described above in communication with a processor 2202. As illustrated, in this embodiment, the continuous monitor 2201 communicates with the processor 2202 over a wired USB link 2203.

FIG. 23 shows a screen shot of an embodiment of a display device which displays the respiration signal and the heart signal in addition to other information to a user located locally or at a remote location. Plot 2301 shows the respiration trace obtained by the monitor 2301 while plot 2302 shows the heart trace obtained by the monitor 2301.

An example configuration of the system 100 can include a continuous physiological monitor including one or more antennas configured to operate in a radio frequency range of 2.4-2.5 GHz, a direct-conversion or a homodyne receiver and an anti-aliasing filter. Various embodiments include either a high-pass filter or a DC-cancellation circuit. In various embodiments, the system 100 can include a processor configured to execute a linear demodulation algorithm. In some embodiments, the processor can also be configured to execute the non-cardiopulmonary motion detection algorithm and/or a rate estimation algorithm. In some embodiments, multiple receive antennas and multiple receivers will be used such that the DOA algorithm described can be executed by the processor for separation and/or tracking purposes. In various embodiments, the rate estimation algorithm described above can be used herein to estimate the rate of respiration or cardiac activity. For example, in various embodiments, a frequency domain rate estimation algorithm, a time domain rate estimation algorithm, a peak detection algorithm or a combination of these can be used. In various embodiments, the accuracy of the determined respiration or cardiac activity can be improved by employing the methods listed above as disclosed in U.S. Provisional App. No. 61/204,881 which is incorporated herein by reference in its entirety. In some embodiments, the rate estimation algorithm can be performed periodically (e.g., every 10 seconds, every 20 seconds, every 30 seconds, etc.).

In various embodiments, the continuous physiological monitor can include an activity monitor configured to provide an indication when and for how long the target subject performs a non-respiratory movement. In some embodiments, the activity monitor can be configured to provide an activity index that can provide an indication of the frequency and duration of motion over a measurement period. In various embodiments, provided with multiple antennas, DOA processing can enable determination of a subject's position and the frequency with which the subject changes position. For example, it is possible to determine whether the subject is rolling to the left, rolling to the right, or moving without changing position. FIG. 24 is a screen shot of a display device or unit illustrating the respiratory rate, activity indicator and position of a sleeping subject. Plot 2401 illustrates the breaths/minute as a function of time for the subject. Plot 2402 illustrates activity of the sleeping subject while plot 2403 shows the position of the subject while sleeping.

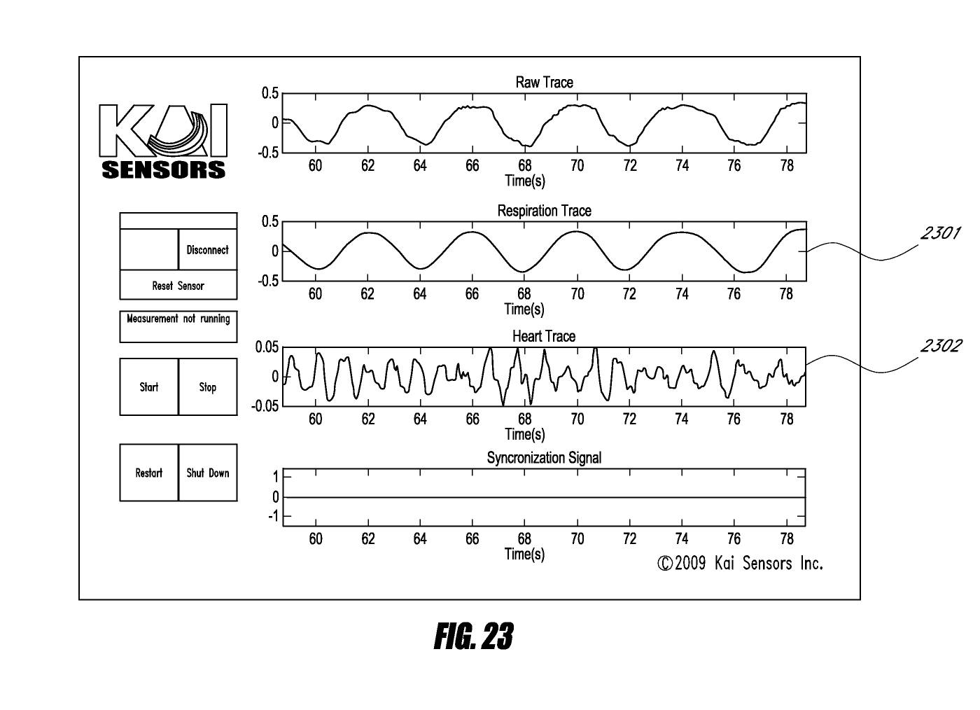

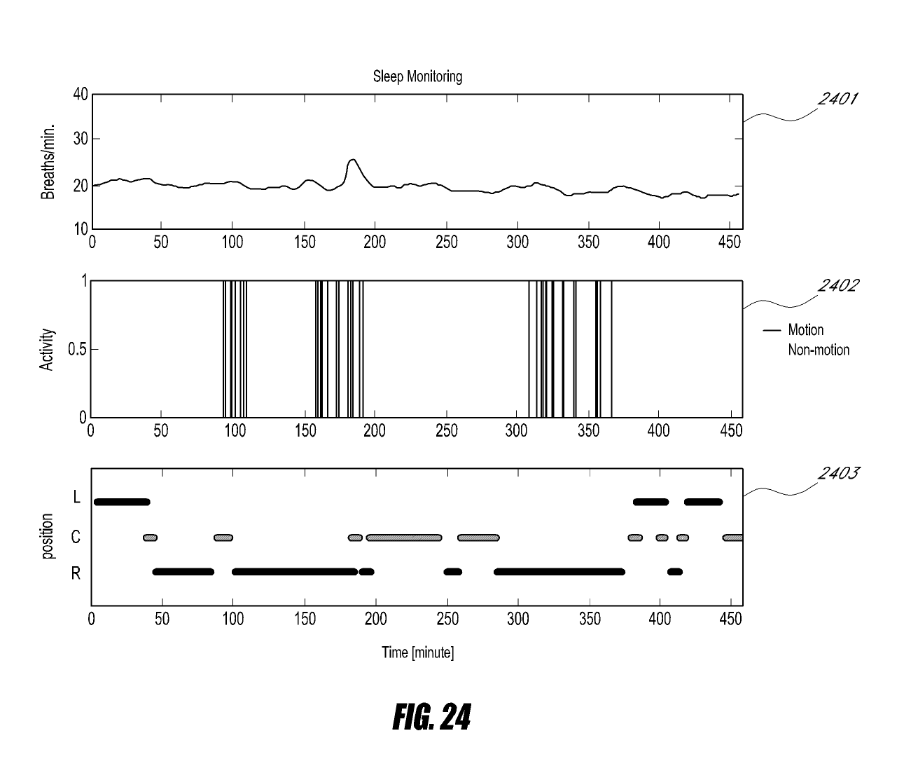

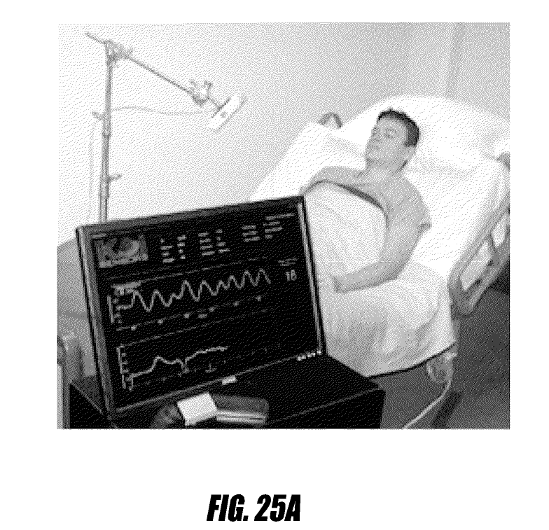

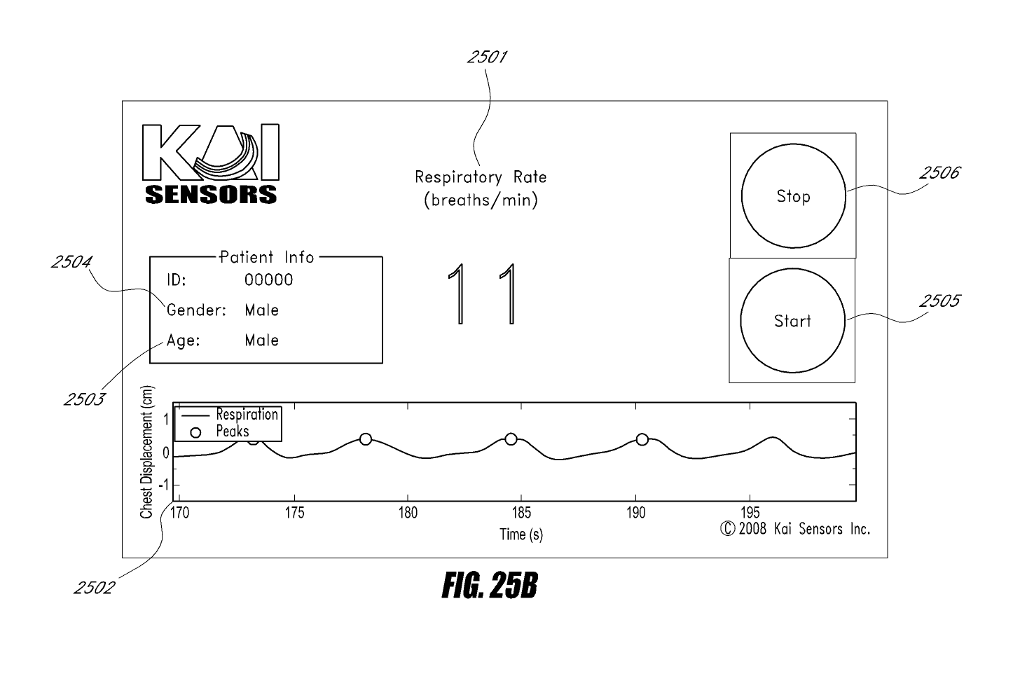

In various embodiments, the vital signs information (e.g., respiration rate or heart rate) can be buffered and plotted to provide historical data for the subject. FIG. 25A shows the application of the system in a hospital environment to measure the respiratory and/or cardiac activity of a patient. FIG. 25B is a screenshot of the display device illustrated in FIG. 25A. In some embodiments, the display device can display the respiratory or respiration rate 2501 and a waveform indicative of the respiratory activity 2502 (e.g., displacement of the chest over time). The display device can provide additional information related to the patient 2503 and 2504 (e.g., age, gender, etc.). The display device can also include a start and a stop button 2505 and 2506. In various embodiments, the display device can be a part of a device operated by health care professionals. FIGS. 26A and 26B illustrate screen shots of a display device that can be used for viewing the vital signs provided by the device. FIG. 26A shows an embodiment of a display device that displays a respiration rate 2601, average respiration rate over time 2602 and waveforms related to respiratory activity 2603 (e.g., chest displacement). FIG. 26B shows an embodiment of a display device that displays a respiration rate 2604, waveforms indicative of respiration activity 2605 and cardiac activity 2606 and a heart rate 2607.

An example system configuration includes a system configured to detect paradoxical breathing. The system includes a single antenna configured to operate in the radio frequency range of approximately 2.4 GHz, a direct conversion or homodyne receiver, and a DC-cancellation circuit. In various embodiments, the system can be configured to detect paradoxical breathing. In some embodiments, the system 100 can also include algorithms to estimate the rate of a respiratory activity or cardiac activity.

In various embodiments, the system 100 can include a continuous-wave radar transceiver with a direct conversion or homodyne receiver as described above with reference to FIGS. 17, 18, 19 and 20. As discussed above, advantages of this approach are the simplicity of the system and the ability to cancel or reduce phase noise. In various embodiments, the transceiver operates in a frequency range including, but not limited to, the 2.4 GHz-2.5 GHz ISM band. As discussed above, in various embodiments, a single signal source can be used to generate both the transmitted signal and the local oscillator signal for the receiver (e.g., source 1701 of FIG. 17). In various embodiments, the homodyne receiver can generate quadrature outputs using complex demodulation. In various embodiments, the quadrature outputs are amplified and anti-alias filtered before being input to a system configured to convert analog signals to digital signals.

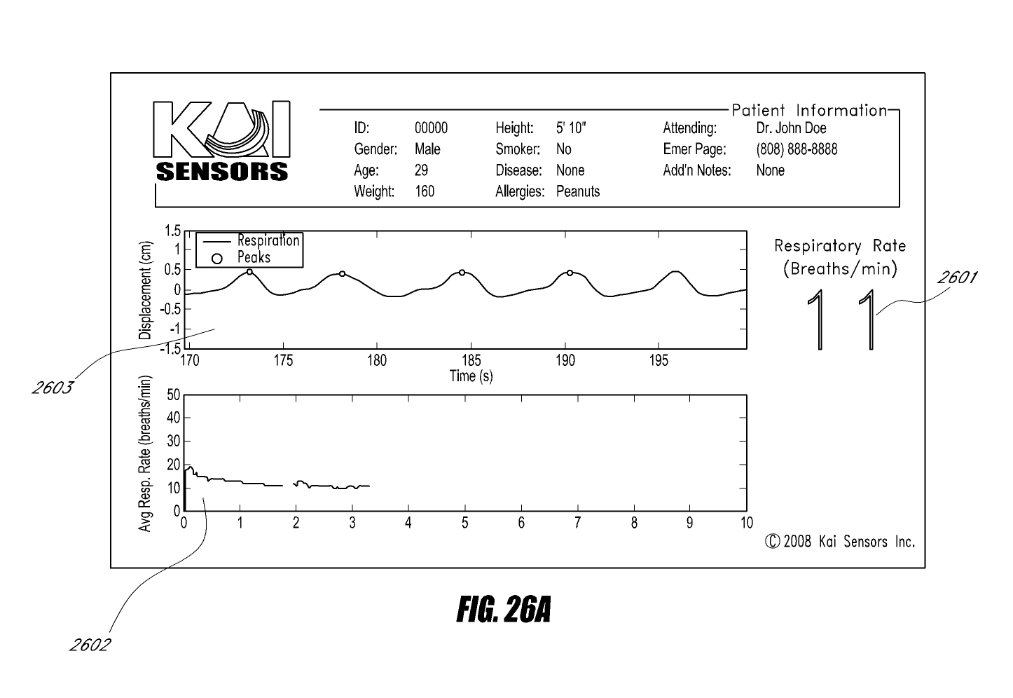

In various embodiments, to improve the dynamic range, the DC offset can be removed or reduced. In various embodiments, a conventional method of using an AC-coupling filter can be used to reduce or remove the DC offset. However, using an AC-coupled filter or a high-pass filtering can remove not only the DC offset itself but can also suppress low frequency components of the signal as well as distort their phase. Consequently, this causes an exponential attenuation of the static signal which is not DC offset, or distorts the phase of the signal. Additionally, a system having AC-coupling can generate or increase the group delay of the filtered signals, which causes a long settling time or a delayed version of the signal. These effects can result in the signal sample being distributed in a ribbon shape rather than an arc in the complex constellation. This distortion can adversely make the paradoxical breathing detection algorithm inaccurate. Some or all of these defects can be eliminated by using a DC cancellation circuit 2700, illustrated in FIG. 27, which is configured to subtract only DC value from the signals without distorting or adversely affecting the rest of the signal components. The DC cancellation circuit 2700 comprises a differential amplifier with gain 2701, an analog-to-digital converter 2702, a digital-to-analog converter 2703 and a DSP/digital control 2704. In various embodiments, the DC cancellation circuit can remove or reduce the DC offset by using feedback loops between ADC and DAC or voltage divider with digital potentiometer. Due to very small phase distortion, settling time, and group delay, systems including DC cancellation can be used to synchronize cardiopulmonary motion or other motion to imaging (e.g., CT scans or MRI) and to synchronize spontaneous respiratory effort to non-invasive or invasive assistive ventilation. The improved phase distortion and settling time also makes it easier to synchronize cardiopulmonary motion to questions asked and other sensors in polygraphs, to stimuli and other sensors for security screening, and for biofeedback applications, as disclosed in U.S. Provisional App. No. 61/204,881 which is incorporated herein by reference in its entirety.

In various embodiments, the system 100 can be configured to include an antenna array that can be used for transmitting and receiving radar signals. In some embodiments, a single antenna can be used for transmitting the radar signal, and an array of antennas can be used for receiving radar signals. The receiver can be configured as a homodyne receiver which is configured to generate quadrature outputs using complex demodulation algorithms. An advantage of this technique as discussed above is elimination of mirror imaging at baseband after down converting the RF signal. In various embodiments, the quadrature outputs are anti-alias filtered and the DC signal is removed or reduced with a DC-cancellation system similar to the one discussed above. The filtered signal is sampled by an analog to digital converter (ADC) and the digital data is processed to isolate physiological motion from noise, interference, and non-physiological motion. The physiological motion signal can be processed to extract the waveforms and parameter(s) of interest.

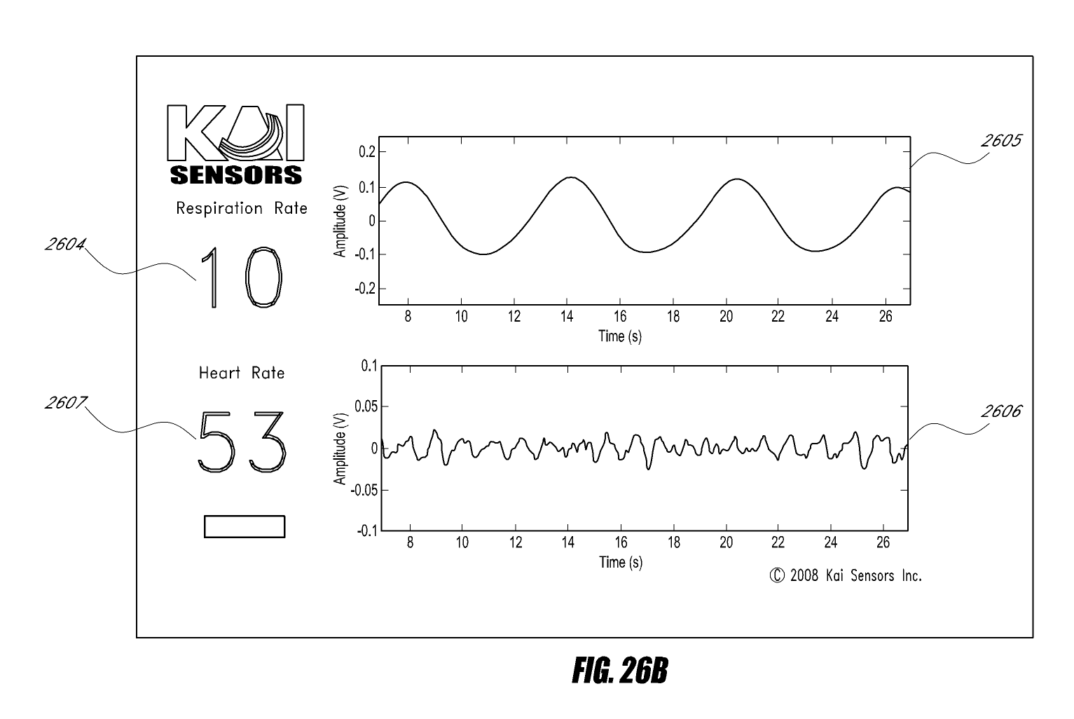

As discussed above, in various embodiments, the system 100 can be configured to detect the presence of or the degree of paradoxical breathing, which is a signature of obstructed breathing, respiratory muscle weakness, or respiratory failure. The system (e.g., a continuous monitor, quadrature continuous-wave Doppler radar system) can monitor the degree of paradoxical breathing based on analysis of the shape of the complex constellation and/or the trace of the plot of the in-phase (I) vs. quadrature (Q) signals from the quadrature radar receiver. An embodiment of a method to determine a paradoxical breathing indicator is illustrated in FIG. 28 and includes:
1. The paradoxical factor can be estimated by multiplying the ratio of the biggest eigenvalue to the second biggest eigenvalue by the ratio of the maximum peak-to-peak value of the signal projected on the principal eigenvector to the maximum peak to peak value of the signal projected on the vector orthogonal to the principal vector, as illustrated in block 2801.
2. The paradox index can be calculated as a cost function performed on the paradoxical factor.
3. If the paradox index is compared with one or more thresholds, it can be interpreted as the absence or presence of paradoxical breathing or the degree of asynchronous respiration.

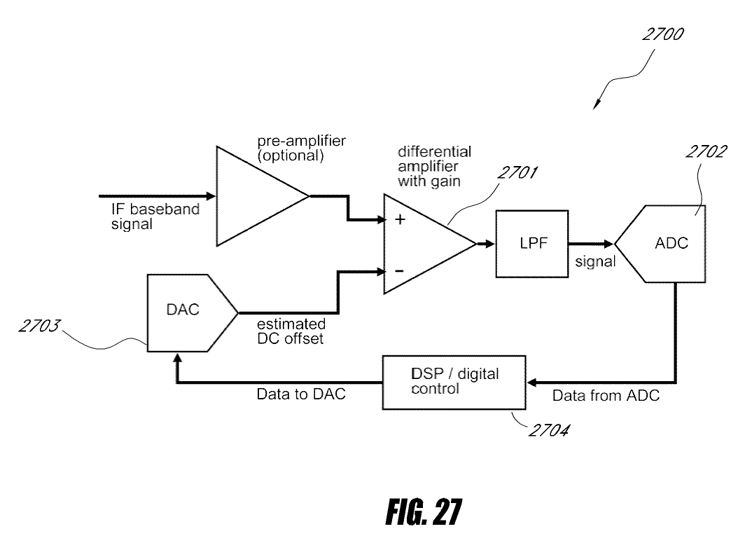

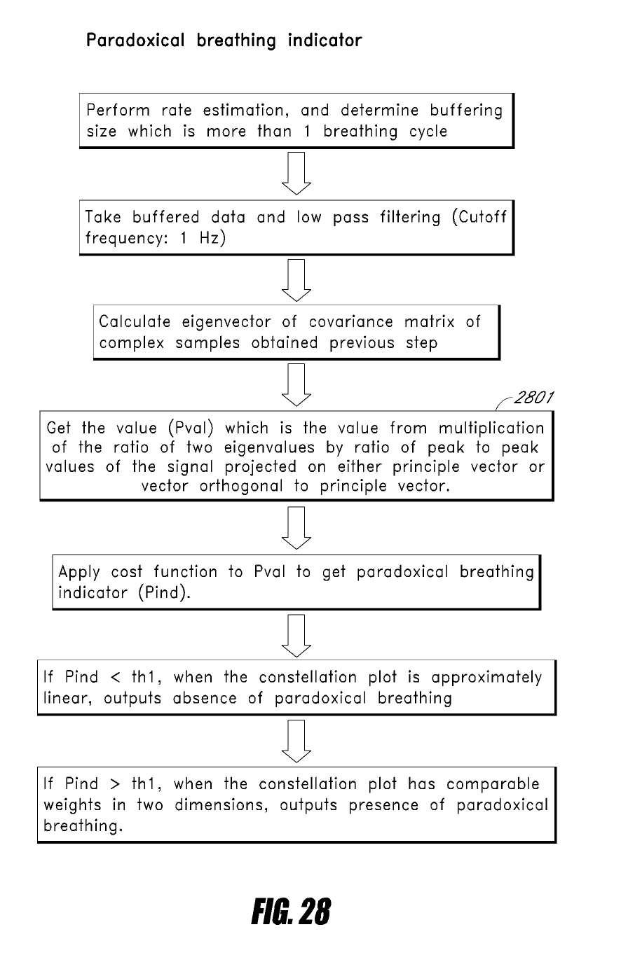

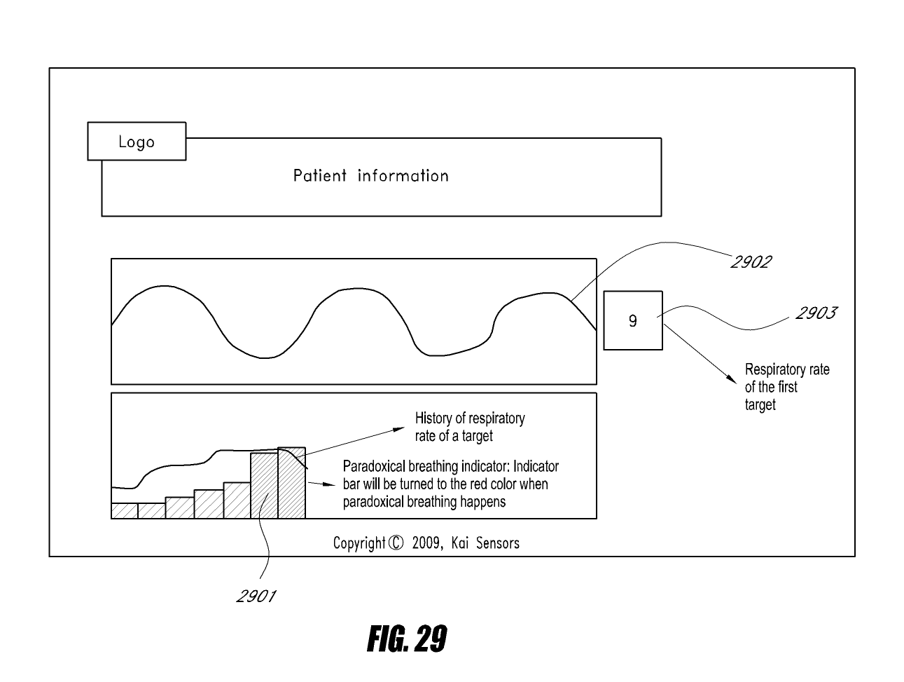

FIGS. 29 and 30 are screen shots of a display device configured to display the output from a system configured to detect paradoxical breathing. Information related to paradoxical breathing can be displayed graphically (e.g., as bars) 2901 and 3001. For example as illustrated in FIGS. 29 and 30, when paradoxical breathing is detected the bars indicating the average respiration rate can change color (e.g., from yellow to red, or green to red, or red to green, etc.). Other information such as respiratory waveform 2902 and 3002 or a respiratory rate 2903 and 3003 can also be displayed. The display of FIG. 30 also shows the tidal volume (amount of air flowing through the nasal passage at each breath) graphically (e.g., as a bar graph) 3004. The color of the bars representing tidal volume can also change colors (e.g., from yellow to red, or green to red) when paradoxical breathing is detected. Other ways of indicating paradoxical breathing can also be used.

An example configuration includes a system 100 configured to operate at a frequency of approximately 2.4 GHz. In some embodiments, the system includes a single antenna configured as a transmitter and three or more antennas configured as a receiver. In various embodiments, the receiver antennas can be spaced half wavelength apart. In various embodiments, a different number of transmitting and receiving antennas can be used. In some embodiments, the system further includes a quadrature direct conversion or homodyne receiver, a high-pass filter or a DC-cancellation circuit or both. The system 100 can further include a processor configured to execute linear demodulation algorithm as disclosed in U.S. Provisional App. No. 61/204,881 which is incorporated herein by reference in its entirety and in U.S. Provisional App. No. 61/137,519 which is incorporated herein by reference in its entirety.

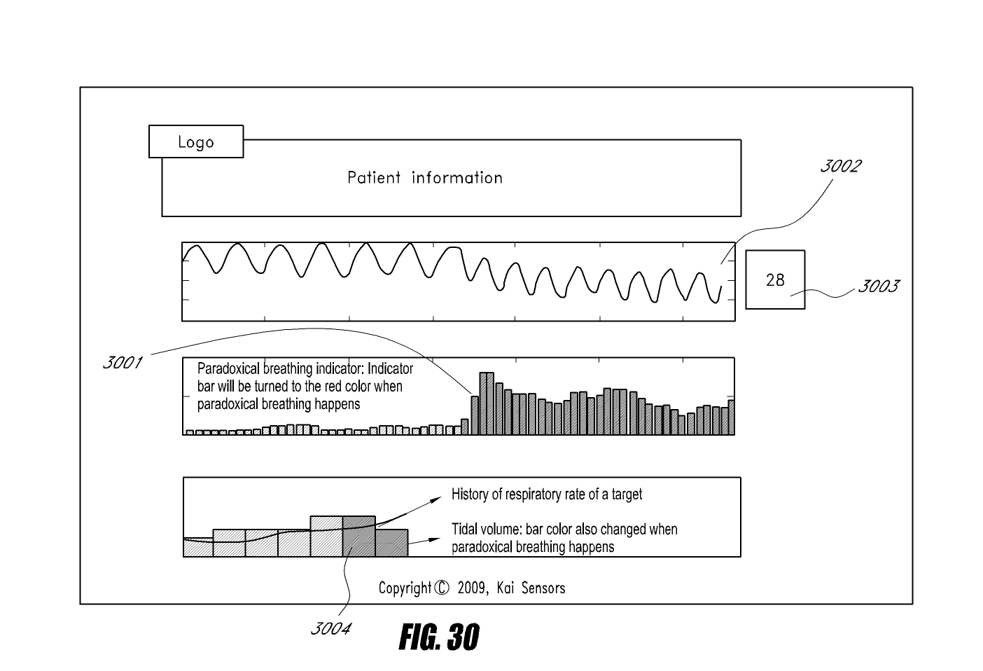

As discussed above, in various embodiments, a homodyne receiver is used for its simplicity and for its phase noise cancellation or reduction property. To eliminate mirror imaging at baseband after down-converting the RF signal, the system includes complex demodulation, which provides quadrature outputs. In various embodiments, an antenna array can be used to transmit and receive radar signals. In some embodiments, a single antenna can be used to transmit, and an array of antennas can be used for receiving. In various embodiments, the system 100 can be configured to execute the Direction of Arrival (DOA) algorithm or processing can be provided with at least two receiver antennas in each plane of interest. In various embodiments, one or more receiver antenna arrays can be used to execute the DOA algorithm. Antenna arrays can be more compactly designed by sharing antennas for different array clusters as illustrated in FIG. 31. The system 3100 illustrated in FIG. 31 comprises a central antenna 3101, an antenna on left 3102 in communication with a receiver 3104 and an antenna on the right 3103 in communication with a receiver 3105. With reference to FIG. 31, the center antenna 3101 belongs to both left and right array clusters and is in communication with both the receiver 3104 and 3105 which results in two independent array clusters composed of two single elements. In one embodiment, this approach can reduce the number of antennas required as compared to a conventional antenna array design wherein each cluster is designed to have two elements, thereby reducing the total area required for the number of antennas. As discussed above, the quadrature outputs can be anti-alias filtered and in various embodiments, the DC signal can be removed either with a high-pass filter or a DC-cancellation system. The filtered signal can be sampled by an analog to digital converter (ADC) followed by signal processing, which can isolate the physiological motion signal from noise, interference, and non-physiological motion. The physiological motion signal can be processed to determine the cardiopulmonary parameter(s) of interest. FIG. 32 illustrates an embodiment of a system including two receiving antennas 3201 and 3202. The system of illustrated in FIG. 32 can be extended to any number of receiving antennas, or can be modified to include only one receiving antenna. In some embodiments, each receiver can have its own antenna.

In various embodiments that include multiple antennas and multiple receivers, DOA algorithm or processing can be used to provide several benefits in the detection of vital signs. When sensing physiological information with a radar system, it is desirable to have a wide antenna beam width to cover the subject in all probable positions. However, the wide beam can cause detection of motion away from the subject, which can affect the measurement. DOA processing from multiple antennas can provide the wide beam width needed to detect and track a subject as well as a way to steer a narrower beam to concentrate the radar signal on the physiological motion and avoid interfering motion from the surrounding. In order to focus the beam on the target, an array antenna configuration can be used as a transceiving antenna. In various embodiments, DOA processing can also null out angles with high amplitude interfering signals.

The radar system 100 can use DOA to separate sources of motion sensed by the radar system based on their differing angles from the antenna. Any of several DOA algorithms can be used for this technique. The signals from the antennas can be processed as an antenna array, which has a narrower beam width than any of the individual antennas. Through processing, the beam of this array can be effectively steered towards the desired source, so the antenna beam is focused on the source and any motion outside the beam will be attenuated according to the antenna pattern in that direction. Additionally, the angle to the target subject can be detected and presented in the interface, either as the angle or as a more general indication of the direction (i.e., straight, left, or right).

The multiple antennas can also be used to detect and track the angle of an interfering motion source. The signals from the antennas can then be combined such that there is a null in the antenna beam pattern in the direction of the interfering motion. This can be used to separate signal sources, by measuring one source while placing a null in the direction of the interfering motion.

One embodiment of an algorithm for separating multi physiological signals is described below and includes:
1. Determining the frequency components of interest f=f₁, f₂, . . . , fₙ. In some embodiments, this can be done by measuring combination of spectral power of multi-channels. A specified cost function can provide output that can distinguish frequency components from the targets' chest motion.
2. Forming a channel matrix H whose entries correspond to fᵢ, f₂, . . . , fₙ. For example, the m^{th} row and n^{th} column of the channel matrix entry can be hₘₙ=sₘₙ(fₙ), corresponding to the receiver antenna m and signal source n, where sₘₙ represents frequency spectrum of the channel.
3. Forming an array vector given by equation (1):

g(θ)=[1exp[jkd sin(θ)] . . . exp[jkd(M−1)sin(θ)]]^{T} (1)
where k is the wavenumber, d=λ/2 is the separation distance between each receiver antenna and θ is the angle from the antenna normal vector to the target, while M is the number of received antennas.
4. Calculating the maximum average power that can be obtained at the angle of the sources and is given by equation (2):

Pₐᵥ(θ)=|H^{H}g(θ)|² (2)
5. Eliminating angles that are separated from each other by an angular distance less than the angular resolution of the multiple receiver antenna array, and identifying at least a first and second angular direction such that each angular direction is separated from each other angular source by an angular distance greater than or equal to an angular resolution of said multiple receiver antenna array.
6. Forming an M×N array matrix A whose ith column is given by the equation (3)

g(θᵢ)=[1exp[jkd sin(θᵢ)] . . . exp[jkd(M−1)sin(θᵢ)]]^{T} (3)
where d=λ/2 and θ are the receive antenna separation and angle respectively, while M is the number of received antennas. In those embodiments where there are other moving objects in the vicinity of the subject which can scatter the radar signal, N denotes the number of moving objects.
7. Including signal separation that can be achieved by steering spatial nulls toward unwanted signal sources by multiplying inverse of matrix A, estimated in step 4, to the channel data (S=A⁻¹Rₓ).

In various embodiments, these approaches can be used as a SIMO (single input multiple output) system, with one transmitter and multiple receiver antennas, or could be implemented as a MIMO (multiple input multiple output) system, with multiple transmitters, each at a different frequency, and multiple receivers. In various embodiments, other DOA algorithms could also be used to separate sources at different angles from the antenna.

In various embodiments, after DOA processing, the subject's vital signs, such as respiratory rate, chest displacement, tidal volume, and/or heart rate can be extracted from the physiological motion waveform and output to the output device.

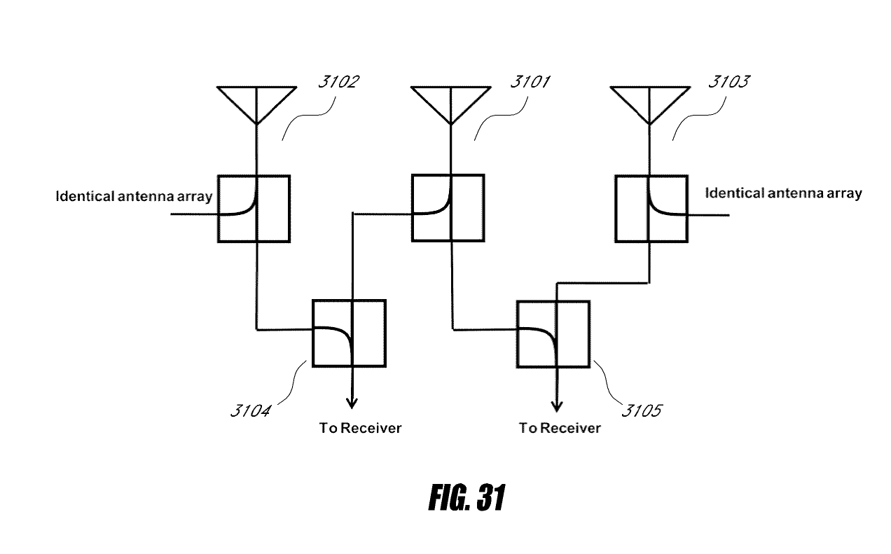

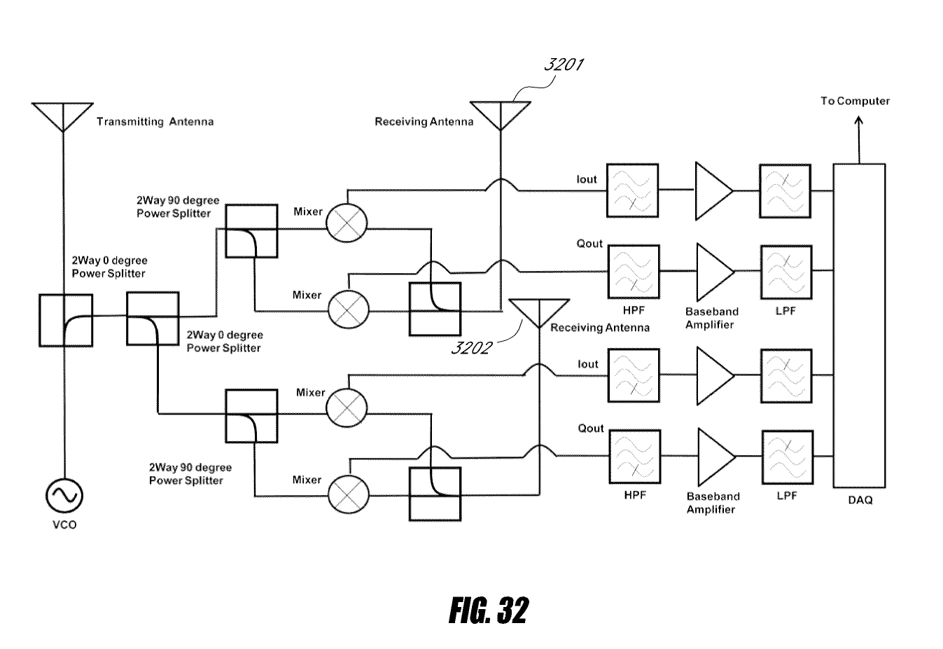

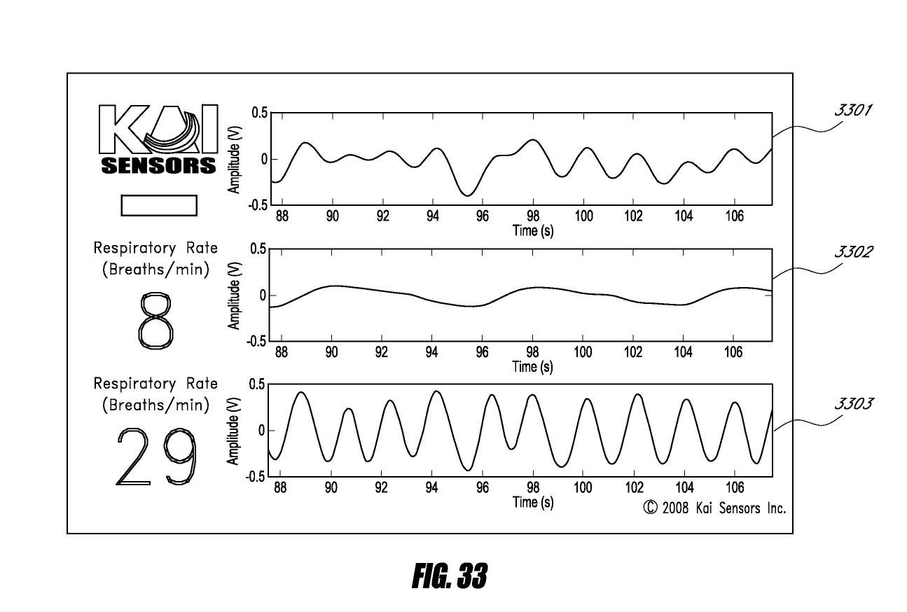

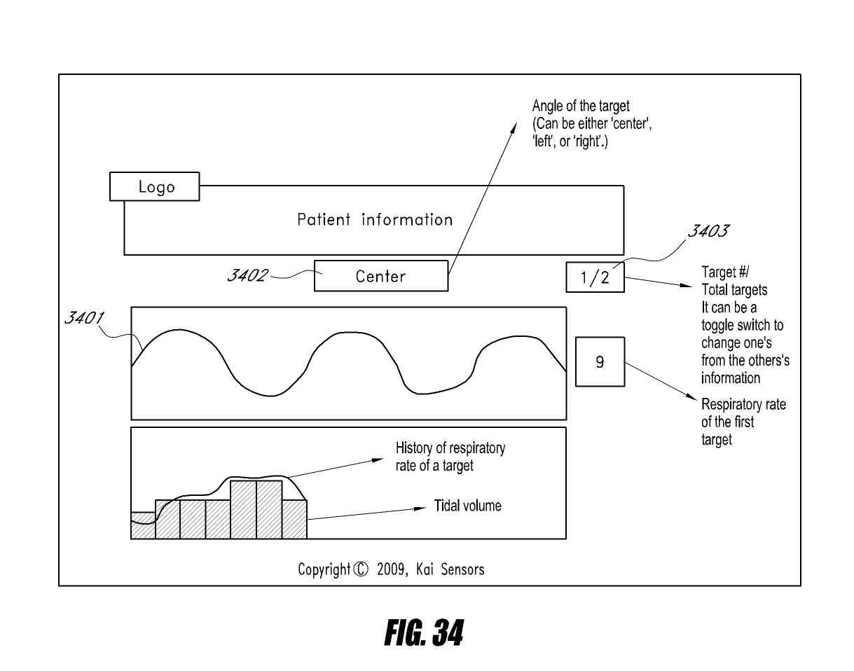

In various embodiments, the vital signs and/or directional information can be buffered and plotted to provide historical data for the subject. FIG. 33 shows the screen shot of a display device configured to output cardiopulmonary information of two people after DOA processing separated their respiratory signals. Plot 3301 shows the baseband signal obtained from both the subjects. Plot 3302 shows a waveform corresponding to a respiratory activity of a first subject while plot 3303 shows a waveform corresponding to a respiratory activity of a second subject. In various embodiments, the display device can be configured to display information related to respiratory activity (e.g., waveform related to respiration, average respiration rate, etc.). In various embodiments, other information such as tidal volume, heart and/or angle or position of the subject can also be displayed. FIG. 34, illustrates a screen shot of a display device configured to display the respiratory waveform 3401 and the tidal volume and a history of respiration rate. In some embodiments, the position of the target with reference to the sensor can also be displayed on the display 3402. In various embodiments, the display can include a control area 3403 to switch between patients. FIG. 35 illustrates a screen shot of a display device configured to display the respiratory motion waveforms for two people. Plot 3501 shows the mixed baseband signal obtained by the system from two subjects. The mixed baseband signal is processed using a DOA algorithm to extract information related to the respiratory activity of the two subjects. Plot 3502 shows the respiratory activity of a first subject positioned about 24 degrees to the right of the system and plot 3503 shows the respiratory activity of a second subject positioned about 13 degrees to the left of the system. A history of the respiratory rates for the two subjects is shown in plot 3504.

An example configuration includes a system 100 configured to operate at approximately 5.8 GHz with a low-IF receiver. In various embodiments, the system further includes a single antenna configured to transmit radar signals and a single antenna configured to receive radar signals. In various embodiments, the system includes a low-IF receiver configured to transform the received signal to a signal including frequencies in the range from a few Hz to a few kHz. For example, in some embodiments, the IF receiver can be configured to transform the received signal to a signal having a frequency in the range for about 1 Hz to 200 kHz. In various embodiments, the system's processor can be configured to execute an arc demodulation algorithm. In various embodiments, the system 100 can be configured as a spot check monitor or a continuous monitor.

In various embodiments, the system includes an oscillator (e.g., a voltage controlled oscillator) configured to operate at approximately 5.8 GHz and a stable crystal oscillator configured to generate radiation in the kHz to MHz range. The signal from the oscillator is split in by a power splitter. The signal from a first output of the power splitter is provided to the transmitting antenna and the signal from a second output of the power splitter is multiplied by the signal from the crystal oscillator to generate a reference signal for the receiver. Since the reference signal will still benefit from the range correlation effect, the phase noise of the reference signal will not adversely affect the residual phase noise; the residual phase noise will be limited by the crystal oscillator, which typically has a very low phase noise. In various embodiments, a low-IF receiver architecture can mitigate problems caused by 1/f noise, channel imbalance, and dc offset with low phase noise. In various embodiments, low-IF signals can be directly sampled by an ADC and down-converted to quadrature baseband signals in the digital domain. Thus, when arctangent demodulation is used, significant changes in the location of the origin, changes in the radius of the circle the arc is on, or changes in the position of the arc on the circle can indicate a change in the relationship between the antenna and subject, which can indicate non-cardiopulmonary motion. As discussed above, non-cardiopulmonary motion can be detected by calculating the inner product of the normalized current vector and the normalized previous vector. A significant change in the relationship between the subject and the antenna is indicated if the value of the inner product is below a threshold. In those embodiments, where arctangent demodulation is used, a change in the RMS error of the fit to the best-fit arc can also indicate non-cardiopulmonary motion or other signal interference.

An example configuration includes a system 100 configured to operate at a radio frequency of approximately 5.8 GHz with a direct-conversion receiver and DC-offset cancellation. In various embodiments, the system 100 includes a single antenna to transmit radiation and a single antenna to receive radiation. In various embodiments, one or more antennas can be used to transmit and/or receive signals. In various embodiments, the system 100 can include a processor configured to execute an arc demodulation algorithm.

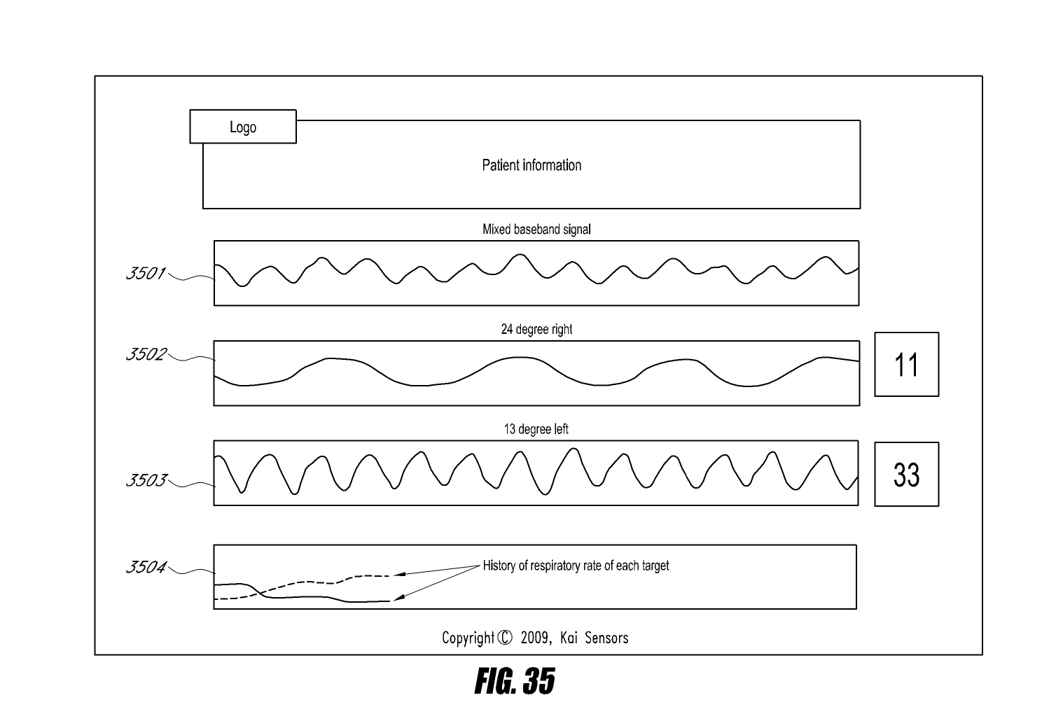

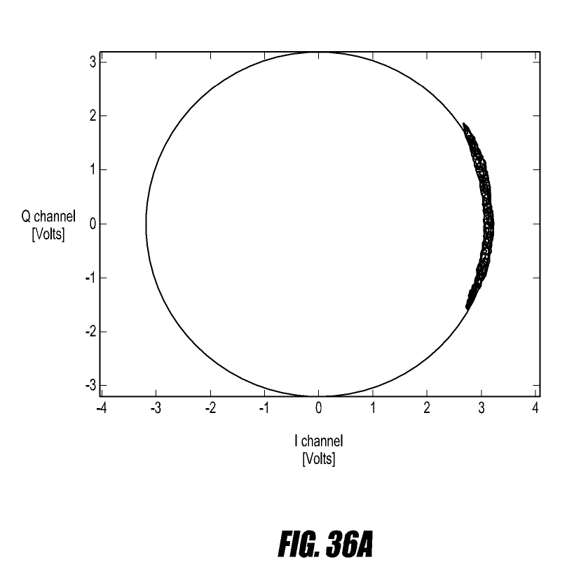

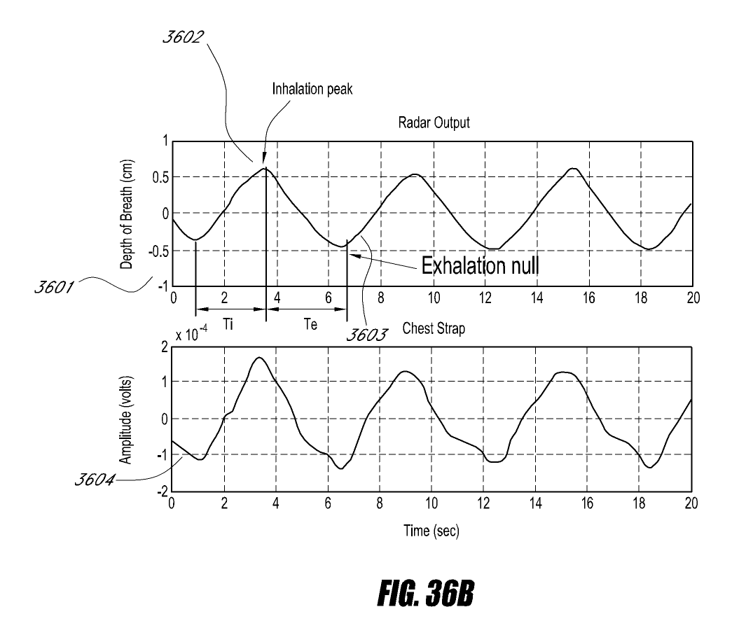

In embodiments using a radio frequency of approximately 5.8 GHz, the phase deviation, can result in non-linear quadrature baseband output or an arc trace rather than a line in the complex constellation as shown in FIG. 36A. Consequently, arc demodulation can be preferred over other demodulation algorithms to obtain accurate signals in systems with 5.8-GHz carriers. Furthermore, DC cancellation rather than AC coupling filter can be preferred to reduce signal distortion, and to enable determination of the origin of the circle where signal samples are scattered with sufficient accuracy. Since arc demodulation can extract phase information from baseband signal which can be linearly proportional to the actual chest motion, it is possible to estimate depth of breath from arc demodulation. The depth of breath information obtained from arc demodulation can also be applied to tidal volume estimation; there can be a linear relationship between the linear chest excursion and the tidal volume. FIG. 36B shows a plot 3601 of the depth of breath versus time. The depth of breath shows an inhalation peak 3602 and an exhalation null 3603. From this plot the tidal volume (amount of air inhaled Ti and amount of air exhaled Te in each respiratory cycle) can be estimated. Plot 3604 shows a corresponding measurement obtained by a conventional sensor. FIG. 36C shows a snapshot of a display device illustrating the tidal volume 3605, a waveform corresponding to the respiratory activity 3606 and a respiratory rate 3607. In various embodiments, as the length of arc increases, the ambiguity in the signal polarity can be reduced which can enable estimation of inhaling and exhaling time duration, which enables estimation of the ratio between inhale time and exhale time. The cardiopulmonary related motion of the body surface can be measured either from a distance or in contact with the body. In those embodiments, wherein the antenna is in contact with the body, methods to isolate body surface reflections from internal reflections can be used and internal body motion can be measured. In various embodiments, other internal cardiopulmonary related changes can also be electromagnetically measured for surface and internal body parts and tissues, including impedance change associated with heart beat.

An example configuration includes a multi-receiver system configured to operate at a radio frequency in the 5.8 GHz band. The system includes a single antenna to transmit the radar signal and four or more antennas to receive the radar signals. In various embodiments, the receiver antennas can be placed a half wavelength apart. In some embodiments, the system 100 can include more than one transmitting antenna and less than four receiving antennas. The system further includes a direct conversion or homodyne receiver for each receiving antenna. In various embodiments, the system 100 can include a DC cancellation circuit to remove or reduce the DC offset. The system 100 can also include a processor configured to execute an arc demodulation algorithm.

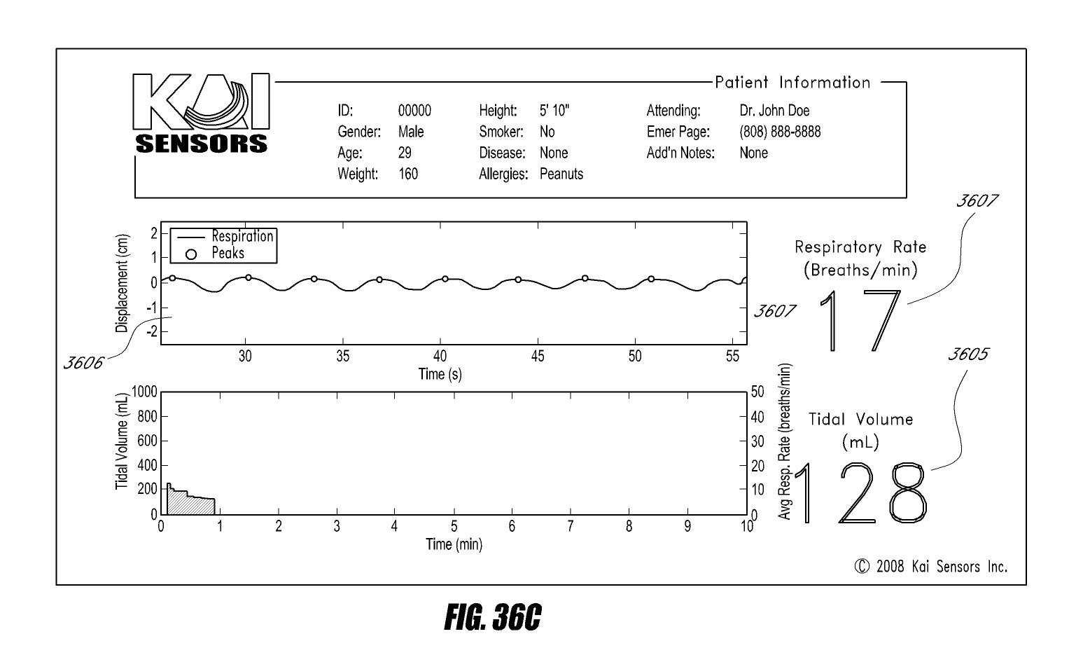

In embodiments of the system configured to operate in a frequency range of approximately 5.8 GHz, it is possible to design and manufacture compact antenna arrays. Thus, in systems configured to operate at approximately 5.8 GHz it is possible to get an increased number of arrayed elements within substantially the same area as a system configured to operate at approximately 2.4 GHz. In other words, it is possible to achieve higher spatial resolution in systems configured to operate at approximately 5.8 GHz as compared to systems configured to operate at approximately 2.4 GHz, with an antenna of the same footprint. FIG. 37 illustrates a schematic layout of an array element including a transmitting antenna 3701 and at least four receiving antennas 3702a-3702d. Thus embodiments of systems configured to operate at approximately 5.8 GHz can be advantageous when used for DOA processing because a given area can include a higher number of antennas as compared to a system configured to operate at approximately 2.4 GHz. An increase in the number of antennas can enable detection and tracking of subjects who are closely spaced (e.g., angular separation between two subjects can be less than 15 degrees with 4 antennas).

The DOA algorithm or processing technique described above can be employed to track subjects in various embodiments of the system. In some embodiments, arc demodulation can be employed after using DOA algorithms to tracking subject or suppress interference from non-cardiopulmonary motion or a cardiopulmonary motion of a second person. After signals from the multiple subjects are separated, non-cardiopulmonary motion detection algorithm can be employed. In various embodiments, the signal from each direction can be demodulated with an arc-based demodulation algorithm, which uses the parameters of the best-fit circle to obtain angular information from the complex constellation. Significant changes in the location of the origin if the best-fit circle, changes in the radius of the best-fit circle, or changes in the angular position of the arc on the circle can indicate a non-cardiopulmonary motion or other signal interference. The processor can then provide cardiopulmonary information on one or more subjects.

In various embodiments, a system 100 including a sensor placed on the body for measuring whether there is respiration and/or heart motion is described. The system 100 can be configured as wearable Microwave Doppler radar which can be placed in contact with a subject (e.g., in contact with a subject's chest). The wearable Microwave Doppler radar can be used to estimate a subject's respiratory rate and heart rate, and/or other vital signs, by detecting the motion of the body surface, motion of internal organs, or a combination of these motions. Various embodiments of this system 100 can operate at approximately 2.4 GHz, approximately 5.8 GHz or some other frequency band. In various embodiments, the system 100 can be configured as a stand alone device or can be integrated with a wireless communication system to communicate with other local devices and/or remote data centers or interfaces as disclosed in U.S. Provisional App. No. 61/194,838 which is incorporated herein by reference in its entirety.

In various embodiments a system comprising a sensor placed on the body for measuring a respiratory activity and/or heart motion is described. The system can comprise a wearable Microwave Doppler radar which can be placed in contact with a subject (e.g., in contact with a subject's chest). The wearable Microwave Doppler radar can be used to estimate a subject's respiratory rate and heart rate, and/or other vital signs, by detecting the motion of the body surface, motion of internal organs, or a combination of these motions. Various embodiments of this system can operate at approximately 2.4 GHz, approximately 5.8 GHz or some other frequency band. In various embodiments, the system can be configured as a stand alone device or can be integrated with a wireless communication system to communicate with other local devices and/or remote data centers or interfaces as disclosed in U.S. Provisional App. No. 61/194,838 which is incorporated herein by reference in its entirety.

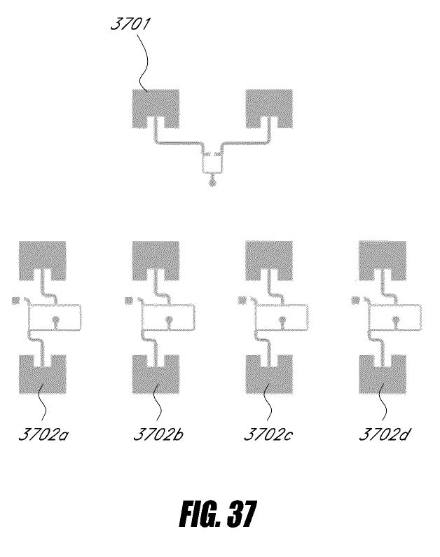

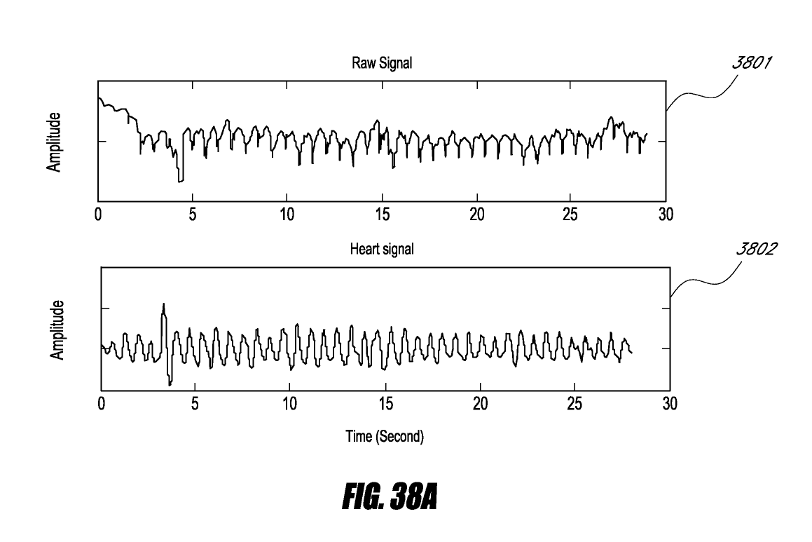

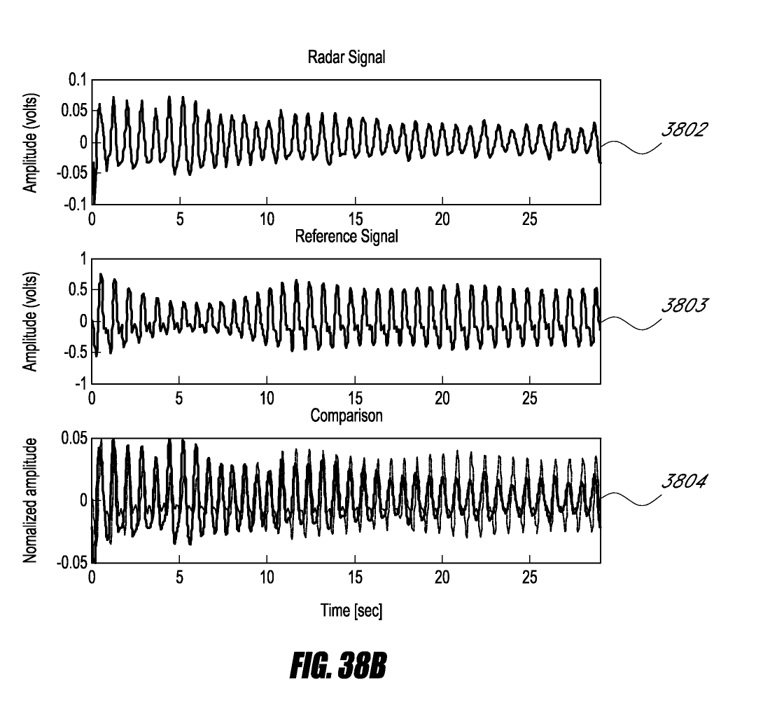

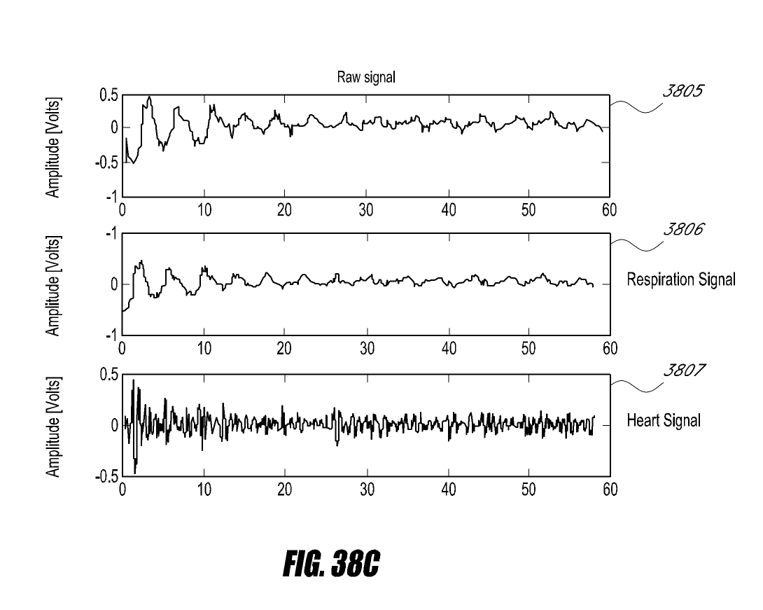

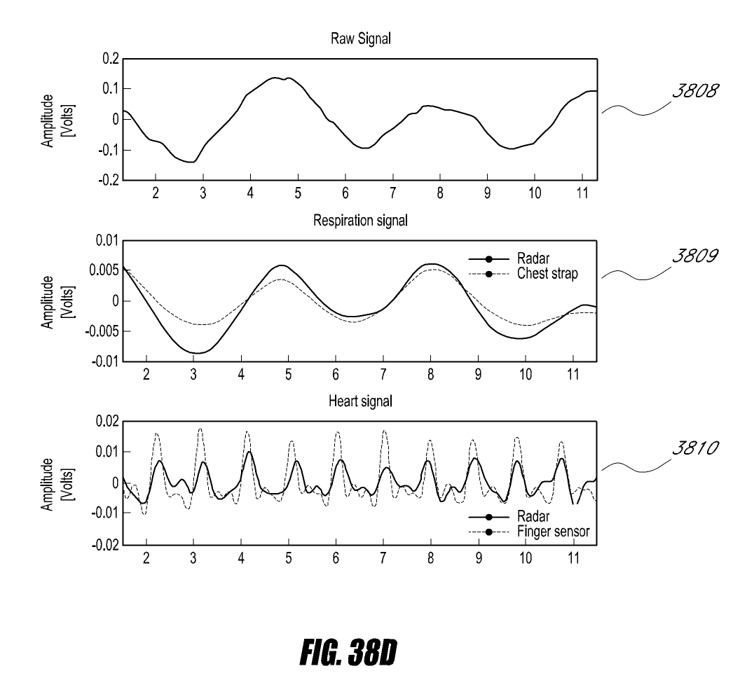

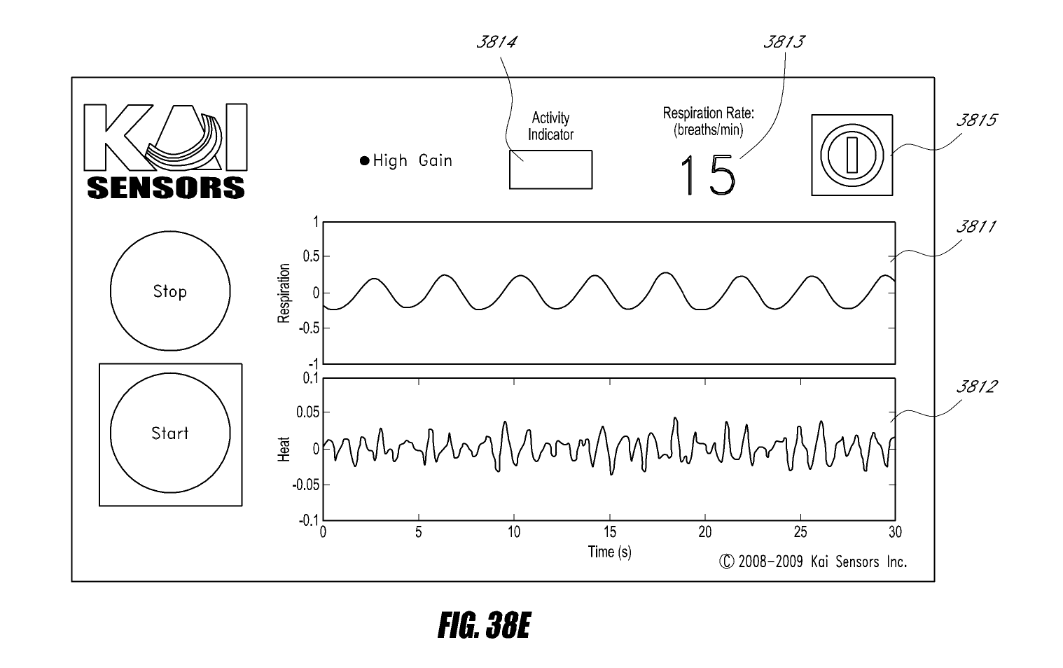

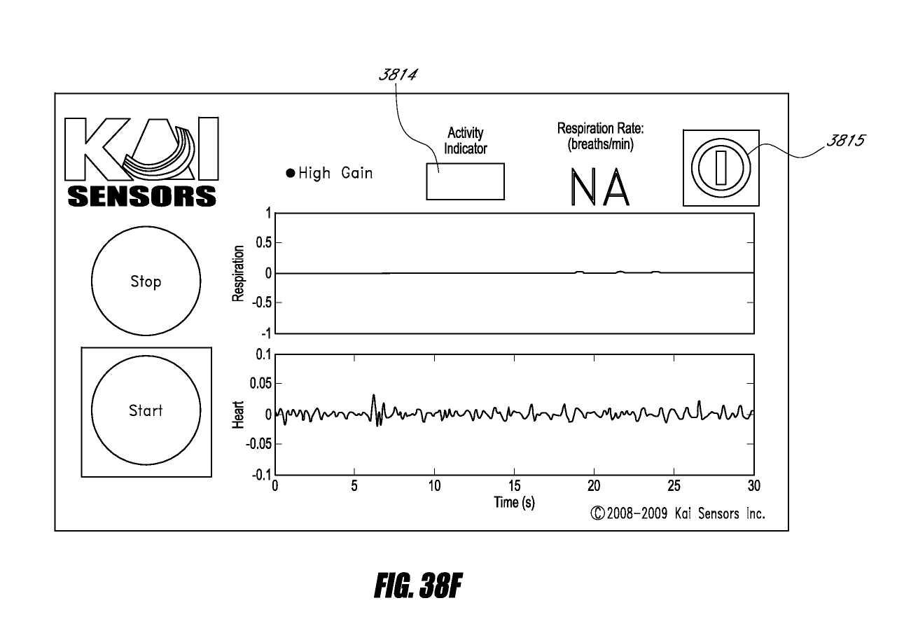

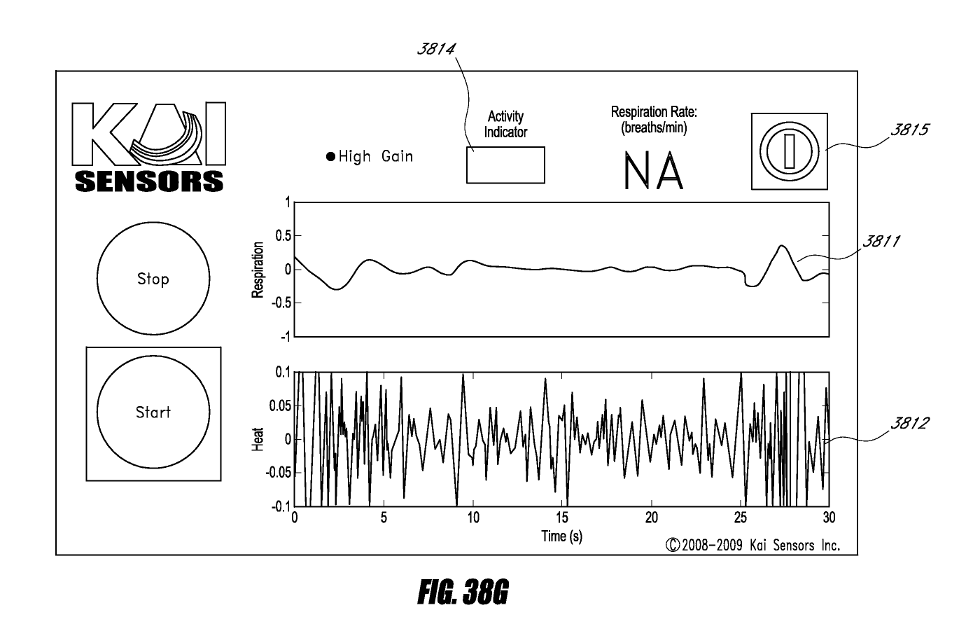

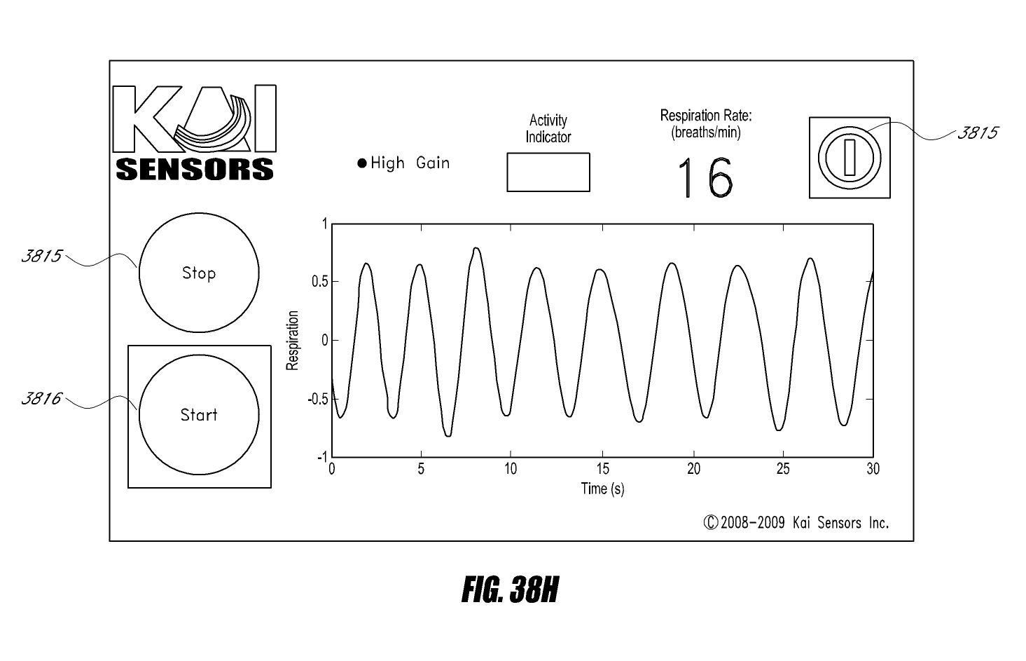

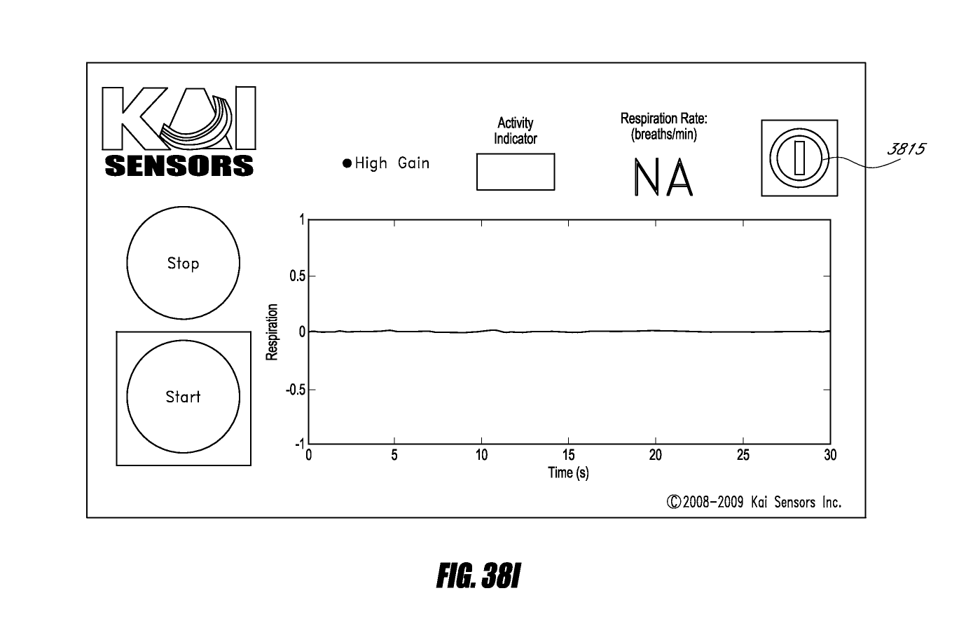

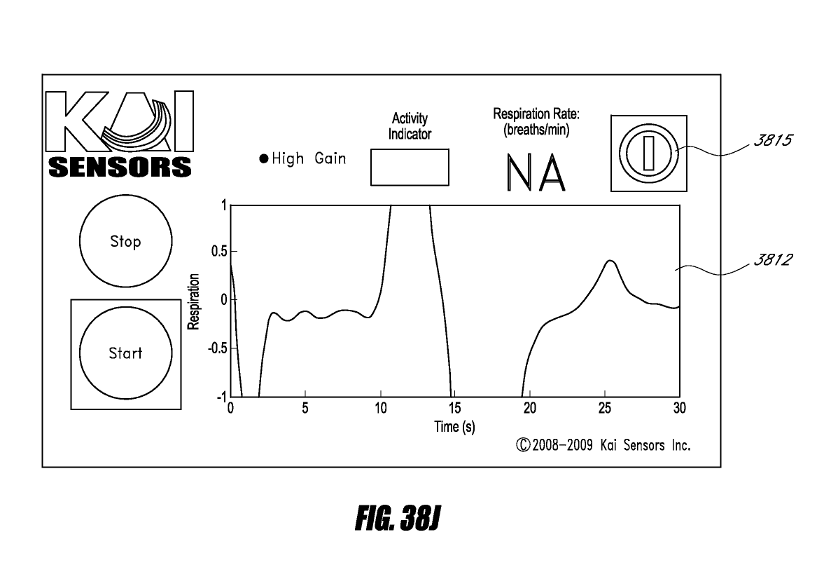

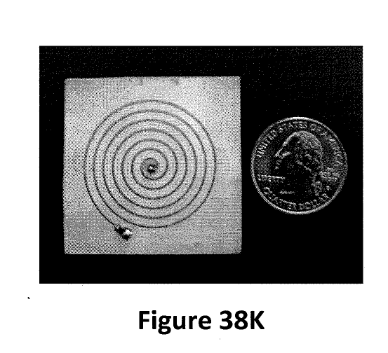

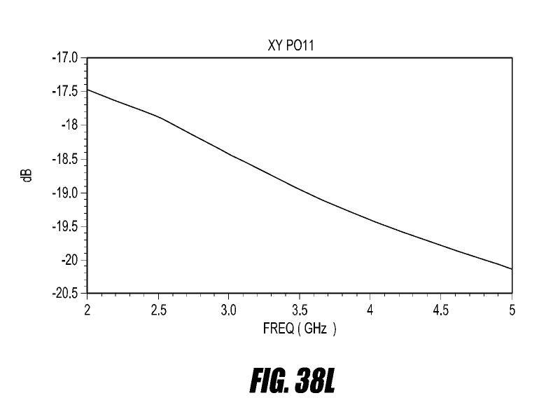

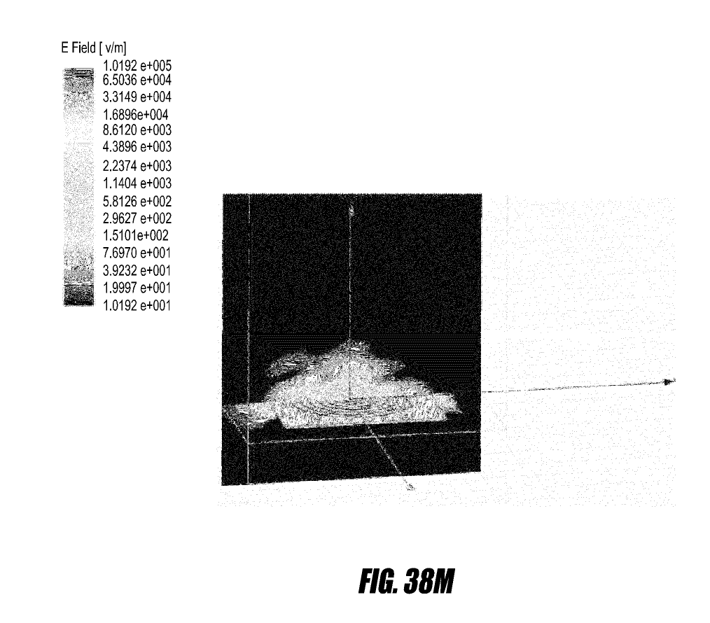

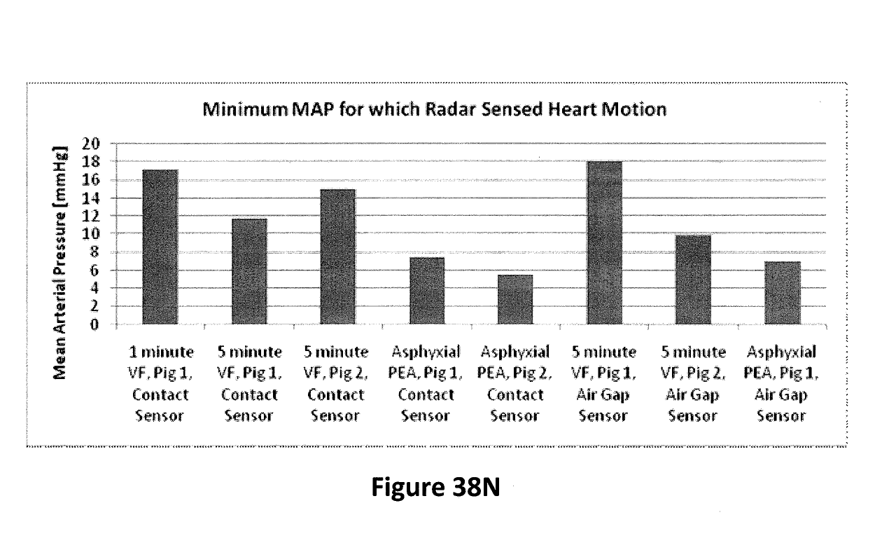

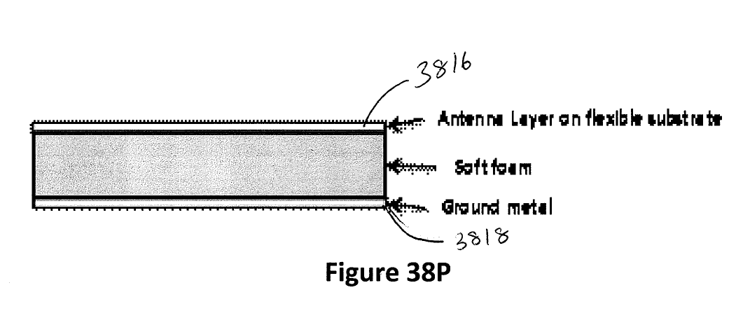

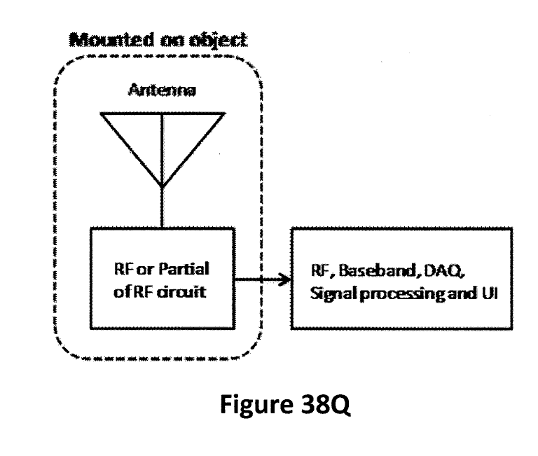

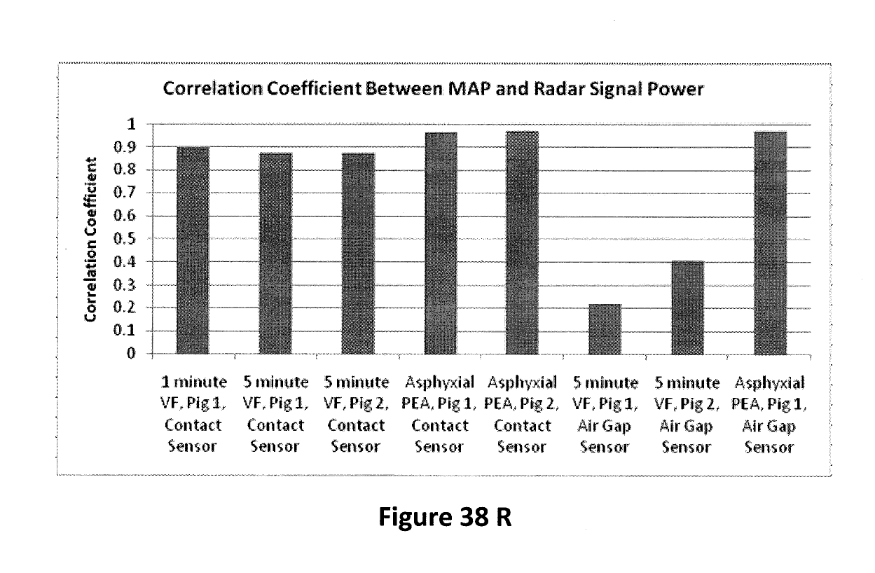

FIG. 38A shows the information related to cardiopulmonary activity when a wearable radar system similar to system 100 is placed in contact with a subject who is holding his/her breath. Plot 3801 illustrates a raw cardiopulmonary signal which has not been processed and plot 3802 illustrates a processed heart signal. FIG. 38B shows the information related to cardiopulmonary activity when a wearable radar system is placed in contact with the subject who is holding his/her breath in comparison to a reference signal. Plot 3802 shows the received radar signal and plot 3803 shows the reference signal. Plot 3804 shows the comparison between the radar signal and the reference signal.

FIG. 38C shows the information related to cardiopulmonary activity when a wearable radar system is placed in contact with a subject who is breathing normally. Plot 3805 shows the unprocessed signal and plot 3806 shows the respiration signal obtained after processing the raw signal. Plot 3807 is a heart signal obtained after processing the raw signal. The heart signal appears irregular due to coupling with breathing and/or harmonics of the breathing signal. However, a substantially accurate heart rate can be measured with the embodiments described in this application.

FIG. 38D shows the information related to cardiopulmonary activity as compared to a reference signal using a non-contact radar-based physiological sensor described above on a subject who is breathing normally. Plot 3808 shows the unprocessed signal and plot 3809 shows the respiration signal obtained after processing the raw signal. Also shown in plot 3809 is the respiration signal measured with a conventional sensor such as a chest strap. Plot 3810 is a heart signal obtained after processing the raw signal as compared to a heart signal obtained using a finger sensor.

FIGS. 38E and 38F are embodiments of a display device configured to display respiration waveform 3811, heart waveform 3812, respiration rate 3813, and indication of activity 3814. In various embodiments, this user interface can be used for detecting the presence of a subject or for detecting whether or not a subject is breathing or a subject's heart is beating. In various embodiments, the display interface can be used for triage and resuscitation as well as detecting a subject's presence. In various embodiments, if activity or respiration or heart is detected, a subject is present; if neither is present, a subject is not detected. In various embodiments, the display interface can be used to detect whether or not a subject's heart is beating and/or the subject is breathing for triage and to determine whether CPR and/or defibrillation and/or other resuscitation is required. In various embodiments, if a subject's presence is detected, for example due to cardiopulmonary activity of the subject then an indication can be provided. For example, the 3815 can turn green if a subject is present. However, if a subject's presence is not detected then, the indicator 3815 can turn red and respiration waveform or respiration rate is not display as shown in FIG. 38F

FIGS. 38G-38J are alternate embodiment of the display device shown in FIGS. 38E and 38F that are configured to display a respiration waveform, a respiration rate, a heart rate, a heart waveform, indication of activity, indication of subject's presence etc. In FIG. 38G, a subject's presence is detected by the heart signal 3812 and the respiration signal 3814 and is indicated by the indicator 3815 turning yellow and/or the activity indicator 3814 glowing. In FIG. 38H, a subject's respiration signal is detected as shown by the respiration waveform 3811 and can be indicated when the activity indicator turns green. Start and Stop controls can be provided on the display as shown by 3816 and 3815 respectively.

In FIG. 38I, no respiration signal is detected and so the indicator 3815 is red. In 38J a respiration signal 3812 is observed which indicates a subject's presence and by the activity indicator turning red.

In some embodiments, the sensor can also detect mechanical physiological motion including cardiopulmonary activity via direct contact with a subject's chest. When the sensor is not in contact, some of the signal emitting from an antenna is reflected on the surface of the chest, and some of the emitted signal can bypass the subject altogether, such that motion in the surrounding environment can interfere with the physiological motion signal. When the sensor is in contact, nearly all of the signal couples with the body, and almost none of the signal by passes the subject. In embodiments where the sensor does not contact the body, an antenna array is used so the antenna radiation pattern has a narrow beam width to enable focusing the transmitted signal in the desired direction to avoid sensing motion in the surrounding environment. In embodiments wherein the sensor contacts the body, nearly all of the transmitted signal couples with the body, so the antenna beam width is not an issue, and it is feasible to detect a cardiopulmonary signal with a single antenna (rather than an array) without any significant interference from the surrounding environment. The use of a single antenna rather than multiple antennas results in a more compact device.

When a sensor is in contact position with a subject's chest, chest motion due to cardiopulmonary activity can be amplitude modulated on the reflected signal. In some embodiments, this amplitude modulated signal, which is proportional to a subject's chest motion, corresponding to his/her cardiopulmonary activity, can be extracted by a low-IF single channel receiver architecture. In various embodiments, once the reflected signal is down converted to the low-IF, the signal will be sampled at higher than Nyquist rate to obtain non-aliased digital signal. In various embodiments, the Hilbert transform performed on the digitized input signal to obtain a complex signal where the in-phase part is the input signal while the quadrature part is the output of Hilbert transform.

In various embodiments, the envelope of the reflected signal, which is proportional to the cardiopulmonary activity, can be obtained by taking the absolute value of the complex value obtained in previous step. This method can achieve a compact device by using a single channel receiver without any concern of imbalance factors. The demodulation circuit is much simpler than that of quadrature architecture.

In some embodiments of a contacting Doppler radar sensor for monitoring or measuring internal cardiopulmonary activity rather than induced chest-surface motion, it is desirable to increase the reflected signal power from the heart relative to the signal from the chest surface. The ratio between these powers can be improved when the RF signal penetrates well into the human body. In some embodiments, a spiral antenna can provide a frequency independent, or broadband antenna, reducing the mismatch between the antenna and the skin when the antenna is in contact with human skin. In some embodiments, better matching can be achieved by covering a spiral antenna with a layer of silicone and/or with a liquid-type gel. In some embodiments, the silicone can be a low-durometer silicone such that it can conform to both the antenna and the skin easily, without any air gaps. In some embodiments, an adhesive can be placed around the antenna or on the silicone surface to tightly adhere the antenna and/or silicone to the skin surface. FIG. 38K illustrates an embodiment of a spiral antenna for contact sensor. In the illustrated embodiments, the width of the line is approximately 0.3 mm and is winding by the function r=aØ where a is approximately 0.35 mm and 0≦Ø≦45 radian. FIG. 38L shows the matching property of this spiral antenna from 2 GHz to 5 GHz. It shows more than −17 dB S11 for the simulated frequency range. FIG. 38M illustrates simulation results of RF signal power coupled through the spiral antenna into the body. It shows that a 2.4 GHz RF signal can penetrate up to 8 mm, with penetration defined as less than −20 dB loss from the maximum field strength at the feedpoint.

Various embodiments of a continuous Doppler radar system can be used to monitor or detect physiological signals including mechanical heart motion (also referred to as heart pulse) and lung motion with contact to the body. In embodiments in which the radar system is collecting reflected signals without contacting a human body or with a small air gap between the antenna and the skin, the received signal is mostly that reflected at the boundary between skin and air. In such embodiments, because the magnitude of the chest motion is highly correlated with internal heart motion, it is feasible to monitor heart's physical motion with an air gap between the antenna and the chest. In embodiments in which the radar antenna is completely in contact with human skin, the radio signal is reflected mostly at an internal interface (for example, the heart muscle wall), which has a higher correlation with the actual heart motion. The reflected signal power and the demodulated heart signal power are proportional to the displacement of heart motion because the sensor-to-heart distance is nearly fixed with a contact sensor. Therefore, the relative pulse power (which is proportional to blood pressure in some cases) can be estimated. With proper calibration, absolute heart motion and/or absolute blood pressure can be estimated from the Doppler-radar based signals. This information can be obtained with a contacting sensor or with a sensor placed on the chest wall that has an air gap between the antenna and the skin.

Embodiments of a contacting sensor designed to measure internal heart motion includes a radar system that is composed of the following: a front end coupler, a radio transmitter and receiver; a baseband signal-conditioning system; an analog-to-digital converter; and a signal processor. Embodiments of radar systems that can be used to sense heart motion include air-gap sensors, contact sensors, and esophageal sensors. In some embodiments of an air-gap sensor, the front-end coupler is an antenna which is designed to transmit a signal through air. In some embodiments of a contacting sensor, the front end coupler is an antenna which is specially designed for impedance matching with the human body. In some embodiments of an esophageal sensor, a coaxial cable with a right-angle connector covered in rubber is inserted in the esophagus with the open end of the connector facing toward the heart. In some embodiments, the parts of the radar system other than the front-end coupler are the same for all air-gap, contacting sensors, and esophageal sensors, such that the front-end coupler can be changed for different types of measurements. In some embodiments, the radar systems can operate at any frequency between 10 MHz and 100 GHz; in some embodiments, sensors can operate in the 2.4-GHz and 5.8-GHz ISM radio bands. The radio transmitter generates and emits a radio signal. The radio receiver collects reflected radio signal and down-converts it to a complex baseband signal, with in-phase and quadrature components, while adding minimal noise. This complex baseband signal that contains cardiopulmonary motion information is amplified and filtered, in the baseband system. In some embodiments, it is AC-coupled in the baseband system, but in other embodiments a DC-coupled signal is digitized. In some embodiments, the conditioned signal is sampled at 1 kHz, which is sufficient to avoid aliasing of heart pulse signal's significant harmonics. In some embodiments, the conditioned signal can be sampled at any frequency between 50 Hz and 100 MHz.

One embodiment of the front-end coupler for the contact sensor is a spiral antenna. In some embodiments, a silicone layer placed between the antenna and the body tends to distribute the RF signal uniformly in the human body. In some embodiments, the silicone bolus also buffers the impedance change between air and the human body, thus providing a matching layer which helps the RF signal to penetrate deeper in the body. In some embodiments, using a silicone layer with a contacting antenna makes it feasible for a radar sensor to get a reflected signal from a broader and deeper body muscle area. In some embodiments, the layer of silicone between the antenna and the body many be 3.5 mm thick. In some embodiments, a gel can provide complete contact between the antenna or silicone layer and the skin surface. In some embodiments, complete contact can provide better impedance matching, resulting in higher power penetration through the body and thus higher reflected power from the heart muscle. In some embodiments, gels of 0% saline, 3% saline, 4% saline, and 10% saline can be used.

In some embodiments, air-gap sensors can be used instead of contacting sensors. In some embodiments, air-gap sensors use a single rectangular patch antenna designed to propagate through air.

In some embodiments, the reflected radio signals received by the antenna are down-converted to a complex baseband signal, which includes cardiopulmonary motion information. In some embodiments, these signals are sampled at 1 kHz and decimated to a 100 Hz signal to increase the signal-to-noise radio (SNR). In some embodiments, the decimated signals are recorded. Subsequently, in some embodiments, these signals are demodulated to get a signal that is proportional to the cardiopulmonary motion acquired by the sensor. In some embodiments, the demodulated signals are filtered by FIR Kaiser windowed filters to isolate the desired heart signals from other signals, resulting in a heart pulse trace. In some embodiments, the relative power of mechanical heart motion during various pathologic stages was calculated using the envelope of the heart pulse trace. In some embodiments, the RMS voltage is used to calculate the pulse power—it is the square root of the mean of the voltage squared. In some embodiments, this pulse power is proportional to mean arterial pressure, and can be used to detect changes in the mean arterial pressure. In some embodiments, with calibration, the pulse power can be used to estimate the mean arterial pressure.

To verify functionality of the radar sensor for monitoring heart motion, the heart wall motion of swine was measured in different pathological conditions including pulseless electrical activity and ventricular fibrillation with two different antennas. During these pathological state, the swine blood pressure and cardiac output dropped significantly. During pulseless electrical activity, the heart has normal electrical activity, while the cardiac output is very low. In these experiments, the system was able to detect heart motion at mean arterial blood pressures as low as 5 mmHg as illustrated in FIG. 38N and had signal power that was proportional to the blood pressure (as illustrated in FIG. 38R). These data indicate that such a system can be used to sense heart motion during pathological states, and can be used in conjunction with an electrocardiograph to detect pulseless electrical activity, which can appear like normal heart beats when the electrocardiograph is used alone.

Various embodiments of a contacting or air-gap radar-based sensor, operating at 2.4 GHz, can be used to sense mechanical heart motion. In some embodiments, the power of the signal from the both the contacting radar-based sensor and the air gap radar-based sensor are proportional to the mean arterial pressure. In some embodiments, the air gap and contacting sensors can detect heart motion at mean arterial pressures below 10 mmHg when positioned directly over the heart.

In some embodiments, a contacting radar-based sensor could be integrated with defibrillation electrodes, to detect heart motion and relative changes in blood pressure during ventricular fibrillation and pulseless electrical activity. This could help to guide decisions of when to deliver a shock for defibrillation, or when chest compressions are required. In some embodiments, this could be integrated in an automated external defibrillator, or a mechanical chest compression device. In some embodiments, the antenna integrated in the defibrillation would be built on a flexible substrate, such that they are flexible and conforming to the skin. In some embodiments, this flexible substrate would include silicone and/or a gel between the antenna and the skin.

In some embodiments, this device could be used in conjunction with ECG to determine the presence of pulseless electrical activity, when the heart motion is very small and the blood pressure is very low, but the heart's electrical activity is normal. This is a case in which an ECG alone cannot detect the need for CPR, but a mechanical measurement could. In some embodiments, one adhesive patch could be placed on the skin, containing both an ECG electrode and a contact radar sensor. In some embodiments, silicone and/or gel would be included between the antenna and the skin, and the adhesive would be around the antenna assembly.

In some embodiments in which a contacting Doppler radar sensor is used for monitoring or measuring internal cardiopulmonary activity, rather than induced skin-surface motion, (as is done with air-gap or non-contact radar-based sensors), it is desirable to increase the reflected signal power from the internal motion relative to the signal from the skin surface. In some embodiments, interference induced by motion of other body parts, such as chest motion due to breathing, can be eliminated or reduced by using a lightweight, conforming antenna or system. In some embodiments, where that sensor is placed on the neck, breathing motion can be much less than the motion of the carotid artery. At the carotid artery and the temporal artery, the blood vessel is near the skin surface, such that it is possible for the signal to penetrate the skin surface and detect the pulse directly from the blood vessel when the sensor is mounted on the neck. In some embodiments, an antenna, antenna array, or a system implemented on a flexible substrate fits and contours to the human neck well, such that the sensor is conformal and comfortable, and does not shift significantly with movement.

In order for the RF signal to penetrate the human body, the antenna should have broad-band matching. The broad-band property of the antenna reduces the mismatch between the antenna and the skin when the antenna is in contact with human skin. In some embodiments, a flexible antenna 3816 with broad band matching can be achieved by using an air gap antenna structure, which has air between the antenna (on the flexible substrate) and the ground metal 3818 as shown in FIG. 38P. The air gap has a low dielectric constant, which facilitates design of a planar antenna with a broad-band match. In some embodiments, the antenna structure is placed on a thin flexible substrate, a layer of soft flexible foam providing an air gap of uniform thickness is placed on top of the substrate with the antenna structure, and then a layer thin metal, such as aluminum or copper foil is placed on the other side of the foam to provide a ground plane. This structure provides a lightweight, flexible, broad-band planar antenna.

In some embodiments, an adhesive can be placed on the broadband antenna such that it adheres directly to the neck. In some embodiments, a low durometer silicone can be placed between the antenna and the adhesive to improve matching between the antenna and the neck, and this can help the RF signal to penetrate deeper into the body. In some embodiments, a gel can be used between the antenna and the body, with adhesive around the edges. In some embodiments, the gel can be water-based. In some embodiments, the water-based gel can include saline. In some embodiments, the saline can be between 1 and 15%. In some embodiments, the saline can be 10%. In some embodiments, both silicone and a gel can be used between the antenna and the body, with adhesive around the edges such that the antenna adheres directly to the neck.

In some embodiments of the arterial sensor, an array of small, inflexible broadband antennas can be placed on a flexible substrate, such that the small, inflexible antennae can each conform to the skin of the neck. The received signal from each element can be combined by a combiner that, in some embodiments, is fabricated on the other side of the antenna ground plane. In some embodiments, when the combiner shares the ground plane with the antennae, the ground metal is thicker than the skin depth of the carrier signal in order to minimize cross talk between antennas and an RF circuit.

In some embodiments, the broad-band antenna can be a spiral antenna. In some embodiments, an array of spiral antennae can be used to make the system robust to positioning on the neck, such that the antenna can be quickly placed on the neck without regard to positioning over the carotid artery. In some embodiments, an elongated spiral, with an ellipse-like shape, can be used to provide robustness to positioning. In some embodiments, one bowtie antenna or an array of bowtie antennae can be used. In some embodiments, one air dielectric rectangle patch antenna or an array of patch antennae can be used. In some embodiments, one annular microstrip antenna or an array of annular antennae can be used.

In some embodiments, antenna(s) and a RF circuit or a partial RF circuit as illustrated in 38Q can be mounted on a subject's body to eliminate or relieve interference with the signal that is induced by cable motion between antenna and RF circuit. In other embodiments, the RF circuit is placed on the shoulder, with a short cable between the antenna and the RF circuit.

In some embodiments, there can be a wireless connection between the RF circuit and the processor and display unit. In some embodiments, this connection can be wired. In some embodiments, the processor and display can be co-located with the RF circuit, all placed on the shoulder or some other body part.

In some embodiments, the sensor is used to sense the carotid arterial pulse during CPR, to provide feedback on the effectiveness of chest compressions; if they are not providing adequate pulses to the carotid artery (and therefore also the brain), an automated CPR device or a healthcare practitioner could adjust the compressions until the device indicates that adequate blood is reaching the brain. In some embodiments, the sensor is used to measure the carotid arterial pulse in an unstable patient, in some instances following defibrillation, to determine if the heart is effectively pumping blood to the brain or not; if a patient has pulseless electrical activity, although electrical signals are being generated by the heart, the patient may not be getting adequate blood flow to the brain, and this sensor could help detect that.

To verify functionality of the radar sensor for monitoring internal organs' motion, heart wall motion of swine were measured in several different pathological conditions with two different antennas. These tests were focused on measuring mechanical motion of the heart; not the expansion of the artery. A spiral antenna was used for the contact sensor to transmit RF signal into the body and to collect the signal reflected from the heart wall. A rectangular patch antenna was used for the air-gap sensor to collect information related to chest surface motion, which correlates with heart motion. The reflected radio signals received by the antenna are down-converted to a complex baseband signal, which includes cardiopulmonary motion information. In some embodiments, these signals are sampled at 1 kHz and decimated to a 100 Hz signal to increase the signal-to-noise radio (SNR). In some embodiments, the decimated signals are recorded. Subsequently, in some embodiments, these signals are demodulated to get a signal that is proportional to the cardiopulmonary motion acquired by the sensor. In some embodiments, the demodulated signals are filtered by FIR Kaiser windowed filters to isolate the desired heart signals from other signals, resulting in a heart pulse trace. In some embodiments, the relative power of mechanical heart motion during various pathologic stages was calculated using the envelope of the heart pulse trace. In some embodiments, the RMS voltage is used to calculate the pulse power—it is the square root of the mean of the voltage squared. This pulse power is proportional to mean arterial pressure, and can be used to detect changes in the mean arterial pressure. With calibration, the pulse power can be used to estimate the mean arterial pressure.

The correlation between mean arterial pressure and radar signal pulse power was high during the ventricular fibrillation measurements. During asphyxial PEA, the heart motion signal power obtained with the contacting sensor closely tracked the mean arterial pressure, increasing at the beginning of the asphyxiation, and decreasing as asphyxiation persisted. The correlation coefficient between the two measurements was 0.97 in both measurements with contacting sensors and the measurement with the air gap sensor. The contacting sensor was able to detect cardiac motion as the mean arterial pressure dropped to values as low as 5.6 mmHg and 7.5 mmHg in the two measurements. The air gap sensor was able to detect cardiac motion as the mean arterial pressure dropped as low as 7 mmHg.

Overall, the power of the pulse signal from the radar sensor correlated well with the mean arterial pressure. The correlation coefficients for all experiments are shown in the bar graphs in FIG. 38R. No correlation with the difference in the systolic-diastolic pressure was found.

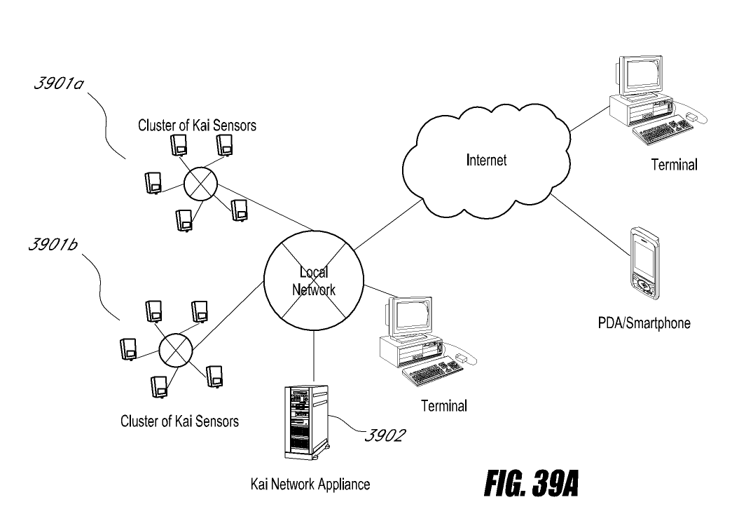

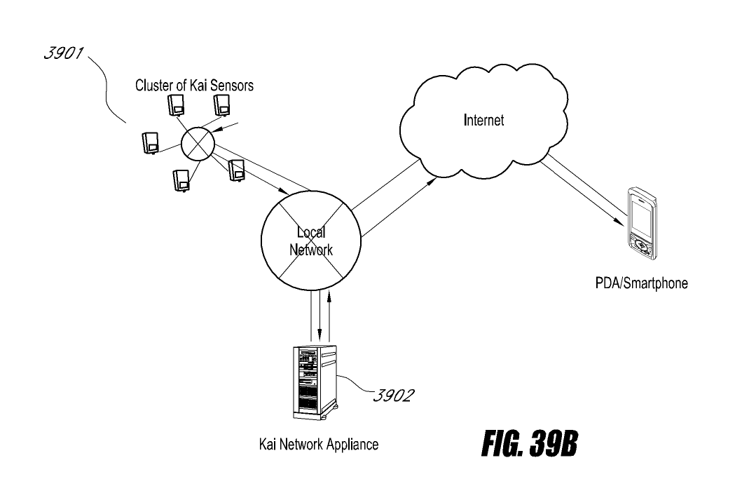

In various embodiments, a sensor network including many “thin” cardio pulmonary sensors works in conjunction with a centralized processing appliance. FIG. 39A describes a centralized topology such that many “thin” non-contact cardiopulmonary sensors form clusters 3901a and 3901b. The sensor clusters can be controlled by a network appliance 3902 where all processing will take place. Embodiments of this topology can be useful where sensors can be deployed in a dense area (i.e., one per hospital bed). In this case, rather than having each sensor be a full fledged cardio pulmonary monitor, each sensor will only possess minimal hardware, in some embodiments, only enough for data acquisition and forwarding a data stream. In various embodiments, each sensor will include a data acquisition module and a network module. In various embodiments, raw data will be streamed to the network appliance 3902 where further processing will be done. In various embodiments described above, the system can process the raw data internally. In various embodiments, processing will include the demodulation of the IQ channels, any DOA processing for tracking, respiration rate, etc. In various embodiments, the calculated statistics and processed data will then reside on the network appliance 3902 or they can be forwarded to an electronic health record server. A remote client can then access this data via a computer, mobile phone, PDA, etc. The data can also be viewed via a terminal locally or remotely in various embodiments. FIG. 39B shows an alternate embodiment of FIG. 39A showing the direction of information travel between the sensor cluster 3901a, the network appliance 3902 and various other components of the network.

The configuration above can also be useful in security applications where information needs to be processed at a centralized location. For example, in home security, the network appliance 3902 can be set to sound an alert if more than the set number of subjects is detected in the home. Another application for the various embodiment of the “thin sensor network” is homeland security, where many people need to be screened quickly such as at ports. A living database can be built and accessed in which biometrics information for certain individuals can be acquired, compared, and analyzed for security purposes.”

Although certain preferred embodiments and examples are disclosed above, inventive subject matter extends beyond the specifically disclosed embodiments to other alternative embodiments and/or uses and to modifications and equivalents thereof. Thus, the scope of the claims appended hereto is not limited by any of the particular embodiments described. For example, in any method or process disclosed herein, the acts or operations of the method or process can be performed in any suitable sequence and are not necessarily limited to any particular disclosed sequence. Various operations can be described as multiple discrete operations in turn, in a manner that can be helpful in understanding certain embodiments; however, the order of description should not be construed to imply that these operations are order dependent. Additionally, the structures, systems, and/or devices described herein can be embodied as integrated components or as separate components. For purposes of comparing various embodiments, certain aspects and advantages of these embodiments are described. Not necessarily all such aspects or advantages are achieved by any particular embodiment. Thus, for example, various embodiments can be carried out in a manner that achieves or optimizes one advantage or group of advantages as taught herein without necessarily achieving other aspects or advantages as can also be taught or suggested herein. Thus, the invention is limited only by the claims that follow.

1.-78. (canceled)

79. A method of determining the regularity of respiration, comprising:
processing one or more frames of a respiratory waveform to obtain information regarding the irregularity or regularity of respiration; said respiratory waveform comprising one or more frames, wherein the one or more frames comprise time sampled values of respiratory signals; and
communicating the information to an output system that is configured to perform an output action.

80. The method of claim 79, wherein the respiratory waveform is obtained by at least one of the group: Doppler radar, ultrawideband radar, impedance pneumography, chest straps, airflow measurements, and load cells.

81. The method of claim 79, wherein information regarding the irregularity or regularity of respiration includes assessment of the irregularity of the breath-to-breath interval or respiratory rate.

82. The method of claim 79, wherein information regarding the irregularity or regularity of respiration includes assessment of the irregularity of the amplitude of a breath or the depth of breath.

83. The method of claim 79, wherein information regarding the irregularity or regularity of respiration includes assessment of irregularity in the amplitude of respiration and irregularity in the breath-to-breath interval.

84. The method of claim 79, wherein information regarding the irregularity or regularity of respiration includes estimation of the cycle length of periodic or Cheyne-Stokes breathing.

85. The method of claim 79, wherein information regarding the irregularity or regularity of respiration includes assessment of the length of apnea in each cycle or the average length of apnea over several cycles.

86. The method of claim 79, wherein information regarding the irregularity of respiration includes the history of irregularity.

87. The method of claim 79, wherein the output of the system comprises one or more of: an indication of regularity or irregularity (a binary state); an integrated regularity index that compiles a variety of information about the regularity of respiration into a signal number or a single bar graph; separate indications of the irregularity of the breath-to-breath interval and the irregularity of the depth of breath; and individual indications of several measures of irregularity.

88. The method of claim 79, wherein processing one or more frames comprises:
performing an auto-correlation function on a subset of frames;
identifying whether major peaks are present;
identifying the number of samples from the center to major peaks, if they are present;
determining whether breathing is regular based on the number of samples to the first major peak and the height of the first major peak; and
identifying the second major peak that is not a multiple of the respiratory period as the period of periodic breathing.

89. The method of claim 88, wherein the subset of frames comprises samples obtained over a time longer than the expected period of respiration.

90. The method of claim 88, wherein the subset of frames comprises samples obtained over a time longer than the expected cycle period of irregular respiration.

91. The method of claim 79, further comprising using a wavelet transform function to create an index of repeating patterns in a respiration signal.

92. The method of claim 81, wherein the irregularity of the breath-to-breath interval is estimated from one or more of: the standard deviation of the breath-to-breath interval, the frequency of apneaic events, the coefficient of variation of the breath-to-breath interval, the standard deviation of the respiratory rate, and the coefficient of variation of the respiratory rate.

93. The method of claim 82, wherein the irregularity of the amplitude of a breath or the depth of breath, or breath duration, is estimated from the standard deviation of the breath depth, the coefficient of variation of the breath depth, the standard deviation of the respiratory signal amplitude, or the coefficient of variation of the respiratory signal amplitude.

94. The method of claim 79, wherein information regarding the irregularity or regularity of respiration includes assessment of whether irregular breathing is periodic.

95. The method of claim 94, wherein assessment of whether irregular breathing is periodic comprises:
estimating each breath-to-breath interval, and storing it with the time point at the end of the interval in which it was calculated;
interpolating between these breath-to-breath intervals to create a waveform;
performing the Fourier transform, performing the autocorrelation function, or calculating the power spectral density of the waveform;
determining whether there are significant peaks of the Fourier transform, the autocorrelation function, or the power spectral density of the waveform; and
determining that if significant peaks exist, the breathing is irregular and periodic.

96. The method of claim 94, wherein assessment of whether irregular breathing is periodic comprises:
interpolating between these breath-to-breath intervals to create a waveform;
identifying peaks of the waveform;
determining the time between the peaks;
calculating the coefficient of variation of the time between the peaks;
determining if the coefficient of variation of the time between the peaks is low, the breathing is irregular and periodic; and
determining if the coefficient of variation of the time between the peaks is low, the breathing is irregular and is not periodic.

97. The method of claim 94, wherein assessment of whether irregular breathing is periodic comprises:
identifying apneaic events;
determining the time of cessation of apneaic events;
estimating the interval between the cessation of each consecutive pair of apneaic events;
determining whether the interval between the cessation of each consecutive pair of apneaic events is consistent by calculating the coefficient of variation of the interval between the events by calculating the coefficient of variation;
determining if the coefficient of variation is below a threshold, breathing is periodic; and
determining if the coefficient of variation is above a threshold, breathing is irregular and not periodic.

98. The method of claim 94, wherein assessment of whether irregular breathing is periodic comprises:
calculating the envelope of the respiratory waveform;
performing the Fourier transform, performing the autocorrelation function, or calculating the power spectral density of the waveform; and
determining whether there are relatively significant peaks of the Fourier transform, the autocorrelation function, or the power spectral density of the waveform.

99. The method of claim 98, wherein the envelope is calculated by interpolating between the peak amplitudes.

100. The method of claim 98, wherein the envelope is calculated by squaring the signal and applying a low-pass filter.

101. The method of claim 87, wherein the integrated respiratory status index comprises a value, that is 0 for regular respiration, and can vary up to 6, with 1 point added for each of the following: irregular breath-breath interval; irregular breath depths; periodic breath-breath interval; periodic breath depth; periodic breath depth cycle time >60 seconds; periodic breath-breath interval cycle time >60 seconds; periodic breathing includes apnea >20 seconds; non-periodic irregular breathing includes apnea >20 seconds more frequently than once every 10 minutes.

102. The method of claim 87, wherein the integrated respiratory status index comprises a value that is 0 for regular respiration that increases by one point for each 20% in the coefficient of variation of the breath-to-breath interval and by one point for each 20% in the coefficient of variation in the depth of breath.

103. The method of claim 79, wherein information regarding the irregularity or regularity of respiration is assessed by:
(a) Estimating the breath-to-breath interval and the depth of breath for each breath as respiration is processed;
(b) Over an interval of 50 breaths, calculating the mean and standard deviation of the breath-breath interval, and the mean and standard deviation of the depth of breath;
(c) Calculating the coefficient of variation of the breath-to-breath interval and the depth of breath. If neither one is above a threshold, the respiration is considered regular. If the coefficient of variation of either the breath-breath interval or the depth of breath is above a threshold, the respiration is considered irregular, and additional processing is performed. In some embodiments, the threshold is 25%;
(d) If the respiration is irregular, determining whether the cycle time is periodic by interpolating between breath-breath intervals and depth of breath estimates, taking a Fourier transform of each waveform, and determining whether a periodic component exists in either waveform. If a periodic component exists in at least one of the waveforms, the cycle time is periodic. If a periodic component does not exist in either waveform, the cycle time is not periodic;
(e) If the cycle time is not periodic, repeating (d) with a longer interval of breaths (150 breaths). If the cycle time is still not periodic, skip to (g);
If the cycle time is periodic, calculating the cycle time finding by peaks in the interpolated breath-breath interval in step (d) and determining the mean time between the peaks, if multiple peaks are not available, extending the interval used for this step;
(g) If the cycle is not periodic, isolating the breath-breath intervals longer than 20 seconds, calculating the number of these intervals divided by the total time interval used for calculation, calculating the mean of these apneaic events;
(h) If the cycle is periodic, determining the length of apnea in each period, and average this number to get the average apnea length per cycle; and
(i) Displaying the data, if respiration is regular, indicating that respiration is “regular”, if respiration is irregular, indicating either “periodic—cycle time X” where X is the cycle time or “irregular,” if apneaic events exist, indicating “—average apnea length Y” and, if respiration is not periodic also indicating “—Z apneaic events/minute”.

104.-129. (canceled)

130. A system for determining the regularity of respiration, the system comprising:
one or more antennas configured to transmit electromagnetic radiation;
one or more antennas configured to receive electromagnetic radiation;
at least one processor configured to process one or more frames of a respiratory waveform to obtain information regarding the irregularity or regularity of respiration, wherein the one or more frames comprise time sampled values of respiratory signals; and
a communications system configured to communicate the obtained information with an output device, said output device configured to perform an output action based on the obtained information.
